(19) United States
(12) Patent Application Publication
(10) Pub. No.: US 2023/0097573 A1
(43) Pub. Date: Mar. 30, 2023

(54) NEONATAL FC RECEPTOR BINDING AFFIMERS

(71) Applicant: LG CHEM, LTD., Seoul (KR)

(72) Inventors: Yeonchul KIM, Seoul (KR); Jaehyung LEE, Seoul (KR); Saem JUNG, Seoul (KR); Joon Hee LEE, Seoul (KR); Gyeong Hyae PARK, Seoul (KR); Kyubong NA, Seoul (KR); Vincent MATTHEW, Amesbury, MA (US); Basran AMRIK, Cambridge (GB); Stanley EMMA, Hitchin (GB); Jenkins EMMA, Stansted (GB); Adam ESTELLE, Hitchin (GB)

(73) Assignee: LG CHEM, LTD., Seoul (KR)

(21) Appl. No.: 17/769,722

(22) PCT Filed: Oct. 16, 2020
(86) PCT No.: PCT/KR2020/014207

§ 371 Date: Apr. 15, 2022

Related U.S. Application Data
(60) Provisional application No. 62/915,790, filed on Oct. 16, 2019.

(51) Int. Cl. C07K 16/28; C12N 15/86; A61P 35/00; A61K 9/00

(52) U.S. Cl. CPC C07K 16/283; C12N 15/86; A61P 35/00; A61K 9/0019; A61K 2039/505

(57) ABSTRACT

Provided herein, in some embodiments, are AFFIMER® polypeptides that binds to the neonatal Fc receptor (FcRn) and extends the half-life of the polypeptides. Also provided herein, in some embodiments, are compositions containing the polypeptides, methods of using the polypeptides, and methods of producing the polypeptides.

### CROSS REFERENCE TO RELATED APPLICATION(S)

This application is a 35 U.S.C. 371 National Phase Entry application from PCT/KR2020/014207 filed on Oct. 16, 2020, which claims priority to and the benefit of U.S. Provisional Patent Application No. 62/915,790 on Oct. 16, 2019, the entire contents of all of which are incorporated herein by reference.

The instant application contains a Sequence Listing which has been submitted via EFS-Web and is hereby incorporated by reference in its entirety. Said Sequence Listing, created on Nov. 23, 2022, is named 3570-821_ST25.txt and is 596,533 bytes in size.

### TECHNICAL FIELD

The present invention relates to a polypeptide comprising an FcRn binding AFFIMER® sequence that binds to human FcRn.

### BACKGROUND ART

The neonatal Fc receptor (FcRn) binds with high affinity to IgG and albumin through non-overlapping sites at a mildly acidic pH (e.g., 5.0-6.5); however, it does not bind IgG or albumin at neutral pH. FcRn expression has been detected nearly ubiquitously in a number of tissues, including epithelial cells, endothelial cells, and cells of hematopoietic origin. It facilitates monitoring of IgG and serum albumin turnover, as its expression is upregulated in response to the proinflammatory cytokine, TNF-α and downregulated in response to IFN-γ FcRn has been used therapeutically to shuttle biologics across mucosal surfaces in order to improve drug absorption or distribution.

### DISCLOSURE

### Technical Problem

The neonatal Fc receptor (FcRn) binds with high affinity to IgG and albumin through non-overlapping sites at a mildly acidic pH (e.g., 5.0-6.5); however, it does not bind IgG or albumin at neutral pH.

### Technical Solution

An object of the present invention is to provide a polypeptide comprising an FcRn binding AFFIMER® sequence that binds to human FcRn.

Another object of the present invention is to provide a pharmaceutical preparations.

Another object of the present invention is to provide a methods that comprise administering to a subject having an autoimmune disease and/or an inflammatory disease.

Another object of the present invention is to provide a provide methods of increasing serum half-life of a therapeutic molecule.

Another object of the present invention is to provide a use of the polynucleotide for targeting FcRn.

A further object of the present invention is to provide a use of the polynucleotide for increasing serum half-life of a therapeutic molecule.

### Advantageous Effects

The present disclosure is based on the generation of AFFIMER® polypeptides that bind to human neonatal Fc receptor (FcRn) to extend, in a controlled manner, the serum half-life of any other therapeutic molecules (e.g., therapeutic AFFIMER® polypeptide, protein, nucleic acid, or drug) to which it is conjugated.

### DESCRIPTION OF DRAWINGS

FIG. 1 Example of LGC01 clones binding in a direct huFcRN ELISA at pH 6.

FIG. 2 Example of differential binding of LGC01 clones at pH 6 and 7.4 in a direct huFcRN ELISA.

FIGS. 3A-3C Analytical SEC-HPLC traces of purified FcRn AFFIMER® monomers and AVA04-FcRn binding AFFIMER® fusion.

FIGS. 4A-4B SDS-PAGE analysis of purified FcRn AFFIMER® monomers and AVA04-FcRn binding AFFIMER® fusion.

FIGS. 5A-5B FcRn binding ELISA showing the binding activity of purified FcRn AFFIMER® monomers and AVA04-FcRn binding AFFIMER® fusion at pH 6 and 7.

FIG. 6 FcRn competition ELISA showing the activity of FcRn AFFIMER® monomers and AVA04-FcRn binding AFFIMER® fusion.

FIG. 7 Flow Cytometry histogram of AFFIMER® clones that have high cell binding affinity at pH 6.0 and various binding affinities at pH 7.4.

FIG. 8 Confirmation of Affimer's cell binding using hFcRn over-expression CHO single clone cell line (pH 6.0 & pH 7.4).

FIG. 9 Demonstration of FcRn mediated recycling of the FcRn binding AFFIMER® polypeptides as determined using the human endothelial cell-based recycling assay.

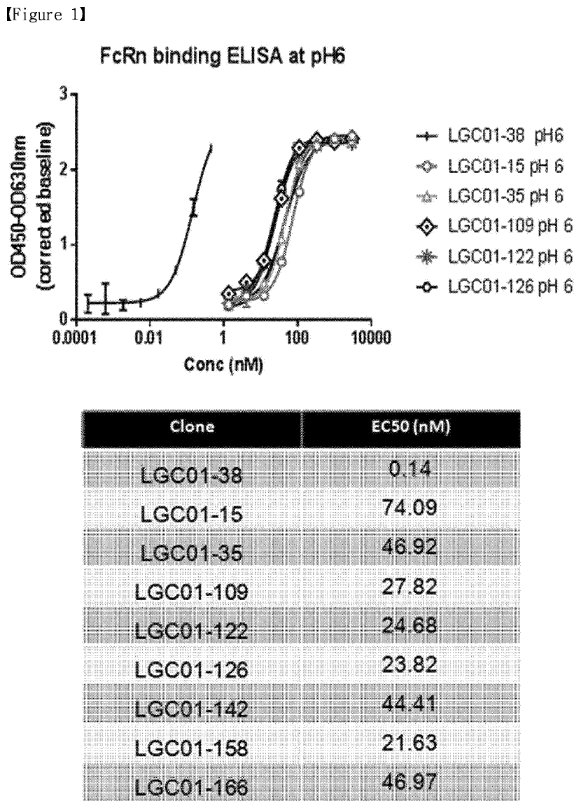

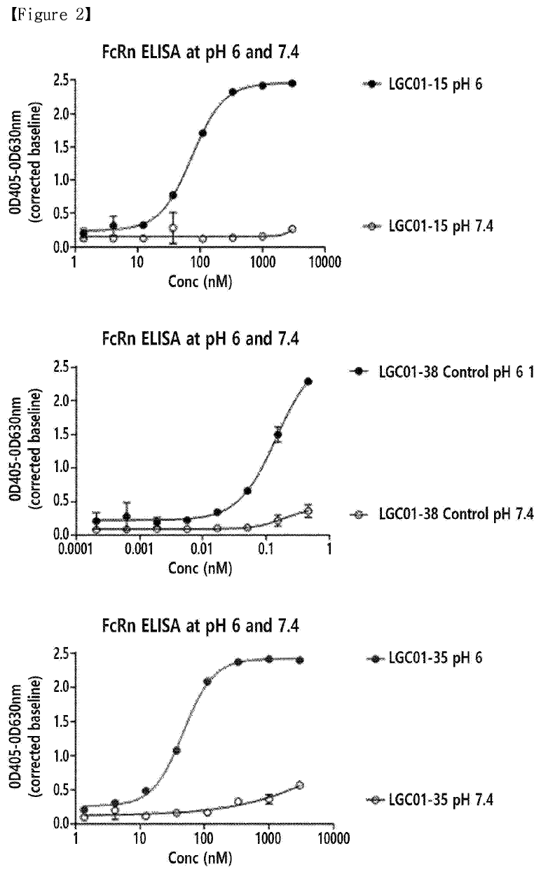

### BEST MODE

Provided herein, in some aspects, is a half-life extension platform based on AFFIMER® polypeptides that bind (e.g., competitively or non-competitively) to neonatal Fc receptor (FcRn, such as human FcRn). A range of human FcRn-binding AFFIMER® polypeptides (referred to as anti-human FcRn AFFIMER® polypeptides), with a range of binding affinities, has been developed. These polypeptides have been shown in in vivo pharmacokinetic (PK) studies to extend, in a controlled manner, the serum half-life of any other AFFIMER® polypeptides to which they are conjugated (e.g., as a single genetic fusion) and can be made, for example, in bacterial cells (e.g., Escherichia coli). The FcRn-binding AFFIMER® polypeptides provided herein can also be used to extend the half-life of other polypeptides, such as therapeutic proteins.

In some aspects, the present invention relates to a polypeptide comprising an FcRn binding AFFIMER® sequence that binds to human FcRn with a K_{d} of 1×10-6M or less at pH 6.0, and (optionally) a K_{d} for binding human FcRn at pH 7.4 that is at least half a log greater than the K_{d} for binding at pH 6.0.

In some embodiments, the FcRn binding AFFIMER® sequence binds to FcRn with a K_{d} of 1×10⁻⁷ M or less at pH 6.0, a K_{d} of 1×10⁻⁸ M or less at pH 6.0, or K_{d} of 1×10⁻⁹ M or less at pH 6.0.

In some embodiments, the polypeptides at pH 6 bind to human FcRn with a K_{d} that is at least one log less than the K_{d} for binding to human FcRn at pH 7.4, at least 1.5 logs less than the K_{d} for binding to human FcRn at pH 7.4, at least 2 logs less than the K_{d} for binding to human FcRn at pH 7.4, or at least 2.5 log less than the K_{d} for binding to human FcRn at pH 7.4

In some embodiments, the FcRn binding AFFIMER® sequence binds to FcRn at pH 7.4 with a K_{d} that is at least one log greater than the K_{d} for binding to FcRn at pH 6.0, at least 1.5 logs greater than the K_{d} for binding to FcRn at pH 6, at least 2 logs greater than the K_{d} for binding to FcRn at pH 6, or at least 2.5 log greater than the IQ for binding to FcRn at pH 6.

In some embodiments, the FcRn binding AFFIMER® polypeptide sequence binds to human FcRn and the protein/polypeptide has a circulating half-life in human patients of at least 7 days, preferably 8, 9, 10, 11, 12, 13, 14, 15, 16, 17, 18, 19, 20 or even 21 days.

In some embodiments, the polypeptide has a serum half-life in human patients of greater than 10 hours, greater than 24 hours, greater than 48 hours, greater than 72 hours, greater than 96 hours, greater than 120 hours, greater than 144 hours, greater than 168 hours, greater than 192 hours, greater than 216 hours, greater than 240 hours, greater than 264 hours, greater than 288 hours, greater than 312 hours, greater than 336 hours or, greater than 360 hours.

In some embodiments, the polypeptide has a serum half-life in human patients of greater than 50%, greater than 60%, greater than 70%, or greater than 80% of the serum half-life of IgG.

In some embodiments, the polypeptide has a serum half-life in human patients of greater than 50%, greater than 60%, greater than 70%, or greater than 80% of the serum half-life of serum albumin.

In certain embodiments, the polypeptide does not inhibit binding of human serum albumin to human FcRn.

In certain embodiments, the polypeptide polypeptide does not inhibit binding of IgG to human FcRn.

In certain embodiments, binding of the polypeptide to human FcRn facilitates transport of the polypeptide from an apical side to a basal side of an epithelial cell layer.

Another aspect relates to a protein comprising an FcRn binding AFFIMER® polypeptide sequence which binds to human FcRn and facilitates transport of the protein across an epithelial tissue barrier.

In certain embodiments, the AFFIMER® polypeptide sequence has an amino acid sequence represented in general formula (I)

FR1-(Xaa)ₙ-FR2-(Xaa)ₘ-FR3 (I),

wherein FR1 is an amino acid sequence having at least 70% identity to MIPGGLSEAK PATPEIQEIV DKVKPQLEEK TNETYGKLEA VQYKTQVLA (SEQ ID NO: 1); FR2 is an amino acid sequence having at least 70% identity to GTNYYIKVRA GDNKYMHLKV FKSL (SEQ ID NO: 2); FR3 is an amino acid sequence having at least 70% identity to EDLVLTGYQV DKNKDDELTG F (SEQ ID NO: 3); Xaa, individually for each occurrence, is an amino acid, n is an integer from 3 to 20, and m is an integer from 3 to 20.

For instance, FR1 can be at least 80%, at least 82%, at least 84%, at least 86%, at least 88%, at least 90%, at least 92%, at least 94%, at least 96%, or at least 98% identity to SEQ ID NO: 1; FR2 has at least 80%, at least 84%, at least 88%, at least 92%, or at least 96% identity to SEQ ID NO: 2; and/or FR3 has at least 80%, at least 85%, at least 90%, or at least 95% identity to SEQ ID NO: 3. In certain embodiments, FR1 comprises the amino acid sequence of SEQ ID NO: 1, FR2 comprises the amino acid sequence of SEQ ID NO: 2, and FR3 comprises the amino acid sequence of SEQ ID NO: 3.

In certain embodiments, the AFFIMER® polypeptide sequence has an amino acid sequence wherein (Xaa)ₙ is an amino acid sequence represented in the general formula

-Xaa-Xaa2-Xaa3-Xaa4-Xaa5-Xaa6-Xaa7-Xaa-Xaa- (SEQ ID NO: 4)

wherein Xaa, Xaa2, Xaa3, Xaa4, Xaa5, Xaa6 and Xaa7, individually for each occurrence, is an amino acid residue, with the caveat that (i) at least two of Xaa2, Xaa3, Xaa4 or Xaa5 are selected from His, Lys or Arg, or (ii) at least two of Xaa4, Xaa5, Xaa6 or Xaa7 are selected from His, Lys or Arg. In certain preferred embodiments, at least three, and preferably four of Xaa2, Xaa3, Xaa4, Xaa5, Xaa6 or Xaa7 are selected from His, Lys or Arg.

In certain embodiments, the AFFIMER® polypeptide sequence has an amino acid sequence wherein (Xaa)ₙ is an amino acid sequence at least 75% identical to the Loop 2 sequence selected from SEQ ID NOs: 6-299 and 1182, and more preferably at least 80%, 85%, 90%, or 95% identical. In certain embodiments, Loop 2 sequence is selected from SEQ ID NOs: 6-299 and 1182.

In certain embodiments, the AFFIMER® polypeptide sequence has an amino acid sequence wherein (Xaa)ₙ, is an amino acid sequence represented in the general formula

-Xaa-Xaa8-Xaa9-Xaa10-Xaa11-Xaa12-Xaa13-Xaa14-Xaa- (SEQ ID NO: 5)

wherein Xaa, Xaa8, Xaa9, Xaa10, Xaa11, Xaa12, Xaa13 and Xaa14, individually for each occurrence, is an amino acid residue, with the caveat that at least three of Xaa8, Xaa9, Xaa10, Xaa11, Xaa12, Xaa13 and Xaa14 are selected from His, Lys or Arg, and at least an additional two of Xaa8, Xaa9, Xaa10, Xaa11, Xaa12, Xaa13 and Xaa14 are selected from His, Lys, Arg, Phe, Tyr or Trp.

In certain embodiments, the AFFIMER® polypeptide sequence has an amino acid sequence wherein (Xaa)ₘ is an amino acid sequence at least 75% identical to the Loop 4 sequence selected from SEQ ID NOs: 300-593 and 1183, and more preferably at least 80%, 85%, 90%, or 95% identical. In certain embodiments, Loop 4 sequence is selected from SEQ ID NOs: 300-593 and 1183.

Another aspect relates to a protein comprising an FcRn binding AFFIMER® polypeptide sequence which binds to human FcRn and which is has an amino acid sequence that is at least 75% identical to an AFFIMER® polypeptide sequence selected from SEQ ID NOs: 594-887 and 1184, and more preferably 90%, 85%, 90% or even 95% identical. In certain embodiments, the FcRn binding AFFIMER® polypeptide sequence which binds to human FcRn and which is has an amino acid sequence that is identical to an AFFIMER® polypeptide sequence selected from SEQ ID NOs: 594-887 and 1184.

Yet another aspect relates to a protein comprising an FcRn binding AFFIMER® polypeptide sequence which binds to human FcRn and has an amino acid sequence that can be encoded by a nucleic acid having a coding sequence that hybridizes to any one of SEQ ID NOs: 888 to 1181 under stringent conditions of 6× sodium chloride/sodium citrate (SSC) at 45° C. followed by a wash in 0.2×SSC at 65° C.

Still another aspect relates to a protein comprising (i) an FcRn binding AFFIMER® polypeptide sequence which binds to human FcRn, and (ii) a heterologous polypeptide covalently associated to the FcRn binding AFFIMER® polypeptide sequence (optionally as a fusion protein or chemically conjugated) which confers a therapeutic activity in human patients.

In some embodiments, the polypeptides further comprise a heterologous polypeptide covalently linked through an amide bond to form a contiguous fusion protein.

In some embodiments, the heterologous polypeptide comprises a therapeutic polypeptide. In certain embodiments, the therapeutic polypeptide is selected from the group consisting of polypeptide hormones, polypeptide cytokines, polypeptide chemokines, growth factors, hemostasis active polypeptides, enzymes, and toxins. In certain embodiments, the therapeutic polypeptide is selected from the group consisting of receptor traps and receptor ligands. In certain embodiments, the therapeutic polypeptide sequence is selected from the group consisting of angiogenic agents and anti-angiogenic agents. In certain embodiments, the therapeutic polypeptide is a neurotransmitter, and optionally wherein the neurotransmitter is Neuropeptide Y. In certain embodiments, the therapeutic polypeptide is an erythropoiesis-stimulating agent, and optionally wherein the erythropoiesis-stimulating agent is erythropoietin or an erythropoietin mimetic. In certain embodiments, the therapeutic polypeptide is an incretin, and optionally wherein the incretin is selected from the group consisting of glucagon, gastric inhibitory peptide (GIP), glucagon-like peptide-1 (GLP-1), glucagon-like peptide-2 (GLP-2), peptide YY (PYY), and oxyntomodulin (OXM). In certain embodiments, the therapeutic polypeptide is an anticancer immune enhancing agent, such as a checkpoint inhibitor, a costimulatory receptor agonist or an iducer of innate immunity. In certain embodiments, the therapeutic polypeptide is an anti-inflammatory immune inhibiting agent, such as a checkpoint agonist, a costimulatory receptor antagonist or an inhibitor of innate immunity.

In some embodiments, the polypeptides extend the serum half-life of the heterologous polypeptide in vivo. For example, the heterologous polypeptide may have an extended half-life that is at least 2-fold, at least 5-fold, at least 10-fold, at least 20-fold, or at least 30-fold greater than the half-life of the heterologous polypeptide not linked to the AFFIMER® polypeptide.

In some embodiments, the polypeptides comprise a loop 2 amino acid sequence of any one of SEQ ID NOs: 6-299 and 1182. In some embodiments, the polypeptides comprise a loop 4 amino acid sequence of any one of SEQ ID NOs: 300-593 and 1183.

In some embodiments, the polypeptides comprise an amino acid sequence having at least 80%, at least 85%, at least 90%, at least 95%, at least 98%, or 100% identity to the sequence of any one of SEQ ID NOs: 594-887 or 1184.

In some embodiments, the polypeptides are encoded by a nucleic acid sequence having at least 70%, at least 75%, at least 80%, at least 85%, at least 90%, at least 95%, at least 98%, or 100% identity to the sequence of any one of SEQ ID NOs: 888-1181.

Other aspects of the present disclosure provide pharmaceutical preparations, e.g., for therapeutic use in a human patient, comprising any of the AFFIMER® polypeptides described herein, and a pharmaceutically acceptable excipient (e.g., carrier, buffer, and/or salt, etc.). In some embodiments, the pharmaceutical composition is formulated for pulmonary delivery. For example, the pharmaceutical composition may be formulated as an intranasal formulation. In other embodiments, the pharmaceutical composition is formulated for topical (e.g., transepithelial) delivery.

Further aspects of the present disclosure provide polynucleotides comprising a sequence encoding the AFFIMER® polypeptides described herein.

In some embodiments, the sequence encoding a polypeptide is operably linked to a transcriptional regulatory sequence. The transcriptional regulatory sequence may be, for example, a promoter or an enhancer. Other transcriptional regulatory sequence are contemplated herein.

In some embodiments, a polynucleotide further comprises an origin of replication, a minichromosome maintenance element (MME), and/or a nuclear localization element. In some embodiments, a polynucleotide further comprise a polyadenylation signal sequence operably linked and transcribed with the sequence encoding the polypeptide. In some embodiments, a polynucleotide further comprises at least one intronic sequence. In some embodiments, a polynucleotide further comprises at least one ribosome binding site transcribed with the sequence encoding the polypeptide.

In some embodiments, a polynucleotide is a deoxyribonucleic acid (DNA). In some embodiments, a polynucleotide is a ribonucleic acid (RNA).

Further aspects of the present disclosure provide viral vectors, plasmids, and/or minicircles comprising the AFFIMER® polypeptides described herein.

Other aspects of the present disclosure provide cells comprising the polypeptides polynucleotides, viral vectors, plasmids, and/or minicircles described herein.

Additional aspects of the present disclosure provide methods that comprise administering to a subject having an autoimmune disease and/or an inflammatory disease a therapeutically effective amount of the AFFIMER® polypeptides described herein.

Still other aspects of the present disclosure provide methods that comprise administering to a subject having a cancer a therapeutically effective amount of the AFFIMER® polypeptides described herein.

Yet other aspects of the present disclosure provide methods of increasing serum half-life of a therapeutic molecule, the method comprising conjugating the AFFIMER® polypeptides described herein to the therapeutic molecule.

Further aspects of the present disclosure provide methods of producing the polypeptides described herein, the methods comprising expressing in a host cell a nucleic acid encoding the polypeptide, and optionally isolating the polypeptide from the host cell.

It should be understood that any one of the AFFIMER® polypeptides described herein may include or exclude a signal sequence (e.g., ˜15-30 amino acids present at the N-terminus of the polypeptide) or a tag sequence (e.g., C-terminal polyhistadine (e.g., HHHHHH (SEQ ID NO: 1185))).

Still yet other aspects of the present disclosure provide use of the polynucleotide for targeting FcRn.

Still yet other aspects of the present disclosure provide use of the polynucleotide for increasing serum half-life of a therapeutic molecule.

### MODE FOR INVENTION

The present disclosure is based on the generation of AFFIMER® polypeptides that bind to human neonatal Fc receptor (FcRn) to extend, in a controlled manner, the serum half-life of any other therapeutic molecules (e.g., therapeutic AFFIMER® polypeptide, protein, nucleic acid, or drug) to which it is conjugated.

Based on naturally occurring proteins (Cystatins) that have been engineered to stably display two loops that create a binding surface, the human FcRn-binding AFFIMER® polypeptides of the present disclosure provide a number of advantages over antibodies, antibody fragments, and other non-antibody molecule-binding proteins. One is the small size of the AFFIMER® polypeptide itself. In its monomeric form it is about 14 kDa, or 1/10th the size of an antibody. This small size gives greater potential for increased tissue penetration, particularly in poorly vascularized and/or fibrotic target tissues (like tumors). AFFIMER® polypeptides have a simple protein structure (versus multi-domain antibodies), and as the AFFIMER® polypeptides do not require disulfide bonds or other post-translational modifications for function, these polypeptides can be manufactured in prokaryotic and eukaryotic systems.

Using libraries of AFFIMER® polypeptides (such as the phage display techniques described in the appended examples) as well as site directed mutagenesis, AFFIMER® polypeptides can be generated with tunable binding kinetics with ideal ranges for therapeutic uses. For instance, the AFFIMER® polypeptides can have high affinity for human FcRn, such as single digit nanomolar or lower K_{d} for monomeric AFFIMER® polypeptides, and picomolar K_{d} and avidity in multi-valent formats. The AFFIMER® polypeptides can be generated with tight binding kinetics for human FcRn, such as slow K_{off} rates in the 10⁻⁴ to 10⁻⁵ (s-1) range, which benefits target tissue localization.

The human FcRn-binding AFFIMER® polypeptides of the present disclosure include AFFIMER® polypeptides with exquisite selectivity.

Moreover, the human FcRn-binding AFFIMER® polypeptides can be readily formatted, allowing formats such as Fc fusions, whole antibody fusions, and in-line multimers to be generated and manufactured with ease.

The lack of need for disulfide bonds and post-translational modifications also permit many embodiments of proteins including the human FcRn-binding AFFIMER® polypeptides to be delivered therapeutically by expression of gene delivery constructs that are introduced into the tissues of a patient, including formats where the protein is delivered systemically (such as expression from muscle tissue) or delivered locally (such as through intratumoral gene delivery).

An AFFIMER® polypeptide (also referred to simply as an AFFIMER®) is a small, highly stable polypeptide (e.g., protein) that is a recombinantly engineered variant of stefin polypeptides. Thus, the term “AFFIMER® polypeptide” may be used interchangeably herein with the term “recombinantly engineered variant of stefin polypeptide”. The term “Affimer” may be used interchangeably with AFFIMER®, etc., and any term may be used without limitation. A stefin polypeptide is a subgroup of proteins in the cystatin superfamily—a family that encompasses proteins containing multiple cystatin-like sequences. The stefin subgroup of the cystatin family is relatively small (˜100 amino acids) single domain proteins. They receive no known post-translational modification, and lack disulfide bonds, suggesting that they will be able to fold identically in a wide range of extracellular and intracellular environments. Stefin A is a monomeric, single chain, single domain protein of 98 amino acids. The structure of stefin A has been solved, facilitating the rational mutation of stefin A into the AFFIMER® polypeptide. The only known biological activity of cystatins is the inhibition of cathepsin activity, has enabled exhaustively testing for residual biological activity of the engineered proteins.

AFFIMER® polypeptides display two peptide loops and an N-terminal sequence that can all be randomized to bind to desired target proteins with high affinity and specificity, in a similar manner to monoclonal antibodies. Stabilization of the two peptides by the stefin A protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the stefin A polypeptide sequence can be carried out, with such variations improving the properties of these affinity reagents, such as increase stability, make them robust across a range of temperatures and pH, for example. In some embodiments, an AFFIMER® polypeptide includes a sequence derived from stefin A, sharing substantial identify with a stefin A wild type sequence, such as human stefin A. In some embodiments, an AFFIMER® polypeptide has an amino acid sequence that shares at least 25%, 35%, 45%, 55% or 60% identity to the sequences corresponding to human stefin A. For example, an AFFIMER® polypeptide may have an amino acid sequence that shares at least 70%, at least 80%, at least 85%, at least 90%, at least 92%, at least 94%, at least 95% identity, e.g., where the sequence variations do not adversely affect the ability of the scaffold to bind to the desired target, and e.g., which do not restore or generate biological functions such as those that are possessed by wild type stefin A, but which are abolished in mutational changes described herein.

As used herein, the term AFFIMER® may be used interchangeably with “recombinantly engineered variant of stefin polypeptide”.

Human Neonatal Fc Receptor (FcRn) Binding AFFIMER® Polypeptides

One aspect of the disclosure provides AFFIMER® polypeptides that bind human neonatal Fc receptor (FcRn) (referred to as anti-human FcRn AFFIMER® polypeptides). Human neonatal Fc receptor, also known as the Brambell receptor, is a protein encoded by the FCGRT gene. This Fc receptor is similar in structure to the MHC class I molecule and also associates with beta-2-microglobulin. FcRn includes a 40 kDa alpha heavy chain that non-covalently associates with the 12 kDa light chain β-2-microgobulin. The FcRn heavy chain comprises three extracellular domains (α1, α2, and α3), a transmembrane domain, and a 44 amino acid cytoplasmic tail. In humans, FcRn has a role in monitoring IgG and serum albumin turnover (Kuo T T et al. mAbs 2011; 3(5):422-430; and Roopenian D C et al. Nature Reviews 2007; 7(9):715-725). Neonatal Fc receptor expression is up-regulated by the proinflammatory cytokine, TNF-α, and down-regulated by IFN-γ. A representative human FcRn sequence is provided by UniProtKB Primary accession number X, and may include other human isoforms thereof.

FcRn-mediated transcytosis of IgG across epithelial cells is possible because FcRn binds IgG at acidic pH (<6.5) but not at neutral or higher pH. Thus, FcRn can bind IgG from the slightly acidic intestinal lumen and ensure efficient, unidirectional transport to the basolateral side where the pH is neutral to slightly basic (Kuo T T et al. Journal of Clinical Immunology 2010; 30(6):777-89).

FcRn extends the half-life of IgG and serum albumin by reducing lysosomal degradation in endothelial cells (Roopenian D C et al. 2007) and bone-marrow derived cells (Akilesh S. et al. Journal of Immunology 2007; 179(7):4580-4588). IgG, serum albumin and other serum proteins are continuously internalized through pinocytosis. Generally, serum proteins are transported from the endosomes to the lysosome, where they are degraded. The two most abundant serum proteins, IgG and serum albumin are bound by FcRn at the slightly acidic pH (<6.5) and recycled to the cell surface where they are released at the neutral pH (>7.0) of blood. In this way IgG and serum albumin avoids lysosomal degradation. This mechanism provides an explanation for the greater serum circulation half-life of IgG and serum albumin (Goebl N A et al. Molecular Biology of the Cell 2008; 19(12):5490-505; and Roopenian D et al. 2007)

Anti-human FcRn AFFIMER® polypeptides comprise an AFFIMER® polypeptide in which at least one of the solvent accessible loops is from the wild-type stefin A protein having amino acid sequences to enable an AFFIMER® polypeptide to bind human FcRn, selectively, and in some embodiments, with K_{d} of 10⁻⁶M or less.

In some embodiments, the polypeptides bind to human FcRn with a K_{d} of 1×10⁻⁹ M to 1×10⁻⁶ M at pH 7.4 to 7.6. In some embodiments, the polypeptides bind to human FcRn with a K_{d} of 1×10⁻⁶ M or less at pH 7.4 to 7.6. In some embodiments, the polypeptides bind to human FcRn with a K_{d} of 1×10⁻⁷ M or less at pH 7.4 to 7.6. In some embodiments, the polypeptides bind to human FcRn with a K_{d} of 1×10⁻⁸ M or less at pH 7.4 to 7.6. In some embodiments, the polypeptides bind to human FcRn with a K_{d} of 1×10⁻⁹ M or less at pH 7.4 to 7.6. In some embodiments, the polypeptides bind to human FcRn with a K_{d} of 1×10⁻⁹ M to 1×10⁻⁶ M at pH 7.4. In some embodiments, the polypeptides bind to human FcRn with a K_{d} of 1×10⁻⁶ M or less at pH 7.4. In some embodiments, the polypeptides bind to human FcRn with a K_{d} of 1×10⁻⁷ M or less at pH 7.4. In some embodiments, the polypeptides bind to human FcRn with a K_{d} of 1×10⁻⁸ M or less at pH 7.4. In some embodiments, the polypeptides bind to human FcRn with a K_{d} of 1×10⁻⁹ M or less at pH 7.4.

In some embodiments, the polypeptides at pH 5.8 to 6.2 bind to human FcRn with a K_{d} of half a log to 2.5 logs less than the K_{d} for binding to human FcRn at pH 7.4 to 7.6. In some embodiments, the polypeptides at pH 5.8 to 6.2 bind to human FcRn with a K_{d} that is at least half a log less than the K_{d} for binding to human FcRn at pH 7.4 to 7.6. In some embodiments, the polypeptides at pH 5.8 to 6.2 bind to human FcRn with a K_{d} that is at least one log less than the K_{d} for binding to human FcRn at pH 7.4 to 7.6. In some embodiments, the polypeptides at pH 5.8 to 6.2 bind to human FcRn with a K_{d} that is at least 1.5 logs less than the K_{d} for binding to human FcRn at pH 7.4 to 7.6. In some embodiments, the polypeptides at pH 5.8 to 6.2 bind to human FcRn with a K_{d} that is at least 2 logs less than the K_{d} for binding to human FcRn at pH 7.4 to 7.6. In some embodiments, the polypeptides at pH 5.8 to 6.2 bind to human FcRn with a IQ that is at least 2.5 log less than the K_{d} for binding to human FcRn at pH 7.4 to 7.6. In some embodiments, the polypeptides at pH 6 bind to human FcRn with a IQ of half a log to 2.5 logs less than the K_{d} for binding to human FcRn at pH 7.4. In some embodiments, the polypeptides at pH 6 bind to human FcRn with a K_{d} that is at least half a log less than the K_{d} for binding to human FcRn at pH 7.4. In some embodiments, the polypeptides at pH 6 bind to human FcRn with a K_{d} that is at least one log less than the K_{d} for binding to human FcRn at pH 7.4. In some embodiments, the polypeptides at pH 6 bind to human FcRn with a K_{d} that is at least 1.5 logs less than the K_{d} for binding to human FcRn at pH 7.4. In some embodiments, the polypeptides at pH 6 bind to human FcRn with a K_{d} that is at least 2 logs less than the K_{d} for binding to human FcRn at pH 7.4. In some embodiments, the polypeptides at pH 6 bind to human FcRn with a K_{d} that is at least 2.5 log less than the K_{d} for binding to human FcRn at pH 7.4

In some embodiments, the polypeptides have a serum half-life in human patients of greater than 10 hours. In some embodiments, the polypeptides have a serum half-life in human patients of greater than 24 hours. In some embodiments, the polypeptides have a serum half-life in human patients of greater than 48 hours. In some embodiments, the polypeptides have a serum half-life in human patients of greater than 72 hours. In some embodiments, the polypeptides have a serum half-life in human patients of greater than 96 hours. In some embodiments, the polypeptides have a serum half-life in human patients of greater than 120 hours. In some embodiments, the polypeptides have a serum half-life in human patients of greater than 144 hours. In some embodiments, the polypeptides have a serum half-life in human patients of greater than 168 hours. In some embodiments, the polypeptides have a serum half-life in human patients of greater than 192 hours. In some embodiments, the polypeptides have a serum half-life in human patients of greater than 216 hours. In some embodiments, the polypeptides have a serum half-life in human patients of greater than 240 hours. In some embodiments, the polypeptides have a serum half-life in human patients of greater than 264 hours. In some embodiments, the polypeptides have a serum half-life in human patients of greater than 288 hours. In some embodiments, the polypeptides have a serum half-life in human patients of greater than 312 hours. In some embodiments, the polypeptides have a serum half-life in human patients of greater than 336 hours. In some embodiments, the polypeptides have a serum half-life in human patients of greater than 360 hours. In some embodiments, the polypeptides have a serum half-life in human patients of 24 to 360 hours, 48 to 360 hours, 72 to 360 hours, 96 to 360 hours, or 120 to 360 hours.

In some embodiments, an anti-human FcRn AFFIMER® polypeptide comprises a loop 2 amino acid sequence selected from any one of SEQ ID NOS: 6-299 and 1182 (Table 1). In some embodiments, an anti-human FcRn AFFIMER® polypeptide comprises a loop 4 amino acid sequence selected from any one of SEQ ID NOS: 300-593 and 1183 (Table 1).

In some embodiments, (Xaa)ₙ comprises an amino acid sequence having at least 80% or at least 90% identity to the amino acid sequence of any one of SEQ ID NOS: 6-299 and 1182. In some embodiments, (Xaa)ₙ comprises an amino acid sequence having 80% to 90% identity to the amino acid sequence of any one of SEQ ID NOS: 6-299 and 1182. In some embodiments, (Xaa)ₙ comprises the amino acid sequence of any one of SEQ ID NOS: 6-299 and 1182.

In some embodiments, (Xaa)ₘ comprises an amino acid sequence having at least 80% or at least 90% identity to the amino acid sequence of any one of SEQ ID NOS: 300-593 and 1183. In some embodiments, (Xaa)ₘ comprises an amino acid sequence having 80% to 90% identity to the amino acid sequence of any one of SEQ ID NOS: 300-593 and 1183. In some embodiments, (Xaa)ₘ comprises the amino acid sequence of any one of SEQ ID NOS: 300-593 and 1183.

TABLE 1
Examples of Anti-FcRn AFFIMER®
Loop Sequences

| | | SEQ | | SEQ |
|---|---|---|---|---|
| | | ID | | ID |
| Name | Loop 2 | NO: | Loop 4 | NO: |
| FcRn-01 | HVIDHKYRH | 6 | KKVNHHYHK | 300 |
| FcRn-02 | LKGHKHHKT | 7 | WQAKHKDGK | 301 |
| FcRn-03 | HNHHKYPHG | 8 | IWSKHNWHW | 302 |
| FcRn-04 | VHKKHHKWF | 9 | KWQVARHDN | 303 |
| FcRn-05 | KRHADHPRV | 10 | AHNYTLVWY | 304 |
| FcRn-06 | QQPKQHGFH | 11 | SSGNKHKHH | 305 |
| FcRn-07 | HHGHRTHSV | 12 | VWAHHKKYY | 306 |
| FcRn-08 | KQHHWDVHR | 13 | KVKHTRIH | 307 |
| FcRn-09 | GGQPAKQHF | 14 | PNKHHHAHK | 308 |
| FcRn-10 | NHVRWKDHD | 15 | FIKRYKLQR | 309 |
| FcRn-11 | HSHHPEHWY | 16 | RKDWHVRKL | 310 |
| FcRn-12 | KVKTHDHQR | 17 | IHQHHSQDW | 311 |
| FcRn-13 | YREVSKRRT | 18 | NQKQGHKHK | 312 |
| FcRn-14 | VTKRAWLKI | 19 | FYAQKRTSY | 313 |
| FcRn-15 | HNHRHYSKG | 20 | AFNDGAVFI | 314 |
| FcRn-16 | KHHHHKHQH | 21 | VFLHNESHQ | 315 |
| FcRn-17 | HPHHVRSSV | 22 | KGHFHTHLV | 316 |
| FcRn-18 | ETPHERHKT | 23 | KRWLKHHAH | 317 |
| FcRn-19 | GTIQHVNQH | 24 | YGHKHHFHW | 318 |
| FcRn-20 | YNVGRKKHR | 25 | VHFFHDQSE | 319 |
| FcRn-21 | RRGPQKSSY | 26 | QKKNRHHQK | 320 |
| FcRn-22 | HDRHQKHWR | 27 | DLRKHKWKS | 321 |
| FcRn-23 | IPHHHKPRV | 28 | SFHHHRHSD | 322 |
| FcRn-24 | KGKHYHSQQ | 29 | EFYQGHWTN | 323 |
| FcRn-25 | HKHKHHHTN | 30 | VGHHWWLKE | 324 |
| FcRn-26 | GRHKHIQVH | 31 | VGTKHLRQS | 325 |
| FcRn-27 | PHQHKLHAH | 32 | KRRRHPSRG | 326 |
| FcRn-28 | RRDHVWHKG | 33 | NHVHNKHIH | 327 |
| FcRn-29 | SHRSHADRR | 34 | TQSHPHRHY | 328 |
| FcRn-30 | SSQNGYQGH | 35 | YRHHHHWHF | 329 |
| FcRn-31 | TEGGKKLRR | 36 | EWTHGKENH | 330 |
| FcRn-32 | KARHHQGHA | 37 | WYQFDGVSF | 331 |
| FcRn-33 | NHSQGRHHI | 38 | KKVRHEYAW | 332 |
| FcRn-34 | KYWKADWYW | 39 | EHSWWRRGH | 333 |
| FcRn-35 | HRQYPPGPH | 40 | YHFHHYYKH | 334 |
| FcRn-36 | RQHHHFYRT | 41 | WQNFHDPFD | 335 |
| FcRn-37 | PQQHQPDPT | 42 | ARQHHHHSH | 336 |
| FcRn-38 | LSFNNYHWH | 43 | KLRHDKLTH | 337 |
| FcRn-39 | HHSKHHHLH | 44 | NHKFQSYQP | 338 |
| FcRn-40 | HKYDRHSFK | 45 | GKHSGARHK | 339 |
| FcRn-41 | KHSRHHHAQY | 46 | NIHHEGKIP | 340 |
| FcRn-42 | RHHHSHFHL | 47 | IRQSSYKVH | 341 |
| FcRn-43 | RNHRHPHGQ | 48 | VQHRWSLHW | 342 |
| FcRn-44 | GHVEQVHFPY | 49 | GHKHHHHWS | 343 |
| FcRn-45 | EPHKHHYHL | 50 | VPGQQPIKN | 344 |
| FcRn-46 | WKKHNWKYK | 51 | WAAKRDWRN | 345 |
| FcRn-47 | IHHHTWGLK | 52 | YGDQPFKRH | 346 |
| FcRn-48 | KPKYHHHDI | 53 | GHHAKPHRW | 347 |
| FcRn-49 | QYWHSHETW | 54 | FLKVRTIRS | 348 |
| FcRn-50 | RKQYHLPWT | 55 | LSQFQTHLW | 349 |
| FcRn-51 | AIHWAHYIL | 56 | VLWRYYYPK | 350 |
| FcRn-52 | DWRKLTLF | 57 | HHQHWHVFP | 351 |
| FcRn-53 | TKSHKFAYH | 58 | IVQEFSLDQW | 352 |
| FcRn-54 | SKYVHWHKF | 59 | WKINNLYHE | 353 |
| FcRn-55 | KEQAAWVLH | 60 | FHYLHHTRS | 354 |
| FcRn-56 | HLQAPRNAY | 61 | KGWRNTHHK | 355 |
| FcRn-57 | GLTHRWRPH | 62 | IWSARSDKL | 356 |
| FcRn-58 | SHHRATDQV | 63 | KAYHTYWHH | 357 |
| FcRn-59 | NKWHIRFAT | 64 | FAQAHHHTQ | 358 |
| FcRn-60 | HIRDSLWIT | 65 | NWQWIPHWA | 359 |
| FcRn-61 | YHISLSFRE | 66 | KLDTLGQQR | 360 |
| FcRn-62 | IHWAGFFRG | 67 | WEWERHWLA | 361 |
| FcRn-63 | YYSERHFYK | 68 | FTLGREGWF | 362 |
| FcRn-64 | RQQQVHVPS | 69 | YRGNTFKIW | 363 |
| FcRn-65 | TKKNQLQGH | 70 | VHSLLQHHD | 364 |
| FcRn-66 | RDIHHHHHW | 71 | YIKRHWSNF | 365 |
| FcRn-67 | QRQYTTKVL | 72 | DNERNQVES | 366 |
| FcRn-68 | YWDWRFVEW | 73 | IGYELFTVK | 367 |
| FcRn-69 | GFSKPFKWY | 74 | YRAWIHWTS | 368 |
| FcRn-70 | IFQERLAGQ | 75 | QIKHSHHAW | 369 |
| FcRn-71 | KYDHHTQSL | 76 | VYAWYWDKW | 370 |
| FcRn-72 | KHAHTPFGP | 77 | AVWWDGRGW | 371 |
| FcRn-73 | SLSRWLWAE | 78 | WHTHKHYQK | 372 |
| FcRn-74 | HQQHTQRYR | 79 | AKLQFGHKH | 373 |
| FcRn-75 | HTISQHVST | 80 | SFRWHRF | 374 |
| FcRn-76 | DQWTWAHSR | 81 | DYHLRHHNH | 375 |
| FcRn-77 | WYRVWRWVW | 82 | VYKYGSENW | 376 |
| FcRn-78 | QKGSTHHNH | 83 | ARSQAGHHN | 377 |
| FcRn-79 | PEGRAGEPS | 84 | EHWWFTFGD | 378 |
| FcRn-80 | HTRHHVTLW | 85 | GWKYAPQVW | 379 |
| FcRn-81 | QRYYKHEYR | 86 | YFKLPPWEE | 380 |
| FcRn-82 | QWFHRREVK | 87 | PVHLHHKQH | 381 |
| FcRn-83 | HHLHATQPP | 88 | NWHIINKYD | 382 |
| FcRn-84 | KHWHQPVAK | 89 | AHWHDWV | 383 |
| FcRn-85 | YTTSHWTIG | 90 | DHHHVQKSH | 384 |
| FcRn-86 | EHHHTQLSN | 91 | KFWQVQQKY | 385 |
| FcRn-87 | HKPHNSKQI | 92 | KPRFNIHHH | 386 |
| FcRn-88 | HHTKHHSRW | 93 | VNHISHAPI | 387 |
| FcRn-89 | FHRHHPIWH | 94 | LKPWEADLW | 388 |
| FcRn-90 | ARVTIDWKA | 95 | YKYPNIHPH | 389 |
| FcRn-91 | KLEQRRSHY | 96 | PKSLFNYQH | 390 |
| FcRn-92 | NIHHVHHQQ | 97 | DGEFHVKQV | 391 |
| FcRn-93 | SHHTIAWYV | 98 | VYPKRQQVE | 392 |
| FcRn-94 | HHQPYYGWQ | 99 | IIDRSKIEK | 393 |
| FcRn-95 | VHRSHHPIK | 100 | SIHSSWKKQ | 394 |
| FcRn-96 | WWSQRVKLF | 101 | NIHKTWDQT | 395 |
| FcRn-97 | HYWKPHDIH | 102 | GKVPFHAFHK | 396 |
| FcRn-98 | TNQPRLYHQ | 103 | FYRLTHGHR | 397 |
| FcRn-99 | WSGKLLKHP | 104 | HIDYKNGRIW | 398 |
| FcRn-100 | HRTSWDHKN | 105 | VFHHQRGGQ | 399 |
| FcRn-101 | PHKQKRHFFN | 106 | WGQSKPAHV | 400 |
| FcRn-102 | HDQHKHDFK | 107 | FHQRFPDHK | 401 |
| FcRn-103 | NRVVHHFHH | 108 | IQAAEGYKH | 402 |
| FcRn-104 | WHKAIRQQF | 109 | FHYQYRHQH | 403 |
| FcRn-105 | TKEWHQHIK | 110 | NKFLHGFEV | 404 |
| FcRn-106 | WYHTHFANA | 111 | FKRHQHGHK | 405 |
| FcRn-107 | TRVHNLSVL | 112 | HYDRAHYFK | 406 |
| FcRn-108 | WNQPYWTTY | 113 | FRWKFHDYK | 407 |
| FcRn-109 | RPHNRDSHR | 114 | DRKHRKHWH | 408 |
| FcRn-110 | GHPRHHWKY | 115 | ATYKYRVDY | 409 |
| FcRn-111 | YPGHHHARD | 116 | YFYHHHWFK | 410 |
| FcRn-112 | IAKHHTWHQ | 117 | YRNHRHHIV | 411 |
| FcRn-113 | HNHGHWHFR | 118 | VQHARHKHY | 412 |
| FcRn-114 | KKFDHYHQK | 119 | KDRHHHNR | 413 |
| FcRn-115 | SKAHRVEHK | 120 | KQHHLYHFK | 414 |
| FcRn-116 | PKKHYHHGI | 121 | VNSFQAHRH | 415 |
| FcRn-117 | NSHRIQHGF | 122 | SHHLHRSAH | 416 |
| FcRn-118 | PHHSHHRLE | 123 | QPTFRHHYT | 417 |
| FcRn-119 | HVHHHREKG | 124 | YSNSRERQW | 418 |
| FcRn-120 | KHKYHHTGH | 125 | GQIHKVRST | 419 |
| FcRn-121 | KYFAPHAPH | 126 | HYHHRHQHS | 420 |
| FcRn-122 | LHHRAHKHL | 127 | YFHREHEHQ | 421 |
| FcRn-123 | AHHGHYGRA | 128 | WHYHHSQWR | 422 |
| FcRn-124 | PEHYSLFKP | 129 | KHHRKHRHW | 423 |
| FcRn-125 | DHRPRHPKH | 130 | AHKHHLGFK | 424 |
| FcRn-126 | KHEVHHHGN | 131 | WHRHGSGFR | 425 |
| FcRn-127 | KSHHHKHRE | 132 | VDRFLHVKK | 426 |
| FcRn-128 | HRHHTHKWT | 133 | WPHSIDYRQ | 427 |
| FcRn-129 | GKHPHHHQN | 134 | KGRYSHHHG | 428 |
| FcRn-130 | WHKHHLRYR | 135 | YPQDKHKVL | 429 |
| FcRn-131 | KTHKEYHHS | 136 | GYRRHQGRG | 430 |
| FcRn-132 | RRHHHQHWS | 137 | ALHDTLHPS | 431 |
| FcRn-133 | THRWHQGSR | 138 | KKPHNHRYY | 432 |
| FcRn-134 | KRGHHHPNH | 139 | AKHHWDTWS | 433 |
| FcRn-135 | HTVPLRKHQ | 140 | VIHHKHRHQ | 434 |
| FcRn-136 | TYRWGHHFH | 141 | KYEQIDRWH | 435 |
| FcRn-137 | FKHHDRGTH | 142 | YRKRHTWFQ | 436 |
| FcRn-138 | TAKKHPKSH | 143 | KVNWHHYRH | 437 |
| FcRn-139 | HYHFSKHHN | 144 | SYHHKHFVK | 438 |
| FcRn-140 | YKHKHGKWR | 145 | WHGHFSKGGVAY | 439 |
| FcRn-141 | VHHKPHKTE | 146 | ATHLKHHNH | 440 |
| FcRn-142 | HGQRYHNKS | 147 | KRKWEHSHK | 441 |
| FcRn-143 | HKHHRHVPS | 148 | DHRHRHWYL | 442 |
| FcRn-144 | HRKHSWSRH | 149 | TKHSHSQLF | 443 |
| FcRn-145 | NRHYHQEYK | 150 | VHKSKHWFY | 444 |
| FcRn-146 | KIKHHHSFK | 151 | SQDHHFHRH | 445 |
| FcRn-147 | QHKRSHRQS | 152 | GHKYSHWSK | 446 |
| FcRn-148 | SVYKWKA | 153 | NKHHHHAHH | 447 |
| FcRn-149 | RKLERTKYH | 154 | HNKYHPHNK | 448 |
| FcRn-150 | TGHKHQFHQ | 155 | KHKHGWFHS | 449 |
| FcRn-151 | WQELGHRVY | 156 | YRRHHDKKH | 450 |
| FcRn-152 | HPHHTDQRH | 157 | EGHRQHAKF | 451 |
| FcRn-153 | FHNHGHPHL | 158 | NSRGHHHHK | 452 |
| FcRn-154 | WNHHHRNKQ | 159 | PHKRPHLYH | 453 |
| FcRn-155 | TRHGHRHYR | 160 | FYDLHPKLS | 454 |
| FcRn-156 | PHHRWHRQH | 161 | IHQHSQKKS | 455 |
| FcRn-157 | NLRHQTEHR | 162 | KRHHRHSHV | 456 |
| FcRn-158 | GHRKHTHLL | 163 | KKSHKAWAW | 457 |
| FcRn-159 | RHSKPQHWP | 164 | KGHKQHHHY | 458 |
| FcRn-160 | PHRSRFHKQ | 165 | WKAERHKHY | 459 |
| FcRn-161 | QRKHFHWDH | 166 | QHRYTHHHT | 460 |
| FcRn-162 | NKHHGQQHN | 167 | SHKVHTHSK | 461 |
| FcRn-163 | KYHHKYKSY | 168 | KHLDQYHPS | 462 |
| FcRn-164 | REWHHQTYY | 169 | SAHKHHHNH | 463 |
| FcRn-165 | RHYHDHHYR | 170 | KYKHQVKQH | 464 |
| FcRn-166 | SHTYRHSTG | 171 | ISHRHRHDI | 465 |
| FcRn-167 | NHRHHHPHF | 172 | NYHAHRSFY | 466 |
| FcRn-168 | HAKTRHHEH | 173 | WFKHHFWHR | 467 |
| FcRn-169 | EPHQKHKRH | 174 | KRKGDFLNY | 468 |
| FcRn-170 | DRRHQHGRH | 175 | HKPWGHHKL | 469 |
| FcRn-171 | HQHRHNLQQ | 176 | QYKHKHWLW | 470 |
| FcRn-172 | KRIHTWHTD | 177 | FKRHHSWHH | 471 |
| FcRn-173 | YHHQPRYQQ | 178 | KDRHHEFRH | 472 |
| FcRn-174 | GIGRHRRRR | 179 | HHHHFHNHR | 473 |
| FcRn-175 | DQHKQHYHF | 180 | SVNQHFKHK | 474 |
| FcRn-176 | GRHHESHKS | 181 | FQHKLHKHH | 475 |
| FcRn-177 | KRHHHWHYS | 182 | DTRYDKWHG | 476 |
| FcRn-178 | NRKGGHRYH | 183 | HVHRVQHSK | 477 |
| FcRn-179 | RKWHGHWHR | 184 | WNYQFKSAS | 478 |
| FcRn-180 | NWKRHHYHR | 185 | QWWFHKHVK | 479 |
| FcRn-181 | TRHHHRNRF | 186 | ISHNPNHYH | 480 |
| FcRn-182 | VKWDFKHFY | 187 | TNLHSPDSP | 481 |
| FcRn-183 | SDDLSPVKW | 188 | FDKYNSHYL | 482 |
| FcRn-184 | RHRQKWPIH | 189 | STHQQKHQW | 483 |
| FcRn-185 | DRHAYHRH | 190 | FHEEIKHWQ | 484 |
| FcRn-186 | HRHHQKHAF | 191 | WRDWNHRFP | 485 |
| FcRn-187 | QKGKHHDYR | 192 | KPHQTKWHH | 486 |
| FcRn-188 | WNKHFYKQG | 193 | RHHRQSHHW | 487 |
| FcRn-189 | KRRHNREFV | 194 | IRHYHADRE | 488 |
| FcRn-190 | TRHVRHWTH | 195 | ASQVPPKHR | 489 |
| FcRn-191 | NRKWQQNHH | 196 | KHKHWHHQL | 490 |
| FcRn-192 | RHREKHQPY | 197 | WEHHRTRWQ | 491 |
| FcRn-193 | YHKHNSKHS | 198 | FKTFKEWHV | 492 |
| FcRn-194 | PAGQHKRKH | 199 | KGHRWHDFK | 493 |
| FcRn-195 | DRHKYPVRV | 200 | KHAWQHHKS | 494 |
| FcRn-196 | GNNNPQGHV | 201 | YKHFKHHWR | 495 |
| FcRn-197 | KQLHHHHYK | 202 | AHRKFFQWH | 496 |
| FcRn-198 | QKHNWHRWH | 203 | WTHRSQVKV | 497 |
| FcRn-199 | YKHLGYWQK | 204 | FQWFKVGVP | 498 |
| FcRn-200 | HQKNFEAWE | 205 | VRYYSKYQW | 499 |
| FcRn-201 | ERVRRRHPP | 206 | NGWHVGHHI | 500 |
| FcRn-202 | HKVHIFREP | 207 | TRFRHYLVT | 501 |
| FcRn-203 | VKSFHVHSH | 208 | SWRNVRPEF | 502 |
| FcRn-204 | WHKDPPPPW | 209 | FGHTFSWRY | 503 |
| FcRn-205 | HRYAHNHFL | 210 | FKHQKFYRD | 504 |
| FcRn-206 | VSHALKTHT | 211 | WRNKWRAQD | 505 |
| FcRn-207 | HQSRAIYVY | 212 | YQKSYFHRH | 506 |
| FcRn-208 | HHTTYHQHH | 213 | WRPRPVHWK | 507 |
| FcRn-209 | TWWRNVQHH | 214 | DPQYKRHGY | 508 |
| FcRn-210 | WNKHNYQHQ | 215 | VPHSVVHYK | 509 |
| FcRn-211 | QHTLRVHTV | 216 | AYSQSFIHH | 510 |
| FcRn-212 | NQHFHQAGH | 217 | FSHSTWRYH | 511 |
| FcRn-213 | RQWTDRVWV | 218 | SKKHQQHW | 512 |
| FcRn-214 | DHDYFHHNK | 219 | AKHPRIHVT | 513 |
| FcRn-215 | YWDVGPGFN | 220 | SPWHHPTHF | 514 |
| FcRn-216 | GIHGHHEYY | 221 | SNWFHHKHR | 515 |
| FcRn-217 | WQRSRYGKY | 222 | AYWPYQKPT | 516 |
| FcRn-218 | YHQQHWRVH | 223 | ILVGYNWHY | 517 |
| FcRn-219 | ATRNSYPRH | 224 | VHSHLPRHP | 518 |
| FcRn-220 | EHHHAHWAT | 225 | LFLHGVHIF | 519 |
| FcRn-221 | KQHQRSFII | 226 | TSLPSEWFQ | 520 |
| FcRn-222 | QFWGHRVEH | 227 | TRHYHQRNR | 521 |
| FcRn-223 | FPSSHRTSY | 228 | YSAHHIRWH | 522 |
| FcRn-224 | SSKYIDHRQ | 229 | ERAQHHTHP | 523 |
| FcRn-225 | YWRHEHSSP | 230 | WKKHHYGHY | 524 |
| FcRn-226 | ERAHYDHHY | 231 | SHHAHHSVQ | 525 |
| FcRn-227 | WRHKAYIYG | 232 | WKHWEHKPQ | 526 |
| FcRn-228 | PQIKEQYNG | 233 | AQVPVLLWY | 527 |
| FcRn-229 | FKKVARDHW | 234 | WVHFYPWQQ | 528 |
| FcRn-230 | AQKHHWHKT | 235 | WHLAHVFYT | 529 |
| FcRn-231 | VSQGHHSWD | 236 | SSHHHKNHH | 530 |
| FcRn-232 | WHLRGHPHY | 237 | TKQPHGVHY | 531 |
| FcRn-233 | HSHHHQPWE | 238 | EHRTHHLGK | 532 |
| FcRn-234 | RRFRVHLHQ | 239 | TNHRQDHPE | 533 |
| FcRn-235 | GRQTKSHQH | 240 | HRKTNWHSY | 534 |
| FcRn-236 | PYSRHHHQL | 241 | SGVHHAAVW | 535 |
| FcRn-237 | VHGDHTRAW | 242 | RYASSYWEW | 536 |
| FcRn-238 | DWQKRGRSW | 243 | NQSGVVVQV | 537 |
| FcRn-239 | YNWERFRKV | 244 | YHNHQHTIH | 538 |
| FcRn-240 | GWSRNVWFW | 245 | KQELGTKTT | 539 |
| FcRn-241 | SQTQHRRHH | 246 | LVPQHHQHQ | 540 |
| FcRn-242 | PNVKHKHRW | 247 | WHDIAGGHY | 541 |
| FcRn-243 | KHPAFHQHS | 248 | RHDLHYHYP | 542 |
| FcRn-244 | PHHHTDWRT | 249 | YWHWKVRRF | 543 |
| FcRn-245 | HTHKILHFH | 250 | DKQRYEDKQ | 544 |
| FcRn-246 | PNHHFFLQF | 251 | QHHHPHRHP | 545 |
| FcRn-247 | RRYIGHNYS | 252 | WHHFHNSYD | 546 |
| FcRn-248 | THYHHQWDP | 253 | IWYSHRPRA | 547 |
| FcRn-249 | DKKHGQYK | 254 | WDDHTLKWY | 548 |
| FcRn-250 | YHIQGVYWR | 255 | IAFWGPKRF | 549 |
| FcRn-251 | SRFKHHVRN | 256 | FPHRNKSDG | 550 |
| FcRn-252 | WHHQHHLLA | 257 | FKRSQQWEW | 551 |
| FcRn-253 | HNKHPSPRV | 258 | KHRYQPTHW | 552 |
| FcRn-254 | TWFHQHEQQ | 259 | YHDIWAWHV | 553 |
| FcRn-255 | WKEWRYHHQ | 260 | DFVKHHLHD | 554 |
| FcRn-256 | FTKHWDRWY | 261 | ISDHVHFGW | 555 |
| FcRn-257 | TRLYDHSVW | 262 | YHHRDHWGW | 556 |
| FcRn-258 | WEYQTHHPA | 263 | EWFTVGGIA | 557 |
| FcRn-259 | VHFRSHRDF | 264 | ERKHAHQHP | 558 |
| FcRn-260 | SRHTHHHRS | 265 | DSNLYNEWN | 559 |
| FcRn-261 | TARYEHAPT | 266 | TAKHSHKKH | 560 |
| FcRn-262 | RHRKESWYV | 267 | NWPHGIDPK | 561 |
| FcRn-263 | DHGYARGHH | 268 | KHIHEHKSE | 562 |
| FcRn-264 | TPHKIWHWH | 269 | TKKFHQHER | 563 |
| FcRn-265 | SYAQHTRLH | 270 | TRHHQHYYL | 564 |
| FcRn-266 | IDHRYHYLH | 271 | WYWTQHHRW | 565 |
| FcRn-267 | HGYNHRKVQ | 272 | YHVWNWRLK | 566 |
| FcRn-268 | GHLKAAPWH | 273 | FHHFRPHHH | 567 |
| FcRn-269 | KEKYASWER | 274 | FLNGKKRHV | 568 |
| FcRn-270 | KGHPHAHPH | 275 | WWKIHGSTV | 569 |
| FcRn-271 | PYRRHEHHQ | 276 | NSDFHHNQQ | 570 |
| FcRn-272 | GFPHWFVHN | 277 | THHLRYHHQ | 571 |
| FcRn-273 | FRRYQSFHY | 278 | FYKYHQVRW | 572 |
| FcRn-274 | PRYRHHVDY | 279 | YSFRDHHWW | 573 |
| FcRn-275 | DYLKRNFRY | 280 | PFYRNHHHE | 574 |
| FcRn-276 | RSHPGKHVH | 281 | FQLNLRWGQ | 575 |
| FcRn-277 | HHHRWAKWL | 282 | VHNFHDIRH | 576 |
| FcRn-278 | AAHHNHWHI | 283 | AQHGHVPFS | 577 |
| FcRn-279 | PVQKHAGSH | 284 | PWHNAEIKH | 578 |
| FcRn-280 | DNWRHWRIW | 285 | AGWSSNKAD | 579 |
| FcRn-281 | PRHHHWAF | 286 | KRQHHDVGQ | 580 |
| FcRn-282 | VSYDDITWV | 287 | NSSYGWLWW | 581 |
| FcRn-283 | PPHPRVQHY | 288 | AFRDHRAPH | 582 |
| FcRn-284 | KQFRHHQHE | 289 | KWWSTQGIV | 583 |
| FcRn-285 | EHHEYHYRY | 290 | FRPVHHIRI | 584 |
| FcRn-286 | HHHHRQHP | 291 | KVGQGVNLG | 585 |
| FcRn-287 | KLHQAHHWH | 292 | EWSNKHYQW | 586 |
| FcRn-288 | EYHHYGTSR | 293 | RQLKHHTNF | 587 |
| FcRn-289 | DNKHIPQRQ | 294 | RNHVAEKYW | 588 |
| FcRn-290 | HKQWQWTIV | 295 | AYKSDKIRK | 589 |
| FcRn-291 | YRIGHGVQH | 296 | YDKPYIVWI | 590 |
| FcRn-292 | DQVRRIPHH | 297 | HDKHPQSWA | 591 |
| FcRn-293 | EGKHEFRFQ | 298 | WDKHRQHLW | 592 |
| FcRn-294 | HYWGRWYKI | 299 | FHAFWHLAY | 593 |
| AVA04-2 | REGRQDWVL | 1182 | WVPFPHQQL | 1183 |
| 51 FX6 | | | | |

In some embodiments, an anti-human FcRn AFFIMER® polypeptide comprises an amino acid sequence selected from any one of SEQ ID NOS: 594-887 and 1184 (Table 2).

In some embodiments, an anti-human FcRn AFFIMER® polypeptide comprises an amino acid sequence having at least 80% or at least 90% identity to the amino acid sequence of any one of SEQ ID NOS: 594-887 and 1184. In some embodiments, an anti-human FcRn AFFIMER® polypeptide comprises an amino acid sequence having 80% to 90% identity to the amino acid sequence of any one of SEQ ID NOS: 594-887 and 1184. In some embodiments, an anti-human FcRn AFFIMER® polypeptide comprises the amino acid sequence of any one of SEQ ID NOS: 594-887 and 1184.

TABLE 2
Examples of Anti-FcRn AFFIMER®Polypeptide Sequences

| | | SEQ |
|---|---|---|
| Name | Protein Sequence | ID NO: |
| FcRn-01 | MIPRGLSEAKPATPEIQEIVDKVKPQLEEKTNETYGKLEAVQYKTQ | 594 |
| | VLAHVIDHKYRHSTNYYIKVRAGDNKYMHLKVFNGPKKVNHHY | |
| | HKADRVLTGYQVDKNKDDELTGF | |
| FcRn-02 | MIPRGLSEAKPATPEIQEIVDKVKPQLEEKTNETYGKLEAVQYKTQ | 595 |
| | VLALKGHKHHKTSTNYYIKVRAGDNKYMHLKVFNGPWQAKHKD | |
| | GKADRVLTGYQVDKNKDDELTGF | |
| FcRn-03 | MIPRGLSEAKPATPEIQEIVDKVKPQLEEKTNETYGKLEAVQYKTQ | 596 |
| | VLAHNHHKYPHGSTNYYIKVRAGDNKYMHLKVFNGPIWSKHNW | |
| | HWADRVLTGYQVDKNKDDELTGF | |
| FcRn-04 | MIPRGLSEAKPATPEIQEIVDKVKPQLEEKTNETYGKLEAVQYKTQ | 597 |
| | VLAVHKKHHKWFSTNYYIKVRAGDNKYMHLKVFNGPKWQVAR | |
| | HDNADRVLTGYQVDKNKDDELTGF | |
| FcRn-05 | MIPRGLSEAKPATPEIQEIVDKVKPQLEEKTNETYGKLEAVQYKTQ | 598 |
| | VLAKRHADHPRVSTNYYIKVRAGDNKYMHLKVFNGPAHNYTLV | |
| | WYADRVLTGYQVDKNKDDELTGF | |
| FcRn-06 | MIPRGLSEAKPATPEIQEIVDKVKPQLEEKTNETYGKLEAVQYKTQ | 599 |
| | VLAQQPKQHGFHSTNYYIKVRAGDNKYMHLKVFNGPSSGNKHKH | |
| | HADRVLTGYQVDKNKDDELTGF | |
| FcRn-07 | MIPRGLSEAKPATPEIQEIVDKVKPQLEEKTNETYGKLEAVQYKTQ | 600 |
| | VLAHHGHRTHSVSTNYYIKVRAGDNKYMHLKVFNGPVWAHHKK | |
| | YYADRVLTGYQVDKNKDDELTGF | |
| FcRn-08 | MIPRGLSEAKPATPEIQEIVDKVKPQLEEKTNETYGKLEAVQYKTQ | 601 |
| | VLAKQHHWDVHRSTNYYIKVRAGDNKYMHLKVFNGPKVKHTRI | |
| | HADRVLTGYQVDKNKDDELTGF | |
| FcRn-09 | MIPRGLSEAKPATPEIQEIVDKVKPQLEEKTNETYGKLEAVQYKTQ | 602 |
| | VLAGGQPAKQHFSTNYYIKVRAGDNKYMHLKVFNGPPNKHHHA | |
| | HKADRVLTGYQVDKNKDDELTGF | |
| FcRn-10 | MIPRGLSEAKPATPEIQEIVDKVKPQLEEKTNETYGKLEAVQYKTQ | 603 |
| | VLANHVRWKDHDSTNYYIKVRAGDNKYMHLKVFNGPFIKRYKLQ | |
| | RADRVLTGYQVDKNKDDELTGF | |
| FcRn-11 | MIPRGLSEAKPATPEIQEIVDKVKPQLEEKTNETYGKLEAVQYKTQ | 604 |
| | VLAHSHHPEHWYSTNYYIKVRAGDNKYMHLKVFNGPRKDWHVR | |
| | KLADRVLTGYQVDKNKDDELTGF | |
| FcRn-12 | MIPRGLSEAKPATPEIQEIVDKVKPQLEEKTNETYGKLEAVQYKTQ | 605 |
| | VLAKVKTHDHQRSTNYYIKVRAGDNKYMHLKVFNGPIHQHHSQD | |
| | WADRVLTGYQVDKNKDDELTGF | |
| FcRn-13 | MIPRGLSEAKPATPEIQEIVDKVKPQLEEKTNETYGKLEAVQYKTQ | 606 |
| | VLAYREVSKRRTSTNYYIKVRAGDNKYMHLKVFNGPNQKQGHKH | |
| | KADRVLTGYQVDKNKDDELTGF | |
| FcRn-14 | MIPRGLSEAKPATPEIQEIVDKVKPQLEEKTNETYGKLEAVQYKTQ | 607 |
| | VLAVTKRAWLKISTNYYIKVRAGDNKYMHLKVFNGPFYAQKRTS | |
| | YADRVLTGYQVDKNKDDELTGF | |
| FcRn-15 | MIPRGLSEAKPATPEIQEIVDKVKPQLEEKTNETYGKLEAVQYKTQ | 608 |
| | VLAHNHRHYSKGSTNYYIKVRAGDNKYMHLKVFNGPAFNDGAVF | |
| | IADRVLTGYQVDKNKDDELTGF | |
| FcRn-16 | MIPRGLSEAKPATPEIQEIVDKVKPQLEEKTNETYGKLEAVQYKTQ | 609 |
| | VLAKHHHHKHQHSTNYYIKVRAGDNKYMHLKVFNGPVFLHNESH | |
| | QADRVLTGYQVDKNKDDELTGF | |
| FcRn-17 | MIPRGLSEAKPATPEIQEIVDKVKPQLEEKTNETYGKLEAVQYKTQ | 610 |
| | VLAHPHHVRSSVSTNYYIKVRAGDNKYMHLKVFNGPKGHFHTHL | |
| | YADRVLTGYQVDKNKDDELTGF | |
| FcRn-18 | MIPRGLSEAKPATPEIQEIVDKVKPQLEEKTNETYGKLEAVQYKTQ | 611 |
| | VLAETPHERHKTSTNYYIKVRAGDNKYMHLKVFNGPKRWLKHHA | |
| | KADRVLTGYQVDKNKDDELTGF | |
| FcRn-19 | MIPRGLSEAKPATPEIQEIVDKVKPQLEEKTNETYGKLEAVQYKTQ | 612 |
| | VLAGTIQHVNQHSTNYYIKVRAGDNKYMHLKVFNGPYGHKHHFH | |
| | WADRVLTGYQVDKNKDDELTGF | |
| FcRn-20 | MIPRGLSEAKPATPEIQEIVDKVKPQLEEKTNETYGKLEAVQYKTQ | 613 |
| | VLAYNVGRKKHRSTNYYIKVRAGDNKYMHLKVFNGPVHFFHDQS | |
| | EADRVLTGYQVDKNKDDELTGF | |
| FcRn-21 | MIPRGLSEAKPATPEIQEIVDKVKPQLEEKTNETYGKLEAVQYKTQ | 614 |
| | VLARRGPQKSSYSTNYYIKVRAGDNKYMHLKVFNGPQKKNRHHQ | |
| | KADRVLTGYQVDKNKDDELTGF | |
| FcRn-22 | MIPRGLSEAKPATPEIQEIVDKVKPQLEEKTNETYGKLEAVQYKTQ | 615 |
| | VLAHDRHQKHWRSTNYYIKVRAGDNKYMHLKVFNGPDLRKHKW | |
| | KSADRVLTGYQVDKNKDDELTGF | |
| FcRn-23 | MIPRGLSEAKPATPEIQEIVDKVKPQLEEKTNETYGKLEAVQYKTQ | 616 |
| | VLAIPHHHKPRVSTNYYIKVRAGDNKYMHLKVFNGPSFHHHRHSD | |
| | ADRVLTGYQVDKNKDDELTGF | |
| FcRn-24 | MIPRGLSEAKPATPEIQEIVDKVKPQLEEKTNETYGKLEAVQYKTQ | 617 |
| | VLAKGKHYHSQQSTNYYIKVRAGDNKYMHLKVFNGPEFYQGHW | |
| | TN ADRVLTGYQVDKNKDDELTGF | |
| FcRn-25 | MIPRGLSEAKPATPEIQEIVDKVKPQLEEKTNETYGKLEAVQYKTQ | 618 |
| | VLAHKHKHHHTNSTNYYIKVRAGDNKYMHLKVFNGPVGHHWW | |
| | LKEADRVLTGYQVDKNKDDELTGF | |
| FcRn-26 | MIPRGLSEAKPATPEIQEIVDKVKPQLEEKTNETYGKLEAVQYKTQ | 619 |
| | VLAGRHKHIQVHSTNYYIKVRAGDNKYMHLKVFNGPVGTKHLRQ | |
| | S ADRVLTGYQVDKNKDDELTGF | |
| FcRn-27 | MIPRGLSEAKPATPEIQEIVDKVKPQLEEKTNETYGKLEAVQYKTQ | 620 |
| | VLAPHQHKLHAHSTNYYIKVRAGDNKYMHLKVFNGPKRRRHPSR | |
| | G ADRVLTGYQVDKNKDDELTGF | |
| FcRn-28 | MIPRGLSEAKPATPEIQEIVDKVKPQLEEKTNETYGKLEAVQYKTQ | 621 |
| | VLARRDHVWHKGSTNYYIKVRAGDNKYMHLKVFNGPNHVHNKH | |
| | IHADRVLTGYQVDKNKDDELTGF | |
| FcRn-29 | MIPRGLSEAKPATPEIQEIVDKVKPQLEEKTNETYGKLEAVQYKTQ | 622 |
| | VLASHRSHADRRSTNYYIKVRAGDNKYMHLKVFNGPTQSHPHRH | |
| | YADRVLTGYQVDKNKDDELTGF | |
| FcRn-30 | MIPRGLSEAKPATPEIQEIVDKVKPQLEEKTNETYGKLEAVQYKTQ | 623 |
| | VLASSQNGYQGHSTNYYIKVRAGDNKYMHLKVFNGPYRHHHHW | |
| | HFADRVLTGYQVDKNKDDELTGF | |
| FcRn-31 | MIPRGLSEAKPATPEIQEIVDKVKPQLEEKTNETYGKLEAVQYKTQ | 624 |
| | VLATEGGKKLRRSTNYYIKVRAGDNKYMHLKVFNGPEWTHGKEN | |
| | HADRVLTGYQVDKNKDDELTGF | |
| FcRn-32 | MIPRGLSEAKPATPEIQEIVDKVKPQLEEKTNETYGKLEAVQYKTQ | 625 |
| | VLAKARHHQGHASTNYYIKVRAGDNKYMHLKVFNGPWYQFDGV | |
| | SFADRVLTGYQVDKNKDDELTGF | |
| FcRn-33 | MIPRGLSEAKPATPEIQEIVDKVKPQLEEKTNETYGKLEAVQYKTQ | 626 |
| | VLANHSQGRHHISTNYYIKVRAGDNKYMHLKVFNGPKKVRHEYA | |
| | WADRVLTGYQVDKNKDDELTGF | |
| FcRn-34 | MIPRGLSEAKPATPEIQEIVDKVKPQLEEKTNETYGKLEAVQYKTQ | 627 |
| | VLAKYWKADWYWSTNYYIKVRAGDNKYMHLKVFNGPEHSWWR | |
| | RGHADRVLTGYQVDKNKDDELTGF | |
| FcRn-35 | MIPRGLSEAKPATPEIQEIVDKVKPQLEEKTNETYGKLEAVQYKTQ | 628 |
| | VLAHRQYPPGPHSTNYYIKVRAGDNKYMHLKVFNGPYHFHHYYK | |
| | HADRVLTGYQVDKNKDDELTGF | |
| FcRn-36 | MIPRGLSEAKPATPEIQEIVDKVKPQLEEKTNETYGKLEAVQYKTQ | 629 |
| | VLARQHHHFYRTSTNYYIKVRAGDNKYMHLKVFNGPWQNFHDPF | |
| | DADRVLTGYQVDKNKDDELTGF | |
| FcRn-37 | MIPRGLSEAKPATPEIQEIVDKVKPQLEEKTNETYGKLEAVQYKTQ | 630 |
| | VLAPQQHQPDPTSTNYYIKVRAGDNKYMHLKVFNGPARQHHHHS | |
| | HADRVLTGYQVDKNKDDELTGF | |
| FcRn-38 | MIPRGLSEAKPATPEIQEIVDKVKPQLEEKTNETYGKLEAVQYKTQ | 631 |
| | VLALSFNNYHWHSTNYYIKVRAGDNKYMHLKVFNGPKLRHDKLT | |
| | HADRVLTGYQVDKNKDDELTGF | |
| FcRn-39 | MIPRGLSEAKPATPEIQEIVDKVKPQLEEKTNETYGKLEAVQYKTQ | 632 |
| | VLAHHSKHHHLHSTNYYIKVRAGDNKYMHLKVFNGPNHKFQSYQ | |
| | PADRVLTGYQVDKNKDDELTGF | |
| FcRn-40 | MIPRGLSEAKPATPEIQEIVDKVKPQLEEKTNETYGKLEAVQYKTQ | 633 |
| | VLAHKYDRHSFKSTNYYIKVRAGDNKYMHLKVFNGPGKHSGARH | |
| | KADRVLTGYQVDKNKDDELTGF | |
| FcRn-41 | MIPRGLSEAKPATPEIQEIVDKVKPQLEEKTNETYGKLEAVQYKTQ | 634 |
| | VLAKHSRHHHAQYTNYYIKVRAGDNKYMHLKVFNGPNIHHEGKI | |
| | PADRVLTGYQVDKNKDDELTGF | |
| FcRn-42 | MIPRGLSEAKPATPEIQEIVDKVKPQLEEKTNETYGKLEAVQYKTQ | 635 |
| | VLARHHHSHFHLSTNYYIKVRAGDNKYMHLKVFNGPIRQSSYKVH | |
| | ADRVLTGYQVDKNKDDELTGF | |
| FcRn-43 | MIPRGLSEAKPATPEIQEIVDKVKPQLEEKTNETYGKLEAVQYKTQ | 636 |
| | VLARNHRHPHGQSTNYYIKVRAGDNKYMHLKVFNGPVQHRWSL | |
| | HWADRVLTGYQVDKNKDDELTGF | |
| FcRn-44 | MIPRGLSEAKPATPEIQEIVDKVKPQLEEKTNETYGKLEAVQYKTQ | 637 |
| | VLAGHVEQVHFPYTNYYIKVRAGDNKYMHLKVFNGPGHKHHHH | |
| | WSADRVLTGYQVDKNKDDELTGF | |
| FcRn-45 | MIPRGLSEAKPATPEIQEIVDKVKPQLEEKTNETYGKLEAVQYKTQ | 638 |
| | VLAEPHKHHYHLSTNYYIKVRAGDNKYMHLKVFNGPVPGQQPIK | |
| | N ADRVLTGYQVDKNKDDELTGF | |
| FcRn-46 | MIPRGLSEAKPATPEIQEIVDKVKPQLEEKTNETYGKLEAVQYKTQ | 639 |
| | VLAWKKHNWKYKSTNYYIKVRAGDNKYMHLKVFNGPWAAKRD | |
| | WRN ADRVLTGYQVDKNKDDELTGF | |
| FcRn-47 | MIPRGLSEAKPATPEIQEIVDKVKPQLEEKTNETYGKLEAVQYKTQ | 640 |
| | VLAIHHHTWGLKSTNYYIKVRAGDNKYMHLKVFNGPYGDQPFKR | |
| | KADRVLTGYQVDKNKDDELTGF | |
| FcRn-48 | MIPRGLSEAKPATPEIQEIVDKVKPQLEEKTNETYGKLEAVQYKTQ | 641 |
| | VLAKPKYHHHDISTNYYIKVRAGDNKYMHLKVFNGPGHHAKPHR | |
| | W ADRVLTGYQVDKNKDDELTGF | |
| FcRn-49 | MIPRGLSEAKPATPEIQEIVDKVKPQLEEKTNETYGKLEAVQYKTQ | 642 |
| | VLAQYWHSHETWSTNYYIKVRAGDNKYMHLKVFNGPFLKVRTIR | |
| | SADRVLTGYQVDKNKDDELTGF | |
| FcRn-50 | MIPRGLSEAKPATPEIQEIVDKVKPQLEEKTNETYGKLEAVQYKTQ | 643 |
| | VLARKQYHLPWTSTNYYIKVRAGDNKYMHLKVFNGPLSQFQTHL | |
| | WADRVLTGYQVDKNEDDELTGF | |
| FcRn-51 | MIPRGLSEAKPATPEIQEIVDKVKPQLEEKTNETYGKLEAVQYKTQ | 644 |
| | VLAAIHWAHYILSTNYYIKVRAGDNKYMHLKVFNGPVLWRYYYP | |
| | KADRVLTGYQVDKNKDDELTGF | |
| FcRn-52 | MIPRGLSEAKPATPEIQEIVDKVKPQLEEKTNETYGKLEAVQYKTQ | 645 |
| | VLADWRKLTLFSTNYYIKVRAGDNKYMHLKVFNGPHHQHWHVF | |
| | PADRVLTGYQVDKNKDDELTGF | |
| FcRn-53 | MIPRGLSEAKPATPEIQEIVDKVKPQLEEKTNETYGKLEAVQYKTQ | 646 |
| | VLATKSHKFAYHSTNYYIKVRAGDNKYMHLKVFNGPIVQEFSLDQ | |
| | WADRVLTGYQVDKNKDDELTGF | |
| FcRn-54 | MIPRGLSEAKPATPEIQEIVDKVKPQLEEKTNETYGKLEAVQYKTQ | 647 |
| | VLASKYVHWHKFSTNYYIKVRAGDNKYMHLKVFNGPWKINNLY | |
| | HEADRVLTGYQVDKNKDDELTGF | |
| FcRn-55 | MIPRGLSEAKPATPEIQEIVDKVKPQLEEKTNETYGKLEAVQYKTQ | 648 |
| | VLAKEQAAWVLHSTNYYIKVRAGDNKYMHLKVFNGPFHYLHHT | |
| | RSADRVLTGYQVDKNKDDELTGF | |
| FcRn-56 | MIPRGLSEAKPATPEIQEIVDKVKPQLEEKTNETYGKLEAVQYKTQ | 649 |
| | VLAHLQAPRNAYSTNYYIKVRAGDNKYMHLKVFNGPKGWRNTH | |
| | HKADRVLTGYQVDKNKDDELTGF | |
| FcRn-57 | MIPRGLSEAKPATPEIQEIVDKVKPQLEEKTNETYGKLEAVQYKTQ | 650 |
| | VLAGLTHRWRPHSTNYYIKVRAGDNKYMHLKVFNGPIWSARSDK | |
| | LADRVLTGYQVDKNKDDELTGF | |
| FcRn-58 | MIPRGLSEAKPATPEIQEIVDKVKPQLEEKTNETYGKLEAVQYKTQ | 651 |
| | VLASHHRATDQVSTNYYIKVRAGDNKYMHLKVFNGPKAYHTYW | |
| | HKADRVLTGYQVDKNKDDELTGF | |
| FcRn-59 | MIPRGLSEAKPATPEIQEIVDKVKPQLEEKTNETYGKLEAVQYKTQ | 652 |
| | VLANKWHIRFATSTNYYIKVRAGDNKYMHLKVFNGPFAQAHHHT | |
| | QADRVLTGYQVDKNKDDELTGF | |
| FcRn-60 | MIPRGLSEAKPATPEIQEIVDKVKPQLEEKTNETYGKLEAVQYKTQ | 653 |
| | VLAHIRDSLWITSTNYYIKVRAGDNKYMHLKVFNGPNWQWIPHW | |
| | AADRVLTGYQVDKNKDDELTGF | |
| FcRn-61 | MIPRGLSEAKPATPEIQEIVDKVKPQLEEKTNETYGKLEAVQYKTQ | 654 |
| | VLAYHISLSFRESTNYYIKVRAGDNKYMHLKVFNGPKLDTLGQQR | |
| | ADRVLTGYQVDKNKDDELTGF | |
| FcRn-62 | MIPRGLSEAKPATPEIQEIVDKVKPQLEEKTNETYGKLEAVQYKTQ | 655 |
| | VLAIHWAGFFRGSTNYYIKVRAGDNKYMHLKVFNGPWEWERHW | |
| | LAADRVLTGYQVDKNKDDELTGF | |
| FcRn-63 | MIPRGLSEAKPATPEIQEIVDKVKPQLEEKTNETYGKLEAVQYKTQ | 656 |
| | VLAYYSERHFYKSTNYYIKVRAGDNKYMHLKVFNGPFTLGREGW | |
| | F ADRVLTGYQVDKNKDDELTGF | |
| FcRn-64 | MIPRGLSEAKPATPEIQEIVDKVKPQLEEKTNETYGKLEAVQYKTQ | 657 |
| | VLARQQQVHVPSSTNYYIKVRAGDNKYMHLKVFNGPYRGNTFKI | |
| | W ADRVLTGYQVDKNKDDELTGF | |
| FcRn-65 | MIPRGLSEAKPATPEIQEIVDKVKPQLEEKTNETYGKLEAVQYKTQ | 658 |
| | VLATKKNQLQGHSTNYYIKVRAGDNKYMHLKVFNGPVHSLLQHH | |
| | D ADRVLTGYQVDKNKDDELTGF | |
| FcRn-66 | MIPRGLSEAKPATPEIQEIVDKVKPQLEEKTNETYGKLEAVQYKTQ | 659 |
| | VLARDIHHHHHWSTNYYIKVRAGDNKYMHLKVFNGPYIKRHWSN | |
| | F ADRVLTGYQVDKNKDDELTGF | |
| FcRn-67 | MIPRGLSEAKPATPEIQEIVDKVKPQLEEKTNETYGKLEAVQYKTQ | 660 |
| | VLAQRQYTTKVLSTNYYIKVRAGDNKYMHLKVFNGPDNERNQVE | |
| | S ADRVLTGYQVDKNKDDELTGF | |
| FcRn-68 | MIPRGLSEAKPATPEIQEIVDKVKPQLEEKTNETYGKLEAVQYKTQ | 661 |
| | VLAYWDWRFVEWSTNYYIKVRAGDNKYMHLKVFNGPIGYELFTV | |
| | KADRVLTGYQVDKNKDDELTGF | |
| FcRn-69 | MIPRGLSEAKPATPEIQEIVDKVKPQLEEKTNETYGKLEAVQYKTQ | 662 |
| | VLAGFSKPFKWYSTNYYIKVRAGDNKYMHLKVFNGPYRAWIHWT | |
| | SADRVLTGYQVDKNKDDELTGF | |
| FcRn-70 | MIPRGLSEAKPATPEIQEIVDKVKPQLEEKTNETYGKLEAVQYKTQ | 663 |
| | VLAIFQERLAGQSTNYYIKVRAGDNKYMHLKVFNGPQIKHSHHA | |
| | WADRVLTGYQVDKNKDDELTGF | |
| FcRn-71 | MIPRGLSEAKPATPEIQEIVDKVKPQLEEKTNETYGKLEAVQYKTQ | 664 |
| | VLAKYDHHTQSLSTNYYIKVRAGDNKYMHLKVFNGPVYAWYWD | |
| | KWADRVLTGYQVDKNKDDELTGF | |
| FcRn-72 | MIPRGLSEAKPATPEIQEIVDKVKPQLEEKTNETYGKLEAVQYKTQ | 665 |
| | VLAKHAHTPFGPSTNYYIKVRAGDNKYMHLKVFNGPAVWWDGR | |
| | GWADRVLTGYQVDKNKDDELTGF | |
| FcRn-73 | MIPRGLSEAKPATPEIQEIVDKVKPQLEEKTNETYGKLEAVQYKTQ | 666 |
| | VLASLSRWLWAESTNYYIKVRAGDNKYMHLKVFNGPWHTHKHY | |
| | QKADRVLTGYQVDKNKDDELTGF | |
| FcRn-74 | MIPRGLSEAKPATPEIQEIVDKVKPQLEEKTNETYGKLEAVQYKTQ | 667 |
| | VLAHQQHTQRYRSTNYYIKVRAGDNKYMHLKVFNGPAKLQFGH | |
| | KHADRVLTGYQVDKNKDDELTGF | |
| FcRn-75 | MIPRGLSEAKPATPEIQEIVDKVKPQLEEKTNETYGKLEAVQYKTQ | 668 |
| | VLAHTISQHVSTNYYIKVRAGDNKYMHLKVFNGPPISFRWHRFAD | |
| | RVLTGYQVDKNKDDELTGF | |
| FcRn-76 | MIPRGLSEAKPATPEIQEIVDKVKPQLEEKTNETYGKLEAVQYKTQ | 669 |
| | VLADQWTWAHSRSTNYYIKVRAGDNKYMHLKVFNGPDYHLRHH | |
| | NHADRVLTGYQVDKNKDDELTGF | |
| FcRn-77 | MIPRGLSEAKPATPEIQEIVDKVKPQLEEKTNETYGKLEAVQYKTQ | 670 |
| | VLAWYRVWRWVWSTNYYIKVRAGDNKYMHLKVFNGPVYKYGS | |
| | ENWADRVLTGYQVDKNKDDELTGF | |
| FcRn-78 | MIPRGLSEAKPATPEIQEIVDKVKPQLEEKTNETYGKLEAVQYKTQ | 671 |
| | VLAQKGSTHHNHSTNYYIKVRAGDNKYMHLKVFNGPARSQAGH | |
| | HNADRVLTGYQVDKNKDDELTGF | |
| FcRn-79 | MIPRGLSEAKPATPEIQEIVDKVKPQLEEKTNETYGKLEAVQYKTQ | 672 |
| | VLAPEGRAGEPSSTNYYIKVRAGDNKYMHLKVFNGPEHWWFTFG | |
| | DADRVLTGYQVDKNKDDELTGF | |
| FcRn-80 | MIPRGLSEAKPATPEIQEIVDKVKPQLEEKTNETYGKLEAVQYKTQ | 673 |
| | VLAHTRHHVTLWSTNYYIKVRAGDNKYMHLKVFNGPGWKYAPQ | |
| | VWADRVLTGYQVDKNKDDELTGF | |
| FcRn-81 | MIPRGLSEAKPATPEIQEIVDKVKPQLEEKTNETYGKLEAVQYKTQ | 674 |
| | VLAQRYYKHEYRSTNYYIKVRAGDNKYMHLKVFNGPYFKLPPWE | |
| | EADRVLTGYQVDKNKDDELTGF | |
| FcRn-82 | MIPRGLSEAKPATPEIQEIVDKVKPQLEEKTNETYGKLEAVQYKTQ | 675 |
| | VLAQWFHRREVKSTNYYIKVRAGDNKYMHLKVFNGPPVHLHHK | |
| | QHADRVLTGYQVDKNKDDELTGF | |
| FcRn-83 | MIPRGLSEAKPATPEIQEIVDKVKPQLEEKTNETYGKLEAVQYKTQ | 676 |
| | VLAHHLHATQPPSTNYYIKVRAGDNKYMHLKVFNGPNWHIINKY | |
| | DADRVLTGYQVDKNKDDELTGF | |
| FcRn-84 | MIPRGLSEAKPATPEIQEIVDKVKPQLEEKTNETYGKLEAVQYKTQ | 677 |
| | VLAKHWHQPVAKSTNYYIKVRAGDNKYMHLKVFNGPAHWHDW | |
| | VADRVLTGYQVDKNKDDELTGF | |
| FcRn-85 | MIPRGLSEAKPATPEIQEIVDKVKPQLEEKTNETYGKLEAVQYKTQ | 678 |
| | VLAYTTSHWTIGSTNYYIKVRAGDNKYMHLKVFNGPDHHHVQKS | |
| | HADRVLTGYQVDKNKDDELTGF | |
| FcRn-86 | MIPRGLSEAKPATPEIQEIVDKVKPQLEEKTNETYGKLEAVQYKTQ | 679 |
| | VLAEHHHTQLSNSTNYYIKVRAGDNKYMHLKVFNGPKFWQVQQ | |
| | KYADRVLTGYQVDKNKDDELTGF | |
| FcRn-87 | MIPRGLSEAKPATPEIQEIVDKVKPQLEEKTNETYGKLEAVQYKTQ | 680 |
| | VLAHKPHNSKQISTNYYIKVRAGDNKYMHLKVFNGPKPRFNIHHH | |
| | ADRVLTGYQVDKNKDDELTGF | |
| FcRn-88 | MIPRGLSEAKPATPEIQEIVDKVKPQLEEKTNETYGKLEAVQYKTQ | 681 |
| | VLAHHTKHHSRWSTNYYIKVRAGDNKYMHLKVFNGPVNHISHAP | |
| | IADRVLTGYQVDKNKDDELTGF | |
| FcRn-89 | MIPRGLSEAKPATPEIQEIVDKVKPQLEEKTNETYGKLEAVQYKTQ | 682 |
| | VLAFHRHHPIWHSTNYYIKVRAGDNKYMHLKVFNGPLKPWEADL | |
| | WADRVLTGYQVDKNKDDELTGF | |
| FcRn-90 | MIPRGLSEAKPATPEIQEIVDKVKPQLEEKTNETYGKLEAVQYKTQ | 683 |
| | VLAARVTIDWKASTNYYIKVRAGDNKYMHLKVFNGPYKYPNIHP | |
| | HADRVLTGYQVDKNKDDELTGF | |
| FcRn-91 | MIPRGLSEAKPATPEIQEIVDKVKPQLEEKTNETYGKLEAVQYKTQ | 684 |
| | VLAKLEQRRSHYSTNYYIKVRAGDNKYMHLKVFNGPPKSLFNYQ | |
| | HADRVLTGYQVDKNKDDELTGF | |
| FcRn-92 | MIPRGLSEAKPATPEIQEIVDKVKPQLEEKTNETYGKLEAVQYKTQ | 685 |
| | VLANIHHVHHQQSTNYYIKVRAGDNKYMHLKVFNGPDGEFHVKQ | |
| | VADRVLTGYQVDKNKDDELTGF | |
| FcRn-93 | MIPRGLSEAKPATPEIQEIVDKVKPQLEEKTNETYGKLEAVQYKTQ | 686 |
| | VLASHHTIAWYVSTNYYIKVRAGDNKYMHLKVFNGPVYPKRQQV | |
| | EADRVLTGYQVDKNKDDELTGF | |
| FcRn-94 | MIPRGLSEAKPATPEIQEIVDKVKPQLEEKTNETYGKLEAVQYKTQ | 687 |
| | VLAHHQPYYGWQSTNYYIKVRAGDNKYMHLKVFNGPIIDRSKIEK | |
| | ADRVLTGYQVDKNKDDELTGF | |
| FcRn-95 | MIPRGLSEAKPATPEIQEIVDKVKPQLEEKTNETYGKLEAVQYKTQ | 688 |
| | VLAVHRSHHPIKSTNYYIKVRAGDNKYMHLKVFNGPSIHSSWKKQ | |
| | ADRVLTGYQVDKNKDDELTGF | |
| FcRn-96 | MIPRGLSEAKPATPEIQEIVDKVKPQLEEKTNETYGKLEAVQYKTQ | 689 |
| | VLAWWSQRVKLFSTNYYIKVRAGDNKYMHLKVFNGPNIHKTWD | |
| | QTADRVLTGYQVDKNKDDELTGF | |
| FcRn-97 | MIPRGLSEAKPATPEIQEIVDKVKPQLEEKTNETYGKLEAVQYKTQ | 690 |
| | VLAHYWKPHDIHSTNYYIKVRAGDNKYMHLKVFNGPGKVPFHAF | |
| | HKADRVLTGYQVDKNKDDELTGF | |
| FcRn-98 | MIPRGLSEAKPATPEIQEIVDKVKPQLEEKTNETYGKLEAVQYKTQ | 691 |
| | VLATNQPRLYHQSTNYYIKVRAGDNKYMHLKVFNGPFYRLTHGH | |
| | RADRVLTGYQVDKNKDDELTGF | |
| FcRn-99 | MIPRGLSEAKPATPEIQEIVDKVKPQLEEKTNETYGKLEAVQYKTQ | 692 |
| | VLAWSGKLLKHPSTNYYIKVRAGDNKYMHLKVFNGPHIDYKNGR | |
| | IWADRVLTGYQVDKNKDDELTGF | |
| FcRn-10 | MIPRGLSEAKPATPEIQEIVDKVKPQLEEKTNETYGKLEAVQYKTQ | 693 |
| 0 | VLAHRTSWDHKNSTNYYIKVRAGDNKYMHLKVFNGPVFHHQRG | |
| | GQADRVLTGYQVDKNKDDELTGF | |
| FcRn-10 | MIPRGLSEAKPATPEIQEIVDKVKPQLEEKTNETYGKLEAVQYKTQ | 694 |
| 1 | VLAPHKQKRHFFNSTNYYIKVRAGDNKYMHLKVFNGPWGQSKPA | |
| | HVADRVLTGYQVDKNKDDELTGF | |
| FcRn-10 | MIPRGLSEAKPATPEIQEIVDKVKPQLEEKTNETYGKLEAVQYKTQ | 695 |
| 2 | VLAHDQHKHDFKSTNYYIKVRAGDNKYMHLKVFNGPFHQRFPDH | |
| | KADRVLTGYQVDKNKDDELTGF | |
| FcRn-10 | MIPRGLSEAKPATPEIQEIVDKVKPQLEEKTNETYGKLEAVQYKTQ | 696 |
| 3 | VLANRVVHHFHHSTNYYIKVRAGDNKYMHLKVFKGPIQAAEGYK | |
| | KADRVLTGYQVDKNKDDELTGF | |
| FcRn-10 | MIPRGLSEAKPATPEIQEIVDKVKPQLEEKTNETYGKLEAVQYKTQ | 697 |
| 4 | VLAWHKAIRQQFSTNYYIKVRAGDNKYMHLKVFNGPFHYQYRHQ | |
| | KADRVLTGYQVDKNKDDELTGF | |
| FcRn-10 | MIPRGLSEAKPATPEIQEIVDKVKPQLEEKTNETYGKLEAVQYKTQ | 698 |
| 5 | VLAWYHTHFANASTNYYIKVRAGDNKYMHLKVFNGPFKRHQHG | |
| | HKADRVLTGYQVDKNKDDELTGF | |
| FcRn-10 | MIPRGLSEAKPATPEIQEIVDKVKPQLEEKTNETYGKLEAVQYKTQ | 699 |
| 6 | VLATKEWHQHIKSTNYYIKVRAGDNKYMHLKVFNGPNKFLHGFE | |
| | VADRVLTGYQVDKNKDDELTGF | |
| FcRn-10 | MIPRGLSEAKPATPEIQEIVDKVKPQLEEKTNETYGKLEAVQYKTQ | 700 |
| 7 | VLATRVHNLSVLSTNYYIKVRAGDNKYMHLKVFNGPHYDRAHYF | |
| | KADRVLTGYQVDKNKDDELTGF | |
| FcRn-10 | MIPRGLSEAKPATPEIQEIVDKVKPQLEEKTNETYGKLEAVQYKTQ | 701 |
| 8 | VLAWNQPYWTTYSTNYYIKVRAGDNKYMHLKVFNGPFRWKFHD | |
| | YKADRVLTGYQVDKNKDDELTGF | |
| FcRn-10 | MIPRGLSEAKPATPEIQEIVDKVKPQLEEKTNETYGKLEAVQYKTQ | 702 |
| 9 | VLARPHNRDSHRSTNYYIKVRAGDNKYMHLKVFNGPDRKHRKH | |
| | WHADRVLTGYQVDKNKDDELTGF | |
| FcRn-11 | MIPRGLSEAKPATPEIQEIVDKVKPQLEEKTNETYGKLEAVQYKTQ | 703 |
| 0 | VLAGHPRHHWKYSTNYYIKVRAGDNKYMHLKVFNGPATYKYRV | |
| | DYADRVLTGYQVDKNKDDELTGF | |
| FcRn-11 | MIPRGLSEAKPATPEIQEIVDKVKPQLEEKTNETYGKLEAVQYKTQ | 704 |
| 1 | VLAYPGHHHARDSTNYYIKVRAGDNKYMHLKVFNGPYFYHHHW | |
| | FKADRVLTGYQVDKNKDDELTGF | |
| FcRn-11 | MIPRGLSEAKPATPEIQEIVDKVKPQLEEKTNETYGKLEAVQYKTQ | 705 |
| 2 | VLAIAKHHTWHQSTNYYIKVRAGDNKYMHLKVFNGPYRNHRHHI | |
| | VADRVLTGYQVDKNKDDELTGF | |
| FcRn-11 | MIPRGLSEAKPATPEIQEIVDKVKPQLEEKTNETYGKLEAVQYKTQ | 706 |
| 3 | VLAHNHGHWHFRSTNYYIKVRAGDNKYMHLKVFNGPVQHARHK | |
| | HYADRVLTGYQVDKNKDDELTGF | |
| FcRn-11 | MIPRGLSEAKPATPEIQEIVDKVKPQLEEKTNETYGKLEAVQYKTQ | 707 |
| 4 | VLAKKFDHYHQKSTNYYIKVRAGDNKYMHLKVFNGPKDRHHHN | |
| | RADRVLTGYQVDKNKDDELTGF | |
| FcRn-11 | MIPRGLSEAKPATPEIQEIVDKVKPQLEEKTNETYGKLEAVQYKTQ | 708 |
| 5 | VLASKAHRVEHKSTNYYIKVRAGDNKYMHLKVFNGPKQHHLYHF | |
| | KADRVLTGYQVDKNKDDELTGF | |
| FcRn-11 | MIPRGLSEAKPATPEIQEIVDKVKPQLEEKTNETYGKLEAVQYKTQ | 709 |
| 6 | VLAPKKHYHHGISTNYYIKVRAGDNKYMHLKVFNGPVNSFQAHR | |
| | KADRVLTGYQVDKNKDDELTGF | |
| FcRn-11 | MIPRGLSEAKPATPEIQEIVDKVKPQLEEKTNETYGKLEAVQYKTQ | 710 |
| 7 | VLANSHRIQHGFSTNYYIKVRAGDNKYMHLKVFNGPSHHLHRSAH | |
| | ADRVLTGYQVDKNKDDELTGF | |
| FcRn-11 | MIPRGLSEAKPATPEIQEIVDKVKPQLEEKTNETYGKLEAVQYKTQ | 711 |
| 8 | VLAPHHSHHRLESTNYYIKVRAGDNKYMHLKVFNGPQPTFRHHY | |
| | T ADRVLTGYQVDKNKDDELTGF | |
| FcRn-11 | MIPRGLSEAKPATPEIQEIVDKVKPQLEEKTNETYGKLEAVQYKTQ | 712 |
| 9 | VLAHVHHHREKGSTNYYIKVRAGDNKYMHLKVFNGPYSNSRERQ | |
| | WADRVLTGYQVDKNKDDELTGF | |
| FcRn-12 | MIPRGLSEAKPATPEIQEIVDKVKPQLEEKTNETYGKLEAVQYKTQ | 713 |
| 0 | VLAKHKYHHTGHSTNYYIKVRAGDNKYMHLKVFNGPGQIHKVRS | |
| | TADRVLTGYQVDKNKDDELTGF | |
| FcRn-12 | MIPRGLSEAKPATPEIQEIVDKVKPQLEEKTNETYGKLEAVQYKTQ | 714 |
| 1 | VLAKYFAPHAPHSTNYYIKVRAGDNKYMHLKVFNGPHYHHRHQH | |
| | SADRVLTGYQVDKNKDDELTGF | |
| FcRn-12 | MIPRGLSEAKPATPEIQEIVDKVKPQLEEKTNETYGKLEAVQYKTQ | 715 |
| 2 | VLALHHRAHKHLSTNYYIKVRAGDNKYMHLKVFNGPYFHREHEH | |
| | QADRVLTGYQVDKNKDDELTGF | |
| FcRn-12 | MIPRGLSEAKPATPEIQEIVDKVKPQLEEKTNETYGKLEAVQYKTQ | 716 |
| 3 | VLAAHHGHYGRASTNYYIKVRAGDNKYMHLKVFNGPWHYHHSQ | |
| | WRADRVLTGYQVDKNKDDELTGF | |
| FcRn-12 | MIPRGLSEAKPATPEIQEIVDKVKPQLEEKTNETYGKLEAVQYKTQ | 717 |
| 4 | VLAPEHYSLFKPSTNYYIKVRAGDNKYMHLKVFNGPKHHRKHRH | |
| | WADRVLTGYQVDKNKDDELTGF | |
| FcRn-12 | MIPRGLSEAKPATPEIQEIVDKVKPQLEEKTNETYGKLEAVQYKTQ | 718 |
| 5 | VLADHRPRHPKHSTNYYIKVRAGDNKYMHLKVFNGPAHKHHLGF | |
| | KADRVLTGYQVDKNKDDELTGF | |
| FcRn-12 | MIPRGLSEAKPATPEIQEIVDKVKPQLEEKTNETYGKLEAVQYKTQ | 719 |
| 6 | VLAKHEVHHHGNSTNYYIKVRAGDNKYMHLKVFNGPWHRHGSG | |
| | FRADRVLTGYQVDKNKDDELTGF | |
| FcRn-12 | MIPRGLSEAKPATPEIQEIVDKVKPQLEEKTNETYGKLEAVQYKTQ | 720 |
| 7 | VLAKSHHHKHRESTNYYIKVRAGDNKYMHLKVFNGPVDRFLHVK | |
| | KADRVLTGYQVDKNKDDELTGF | |
| FcRn-12 | MIPRGLSEAKPATPEIQEIVDKVKPQLEEKTNETYGKLEAVQYKTQ | 721 |
| 8 | VLAHRHHTHKWTSTNYYIKVRAGDNKYMHLKVFNGPWPHSIDYR | |
| | QADRVLTGYQVDKNKDDELTGF | |
| FcRn-12 | MIPRGLSEAKPATPEIQEIVDKVKPQLEEKTNETYGKLEAVQYKTQ | 722 |
| 9 | VLAGKHPHHHQNSTNYYIKVRAGDNKYMHLKVFNGPKGRYSHH | |
| | HGADRVLTGYQVDKNKDDELTGF | |
| FcRn-13 | MIPRGLSEAKPATPEIQEIVDKVKPQLEEKTNETYGKLEAVQYKTQ | 723 |
| 0 | VLAWHKHHLRYRSTNYYIKVRAGDNKYMHLKVFNGPYPQDKHK | |
| | VLADRVLTGYQVDKNKDDELTGF | |
| FcRn-13 | MIPRGLSEAKPATPEIQEIVDKVKPQLEEKTNETYGKLEAVQYKTQ | 724 |
| 1 | VLAKTHKEYHHSSTNYYIKVRAGDNKYMHLKVFNGPGYRRHQGR | |
| | GADRVLTGYQVDKNKDDELTGF | |
| FcRn-13 | MIPRGLSEAKPATPEIQEIVDKVKPQLEEKTNETYGKLEAVQYKTQ | 725 |
| 2 | VLARRHHHQHWSSTNYYIKVRAGDNKYMHLKVFNGPALHDTLHP | |
| | SADRVLTGYQVDKNKDDELTGF | |
| FcRn-13 | MIPRGLSEAKPATPEIQEIVDKVKPQLEEKTNETYGKLEAVQYKTQ | 726 |
| 3 | VLATHRWHQGSRSTNYYIKVRAGDNKYMHLKVFNGPKKPHNHR | |
| | YYADRVLTGYQVDKNKDDELTGF | |
| FcRn-13 | MIPRGLSEAKPATPEIQEIVDKVKPQLEEKTNETYGKLEAVQYKTQ | 727 |
| 4 | VLAKRGHHHPNHSTNYYIKVRAGDNKYMHLKVFNGPAKHHWDT | |
| | WSADRVLTGYQVDKNKDDELTGF | |
| FcRn-13 | MIPRGLSEAKPATPEIQEIVDKVKPQLEEKTNETYGKLEAVQYKTQ | 728 |
| 5 | VLAHTVPLRKHQSTNYYIKVRAGDNKYMHLKVFNGPVIHHKHRH | |
| | QADRVLTGYQVDKNKDDELTGF | |
| FcRn-13 | MIPRGLSEAKPATPEIQEIVDKVKPQLEEKTNETYGKLEAVQYKTQ | 729 |
| 6 | VLATYRWGHHFHSTNYYIKVRAGDNKYMHLKVFNGPKYEQIDR | |
| | WHADRVLTGYQVDKNKDDELTGF | |
| FcRn-13 | MIPRGLSEAKPATPEIQEIVDKVKPQLEEKTNETYGKLEAVQYKTQ | 730 |
| 7 | VLAFKHHDRGTHSTNYYIKVRAGDNKYMHLKVFNGPYRKRHTW | |
| | FQADRVLTGYQVDKNKDDELTGF | |
| FcRn-13 | MIPRGLSEAKPATPEIQEIVDKVKPQLEEKTNETYGKLEAVQYKTQ | 731 |
| 8 | VLATAKKHPKSHSTNYYIKVRAGDNKYMHLKVFNGPKVNWHHY | |
| | RHADRVLTGYQVDKNKDDELTGF | |
| FcRn-13 | MIPRGLSEAKPATPEIQEIVDKVKPQLEEKTNETYGKLEAVQYKTQ | 732 |
| 9 | VLAHYHFSKHHNSTNYYIKVRAGDNKYMHLKVFNGPSYHHKHFV | |
| | KADRVLTGYQVDKNKDDELTGF | |
| FcRn-14 | MIPRGLSEAKPATPEIQEIVDKVKPQLEEKTNETYGKLEAVQYKTQ | 733 |
| 0 | VLAYKHKHGKWRSTNYYIKVRAGDNKYMHLKVFNGPWHGHFSK | |
| | GGVAYADRVLTGYQVDKNKDDELTGF | |
| FcRn-14 | MIPRGLSEAKPATPEIQEIVDKVKPQLEEKTNETYGKLEAVQYKTQ | 734 |
| 1 | VLAVHHKPHKTESTNYYIKVRAGDNKYMHLKVFNGPATHLKHHN | |
| | KADRVLTGYQVDKNKDDELTGF | |
| FcRn-14 | MIPRGLSEAKPATPEIQEIVDKVKPQLEEKTNETYGKLEAVQYKTQ | 735 |
| 2 | VLAHGQRYHNKSSTNYYIKVRAGDNKYMHLKVFNGPKRKWEHS | |
| | HKADRVLTGYQVDKNKDDELTGF | |
| FcRn-14 | MIPRGLSEAKPATPEIQEIVDKVKPQLEEKTNETYGKLEAVQYKTQ | 736 |
| 3 | VLAHKHHRHVPSSTNYYIKVRAGDNKYMHLKVFNGPDHRHRHW | |
| | YLADRVLTGYQVDKNKDDELTGF | |
| FcRn-14 | MIPRGLSEAKPATPEIQEIVDKVKPQLEEKTNETYGKLEAVQYKTQ | 737 |
| 4 | VLAHRKHSWSRHSTNYYIKVRAGDNKYMHLKVFNGPTKHSHSQL | |
| | FADRVLTGYQVDKNKDDELTGF | |
| FcRn-14 | MIPRGLSEAKPATPEIQEIVDKVKPQLEEKTNETYGKLEAVQYKTQ | 738 |
| 5 | VLANRHYHQEYKSTNYYIKVRAGDNKYMHLKVFNGPVHKSKHW | |
| | FYADRVLTGYQVDKNKDDELTGF | |
| FcRn-14 | MIPRGLSEAKPATPEIQEIVDKVKPQLEEKTNETYGKLEAVQYKTQ | 739 |
| 6 | VLAKIKHHHSFKSTNYYIKVRAGDNKYMHLKVFNGPSQDHHFHR | |
| | KADRVLTGYQVDKNKDDELTGF | |
| FcRn-14 | MIPRGLSEAKPATPEIQEIVDKVKPQLEEKTNETYGKLEAVQYKTQ | 740 |
| 7 | VLAQHKRSHRQSSTNYYIKVRAGDNKYMHLKVFNGPGHKYSHWS | |
| | KADRVLTGYQVDKNKDDELTGF | |
| FcRn-14 | MIPRGLSEAKPATPEIQEIVDKVKPQLEEKTNETYGKLEAVQYKTQ | 741 |
| 8 | VLASVYKWKASTNYYIKVRAGDNKYMHLKVFNGPNKHHHHAHH | |
| | ADRVLTGYQVDKNKDDELTGF | |
| FcRn-14 | MIPRGLSEAKPATPEIQEIVDKVKPQLEEKTNETYGKLEAVQYKTQ | 742 |
| 9 | VLARKLERTKYHSTNYYIKVRAGDNKYMHLKVFNGPHNKYHPHN | |
| | KADRVLTGYQVDKNKDDELTGF | |
| FcRn-15 | MIPRGLSEAKPATPEIQEIVDKVKPQLEEKTNETYGKLEAVQYKTQ | 743 |
| 0 | VLATGHKHQFHQSTNYYIKVRAGDNKYMHLKVFNGPKHKHGWF | |
| | HSADRVLTGYQVDKNKDDELTGF | |
| FcRn-15 | MIPRGLSEAKPATPEIQEIVDKVKPQLEEKTNETYGKLEAVQYKTQ | 744 |
| 1 | VLAWQELGHRVYSTNYYIKVRAGDNKYMHLKVFNGPYRRHHDK | |
| | KHADRVLTGYQVDKNKDDELTGF | |
| FcRn-15 | MIPRGLSEAKPATPEIQEIVDKVKPQLEEKTNETYGKLEAVQYKTQ | 745 |
| 2 | VLAHPHHTDQRHSTNYYIKVRAGDNKYMHLKVFNGPEGHRQHA | |
| | KFADRVLTGYQVDKNKDDELTGF | |
| FcRn-15 | MIPRGLSEAKPATPEIQEIVDKVKPQLEEKTNETYGKLEAVQYKTQ | 746 |
| 3 | VLAFHNHGHPHLSTNYYIKVRAGDNKYMHLKVFNGPNSRGHHHH | |
| | KADRVLTGYQVDKNKDDELTGF | |
| FcRn-15 | MIPRGLSEAKPATPEIQEIVDKVKPQLEEKTNETYGKLEAVQYKTQ | 747 |
| 4 | VLAWNHHHRNKQSTNYYIKVRAGDNKYMHLKVFNGPPHKRPHL | |
| | YHADRVLTGYQVDKNKDDELTGF | |
| FcRn-15 | MIPRGLSEAKPATPEIQEIVDKVKPQLEEKTNETYGKLEAVQYKTQ | 748 |
| 5 | VLATRHGHRHYRSTNYYIKVRAGDNKYMHLKVFNGPFYDLHPKL | |
| | SADRVLTGYQVDKNKDDELTGF | |
| FcRn-15 | MIPRGLSEAKPATPEIQEIVDKVKPQLEEKTNETYGKLEAVQYKTQ | 749 |
| 6 | VLAPHHRWHRQHSTNYYIKVRAGDNKYMHLKVFNGPIHQHSQKK | |
| | SADRVLTGYQVDKNKDDELTGF | |
| FcRn-15 | MIPRGLSEAKPATPEIQEIVDKVKPQLEEKTNETYGKLEAVQYKTQ | 750 |
| 7 | VLANLRHQTEHRSTNYYIKVRAGDNKYMHLKVFNGPKRHHRHSH | |
| | VADRVLTGYQVDKNKDDELTGF | |
| FcRn-15 | MIPRGLSEAKPATPEIQEIVDKVKPQLEEKTNETYGKLEAVQYKTQ | 751 |
| 8 | VLAGHRKHTHLLSTNYYIKVRAGDNKYMHLKVFNGPKKSHKAW | |
| | AWADRVLTGYQVDKNKDDELTGF | |
| FcRn-15 | MIPRGLSEAKPATPEIQEIVDKVKPQLEEKTNETYGKLEAVQYKTQ | 752 |
| 9 | VLARHSKPQHWPSTNYYIKVRAGDNKYMHLKVFNGPKGHKQHH | |
| | HYADRVLTGYQVDKNKDDELTGF | |
| FcRn-16 | MIPRGLSEAKPATPEIQEIVDKVKPQLEEKTNETYGKLEAVQYKTQ | 753 |
| 0 | VLAPHRSRFHKQSTNYYIKVRAGDNKYMHLKVFNGPWKAERHKH | |
| | YADRVLTGYQVDKNKDDELTGF | |
| FcRn-16 | MIPRGLSEAKPATPEIQEIVDKVKPQLEEKTNETYGKLEAVQYKTQ | 754 |
| 1 | VLAQRKHFHWDHSTNYYIKVRAGDNKYMHLKVFNGPQHRYTHH | |
| | HTADRVLTGYQVDKNKDDELTGF | |
| FcRn-16 | MIPRGLSEAKPATPEIQEIVDKVKPQLEEKTNETYGKLEAVQYKTQ | 755 |
| 2 | VLANKHHGQQHNSTNYYIKVRAGDNKYMHLKVFNGPSHKVHTH | |
| | SKADRVLTGYQVDKNKDDELTGF | |
| FcRn-16 | MIPRGLSEAKPATPEIQEIVDKVKPQLEEKTNETYGKLEAVQYKTQ | 756 |
| 3 | VLAKYHHKYKSYSTNYYIKVRAGDNKYMHLKVFNGPKHLDQYH | |
| | PSADRVLTGYQVDKNKDDELTGF | |
| FcRn-16 | MIPRGLSEAKPATPEIQEIVDKVKPQLEEKTNETYGKLEAVQYKTQ | 757 |
| 4 | VLAREWHHQTYYSTNYYIKVRAGDNKYMHLKVFNGPSAHKHHH | |
| | NHADRVLTGYQVDKNKDDELTGF | |
| FcRn-16 | MIPRGLSEAKPATPEIQEIVDKVKPQLEEKTNETYGKLEAVQYKTQ | 758 |
| 5 | VLARHYHDHHYRSTNYYIKVRAGDNKYMHLKVFNGPKYKHQVK | |
| | QHADRVLTGYQVDKNKDDELTGF | |
| FcRn-16 | MIPRGLSEAKPATPEIQEIVDKVKPQLEEKTNETYGKLEAVQYKTQ | 759 |
| 6 | VLASHTYRHSTGSTNYYIKVRAGDNKYMHLKVFNGPISHRHRHDI | |
| | ADRVLTGYQVDKNKDDELTGF | |
| FcRn-16 | MIPRGLSEAKPATPEIQEIVDKVKPQLEEKTNETYGKLEAVQYKTQ | 760 |
| 7 | VLANHRHHHPHFSTNYYIKVRAGDNKYMHLKVFNGPNYHAHRSF | |
| | YADRVLTGYQVDKNKDDELTGF | |
| FcRn-16 | MIPRGLSEAKPATPEIQEIVDKVKPQLEEKTNETYGKLEAVQYKTQ | 761 |
| 8 | VLAHAKTRHHEHSTNYYIKVRAGDNKYMHLKVFNGPWFKHHFW | |
| | HRADRVLTGYQVDKNKDDELTGF | |
| FcRn-16 | MIPRGLSEAKPATPEIQEIVDKVKPQLEEKTNETYGKLEAVQYKTQ | 762 |
| 9 | VLAEPHQKHKRHSTNYYIKVRAGDNKYMHLKVFNGPKRKGDFLN | |
| | YADRVLTGYQVDKNKDDELTGF | |
| FcRn-17 | MIPRGLSEAKPATPEIQEIVDKVKPQLEEKTNETYGKLEAVQYKTQ | 763 |
| 0 | VLADRRHQHGRHSTNYYIKVRAGDNKYMHLKVFNGPHKPWGHH | |
| | KLADRVLTGYQVDKNKDDELTGF | |
| FcRn-17 | MIPRGLSEAKPATPEIQEIVDKVKPQLEEKTNETYGKLEAVQYKTQ | 764 |
| 1 | VLAHQHRHNLQQSTNYYIKVRAGDNKYMHLKVFNGPQYKHKHW | |
| | LWADRVLTGYQVDKNKDDELTGF | |
| FcRn-17 | MIPRGLSEAKPATPEIQEIVDKVKPQLEEKTNETYGKLEAVQYKTQ | 765 |
| 2 | VLAKRIHTWHTDSTNYYIKVRAGDNKYMHLKVFNGPFKRHHSWH | |
| | HADRVLTGYQVDKNKDDELTGF | |
| FcRn-17 | MIPRGLSEAKPATPEIQEIVDKVKPQLEEKTNETYGKLEAVQYKTQ | 766 |
| 3 | VLAYHHQPRYQQSTNYYIKVRAGDNKYMHLKVFNGPKDRHHEFR | |
| | HADRVLTGYQVDKNKDDELTGF | |
| FcRn-17 | MIPRGLSEAKPATPEIQEIVDKVKPQLEEKTNETYGKLEAVQYKTQ | 767 |
| 4 | VLAGIGRHRRRRSTNYYIKVRAGDNKYMHLKVFNGPHHHHFHNH | |
| | RADRVLTGYQVDKNKDDELTGF | |
| FcRn-17 | MIPRGLSEAKPATPEIQEIVDKVKPQLEEKTNETYGKLEAVQYKTQ | 768 |
| 5 | VLADQHKQHYHFSTNYYIKVRAGDNKYMHLKVFNGPSVNQHFK | |
| | HKADRVLTGYQVDKNKDDELTGF | |
| FcRn-17 | MIPRGLSEAKPATPEIQEIVDKVKPQLEEKTNETYGKLEAVQYKTQ | 769 |
| 6 | VLAGRHHESHKSSTNYYIKVRAGDNKYMHLKVFNGPFQHKLHKH | |
| | HADRVLTGYQVDKNKDDELTGF | |
| FcRn-17 | MIPRGLSEAKPATPEIQEIVDKVKPQLEEKTNETYGKLEAVQYKTQ | 770 |
| 7 | VLAKRHHHWHYSSTNYYIKVRAGDNKYMHLKVFNGPDTRYDKW | |
| | HGADRVLTGYQVDKNKDDELTGF | |
| FcRn-17 | MIPRGLSEAKPATPEIQEIVDKVKPQLEEKTNETYGKLEAVQYKTQ | 771 |
| 8 | VLANRKGGHRYHSTNYYIKVRAGDNKYMHLKVFNGPHVHRVQH | |
| | SKADRVLTGYQVDKNKDDELTGF | |
| FcRn-17 | MIPRGLSEAKPATPEIQEIVDKVKPQLEEKTNETYGKLEAVQYKTQ | 772 |
| 9 | VLARKWHGHWHRSTNYYIKVRAGDNKYMHLKVFNGPWNYQFK | |
| | SASADRVLTGYQVDKNKDDELTGF | |
| FcRn-18 | MIPRGLSEAKPATPEIQEIVDKVKPQLEEKTNETYGKLEAVQYKTQ | 773 |
| 0 | VLANWKRHHYHRSTNYYIKVRAGDNKYMHLKVFNGPQWWFHK | |
| | HVKADRVLTGYQVDKNKDDELTGF | |
| FcRn-18 | MIPRGLSEAKPATPEIQEIVDKVKPQLEEKTNETYGKLEAVQYKTQ | 774 |
| 1 | VLATRHHHRNRFSTNYYIKVRAGDNKYMHLKVFNGPISHNPNHY | |
| | HADRVLTGYQVDKNKDDELTGF | |
| FcRn-18 | MIPRGLSEAKPATPEIQEIVDKVKPQLEEKTNETYGKLEAVQYKTQ | 775 |
| 2 | VLAVKWDFKHFYSTNYYIKVRAGDNKYMHLKVFNGPTNLHSPDS | |
| | PADRVLTGYQVDKNKDDELTGF | |
| FcRn-18 | MIPRGLSEAKPATPEIQEIVDKVKPQLEEKTNETYGKLEAVQYKTQ | 776 |
| 3 | VLASDDLSPVKWSTNYYIKVRAGDNKYMHLKVFNGPFDKYNSHY | |
| | LADRVLTGYQVDKNKDDELTGF | |
| FcRn-18 | MIPRGLSEAKPATPEIQEIVDKVKPQLEEKTNETYGKLEAVQYKTQ | 777 |
| 4 | VLARHRQKWPIHSTNYYIKVRAGDNKYMHLKVFNGPSTHQQKHQ | |
| | WADRVLTGYQVDKNKDDELTGF | |
| FcRn-18 | MIPRGLSEAKPATPEIQEIVDKVKPQLEEKTNETYGKLEAVQYKTQ | 778 |
| 5 | VLADRHAYHRHSTNYYIKVRAGDNKYMHLKVFNGPFHEEIKHWQ | |
| | ADRVLTGYQVDKNKDDELTGF | |
| FcRn-18 | MIPRGLSEAKPATPEIQEIVDKVKPQLEEKTNETYGKLEAVQYKTQ | 779 |
| 6 | VLAHRHHQKHAFSTNYYIKVRAGDNKYMHLKVFNGPWRDWNHR | |
| | FPADRVLTGYQVDKNKDDELTGF | |
| FcRn-18 | MIPRGLSEAKPATPEIQEIVDKVKPQLEEKTNETYGKLEAVQYKTQ | 780 |
| 7 | VLAQKGKHHDYRSTNYYIKVRAGDNKYMHLKVFNGPKPHQTKW | |
| | HHADRVLTGYQVDKNKDDELTGF | |
| FcRn-18 | MIPRGLSEAKPATPEIQEIVDKVKPQLEEKTNETYGKLEAVQYKTQ | 781 |
| 8 | VLAWNKHFYKQGSTNYYIKVRAGDNKYMHLKVFNGPRHHRQSH | |
| | HWADRVLTGYQVDKNKDDELTGF | |
| FcRn-18 | MIPRGLSEAKPATPEIQEIVDKVKPQLEEKTNETYGKLEAVQYKTQ | 782 |
| 9 | VLAKRRHNREFVSTNYYIKVRAGDNKYMHLKVFNGPIRHYHADR | |
| | EADRVLTGYQVDKNKDDELTGF | |
| FcRn-19 | MIPRGLSEAKPATPEIQEIVDKVKPQLEEKTNETYGKLEAVQYKTQ | 783 |
| 0 | VLATRHVRHWTHSTNYYIKVRAGDNKYMHLKVFNGPASQVPPKH | |
| | RADRVLTGYQVDKNKDDELTGF | |
| FcRn-19 | MIPRGLSEAKPATPEIQEIVDKVKPQLEEKTNETYGKLEAVQYKTQ | 784 |
| 1 | VLANRKWQQNHHSTNYYIKVRAGDNKYMHLKVFNGPKHKHWH | |
| | HQLADRVLTGYQVDKNKDDELTGF | |
| FcRn-19 | MIPRGLSEAKPATPEIQEIVDKVKPQLEEKTNETYGKLEAVQYKTQ | 785 |
| 2 | VLARHREKHQPYSTNYYIKVRAGDNKYMHLKVFNGPWEHHRTR | |
| | WQADRVLTGYQVDKNKDDELTGF | |
| FcRn-19 | MIPRGLSEAKPATPEIQEIVDKVKPQLEEKTNETYGKLEAVQYKTQ | 786 |
| 3 | VLA YHKHNS KHS STN YYIKVRAGDNKYMHLKVFNGPFKTFKEWH | |
| | VADRVLTGYQVDKNKDDELTGF | |
| FcRn-19 | MIPRGLSEAKPATPEIQEIVDKVKPQLEEKTNETYGKLEAVQYKTQ | 787 |
| 4 | VLAPAGQHKRKHSTNYYIKVRAGDNKYMHLKVFNGPKGHRWHD | |
| | FKADRVLTGYQVDKNKDDELTGF | |
| FcRn-19 | MIPRGLSEAKPATPEIQEIVDKVKPQLEEKTNETYGKLEAVQYKTQ | 788 |
| 5 | VLADRHKYPVRVSTNYYIKVRAGDNKYMHLKVFNGPKHAWQHH | |
| | KSADRVLTGYQVDKNKDDELTGF | |
| FcRn-19 | MIPRGLSEAKPATPEIQEIVDKVKPQLEEKTNETYGKLEAVQYKTQ | 789 |
| 6 | VLAGNNNPQGHVSTNYYIKVRAGDNKYMHLKVFNGPYKHFKHH | |
| | WRADRVLTGYQVDKNKDDELTGF | |
| FcRn-19 | MIPRGLSEAKPATPEIQEIVDKVKPQLEEKTNETYGKLEAVQYKTQ | 790 |
| 7 | VLAKQLHHHHYKSTNYYIKVRAGDNKYMHLKVFNGPAHRKFFQ | |
| | WHADRVLTGYQVDKNKDDELTGF | |
| FcRn-19 | MIPRGLSEAKPATPEIQEIVDKVKPQLEEKTNETYGKLEAVQYKTQ | 791 |
| 8 | VLAQKHNWHRWHSTNYYIKVRAGDNKYMHLKVFNGPWTHRSQ | |
| | VKVADRVLTGYQVDKNKDDELTGF | |
| FcRn-19 | MIPRGLSEAKPATPEIQEIVDKVKPQLEEKTNETYGKLEAVQYKTQ | 792 |
| 9 | VLAYKHLGYWQKSTNYYIKVRAGDNKYMHLKVFNGPFQWFKVG | |
| | VPADRVLTGYQVDKNKDDELTGF | |
| FcRn-20 | MIPRGLSEAKPATPEIQEIVDKVKPQLEEKTNETYGKLEAVQYKTQ | 793 |
| 0 | VLAHQKNFEAWESTNYYIKVRAGDNKYMHLKVFNGPVRYYSKY | |
| | QWADRVLTGYQVDKNKDDELTGF | |
| FcRn-20 | MIPRGLSEAKPATPEIQEIVDKVKPQLEEKTNETYGKLEAVQYKTQ | 794 |
| 1 | VLAERVRRRHPPSTNYYIKVRAGDNKYMHLKVFNGPNGWHVGH | |
| | HIADRVLTGYQVDKNKDDELTGF | |
| FcRn-20 | MIPRGLSEAKPATPEIQEIVDKVKPQLEEKTNETYGKLEAVQYKTQ | 795 |
| 2 | VLAHKVHIFREPSTNYYIKVRAGDNKYMHLKVFNGPTRFRHYLVT | |
| | ADRVLTGYQVDKNKDDELTGF | |
| FcRn-20 | MIPRGLSEAKPATPEIQEIVDKVKPQLEEKTNETYGKLEAVQYKTQ | 796 |
| 3 | VLAVKSFHVHSHSTNYYIKVRAGDNKYMHLKVFNGPSWRNVRPE | |
| | F ADRVLTGYQVDKNKDDELTGF | |
| FcRn-20 | MIPRGLSEAKPATPEIQEIVDKVKPQLEEKTNETYGKLEAVQYKTQ | 797 |
| 4 | VLAWHKDPPPPWSTNYYIKVRAGDNKYMHLKVFNGPFGHTFSWR | |
| | YADRVLTGYQVDKNKDDELTGF | |
| FcRn-20 | MIPRGLSEAKPATPEIQEIVDKVKPQLEEKTNETYGKLEAVQYKTQ | 798 |
| 5 | VLAHRYAHNHFLSTNYYIKVRAGDNKYMHLKVFNGPFKHQKFYR | |
| | D ADRVLTGYQVDKNKDDELTGF | |
| FcRn-20 | MIPRGLSEAKPATPEIQEIVDKVKPQLEEKTNETYGKLEAVQYKTQ | 799 |
| 6 | VLAVSHALKTHTSTNYYIKVRAGDNKYMHLKVFNGPWRNKWRA | |
| | QDADRVLTGYQVDKNKDDELTGF | |
| FcRn-20 | MIPRGLSEAKPATPEIQEIVDKVKPQLEEKTNETYGKLEAVQYKTQ | 800 |
| 7 | VLAHQSRAIYVYSTNYYIKVRAGDNKYMHLKVFNGPYQKSYFHR | |
| | HADRVLTGYQVDKNKDDELTGF | |
| FcRn-20 | MIPRGLSEAKPATPEIQEIVDKVKPQLEEKTNETYGKLEAVQYKTQ | 801 |
| 8 | VLAHHTTYHQHHSTNYYIKVRAGDNKYMHLKVFNGPWRPRPVH | |
| | WKADRVLTGYQVDKNKDDELTGF | |
| FcRn-20 | MIPRGLSEAKPATPEIQEIVDKVKPQLEEKTNETYGKLEAVQYKTQ | 802 |
| 9 | VLATWWRNVQHHSTNYYIKVRAGDNKYMHLKVFNGPDPQYKRH | |
| | GYADRVLTGYQVDKNKDDELTGF | |
| FcRn-21 | MIPRGLSEAKPATPEIQEIVDKVKPQLEEKTNETYGKLEAVQYKTQ | 803 |
| 0 | VLAWNKHNYQHQSTNYYIKVRAGDNKYMHLKVFNGPVPHSVVH | |
| | YKADRVLTGYQVDKNKDDELTGF | |
| FcRn-21 | MIPRGLSEAKPATPEIQEIVDKVKPQLEEKTNETYGKLEAVQYKTQ | 804 |
| 1 | VLAQHTLRVHTVSTNYYIKVRAGDNKYMHLKVFNGPAYSQSFIHH | |
| | ADRVLTGYQVDKNKDDELTGF | |
| FcRn-21 | MIPRGLSEAKPATPEIQEIVDKVKPQLEEKTNETYGKLEAVQYKTQ | 805 |
| 2 | VLANQHFHQAGHSTNYYIKVRAGDNKYMHLKVFNGPFSHSTWRY | |
| | HADRVLTGYQVDKNKDDELTGF | |
| FcRn-21 | MIPRGLSEAKPATPEIQEIVDKVKPQLEEKTNETYGKLEAVQYKTQ | 806 |
| 3 | VLARQWTDRVWVSTNYYIKVRAGDNKYMHLKVFNGPSKKHQQH | |
| | W ADRVLTGYQVDKNKDDELTGF | |
| FcRn-21 | MIPRGLSEAKPATPEIQEIVDKVKPQLEEKTNETYGKLEAVQYKTQ | 807 |
| 4 | VLADHDYFHHNKSTNYYIKVRAGDNKYMHLKVFNGPAKHPRIHV | |
| | T ADRVLTGYQVDKNKDDELTGF | |
| FcRn-21 | MIPRGLSEAKPATPEIQEIVDKVKPQLEEKTNETYGKLEAVQYKTQ | 808 |
| 5 | VLAYWDVGPGFNSTNYYIKVRAGDNKYMHLKVFNGPSPWHHPT | |
| | HFADRVLTGYQVDKNKDDELTGF | |
| FcRn-21 | MIPRGLSEAKPATPEIQEIVDKVKPQLEEKTNETYGKLEAVQYKTQ | 809 |
| 6 | VLAGIHGHHEYYSTNYYIKVRAGDNKYMHLKVFNGPSNWFHHKH | |
| | RADRVLTGYQVDKNKDDELTGF | |
| FcRn-21 | MIPRGLSEAKPATPEIQEIVDKVKPQLEEKTNETYGKLEAVQYKTQ | 810 |
| 7 | VLAWQRSRYGKYSTNYYIKVRAGDNKYMHLKVFNGPAYWPYQK | |
| | PTADRVLTGYQVDKNKDDELTGF | |
| FcRn-21 | MIPRGLSEAKPATPEIQEIVDKVKPQLEEKTNETYGKLEAVQYKTQ | 811 |
| 8 | VLAYHQQHWRVHSTNYYIKVRAGDNKYMHLKVFNGPILVGYNW | |
| | HY ADRVLTGYQVDKNKDDELTGF | |
| FcRn-21 | MIPRGLSEAKPATPEIQEIVDKVKPQLEEKTNETYGKLEAVQYKTQ | 812 |
| 9 | VLAATRNSYPRHSTNYYIKVRAGDNKYMHLKVFNGPVHSHLPRH | |
| | PADRVLTGYQVDKNKDDELTGF | |
| FcRn-22 | MIPRGLSEAKPATPEIQEIVDKVKPQLEEKTNETYGKLEAVQYKTQ | 813 |
| 0 | VLAEHHHAHWATSTNYYIKVRAGDNKYMHLKVFNGPLFLHGVHI | |
| | FADRVLTGYQVDKNKDDELTGF | |
| FcRn-22 | MIPRGLSEAKPATPEIQEIVDKVKPQLEEKTNETYGKLEAVQYKTQ | 814 |
| 1 | VLAKQHQRSFIISTNYYIKVRAGDNKYMHLKVFNGPTSLPSEWFQ | |
| | ADRVLTGYQVDKNKDDELTGF | |
| FcRn-22 | MIPRGLSEAKPATPEIQEIVDKVKPQLEEKTNETYGKLEAVQYKTQ | 815 |
| 2 | VLAQFWGHRVEHSTNYYIKVRAGDNKYMHLKVFNGPTRHYHQR | |
| | NRADRVLTGYQVDKNKDDELTGF | |
| FcRn-22 | MIPRGLSEAKPATPEIQEIVDKVKPQLEEKTNETYGKLEAVQYKTQ | 816 |
| 3 | VLAFPSSHRTSYSTNYYIKVRAGDNKYMHLKVFNGPYSAHHIRWH | |
| | ADRVLTGYQVDKNKDDELTGF | |
| FcRn-22 | MIPRGLSEAKPATPEIQEIVDKVKPQLEEKTNETYGKLEAVQYKTQ | 817 |
| 4 | VLASSKYIDHRQSTNYYIKVRAGDNKYMHLKVFNGPERAQHHTH | |
| | PADRVLTGYQVDKNKDDELTGF | |
| FcRn-22 | MIPRGLSEAKPATPEIQEIVDKVKPQLEEKTNETYGKLEAVQYKTQ | 818 |
| 5 | VLAYWRHEHSSPSTNYYIKVRAGDNKYMHLKVFNGPWKKHHYG | |
| | HY ADRVLTGYQVDKNKDDELTGF | |
| FcRn-22 | MIPRGLSEAKPATPEIQEIVDKVKPQLEEKTNETYGKLEAVQYKTQ | 819 |
| 6 | VLAERAHYDHHYSTNYYIKVRAGDNKYMHLKVFNGPSHHAHHS | |
| | VQADRVLTGYQVDKNKDDELTGF | |
| FcRn-22 | MIPRGLSEAKPATPEIQEIVDKVKPQLEEKTNETYGKLEAVQYKTQ | 820 |
| 7 | VLAWRHKAYIYGSTNYYIKVRAGDNKYMHLKVFNGPWKHWEHK | |
| | PQADRVLTGYQVDKNKDDELTGF | |
| FcRn-22 | MIPRGLSEAKPATPEIQEIVDKVKPQLEEKTNETYGKLEAVQYKTQ | 821 |
| 8 | VLAPQIKEQYNGSTNYYIKVRAGDNKYMHLKVFNGPAQVPVLLW | |
| | YADRVLTGYQVDKNKDDELTGF | |
| FcRn-22 | MIPRGLSEAKPATPEIQEIVDKVKPQLEEKTNETYGKLEAVQYKTQ | 822 |
| 9 | VLAFKKVARDHWSTNYYIKVRAGDNKYMHLKVFNGPWVHFYPW | |
| | QQADRVLTGYQVDKNKDDELTGF | |
| FcRn-23 | MIPRGLSEAKPATPEIQEIVDKVKPQLEEKTNETYGKLEAVQYKTQ | 823 |
| 0 | VLAAQKHHWHKTSTNYYIKVRAGDNKYMHLKVFNGPWHLAHVF | |
| | YTADRVLTGYQVDKNKDDELTGF | |
| FcRn-23 | MIPRGLSEAKPATPEIQEIVDKVKPQLEEKTNETYGKLEAVQYKTQ | 824 |
| 1 | VLAVSQGHHSWDSTNYYIKVRAGDNKYMHLKVFNGPSSHHHKN | |
| | HHADRVLTGYQVDKNKDDELTGF | |
| FcRn-23 | MIPRGLSEAKPATPEIQEIVDKVKPQLEEKTNETYGKLEAVQYKTQ | 825 |
| 2 | VLAWHLRGHPHYSTNYYIKVRAGDNKYMHLKVFNGPTKQPHGV | |
| | HYADRVLTGYQVDKNKDDELTGF | |
| FcRn-23 | MIPRGLSEAKPATPEIQEIVDKVKPQLEEKTNETYGKLEAVQYKTQ | 826 |
| 3 | VLAHSHHHQPWESTNYYIKVRAGDNKYMHLKVFNGPEHRTHHLG | |
| | KADRVLTGYQVDKNKDDELTGF | |
| FcRn-23 | MIPRGLSEAKPATPEIQEIVDKVKPQLEEKTNETYGKLEAVQYKTQ | 827 |
| 4 | VLARRFRVHLHQSTNYYIKVRAGDNKYMHLKVFNGPTNHRQDHP | |
| | EADRVLTGYQVDKNKDDELTGF | |
| FcRn-23 | MIPRGLSEAKPATPEIQEIVDKVKPQLEEKTNETYGKLEAVQYKTQ | 828 |
| 5 | VLAGRQTKSHQHSTNYYIKVRAGDNKYMHLKVFNGPHRKTNWH | |
| | SYADRVLTGYQVDKNKDDELTGF | |
| FcRn-23 | MIPRGLSEAKPATPEIQEIVDKVKPQLEEKTNETYGKLEAVQYKTQ | 829 |
| 6 | VLAPYSRHHHQLSTNYYIKVRAGDNKYMHLKVFNGPSGVHHAAV | |
| | WADRVLTGYQVDKNKDDELTGF | |
| FcRn-23 | MIPRGLSEAKPATPEIQEIVDKVKPQLEEKTNETYGKLEAVQYKTQ | 830 |
| 7 | VLAVHGDHTRAWSTNYYIKVRAGDNKYMHLKVFNGPRYASSYW | |
| | EWADRVLTGYQVDKNKDDELTGF | |
| FcRn-23 | MIPRGLSEAKPATPEIQEIVDKVKPQLEEKTNETYGKLEAVQYKTQ | 831 |
| 8 | VLADWQKRGRSWSTNYYIKVRAGDNKYMHLKVFNGPNQSGVVV | |
| | QVADRVLTGYQVDKNKDDELTGF | |
| FcRn-23 | MIPRGLSEAKPATPEIQEIVDKVKPQLEEKTNETYGKLEAVQYKTQ | 832 |
| 9 | VLAYNWERFRKVSTNYYIKVRAGDNKYMHLKVFNGPYHNHQHTI | |
| | HADRVLTGYQVDKNKDDELTGF | |
| FcRn-24 | MIPRGLSEAKPATPEIQEIVDKVKPQLEEKTNETYGKLEAVQYKTQ | 833 |
| 0 | VLAGWSRNVWFWSTNYYIKVRAGDNKYMHLKVFNGPKQELGTK | |
| | TTADRVLTGYQVDKNKDDELTGF | |
| FcRn-24 | MIPRGLSEAKPATPEIQEIVDKVKPQLEEKTNETYGKLEAVQYKTQ | 834 |
| 1 | VLASQTQHRRHHSTNYYIKVRAGDNKYMHLKVFNGPLVPQHHQH | |
| | QADRVLTGYQVDKNKDDELTGF | |
| FcRn-24 | MIPRGLSEAKPATPEIQEIVDKVKPQLEEKTNETYGKLEAVQYKTQ | 835 |
| 2 | VLAPNVKHKHRWSTNYYIKVRAGDNKYMHLKVFNGPWHDIAGG | |
| | HYADRVLTGYQVDKNKDDELTGF | |
| FcRn-24 | MIPRGLSEAKPATPEIQEIVDKVKPQLEEKTNETYGKLEAVQYKTQ | 836 |
| 3 | VLAKHPAFHQHSSTNYYIKVRAGDNKYMHLKVFNGPRHDLHYHY | |
| | PADRVLTGYQVDKNKDDELTGF | |
| FcRn-24 | MIPRGLSEAKPATPEIQEIVDKVKPQLEEKTNETYGKLEAVQYKTQ | 837 |
| 4 | VLAPHHHTDWRTSTNYYIKVRAGDNKYMHLKVFNGPYWHWKVR | |
| | RFADRVLTGYQVDKNKDDELTGF | |
| FcRn-24 | MIPRGLSEAKPATPEIQEIVDKVKPQLEEKTNETYGKLEAVQYKTQ | 838 |
| 5 | VLAHTHKILHFHSTNYYIKVRAGDNKYMHLKVFNGPDKQRYEDK | |
| | QADRVLTGYQVDKNKDDELTGF | |
| FcRn-24 | MIPRGLSEAKPATPEIQEIVDKVKPQLEEKTNETYGKLEAVQYKTQ | 839 |
| 6 | VLAPNHHFFLQFSTNYYIKVRAGDNKYMHLKVFNGPQHHHPHRH | |
| | PADRVLTGYQVDKNKDDELTGF | |
| FcRn-24 | MIPRGLSEAKPATPEIQEIVDKVKPQLEEKTNETYGKLEAVQYKTQ | 840 |
| 7 | VLARRYIGHNYSSTNYYIKVRAGDNKYMHLKVFNGPWHHFHNSY | |
| | DADRVLTGYQVDKNKDDELTGF | |
| FcRn-24 | MIPRGLSEAKPATPEIQEIVDKVKPQLEEKTNETYGKLEAVQYKTQ | 841 |
| 8 | VLATHYHHQWDPSTNYYIKVRAGDNKYMHLKVFNGPIWYSHRPR | |
| | AADRVLTGYQVDKNKDDELTGF | |
| FcRn-24 | MIPRGLSEAKPATPEIQEIVDKVKPQLEEKTNETYGKLEAVQYKTQ | 842 |
| 9 | VLADKKHGQYKSTNYYIKVRAGDNKYMHLKVFNGPWDDHTLK | |
| | WYADRVLTGYQVDKNKDDELTGF | |
| FcRn-25 | MIPRGLSEAKPATPEIQEIVDKVKPQLEEKTNETYGKLEAVQYKTQ | 843 |
| 0 | VLAYHIQGVYWRSTNYYIKVRAGDNKYMHLKVFNGPIAFWGPKR | |
| | FADRVLTGYQVDKNKDDELTGF | |
| FcRn-25 | MIPRGLSEAKPATPEIQEIVDKVKPQLEEKTNETYGKLEAVQYKTQ | 844 |
| 1 | VLASRFKHHVRNSTNYYIKVRAGDNKYMHLKVFNGPFPHRNKSD | |
| | GADRVLTGYQVDKNKDDELTGF | |
| FcRn-25 | MIPRGLSEAKPATPEIQEIVDKVKPQLEEKTNETYGKLEAVQYKTQ | 845 |
| 2 | VLAWHHQHHLLASTNYYIKVRAGDNKYMHLKVFNGPFKRSQQW | |
| | EWADRVLTGYQVDKNKDDELTGF | |
| FcRn-25 | MIPRGLSEAKPATPEIQEIVDKVKPQLEEKTNETYGKLEAVQYKTQ | 846 |
| 3 | VLAHNKHPSPRVSTNYYIKVRAGDNKYMHLKVFNGPKHRYQPTH | |
| | WADRVLTGYQVDKNKDDELTGF | |
| FcRn-25 | MIPRGLSEAKPATPEIQEIVDKVKPQLEEKTNETYGKLEAVQYKTQ | 847 |
| 4 | VLATWFHQHEQQSTNYYIKVRAGDNKYMHLKVFNGPYHDIWAW | |
| | HVADRVLTGYQVDKNKDDELTGF | |
| FcRn-25 | MIPRGLSEAKPATPEIQEIVDKVKPQLEEKTNETYGKLEAVQYKTQ | 848 |
| 5 | VLAWKEWRYHHQSTNYYIKVRAGDNKYMHLKVFNGPDFVKHHL | |
| | HDADRVLTGYQVDKNKDDELTGF | |
| FcRn-25 | MIPRGLSEAKPATPEIQEIVDKVKPQLEEKTNETYGKLEAVQYKTQ | 849 |
| 6 | VLAFTKHWDRWYSTNYYIKVRAGDNKYMHLKVFNGPISDHVHFG | |
| | WADRVLTGYQVDKNKDDELTGF | |
| FcRn-25 | MIPRGLSEAKPATPEIQEIVDKVKPQLEEKTNETYGKLEAVQYKTQ | 850 |
| 7 | VLATRLYDHSVWSTNYYIKVRAGDNKYMHLKVFNGPYHHRDHW | |
| | GWADRVLTGYQVDKNKDDELTGF | |
| FcRn-25 | MIPRGLSEAKPATPEIQEIVDKVKPQLEEKTNETYGKLEAVQYKTQ | 851 |
| 8 | VLAWEYQTHHPASTNYYIKVRAGDNKYMHLKVFNGPEWFTVGGI | |
| | AADRVLTGYQVDKNKDDELTGF | |
| FcRn-25 | MIPRGLSEAKPATPEIQEIVDKVKPQLEEKTNETYGKLEAVQYKTQ | 852 |
| 9 | VLAVHFRSHRDFSTNYYIKVRAGDNKYMHLKVFNGPERKHAHQH | |
| | PADRVLTGYQVDKNKDDELTGF | |
| FcRn-26 | MIPRGLSEAKPATPEIQEIVDKVKPQLEEKTNETYGKLEAVQYKTQ | 853 |
| 0 | VLASRHTHHHRSSTNYYIKVRAGDNKYMHLKVFNGPDSNLYNEW | |
| | NADRVLTGYQVDKNKDDELTGF | |
| FcRn-26 | MIPRGLSEAKPATPEIQEIVDKVKPQLEEKTNETYGKLEAVQYKTQ | 854 |
| 1 | VLATARYEHAPTSTNYYIKVRAGDNKYMHLKVFNGPTAKHSHKK | |
| | HADRVLTGYQVDKNKDDELTGF | |
| FcRn-26 | MIPRGLSEAKPATPEIQEIVDKVKPQLEEKTNETYGKLEAVQYKTQ | 855 |
| 2 | VLARHRKESWYVSTNYYIKVRAGDNKYMHLKVFNGPNWPHGIDP | |
| | KADRVLTGYQVDKNKDDELTGF | |
| FcRn-26 | MIPRGLSEAKPATPEIQEIVDKVKPQLEEKTNETYGKLEAVQYKTQ | 856 |
| 3 | VLADHGYARGHHSTNYYIKVRAGDNKYMHLKVFNGPKHIHEHKS | |
| | EADRVLTGYQVDKNKDDELTGF | |
| FcRn-26 | MIPRGLSEAKPATPEIQEIVDKVKPQLEEKTNETYGKLEAVQYKTQ | 857 |
| 4 | VLATPHKIWHWHSTNYYIKVRAGDNKYMHLKVFNGPTKKFHQHE | |
| | RADRVLTGYQVDKNKDDELTGF | |
| FcRn-26 | MIPRGLSEAKPATPEIQEIVDKVKPQLEEKTNETYGKLEAVQYKTQ | 858 |
| 5 | VLASYAQHTRLHSTNYYIKVRAGDNKYMHLKVFNGPTRHHQHYY | |
| | EADRVLTGYQVDKNKDDELTGF | |
| FcRn-26 | MIPRGLSEAKPATPEIQEIVDKVKPQLEEKTNETYGKLEAVQYKTQ | 859 |
| 6 | VLAIDHRYHYLHSTNYYIKVRAGDNKYMHLKVFNGPWYWTQHH | |
| | RWADRVLTGYQVDKNKDDELTGF | |
| FcRn-26 | MIPRGLSEAKPATPEIQEIVDKVKPQLEEKTNETYGKLEAVQYKTQ | 860 |
| 7 | VLAHGYNHRKVQSTNYYIKVRAGDNKYMHLKVFNGPYHVWNW | |
| | RLKADRVLTGYQVDKNKDDELTGF | |
| FcRn-26 | MIPRGLSEAKPATPEIQEIVDKVKPQLEEKTNETYGKLEAVQYKTQ | 861 |
| 8 | VLAGHLKAAPWHSTNYYIKVRAGDNKYMHLKVFNGPFHHFRPHH | |
| | HADRVLTGYQVDKNKDDELTGF | |
| FcRn-26 | MIPRGLSEAKPATPEIQEIVDKVKPQLEEKTNETYGKLEAVQYKTQ | 862 |
| 9 | VLAKEKYASWERSTNYYIKVRAGDNKYMHLKVFNGPFLNGKKRH | |
| | VADRVLTGYQVDKNKDDELTGF | |
| FcRn-27 | MIPRGLSEAKPATPEIQEIVDKVKPQLEEKTNETYGKLEAVQYKTQ | 863 |
| 0 | VLAKGHPHAHPHSTNYYIKVRAGDNKYMHLKVFNGPWWKIHGST | |
| | VADRVLTGYQVDKNKDDELTGF | |
| FcRn-27 | MIPRGLSEAKPATPEIQEIVDKVKPQLEEKTNETYGKLEAVQYKTQ | 864 |
| 1 | VLAPYRRHEHHQSTNYYIKVRAGDNKYMHLKVFNGPNSDFHHNQ | |
| | QADRVLTGYQVDKNKDDELTGF | |
| FcRn-27 | MIPRGLSEAKPATPEIQEIVDKVKPQLEEKTNETYGKLEAVQYKTQ | 865 |
| 2 | VLAGFPHWFVHNSTNYYIKVRAGDNKYMHLKVFNGPTHHLRYHH | |
| | QADRVLTGYQVDKNKDDELTGF | |
| FcRn-27 | MIPRGLSEAKPATPEIQEIVDKVKPQLEEKTNETYGKLEAVQYKTQ | 866 |
| 3 | VLAFRRYQSFHYSTNYYIKVRAGDNKYMHLKVFNGPFYKYHQVR | |
| | WADRVLTGYQVDKNKDDELTGF | |
| FcRn-27 | MIPRGLSEAKPATPEIQEIVDKVKPQLEEKTNETYGKLEAVQYKTQ | 867 |
| 4 | VLAPRYRHHVDYSTNYYIKVRAGDNKYMHLKVFNGPYSFRDHH | |
| | WWADRVLTGYQVDKNKDDELTGF | |
| FcRn-27 | MIPRGLSEAKPATPEIQEIVDKVKPQLEEKTNETYGKLEAVQYKTQ | 868 |
| 5 | VLADYLKRNFRYSTNYYIKVRAGDNKYMHLKVFNGPPFYRNHHH | |
| | EADRVLTGYQVDKNKDDELTGF | |
| FcRn-27 | MIPRGLSEAKPATPEIQEIVDKVKPQLEEKTNETYGKLEAVQYKTQ | 869 |
| 6 | VLARSHPGKHVHSTNYYIKVRAGDNKYMHLKVFNGPFQLNLRWG | |
| | QADRVLTGYQVDKNKDDELTGF | |
| FcRn-27 | MIPRGLSEAKPATPEIQEIVDKVKPQLEEKTNETYGKLEAVQYKTQ | 870 |
| 7 | VLAHHHRWAKWLSTNYYIKVRAGDNKYMHLKVFNGPVHNFHDI | |
| | RHADRVLTGYQVDKNKDDELTGF | |
| FcRn-27 | MIPRGLSEAKPATPEIQEIVDKVKPQLEEKTNETYGKLEAVQYKTQ | 871 |
| 8 | VLAAAHHNHWHISTNYYIKVRAGDNKYMHLKVFNGPAQHGHVP | |
| | FSADRVLTGYQVDKNKDDELTGF | |
| FcRn-27 | MIPRGLSEAKPATPEIQEIVDKVKPQLEEKTNETYGKLEAVQYKTQ | 872 |
| 9 | VLAPVQKHAGSHSTNYYIKVRAGDNKYMHLKVFNGPPWHNAEIK | |
| | HADRVLTGYQVDKNKDDELTGF | |
| FcRn-28 | MIPRGLSEAKPATPEIQEIVDKVKPQLEEKTNETYGKLEAVQYKTQ | 873 |
| 0 | VLADNWRHWRIWSTNYYIKVRAGDNKYMHLKVFNGPAGWSSNK | |
| | ADADRVLTGYQVDKNKDDELTGF | |
| FcRn-28 | MIPRGLSEAKPATPEIQEIVDKVKPQLEEKTNETYGKLEAVQYKTQ | 874 |
| 1 | VLAPRHHHWAFSTNYYIKVRAGDNKYMHLKVFNGPKRQHHDVG | |
| | QADRVLTGYQVDKNKDDELTGF | |
| FcRn-28 | MIPRGLSEAKPATPEIQEIVDKVKPQLEEKTNETYGKLEAVQYKTQ | 875 |
| 2 | VLAVSYDDITWVSTNYYIKVRAGDNKYMHLKVFNGPNSSYGWL | |
| | WWADRVLTGYQVDKNKDDELTGF | |
| FcRn-28 | MIPRGLSEAKPATPEIQEIVDKVKPQLEEKTNETYGKLEAVQYKTQ | 876 |
| 3 | VLAPPHPRVQHYSTNYYIKVRAGDNKYMHLKVFNGPAFRDHRAP | |
| | HADRVLTGYQVDKNKDDELTGF | |
| FcRn-28 | MIPRGLSEAKPATPEIQEIVDKVKPQLEEKTNETYGKLEAVQYKTQ | 877 |
| 4 | VLAKQFRHHQHESTNYYIKVRAGDNKYMHLKVFNGPKWWSTQGI | |
| | VADRVLTGYQVDKNKDDELTGF | |
| FcRn-28 | MIPRGLSEAKPATPEIQEIVDKVKPQLEEKTNETYGKLEAVQYKTQ | 878 |
| 5 | VLAEHHEYHYRYSTNYYIKVRAGDNKYMHLKVFNGPFRPVHHIRI | |
| | ADRVLTGYQVDKNKDDELTGF | |
| FcRn-28 | MIPRGLSEAKPATPEIQEIVDKVKPQLEEKTNETYGKLEAVQYKTQ | 879 |
| 6 | VLAHHHHRQHPSTNYYIKVRAGDNKYMHLKVFNGPKVGQGVNL | |
| | G ADRVLTGYQVDKNKDDELTGF | |
| FcRn-28 | MIPRGLSEAKPATPEIQEIVDKVKPQLEEKTNETYGKLEAVQYKTQ | 880 |
| 7 | VLAKLHQAHHWHSTNYYIKVRAGDNKYMHLKVFNGPEWSNKHY | |
| | QWADRVLTGYQVDKNKDDELTGF | |
| FcRn-28 | MIPRGLSEAKPATPEIQEIVDKVKPQLEEKTNETYGKLEAVQYKTQ | 881 |
| 8 | VLAEYHHYGTSRSTNYYIKVRAGDNKYMHLKVFNGPRQLKHHTN | |
| | F ADRVLTGYQVDKNKDDELTGF | |
| FcRn-28 | MIPRGLSEAKPATPEIQEIVDKVKPQLEEKTNETYGKLEAVQYKTQ | 882 |
| 9 | VLADNKHIPQRQSTNYYIKVRAGDNKYMHLKVFNGPRNHVAEKY | |
| | WADRVLTGYQVDKNKDDELTGF | |
| FcRn-29 | MIPRGLSEAKPATPEIQEIVDKVKPQLEEKTNETYGKLEAVQYKTQ | 883 |
| 0 | VLAHKQWQWTIVSTNYYIKVRAGDNKYMHLKVFNGPAYKSDKIR | |
| | KADRVLTGYQVDKNKDDELTGF | |
| FcRn-29 | MIPRGLSEAKPATPEIQEIVDKVKPQLEEKTNETYGKLEAVQYKTQ | 884 |
| 1 | VLAYRIGHGVQHSTNYYIKVRAGDNKYMHLKVFNGPYDKPYIVW | |
| | IADRVLTGYQVDKNKDDELTGF | |
| FcRn-29 | MIPRGLSEAKPATPEIQEIVDKVKPQLEEKTNETYGKLEAVQYKTQ | 885 |
| 2 | VLADQVRRIPHHSTNYYIKVRAGDNKYMHLKVFNGPHDKHPQSW | |
| | AADRVLTGYQVDKNKDDELTGF | |
| FcRn-29 | MIPRGLSEAKPATPEIQEIVDKVKPQLEEKTNETYGKLEAVQYKTQ | 886 |
| 3 | VLAEGKHEFRFQSTNYYIKVRAGDNKYMHLKVFNGPWDKHRQHL | |
| | WADRVLTGYQVDKNKDDELTGF | |
| FcRn-29 | MIPRGLSEAKPATPEIQEIVDKVKPQLEEKTNETYGKLEAVQYKTQ | 887 |
| 4 | VLAHYWGRWYKISTNYYIKVRAGDNKYMHLKVFNGPFHAFWHL | |
| | AYADRVLTGYQVDKNKDDELTGF | |
| AVA04- | MIPRGLSEAKPATPEIQEIVDKVKPQLEEKTNETYGKLEAVQYKTQ | 1184 |
| 251 FX6 | VLALSFNNYHWHSTNYYIKVRAGDNKYMHLKVFNGPKLRHDKLT | |
| | HADRVLTGYQVDKNKDDELTGFAEAAAKEAAAKEAAAKEAAAK | |
| | EAAAKEAAAKMIPRGLSEAKPATPEIQEIVDKVKPQLEEKTNETYG | |
| | KLEAVQYKTQVLAREGRQDWVLSTNYYIKVRAGDNKYMHLKVF | |
| | NGPWVPFPHQQLADRVLTGYQVDKNKDDELTGFAEAAAKEAAA | |
| | KEAAAKEAAAKEAAAKEAAAKMIPRGLSEAKPATPEIQEIVDKVK | |
| | PQLEEKTNETYGKLEAVQYKTQVLAREGRQDWVLSTNYYIKVRA | |
| | GDNKYMHLKVFNGPWVPFPHQQLADRVLTGYQVDKNKDDELTG | |
| | F | |

TABLE 3
Examples of FeRn Binding AFFIMER® Polynucleotide Sequences

| | | SEQ |
|---|---|---|
| | | ID |
| Name | DNA Sequence | NO: |
| FcRn-01 | ATGATCCCGCGTGGCCTGTCTGAAGCTAAACCAGCAACTCCGGAAA | 888 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATGTTATCGATCATAAATACCGTCATTCCACCAA | |
| | CTATTACATTAAGGTTCGTGCCGGTGACAATAAGTATATGCACCTG | |
| | AAAGTGTTCAACGGCCCGAAAAAAGTTAACCATCATTACCATAAA | |
| | GCGGACCGTGTTCTGACCGGTTACCAGGTTGACAAGAACAAAGAT | |
| | GACGAGCTGACGGGTTTC | |
| FcRn-02 | ATGATCCCGCGTGGCCTGTCTGAAGCTAAACCAGCAACTCCGGAAA | 889 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACTGAAAGGTCATAAACATCATAAAACCTCCACCA | |
| | ACTATTACATTAAGGTTCGTGCCGGTGACAATAAGTATATGCACCT | |
| | GAAAGTGTTCAACGGCCCGTGGCAGGCAAAACATAAAGATGGTAA | |
| | AGCGGACCGTGTTCTGACCGGTTACCAGGTTGACAAGAACAAAGA | |
| | TGACGAGCTGACGGGTTTC | |
| FcRn-03 | ATGATCCCGCGTGGCCTGTCTGAAGCTAAACCAGCAACTCCGGAAA | 890 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAACCATCATAAATACCCACATGGTTCCACCAA | |
| | CTATTACATTAAGGTTCGTGCCGGTGACAATAAGTATATGCACCTG | |
| | AAAGTGTTCAACGGCCCGATCTGGTCTAAACATAACTGGCATTGGG | |
| | CGGACCGTGTTCTGACCGGTTACCAGGTTGACAAGAACAAAGATG | |
| | ACGAGCTGACGGGTTTC | |
| FcRn-04 | ATGATCCCGCGTGGCCTGTCTGAAGCTAAACCAGCAACTCCGGAAA | 891 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTCATAAAAAACATCATAAATGGTTTTCCACCAA | |
| | CTATTACATTAAGGTTCGTGCCGGTGACAATAAGTATATGCACCTG | |
| | AAAGTGTTCAACGGCCCGAAATGGCAGGTTGCACGTCATGATAAC | |
| | GCGGACCGTGTTCTGACCGGTTACCAGGTTGACAAGAACAAAGAT | |
| | GACGAGCTGACGGGTTTC | |
| FcRn-05 | ATGATCCCGCGTGGCCTGTCTGAAGCTAAACCAGCAACTCCGGAAA | 892 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACGTCATGCAGATCATCCACGTGTTTCCACCAA | |
| | CTATTACATTAAGGTTCGTGCCGGTGACAATAAGTATATGCACCTG | |
| | AAAGTGTTCAACGGCCCGGCACATAACTACACCCTGGTTTGGTACG | |
| | CGGACCGTGTTCTGACCGGTTACCAGGTTGACAAGAACAAAGATG | |
| | ACGAGCTGACGGGTTTC | |
| FcRn-06 | ATGATCCCGCGTGGCCTGTCTGAAGCTAAACCAGCAACTCCGGAAA | 893 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGCAGCCAAAACAGCATGGTTTTCATTCCACCAA | |
| | CTATTACATTAAGGTTCGTGCCGGTGACAATAAGTATATGCACCTG | |
| | AAAGTGTTCAACGGCCCGTCTTCTGGTAACAAACATAAACATCATG | |
| | CGGACCGTGTTCTGACCGGTTACCAGGTTGACAAGAACAAAGATG | |
| | ACGAGCTGACGGGTTTC | |
| FcRn-07 | ATGATCCCGCGTGGCCTGTCTGAAGCTAAACCAGCAACTCCGGAAA | 894 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATGGTCATCGTACCCATTCTGTTTCCACCAA | |
| | CTATTACATTAAGGTTCGTGCCGGTGACAATAAGTATATGCACCTG | |
| | AAAGTGTTCAACGGCCCGGTTTGGGCACATCATAAAAAATACTACG | |
| | CGGACCGTGTTCTGACCGGTTACCAGGTTGACAAGAACAAAGATG | |
| | ACGAGCTGACGGGTTTC | |
| FcRn-08 | ATGATCCCGCGTGGCCTGTCTGAAGCTAAACCAGCAACTCCGGAAA | 895 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACAGCATCATTGGGATGTTCATCGTTCCACCAA | |
| | CTATTACATTAAGGTTCGTGCCGGTGACAATAAGTATATGCACCTG | |
| | AAAGTGTTCAACGGCCCGAAAGTTAAACATACCCGTATCCATGCGG | |
| | ACCGTGTTCTGACCGGTTACCAGGTTGACAAGAACAAAGATGACG | |
| | AGCTGACGGGTTTC | |
| FcRn-09 | ATGATCCCGCGTGGCCTGTCTGAAGCTAAACCAGCAACTCCGGAAA | 896 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTGGTCAGCCAGCAAAACAGCATTTTTCCACCAA | |
| | CTATTACATTAAGGTTCGTGCCGGTGACAATAAGTATATGCACCTG | |
| | AAAGTGTTCAACGGCCCGCCAAACAAACATCATCATGCACATAAA | |
| | GCGGACCGTGTTCTGACCGGTTACCAGGTTGACAAGAACAAAGAT | |
| | GACGAGCTGACGGGTTTC | |
| FcRn-10 | ATGATCCCGCGTGGCCTGTCTGAAGCTAAACCAGCAACTCCGGAAA | 897 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCATGTTCGTTGGAAAGATCATGATTCCACCAA | |
| | CTATTACATTAAGGTTCGTGCCGGTGACAATAAGTATATGCACCTG | |
| | AAAGTGTTCAACGGCCCGTTTATCAAACGTTACAAACTGCAGCGTG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 898 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATTCTCATCATCCAGAACATTGGTACTCCACCAA | |
| | CTATTACATTAAGGTTCGTGCCGGTGACAATAAGTATATGCACCTG | |
| | AAAGTGTTCAACGGCCCGCGTAAAGATTGGCATGTTCGTAAACTGG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 899 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAGTTAAAACCCATGATCATCAGCGTTCCACCAA | |
| | CTATTACATTAAGGTTCGTGCCGGTGACAATAAGTATATGCACCTG | |
| | AAAGTGTTCAACGGCCCGATCCATCAGCATCATTCTCAGGATTGGG | |
| | CGGACCGTGTTCTGACCGGTTACCAGGTTGACAAGAACAAAGATG | |
| | ACGAGCTGACGGGTTTC | |
| FcRn-13 | ATGATCCCGCGTGGCCTGTCTGAAGCTAAACCAGCAACTCCGGAAA | 900 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCGTGAAGTTTCTAAACGTCGTACCTCCACCAA | |
| | CTATTACATTAAGGTTCGTGCCGGTGACAATAAGTATATGCACCTG | |
| | AAAGTGTTCAACGGCCCGAACCAGAAACAGGGTCATAAACATAAA | |
| | GCGGACCGTGTTCTGACCGGTTACCAGGTTGACAAGAACAAAGAT | |
| | GACGAGCTGACGGGTTTC | |
| FcRn-14 | ATGATCCCGCGTGGCCTGTCTGAAGCTAAACCAGCAACTCCGGAAA | 901 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTACCAAACGTGCATGGCTGAAAATCTCCACCAA | |
| | CTATTACATTAAGGTTCGTGCCGGTGACAATAAGTATATGCACCTG | |
| | AAAGTGTTCAACGGCCCGTTTTACGCACAGAAACGTACCTCTTACG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 902 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAACCATCGTCATTACTCTAAAGGTTCCACCAA | |
| | CTATTACATTAAGGTTCGTGCCGGTGACAATAAGTATATGCACCTG | |
| | AAAGTGTTCAACGGCCCGGCATTTAACGATGGTGCAGTTTTTATCG | |
| | CGGACCGTGTTCTGACCGGTTACCAGGTTGACAAGAACAAAGATG | |
| | ACGAGCTGACGGGTTTC | |
| FcRn-16 | ATGATCCCGCGTGGCCTGTCTGAAGCTAAACCAGCAACTCCGGAAA | 903 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACATCATCATCATAAACATCAGCATTCCACCAA | |
| | CTATTACATTAAGGTTCGTGCCGGTGACAATAAGTATATGCACCTG | |
| | AAAGTGTTCAACGGCCCGGTTTTTCTGCATAACGAATCTCATCAGG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 904 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCCACATCATGTTCGTTCTTCTGTTTCCACCAAC | |
| | TATTACATTAAGGTTCGTGCCGGTGACAATAAGTATATGCACCTGA | |
| | AAGTGTTCAACGGCCCGAAAGGTCATTTTCATACCCATCTGGTTGC | |
| | GGACCGTGTTCTGACCGGTTACCAGGTTGACAAGAACAAAGATGA | |
| | CGAGCTGACGGGTTTC | |
| FcRn-18 | ATGATCCCGCGTGGCCTGTCTGAAGCTAAACCAGCAACTCCGGAAA | 905 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAAACCCCACATGAACGTCATAAAACCTCCACCA | |
| | ACTATTACATTAAGGTTCGTGCCGGTGACAATAAGTATATGCACCT | |
| | GAAAGTGTTCAACGGCCCGAAACGTTGGCTGAAACATCATGCACA | |
| | TGCGGACCGTGTTCTGACCGGTTACCAGGTTGACAAGAACAAAGAT | |
| | GACGAGCTGACGGGTTTC | |
| FcRn-19 | ATGATCCCGCGTGGCCTGTCTGAAGCTAAACCAGCAACTCCGGAAA | 906 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTACCATCCAGCATGTTAACCAGCATTCCACCAA | |
| | CTATTACATTAAGGTTCGTGCCGGTGACAATAAGTATATGCACCTG | |
| | AAAGTGTTCAACGGCCCGTACGGTCATAAACATCATTTTCATTGGG | |
| | CGGACCGTGTTCTGACCGGTTACCAGGTTGACAAGAACAAAGATG | |
| | ACGAGCTGACGGGTTTC | |
| FcRn-20 | ATGATCCCGCGTGGCCTGTCTGAAGCTAAACCAGCAACTCCGGAAA | 907 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACAACGTTGGTCGTAAAAAACATCGTTCCACCAA | |
| | CTATTACATTAAGGTTCGTGCCGGTGACAATAAGTATATGCACCTG | |
| | AAAGTGTTCAACGGCCCGGTTCATTTTTTTCATGATCAGTCTGAAG | |
| | CGGACCGTGTTCTGACCGGTTACCAGGTTGACAAGAACAAAGATG | |
| | ACGAGCTGACGGGTTTC | |
| FcRn-21 | ATGATCCCGCGTGGCCTGTCTGAAGCTAAACCAGCAACTCCGGAAA | 908 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCGTGGTCCACAGAAATCTTCTTACTCCACCAA | |
| | CTATTACATTAAGGTTCGTGCCGGTGACAATAAGTATATGCACCTG | |
| | AAAGTGTTCAACGGCCCGCAGAAAAAAAACCGTCATCATCAGAAA | |
| | GCGGACCGTGTTCTGACCGGTTACCAGGTTGACAAGAACAAAGAT | |
| | GACGAGCTGACGGGTTTC | |
| FcRn-22 | ATGATCCCGCGTGGCCTGTCTGAAGCTAAACCAGCAACTCCGGAAA | 909 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATGATCGTCATCAGAAACATTGGCGTTCCACCAA | |
| | CTATTACATTAAGGTTCGTGCCGGTGACAATAAGTATATGCACCTG | |
| | AAAGTGTTCAACGGCCCGGATCTGCGTAAACATAAATGGAAATCT | |
| | GCGGACCGTGTTCTGACCGGTTACCAGGTTGACAAGAACAAAGAT | |
| | GACGAGCTGACGGGTTTC | |
| FcRn-23 | ATGATCCCGCGTGGCCTGTCTGAAGCTAAACCAGCAACTCCGGAAA | 910 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAATCCCACATCATCATAAACCACGTGTTTCCACCAA | |
| | CTATTACATTAAGGTTCGTGCCGGTGACAATAAGTATATGCACCTG | |
| | AAAGTGTTCAACGGCCCGTCTTTTCATCATCATCGTCATTCTGATGC | |
| | GGACCGTGTTCTGACCGGTTACCAGGTTGACAAGAACAAAGATGA | |
| | CGAGCTGACGGGTTTC | |
| FcRn-24 | ATGATCCCGCGTGGCCTGTCTGAAGCTAAACCAGCAACTCCGGAAA | 911 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAGGTAAACATTACCATTCTCAGCAGTCCACCAA | |
| | CTATTACATTAAGGTTCGTGCCGGTGACAATAAGTATATGCACCTG | |
| | AAAGTGTTCAACGGCCCGGAATTTTACCAGGGTCATTGGACCAACG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 912 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAAACATAAACATCATCATACCAACTCCACCAA | |
| | CTATTACATTAAGGTTCGTGCCGGTGACAATAAGTATATGCACCTG | |
| | AAAGTGTTCAACGGCCCGGTTGGTCATCATTGGTGGCTGAAAGAAG | |
| | CGGACCGTGTTCTGACCGGTTACCAGGTTGACAAGAACAAAGATG | |
| | ACGAGCTGACGGGTTTC | |
| FcRn-26 | ATGATCCCGCGTGGCCTGTCTGAAGCTAAACCAGCAACTCCGGAAA | 913 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCGTCATAAACATATCCAGGTTCATTCCACCAA | |
| | CTATTACATTAAGGTTCGTGCCGGTGACAATAAGTATATGCACCTG | |
| | AAAGTGTTCAACGGCCCGGTTGGTACCAAACATCTGCGTCAGTCTG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 914 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACATCAGCATAAACTGCATGCACATTCCACCAA | |
| | CTATTACATTAAGGTTCGTGCCGGTGACAATAAGTATATGCACCTG | |
| | AAAGTGTTCAACGGCCCGAAACGTCGTCGTCATCCATCTCGTGGTG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 915 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCGTGATCATGTTTGGCATAAAGGTTCCACCAA | |
| | CTATTACATTAAGGTTCGTGCCGGTGACAATAAGTATATGCACCTG | |
| | AAAGTGTTCAACGGCCCGAACCATGTTCATAACAAACATATCCATG | |
| | CGGACCGTGTTCTGACCGGTTACCAGGTTGACAAGAACAAAGATG | |
| | ACGAGCTGACGGGTTTC | |
| FcRn-29 | ATGATCCCGCGTGGCCTGTCTGAAGCTAAACCAGCAACTCCGGAAA | 916 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCATCGTTCTCATGCAGATCGTCGTTCCACCAA | |
| | CTATTACATTAAGGTTCGTGCCGGTGACAATAAGTATATGCACCTG | |
| | AAAGTGTTCAACGGCCCGACCCAGTCTCATCCACATCGTCATTACG | |
| | CGGACCGTGTTCTGACCGGTTACCAGGTTGACAAGAACAAAGATG | |
| | ACGAGCTGACGGGTTTC | |
| FcRn-30 | ATGATCCCGCGTGGCCTGTCTGAAGCTAAACCAGCAACTCCGGAAA | 917 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTTCTCAGAACGGTTACCAGGGTCATTCCACCAA | |
| | CTATTACATTAAGGTTCGTGCCGGTGACAATAAGTATATGCACCTG | |
| | AAAGTGTTCAACGGCCCGTACCGTCATCATCATCATTGGCATTTTG | |
| | CGGACCGTGTTCTGACCGGTTACCAGGTTGACAAGAACAAAGATG | |
| | ACGAGCTGACGGGTTTC | |
| FcRn-31 | ATGATCCCGCGTGGCCTGTCTGAAGCTAAACCAGCAACTCCGGAAA | 918 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCGAAGGTGGTAAAAAACTGCGTCGTTCCACCA | |
| | ACTATTACATTAAGGTTCGTGCCGGTGACAATAAGTATATGCACCT | |
| | GAAAGTGTTCAACGGCCCGGAATGGACCCATGGTAAAGAAAACCA | |
| | TGCGGACCGTGTTCTGACCGGTTACCAGGTTGACAAGAACAAAGAT | |
| | GACGAGCTGACGGGTTTC | |
| FcRn-32 | ATGATCCCGCGTGGCCTGTCTGAAGCTAAACCAGCAACTCCGGAAA | 919 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAGCACGTCATCATCAGGGTCATGCATCCACCA | |
| | ACTATTACATTAAGGTTCGTGCCGGTGACAATAAGTATATGCACCT | |
| | GAAAGTGTTCAACGGCCCGTGGTACCAGTTTGATGGTGTTTCTTTT | |
| | GCGGACCGTGTTCTGACCGGTTACCAGGTTGACAAGAACAAAGAT | |
| | GACGAGCTGACGGGTTTC | |
| FcRn-33 | ATGATCCCGCGTGGCCTGTCTGAAGCTAAACCAGCAACTCCGGAAA | 920 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCATTCTCAGGGTCGTCATCATATCTCCACCAA | |
| | CTATTACATTAAGGTTCGTGCCGGTGACAATAAGTATATGCACCTG | |
| | AAAGTGTTCAACGGCCCGAAAAAAGTTCGTCATGAATACGCATGG | |
| | GCGGACCGTGTTCTGACCGGTTACCAGGTTGACAAGAACAAAGAT | |
| | GACGAGCTGACGGGTTTC | |
| FcRn-34 | ATGATCCCGCGTGGCCTGTCTGAAGCTAAACCAGCAACTCCGGAAA | 921 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAATACTGGAAAGCAGATTGGTACTGGTCCACCA | |
| | ACTATTACATTAAGGTTCGTGCCGGTGACAATAAGTATATGCACCT | |
| | GAAAGTGTTCAACGGCCCGGAACATTCTTGGTGGCGTCGTGGTCAT | |
| | GCGGACCGTGTTCTGACCGGTTACCAGGTTGACAAGAACAAAGAT | |
| | GACGAGCTGACGGGTTTC | |
| FcRn-35 | ATGATCCCGCGTGGCCTGTCTGAAGCTAAACCAGCAACTCCGGAAA | 922 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCGTCAGTACCCACCAGGTCCACATTCCACCAA | |
| | CTATTACATTAAGGTTCGTGCCGGTGACAATAAGTATATGCACCTG | |
| | AAAGTGTTCAACGGCCCGTACCATTTTCATCATTACTACAAACATG | |
| | CGGACCGTGTTCTGACCGGTTACCAGGTTGACAAGAACAAAGATG | |
| | ACGAGCTGACGGGTTTC | |
| FcRn-36 | ATGATCCCGCGTGGCCTGTCTGAAGCTAAACCAGCAACTCCGGAAA | 923 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCAGCATCATCATTTTTACCGTACCTCCACCAA | |
| | CTATTACATTAAGGTTCGTGCCGGTGACAATAAGTATATGCACCTG | |
| | AAAGTGTTCAACGGCCCGTGGCAGAACTTTCATGATCCATTTGATG | |
| | CGGACCGTGTTCTGACCGGTTACCAGGTTGACAAGAACAAAGATG | |
| | ACGAGCTGACGGGTTTC | |
| FcRn-37 | ATGATCCCGCGTGGCCTGTCTGAAGCTAAACCAGCAACTCCGGAAA | 924 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACAGCAGCATCAGCCAGATCCAACCTCCACCA | |
| | ACTATTACATTAAGGTTCGTGCCGGTGACAATAAGTATATGCACCT | |
| | GAAAGTGTTCAACGGCCCGGCACGTCAGCATCATCATCATTCTCAT | |
| | GCGGACCGTGTTCTGACCGGTTACCAGGTTGACAAGAACAAAGAT | |
| | GACGAGCTGACGGGTTTC | |
| FcRn-38 | ATGATCCCGCGTGGCCTGTCTGAAGCTAAACCAGCAACTCCGGAAA | 925 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACTGTCTTTTAACAACTACCATTGGCATTCCACCAA | |
| | CTATTACATTAAGGTTCGTGCCGGTGACAATAAGTATATGCACCTG | |
| | AAAGTGTTCAACGGCCCGAAACTGCGTCATGATAAACTGACCCATG | |
| | CGGACCGTGTTCTGACCGGTTACCAGGTTGACAAGAACAAAGATG | |
| | ACGAGCTGACGGGTTTC | |
| FcRn-39 | ATGATCCCGCGTGGCCTGTCTGAAGCTAAACCAGCAACTCCGGAAA | 926 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATTCTAAACATCATCATCTGCATTCCACCAA | |
| | CTATTACATTAAGGTTCGTGCCGGTGACAATAAGTATATGCACCTG | |
| | AAAGTGTTCAACGGCCCGAACCATAAATTTCAGTCTTACCAGCCAG | |
| | CGGACCGTGTTCTGACCGGTTACCAGGTTGACAAGAACAAAGATG | |
| | ACGAGCTGACGGGTTTC | |
| FcRn-40 | ATGATCCCGCGTGGCCTGTCTGAAGCTAAACCAGCAACTCCGGAAA | 927 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAAATACGATCGTCATTCTTTTAAATCCACCAA | |
| | CTATTACATTAAGGTTCGTGCCGGTGACAATAAGTATATGCACCTG | |
| | AAAGTGTTCAACGGCCCGGGTAAACATTCTGGTGCACGTCATAAAG | |
| | CGGACCGTGTTCTGACCGGTTACCAGGTTGACAAGAACAAAGATG | |
| | ACGAGCTGACGGGTTTC | |
| FcRn-41 | ATGATCCCGCGTGGCCTGTCTGAAGCTAAACCAGCAACTCCGGAAA | 928 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACATTCTCGTCATCATCATGCACAGTACACCAA | |
| | CTATTACATTAAGGTTCGTGCCGGTGACAATAAGTATATGCACCTG | |
| | AAAGTGTTCAACGGCCCGAACATCCATCATGAAGGTAAAATCCCA | |
| | GCGGACCGTGTTCTGACCGGTTACCAGGTTGACAAGAACAAAGAT | |
| | GACGAGCTGACGGGTTTC | |
| FcRn-42 | ATGATCCCGCGTGGCCTGTCTGAAGCTAAACCAGCAACTCCGGAAA | 929 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCATCATCATTCTCATTTTCATCTGTCCACCAAC | |
| | TATTACATTAAGGTTCGTGCCGGTGACAATAAGTATATGCACCTGA | |
| | AAGTGTTCAACGGCCCGATCCGTCAGTCTTCTTACAAAGTTCATGC | |
| | GGACCGTGTTCTGACCGGTTACCAGGTTGACAAGAACAAAGATGA | |
| | CGAGCTGACGGGTTTC | |
| FcRn-43 | ATGATCCCGCGTGGCCTGTCTGAAGCTAAACCAGCAACTCCGGAAA | 930 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTAACCATCGTCATCCACATGGTCAGTCCACCAA | |
| | CTATTACATTAAGGTTCGTGCCGGTGACAATAAGTATATGCACCTG | |
| | AAAGTGTTCAACGGCCCGGTTCAGCATCGTTGGTCTCTGCATTGGG | |
| | CGGACCGTGTTCTGACCGGTTACCAGGTTGACAAGAACAAAGATG | |
| | ACGAGCTGACGGGTTTC | |
| FcRn-44 | ATGATCCCGCGTGGCCTGTCTGAAGCTAAACCAGCAACTCCGGAAA | 931 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCATGTTGAACAGGTTCATTTTCCATACACCAA | |
| | CTATTACATTAAGGTTCGTGCCGGTGACAATAAGTATATGCACCTG | |
| | AAAGTGTTCAACGGCCCGGGTCATAAACATCATCATCATTGGTCTG | |
| | CGGACCGTGTTCTGACCGGTTACCAGGTTGACAAGAACAAAGATG | |
| | ACGAGCTGACGGGTTTC | |
| FcRn-45 | ATGATCCCGCGTGGCCTGTCTGAAGCTAAACCAGCAACTCCGGAAA | 932 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAACCACATAAACATCATTACCATCTGTCCACCAA | |
| | CTATTACATTAAGGTTCGTGCCGGTGACAATAAGTATATGCACCTG | |
| | AAAGTGTTCAACGGCCCGGTTCCAGGTCAGCAGCCAATCAAAAAC | |
| | GCGGACCGTGTTCTGACCGGTTACCAGGTTGACAAGAACAAAGAT | |
| | GACGAGCTGACGGGTTTC | |
| FcRn-46 | ATGATCCCGCGTGGCCTGTCTGAAGCTAAACCAGCAACTCCGGAAA | 933 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGAAAAAACATAACTGGAAATACAAATCCACCA | |
| | ACTATTACATTAAGGTTCGTGCCGGTGACAATAAGTATATGCACCT | |
| | GAAAGTGTTCAACGGCCCGTGGGCAGCAAAACGTGATTGGCGTAA | |
| | CGCGGACCGTGTTCTGACCGGTTACCAGGTTGACAAGAACAAAGA | |
| | TGACGAGCTGACGGGTTTC | |
| FcRn-47 | ATGATCCCGCGTGGCCTGTCTGAAGCTAAACCAGCAACTCCGGAAA | 934 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAATCCATCATCATACCTGGGGTCTGAAATCCACCAA | |
| | CTATTACATTAAGGTTCGTGCCGGTGACAATAAGTATATGCACCTG | |
| | AAAGTGTTCAACGGCCCGTACGGTGATCAGCCATTTAAACGTCATG | |
| | CGGACCGTGTTCTGACCGGTTACCAGGTTGACAAGAACAAAGATG | |
| | ACGAGCTGACGGGTTTC | |
| FcRn-48 | ATGATCCCGCGTGGCCTGTCTGAAGCTAAACCAGCAACTCCGGAAA | 935 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACCAAAATACCATCATCATGATATCTCCACCAA | |
| | CTATTACATTAAGGTTCGTGCCGGTGACAATAAGTATATGCACCTG | |
| | AAAGTGTTCAACGGCCCGGGTCATCATGCAAAACCACATCGTTGGG | |
| | CGGACCGTGTTCTGACCGGTTACCAGGTTGACAAGAACAAAGATG | |
| | ACGAGCTGACGGGTTTC | |
| FcRn-49 | ATGATCCCGCGTGGCCTGTCTGAAGCTAAACCAGCAACTCCGGAAA | 936 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGTACTGGCATTCTCATGAAACCTGGTCCACCAA | |
| | CTATTACATTAAGGTTCGTGCCGGTGACAATAAGTATATGCACCTG | |
| | AAAGTGTTCAACGGCCCGTTTCTGAAAGTTCGTACCATCCGTTCTG | |
| | CGGACCGTGTTCTGACCGGTTACCAGGTTGACAAGAACAAAGATG | |
| | ACGAGCTGACGGGTTTC | |
| FcRn-50 | ATGATCCCGCGTGGCCTGTCTGAAGCTAAACCAGCAACTCCGGAAA | 937 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTAAACAGTACCATCTGCCATGGACCTCCACCAA | |
| | CTATTACATTAAGGTTCGTGCCGGTGACAATAAGTATATGCACCTG | |
| | AAAGTGTTCAACGGCCCGCTGTCTCAGTTTCAGACCCATCTGTGGG | |
| | CGGACCGTGTTCTGACCGGTTACCAGGTTGACAAGAACGAAGATG | |
| | ACGAGCTGACGGGTTTC | |
| FcRn-51 | ATGATCCCGCGTGGCCTGTCTGAAGCTAAACCAGCAACTCCGGAAA | 938 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGCAATCCATTGGGCACATTACATCCTGTCCACCAA | |
| | CTATTACATTAAGGTTCGTGCCGGTGACAATAAGTATATGCACCTG | |
| | AAAGTGTTCAACGGCCCGGTTCTGTGGCGTTACTACTACCCAAAAG | |
| | CGGACCGTGTTCTGACCGGTTACCAGGTTGACAAGAACAAAGATG | |
| | ACGAGCTGACGGGTTTC | |
| FcRn-52 | ATGATCCCGCGTGGCCTGTCTGAAGCTAAACCAGCAACTCCGGAAA | 939 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATTGGCGTAAACTGACCCTGTTTTCCACCAACTA | |
| | TTACATTAAGGTTCGTGCCGGTGACAATAAGTATATGCACCTGAAA | |
| | GTGTTCAACGGCCCGCATCATCAGCATTGGCATGTTTTTCCAGCGG | |
| | ACCGTGTTCTGACCGGTTACCAGGTTGACAAGAACAAAGATGACG | |
| | AGCTGACGGGTTTC | |
| FcRn-53 | ATGATCCCGCGTGGCCTGTCTGAAGCTAAACCAGCAACTCCGGAAA | 940 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCAAATCTCATAAATTTGCATACCATTCCACCAA | |
| | CTATTACATTAAGGTTCGTGCCGGTGACAATAAGTATATGCACCTG | |
| | AAAGTGTTCAACGGCCCGATCGTTCAGGAATTTTCTCTGGATCAGT | |
| | GGGCGGACCGTGTTCTGACCGGTTACCAGGTTGACAAGAACAAAG | |
| | ATGACGAGCTGACGGGTTTC | |
| FcRn-54 | ATGATCCCGCGTGGCCTGTCTGAAGCTAAACCAGCAACTCCGGAAA | 941 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTAAATACGTTCATTGGCATAAATTTTCCACCAA | |
| | CTATTACATTAAGGTTCGTGCCGGTGACAATAAGTATATGCACCTG | |
| | AAAGTGTTCAACGGCCCGTGGAAAATCAACAACCTGTACCATGAA | |
| | GCGGACCGTGTTCTGACCGGTTACCAGGTTGACAAGAACAAAGAT | |
| | GACGAGCTGACGGGTTTC | |
| FcRn-55 | ATGATCCCGCGTGGCCTGTCTGAAGCTAAACCAGCAACTCCGGAAA | 942 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAGAACAGGCAGCATGGGTTCTGCATTCCACCA | |
| | ACTATTACATTAAGGTTCGTGCCGGTGACAATAAGTATATGCACCT | |
| | GAAAGTGTTCAACGGCCCGTTTCATTACCTGCATCATACCCGTTCT | |
| | GCGGACCGTGTTCTGACCGGTTACCAGGTTGACAAGAACAAAGAT | |
| | GACGAGCTGACGGGTTTC | |
| FcRn-56 | ATGATCCCGCGTGGCCTGTCTGAAGCTAAACCAGCAACTCCGGAAA | 943 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCTGCAGGCACCACGTAACGCATACTCCACCAA | |
| | CTATTACATTAAGGTTCGTGCCGGTGACAATAAGTATATGCACCTG | |
| | AAAGTGTTCAACGGCCCGAAAGGTTGGCGTAACACCCATCATAAA | |
| | GCGGACCGTGTTCTGACCGGTTACCAGGTTGACAAGAACAAAGAT | |
| | GACGAGCTGACGGGTTTC | |
| FcRn-57 | ATGATCCCGCGTGGCCTGTCTGAAGCTAAACCAGCAACTCCGGAAA | 944 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCTGACCCATCGTTGGCGTCCACATTCCACCAA | |
| | CTATTACATTAAGGTTCGTGCCGGTGACAATAAGTATATGCACCTG | |
| | AAAGTGTTCAACGGCCCGATCTGGTCTGCACGTTCTGATAAACTGG | |
| | CGGACCGTGTTCTGACCGGTTACCAGGTTGACAAGAACAAAGATG | |
| | ACGAGCTGACGGGTTTC | |
| FcRn-58 | ATGATCCCGCGTGGCCTGTCTGAAGCTAAACCAGCAACTCCGGAAA | 945 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCATCATCGTGCAACCGATCAGGTTTCCACCAA | |
| | CTATTACATTAAGGTTCGTGCCGGTGACAATAAGTATATGCACCTG | |
| | AAAGTGTTCAACGGCCCGAAAGCATACCATACCTACTGGCATCATG | |
| | CGGACCGTGTTCTGACCGGTTACCAGGTTGACAAGAACAAAGATG | |
| | ACGAGCTGACGGGTTTC | |
| FcRn-59 | ATGATCCCGCGTGGCCTGTCTGAAGCTAAACCAGCAACTCCGGAAA | 946 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACAAATGGCATATCCGTTTTGCAACCTCCACCAA | |
| | CTATTACATTAAGGTTCGTGCCGGTGACAATAAGTATATGCACCTG | |
| | AAAGTGTTCAACGGCCCGTTTGCACAGGCACATCATCATACCCAGG | |
| | CGGACCGTGTTCTGACCGGTTACCAGGTTGACAAGAACAAAGATG | |
| | ACGAGCTGACGGGTTTC | |
| FcRn-60 | ATGATCCCGCGTGGCCTGTCTGAAGCTAAACCAGCAACTCCGGAAA | 947 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATATCCGTGATTCTCTGTGGATCACCTCCACCAA | |
| | CTATTACATTAAGGTTCGTGCCGGTGACAATAAGTATATGCACCTG | |
| | AAAGTGTTCAACGGCCCGAACTGGCAGTGGATCCCACATTGGGCA | |
| | GCGGACCGTGTTCTGACCGGTTACCAGGTTGACAAGAACAAAGAT | |
| | GACGAGCTGACGGGTTTC | |
| FcRn-61 | ATGATCCCGCGTGGCCTGTCTGAAGCTAAACCAGCAACTCCGGAAA | 948 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCATATCTCTCTGTCTTTTCGTGAATCCACCAAC | |
| | TATTACATTAAGGTTCGTGCCGGTGACAATAAGTATATGCACCTGA | |
| | AAGTGTTCAACGGCCCGAAACTGGATACCCTGGGTCAGCAGCGTG | |
| | CGGACCGTGTTCTGACCGGTTACCAGGTTGACAAGAACAAAGATG | |
| | ACGAGCTGACGGGTTTC | |
| FcRn-62 | ATGATCCCGCGTGGCCTGTCTGAAGCTAAACCAGCAACTCCGGAAA | 949 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAATCCATTGGGCAGGTTTTTTTCGTGGTTCCACCAA | |
| | CTATTACATTAAGGTTCGTGCCGGTGACAATAAGTATATGCACCTG | |
| | AAAGTGTTCAACGGCCCGTGGGAATGGGAACGTCATTGGCTGGCA | |
| | GCGGACCGTGTTCTGACCGGTTACCAGGTTGACAAGAACAAAGAT | |
| | GACGAGCTGACGGGTTTC | |
| FcRn-63 | ATGATCCCGCGTGGCCTGTCTGAAGCTAAACCAGCAACTCCGGAAA | 950 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACTACTCTGAACGTCATTTTTACAAATCCACCAA | |
| | CTATTACATTAAGGTTCGTGCCGGTGACAATAAGTATATGCACCTG | |
| | AAAGTGTTCAACGGCCCGTTTACCCTGGGTCGTGAAGGTTGGTTTG | |
| | CGGACCGTGTTCTGACCGGTTACCAGGTTGACAAGAACAAAGATG | |
| | ACGAGCTGACGGGTTTC | |
| FcRn-64 | ATGATCCCGCGTGGCCTGTCTGAAGCTAAACCAGCAACTCCGGAAA | 951 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCAGCAGCAGGTTCATGTTCCATCTTCCACCAA | |
| | CTATTACATTAAGGTTCGTGCCGGTGACAATAAGTATATGCACCTG | |
| | AAAGTGTTCAACGGCCCGTACCGTGGTAACACCTTTAAAATCTGGG | |
| | CGGACCGTGTTCTGACCGGTTACCAGGTTGACAAGAACAAAGATG | |
| | ACGAGCTGACGGGTTTC | |
| FcRn-65 | ATGATCCCGCGTGGCCTGTCTGAAGCTAAACCAGCAACTCCGGAAA | 952 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCAAAAAAAACCAGCTGCAGGGTCATTCCACCA | |
| | ACTATTACATTAAGGTTCGTGCCGGTGACAATAAGTATATGCACCT | |
| | GAAAGTGTTCAACGGCCCGGTTCATTCTCTGCTGCAGCATCATGAT | |
| | GCGGACCGTGTTCTGACCGGTTACCAGGTTGACAAGAACAAAGAT | |
| | GACGAGCTGACGGGTTTC | |
| FcRn-66 | ATGATCCCGCGTGGCCTGTCTGAAGCTAAACCAGCAACTCCGGAAA | 953 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTGATATCCATCATCATCATCATTGGTCCACCAA | |
| | CTATTACATTAAGGTTCGTGCCGGTGACAATAAGTATATGCACCTG | |
| | AAAGTGTTCAACGGCCCGTACATCAAACGTCATTGGTCTAACTTTG | |
| | CGGACCGTGTTCTGACCGGTTACCAGGTTGACAAGAACAAAGATG | |
| | ACGAGCTGACGGGTTTC | |
| FcRn-67 | ATGATCCCGCGTGGCCTGTCTGAAGCTAAACCAGCAACTCCGGAAA | 954 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGCGTCAGTACACCACCAAGGTTCTGTCCACCAA | |
| | CTATTACATTAAGGTTCGTGCCGGTGACAATAAGTATATGCACCTG | |
| | AAAGTGTTCAACGGCCCGGATAACGAACGTAACCAGGTTGAATCT | |
| | GCGGACCGTGTTCTGACCGGTTACCAGGTTGACAAGAACAAAGAT | |
| | GACGAGCTGACGGGTTTC | |
| FcRn-68 | ATGATCCCGCGTGGCCTGTCTGAAGCTAAACCAGCAACTCCGGAAA | 955 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACTGGGATTGGCGTTTTGTTGAATGGTCCACCAA | |
| | CTATTACATTAAGGTTCGTGCCGGTGACAATAAGTATATGCACCTG | |
| | AAAGTGTTCAACGGCCCGATCGGTTACGAACTGTTTACCGTTAAAG | |
| | CGGACCGTGTTCTGACCGGTTACCAGGTTGACAAGAACAAAGATG | |
| | ACGAGCTGACGGGTTTC | |
| FcRn-69 | ATGATCCCGCGTGGCCTGTCTGAAGCTAAACCAGCAACTCCGGAAA | 956 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTTTTTCTAAACCATTTAAATGGTACTCCACCAA | |
| | CTATTACATTAAGGTTCGTGCCGGTGACAATAAGTATATGCACCTG | |
| | AAAGTGTTCAACGGCCCGTACCGTGCATGGATCCATTGGACCTCTG | |
| | CGGACCGTGTTCTGACCGGTTACCAGGTTGACAAGAACAAAGATG | |
| | ACGAGCTTACGGGTTTC | |
| FcRn-70 | ATGATCCCGCGTGGCCTGTCTGAAGCTAAACCAGCAACTCCGGAAA | 957 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAATCTTTCAGGAACGTCTGGCAGGTCAGTCCACCAA | |
| | CTATTACATTAAGGTTCGTGCCGGTGACAATAAGTATATGCACCTG | |
| | AAAGTGTTCAACGGCCCGCAGATCAAACATTCTCATCATGCATGGG | |
| | CGGACCGTGTTCTGACCGGTTACCAGGTTGACAAGAACAAAGATG | |
| | ACGAGCTGACGGGTTTC | |
| FcRn-71 | ATGATCCCGCGTGGCCTGTCTGAAGCTAAACCAGCAACTCCGGAAA | 958 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAATACGATCATCATACCCAGTCTCTGTCCACCAA | |
| | CTATTACATTAAGGTTCGTGCCGGTGACAATAAGTATATGCACCTG | |
| | AAAGTGTTCAACGGCCCGGTTTACGCATGGTACTGGGATAAATGGG | |
| | CGGACCGTGTTCTGACCGGTTACCAGGTTGACAAGAACAAAGATG | |
| | ACGAGCTGACGGGTTTC | |
| FcRn-72 | ATGATCCCGCGTGGCCTGTCTGAAGCTAAACCAGCAACTCCGGAAA | 959 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACATGCACATACCCCATTTGGTCCATCCACCAA | |
| | CTATTACATTAAGGTTCGTGCCGGTGACAATAAGTATATGCACCTG | |
| | AAAGTGTTCAACGGCCCGGCAGTTTGGTGGGATGGTCGTGGTTGGG | |
| | CGGACCGTGTTCTGACCGGTTACCAGGTTGACAAGAACAAAGATG | |
| | ACGAGCTGACGGGTTTC | |
| FcRn-73 | ATGATCCCGCGTGGCCTGTCTGAAGCTAAACCAGCAACTCCGGAAA | 960 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCTGTCTCGTTGGCTGTGGGCAGAATCCACCAA | |
| | CTATTACATTAAGGTTCGTGCCGGTGACAATAAGTATATGCACCTG | |
| | AAAGTGTTCAACGGCCCGTGGCATACCCATAAACATTACCAGAAA | |
| | GCGGACCGTGTTCTGACCGGTTACCAGGTTGACAAGAACAAAGAT | |
| | GACGAGCTGACGGGTTTC | |
| FcRn-74 | ATGATCCCGCGTGGCCTGTCTGAAGCTAAACCAGCAACTCCGGAAA | 961 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCAGCAGCATACCCAGCGTTACCGTTCCACCAA | |
| | CTATTACATTAAGGTTCGTGCCGGTGACAATAAGTATATGCACCTG | |
| | AAAGTGTTCAACGGCCCGGCAAAACTGCAGTTTGGTCATAAACATG | |
| | CGGACCGTGTTCTGACCGGTTACCAGGTTGACAAGAACAAAGATG | |
| | ACGAGCTGACGGGTTTC | |
| FcRn-75 | ATGATCCCGCGTGGCCTGTCTGAAGCTAAACCAGCAACTCCGGAAA | 962 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATACCATCTCTCAGCATGTTTCCACCAACTATTA | |
| | CATTAAGGTTCGTGCCGGTGACAATAAGTATATGCACCTGAAAGTG | |
| | TTCAACGGCCCGCCAATCTCTTTTCGTTGGCATCGTTTTGCGGACCG | |
| | TGTTCTGACCGGTTACCAGGTTGACAAGAACAAAGATGACGAGCT | |
| | GACGGGTTTC | |
| FcRn-76 | ATGATCCCGCGTGGCCTGTCTGAAGCTAAACCAGCAACTCCGGAAA | 963 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCAGTGGACCTGGGCACATTCTCGTTCCACCAA | |
| | CTATTACATTAAGGTTCGTGCCGGTGACAATAAGTATATGCACCTG | |
| | AAAGTGTTCAACGGCCCGGATTACCATCTGCGTCATCATAACCATG | |
| | CGGACCGTGTTCTGACCGGTTACCAGGTTGACAAGAACAAAGATG | |
| | ACGAGCTGACGGGTTTC | |
| FcRn-77 | ATGATCCCGCGTGGCCTGTCTGAAGCTAAACCAGCAACTCCGGAAA | 964 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGTACCGTGTTTGGCGTTGGGTTTGGTCCACCAA | |
| | CTATTACATTAAGGTTCGTGCCGGTGACAATAAGTATATGCACCTG | |
| | AAAGTGTTCAACGGCCCGGTTTACAAATACGGTTCTGAAAACTGGG | |
| | CGGACCGTGTTCTGACCGGTTACCAGGTTGACAAGAACAAAGATG | |
| | ACGAGCTGACGGGTTTC | |
| FcRn-78 | ATGATCCCGCGTGGCCTGTCTGAAGCTAAACCAGCAACTCCGGAAA | 965 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGAAAGGTTCTACCCATCATAACCATTCCACCAA | |
| | CTATTACATTAAGGTTCGTGCCGGTGACAATAAGTATATGCACCTG | |
| | AAAGTGTTCAACGGCCCGGCACGTTCTCAGGCAGGTCATCATAACG | |
| | CGGACCGTGTTCTGACCGGTTACCAGGTTGACAAGAACAAAGATG | |
| | ACGAGCTGACGGGTTTC | |
| FcRn-79 | ATGATCCCGCGTGGCCTGTCTGAAGCTAAACCAGCAACTCCGGAAA | 966 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAGAAGGTCGTGCAGGTGAACCATCTTCCACCA | |
| | ACTATTACATTAAGGTTCGTGCCGGTGACAATAAGTATATGCACCT | |
| | GAAAGTGTTCAACGGCCCGGAACATTGGTGGTTTACCTTTGGTGAT | |
| | GCGGACCGTGTTCTGACCGGTTACCAGGTTGACAAGAACAAAGAT | |
| | GACGAGCTGACGGGTTTC | |
| FcRn-80 | ATGATCCCGCGTGGCCTGTCTGAAGCTAAACCAGCAACTCCGGAAA | 967 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATACCCGTCATCATGTTACCCTGTGGTCCACCAA | |
| | CTATTACATTAAGGTTCGTGCCGGTGACAATAAGTATATGCACCTG | |
| | AAAGTTTTCAACGGCCCGGGTTGGAAATACGCACCACAGGTTTGGG | |
| | CGGACCGTGTTCTGACCGGTTACCAGGTTGACAAGAACAAAGATG | |
| | ACGAGCTGACGGGTTTC | |
| FcRn-81 | ATGATCCCGCGTGGCCTGTCTGAAGCTAAACCAGCAACTCCGGAAA | 968 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGCGGTACTACAAACATGAATACCGTTCCACCA | |
| | ACTATTACATTAAGGTTCGTGCCGGTGACAATAAGTATATGCACCT | |
| | GAAAGTGTTCAACGGCCCGTACTTTAAACTGCCACCATGGGAAGA | |
| | AGCGGACCGTGTTCTGACCGGTTACCAGGTTGACAAGAACAAAGA | |
| | TGACGAGCTGACGGGTTTC | |
| FcRn-82 | ATGATCCCGCGTGGCCTGTCTGAAGCTAAACCAGCAACTCCGGAAA | 969 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGTGGTTTCATCGTCGTGAAGTTAAATCCACCAA | |
| | CTATTACATTAAGGTTCGTGCCGGTGACAATAAGTATATGCACCTG | |
| | AAAGTGTTCAACGGCCCGCCAGTTCATCTGCATCATAAACAGCATG | |
| | CGGACCGTGTTCTGACCGGTTACCAGGTTGACAAGAACAAAGATG | |
| | ACGAGCTGACGGGTTTC | |
| FcRn-83 | ATGATCCCGCGTGGCCTGTCTGAAGCTAAACCAGCAACTCCGGAAA | 970 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATCTGCATGCAACCCAGCCACCATCCACCAA | |
| | CTATTACATTAAGGTTCGTGCCGGTGACAATAAGTATATGCACCTG | |
| | AAAGTGTTCAACGGCCCGAACTGGCATATCATCAACAAATACGAT | |
| | GCGGACCGTGTTCTGACCGGTTACCAGGTTGACAAGAACAAAGAT | |
| | GACGAGCTGACGGGTTTC | |
| FcRn-84 | ATGATCCCGCGTGGCCTGTCTGAAGCTAAACCAGCAACTCCGGAAA | 971 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACATTGGCATCAGCCAGTTGCAAAATCCACCA | |
| | ACTATTACATTAAGGTTCGTGCCGGTGACAATAAGTATATGCACCT | |
| | GAAAGTGTTCAACGGCCCGGCACATTGGCATGATTGGGTTGCGGAC | |
| | CGTGTTCTGACCGGTTACCAGGTTGACAAGAACAAAGATGACGAG | |
| | CTGACGGGTTTC | |
| FcRn-85 | ATGATCCCGCGTGGCCTGTCTGAAGCTAAACCAGCAACTCCGGAAA | 972 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACACCACCTCTCATTGGACCATCGGTTCCACCAA | |
| | CTATTACATTAAGGTTCGTGCCGGTGACAATAAGTATATGCACCTG | |
| | AAAGTGTTCAACGGCCCGGATCATCATCATGTTCAGAAATCTCATG | |
| | CGGACCGTGTTCTGACCGGTTACCAGGTTGACAAGAACAAAGATG | |
| | ACGAGCTGACGGGTTTC | |
| FcRn-86 | ATGATCCCGCGTGGCCTGTCTGAAGCTAAACCAGCAACTCCGGAAA | 973 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAACATCATCATACCCAGCTGTCTAACTCCACCAA | |
| | CTATTACATTAAGGTTCGTGCCGGTGACAATAAGTATATGCACCTG | |
| | AAAGTGTTCAACGGCCCGAAATTTTGGCAGGTTCAGCAGAAATAC | |
| | GCGGACCGTGTTCTGACCGGTTACCAGGTTGACAAGAACAAAGAT | |
| | GACGAGCTGACGGGTTTC | |
| FcRn-87 | ATGATCCCGCGTGGCCTGTCTGAAGCTAAACCAGCAACTCCGGAAA | 974 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAAACCACATAACTCTAAACAGATCTCCACCAA | |
| | CTATTACATTAAGGTTCGTGCCGGTGACAATAAGTATATGCACCTG | |
| | AAAGTGTTCAACGGCCCGAAACCACGTTTTAACATCCATCATCATG | |
| | CGGACCGTGTTCTGACCGGTTACCAGGTTGACAAGAACAAAGATG | |
| | ACGAGCTGACGGGTTTC | |
| FcRn-88 | ATGATCCCGCGTGGCCTGTCTGAAGCTAAACCAGCAACTCCGGAAA | 975 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATACCAAACATCATTCTCGTTGGTCCACCAA | |
| | CTATTACATTAAGGTTCGTGCCGGTGACAATAAGTATATGCACCTG | |
| | AAAGTGTTCAACGGCCCGGTTAACCATATCTCTCATGCACCAATCG | |
| | CGGACCGTGTTCTGACCGGTTACCAGGTTGACAAGAACAAAGATG | |
| | ACGAGCTGACGGGTTTC | |
| FcRn-89 | ATGATCCCGCGTGGCCTGTCTGAAGCTAAACCAGCAACTCCGGAAA | 976 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATTTCATCGTCATCATCCAATCTGGCATTCCACCAA | |
| | CTATTACATTAAGGTTCGTGCCGGTGACAATAAGTATATGCACCTG | |
| | AAAGTGTTCAACGGCCCGCTGAAACCATGGGAAGCAGATCTGTGG | |
| | GCGGACCGTGTTCTGACCGGTTACCAGGTTGACAAGAACAAAGAT | |
| | GACGAGCTGACGGGTTTC | |
| FcRn-90 | ATGATCCCGCGTGGCCTGTCTGAAGCTAAACCAGCAACTCCGGAAA | 977 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGCACGTGTTACCATCGATTGGAAAGCATCCACCAA | |
| | CTATTACATTAAGGTTCGTGCCGGTGACAATAAGTATATGCACCTG | |
| | AAAGTGTTCAACGGCCCGTACAAATACCCAAACATCCATCCACATG | |
| | CGGACCGTGTTCTGACCGGTTACCAGGTTGACAAGAACAAAGATG | |
| | ACGAGCTGACGGGTTTC | |
| FcRn-91 | ATGATCCCGCGTGGCCTGTCTGAAGCTAAACCAGCAACTCCGGAAA | 978 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACTGGAACAGCGTCGTTCTCATTACTCCACCAA | |
| | CTATTACATTAAGGTTCGTGCCGGTGACAATAAGTATATGCACCTG | |
| | AAAGTGTTCAACGGCCCGCCAAAATCTCTGTTTAACTACCAGCATG | |
| | CGGACCGTGTTCTGACCGGTTACCAGGTTGACAAGAACAAAGATG | |
| | ACGAGCTGACGGGTTTC | |
| FcRn-92 | ATGATCCCGCGTGGCCTGTCTGAAGCTAAACCAGCAACTCCGGAAA | 979 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACATCCATCATGTTCATCATCAGCAGTCCACCAA | |
| | CTATTACATTAAGGTTCGTGCCGGTGACAATAAGTATATGCACCTG | |
| | AAAGTGTTCAACGGCCCGGATGGTGAATTTCATGTTAAACAGGTTG | |
| | CGGACCGTGTTCTGACCGGTTACCAGGTTGACAAGAACAAAGATG | |
| | ACGAGCTGACGGGTTTC | |
| FcRn-93 | ATGATCCCGCGTGGCCTGTCTGAAGCTAAACCAGCAACTCCGGAAA | 980 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCATCATACCATCGCATGGTACGTTTCCACCAA | |
| | CTATTACATTAAGGTTCGTGCCGGTGACAATAAGTATATGCACCTG | |
| | AAAGTGTTCAACGGCCCGGTTTACCCAAAACGTCAGCAGGTTGAA | |
| | GCGGACCGTGTTCTGACCGGTTACCAGGTTGACAAGAACAAAGAT | |
| | GACGAGCTGACGGGTTTC | |
| FcRn-94 | ATGATCCCGCGTGGCCTGTCTGAAGCTAAACCAGCAACTCCGGAAA | 981 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATCAGCCATACTACGGTTGGCAGTCCACCAA | |
| | CTATTACATTAAGGTTCGTGCCGGTGACAATAAGTATATGCACCTG | |
| | AAAGTGTTCAACGGCCCGATCATCGATCGTTCTAAAATCGAAAAAG | |
| | CGGACCGTGTTCTGACCGGTTACCAGGTTGACAAGAACAAAGATG | |
| | ACGAGCTGACGGGTTTC | |
| FcRn-95 | ATGATCCCGCGTGGCCTGTCTGAAGCTAAACCAGCAACTCCGGAAA | 982 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTCATCGTTCTCATCATCCAATCAAATCCACCAA | |
| | CTATTACATTAAGGTTCGTGCCGGTGACAATAAGTATATGCACCTG | |
| | AAAGTGTTCAACGGCCCGTCTATCCATTCTTCTTGGAAAAAACAGG | |
| | CGGACCGTGTTCTGACCGGTTACCAGGTTGACAAGAACAAAGATG | |
| | ACGAGCTGACGGGTTTC | |
| FcRn-96 | ATGATCCCGCGTGGCCTGTCTGAAGCTAAACCAGCAACTCCGGAAA | 983 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGTGGTCTCAGCGTGTTAAACTGTTTTCCACCAA | |
| | CTATTACATTAAGGTTCGTGCCGGTGACAATAAGTATATGCACCTG | |
| | AAAGTGTTCAACGGCCCGAACATCCATAAAACCTGGGATCAGACC | |
| | GCGGACCGTGTTCTGACCGGTTACCAGGTTGACAAGAACAAAGAT | |
| | GACGAGCTGACGGGTTTC | |
| FcRn-97 | ATGATCCCGCGTGGCCTGTCTGAAGCTAAACCAGCAACTCCGGAAA | 984 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATTACTGGAAACCACATGATATCCATTCCACCAA | |
| | CTATTACATTAAGGTTCGTGCCGGTGACAATAAGTATATGCACCTG | |
| | AAAGTGTTCAACGGCCCGGGTAAAGTTCCATTTCATGCATTTCATA | |
| | AAGCGGACCGTGTTCTGACCGGTTACCAGGTTGACAAGAACAAAG | |
| | ATGACGAGCTGACGGGTTTC | |
| FcRn-98 | ATGATCCCGCGTGGCCTGTCTGAAGCTAAACCAGCAACTCCGGAAA | 985 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCAACCAGCCACGTCTGTACCATCAGTCCACCAA | |
| | CTATTACATTAAGGTTCGTGCCGGTGACAATAAGTATATGCACCTG | |
| | AAAGTGTTCAACGGCCCGTTTTACCGTCTGACCCATGGTCATCGTG | |
| | CGGACCGTGTTCTGACCGGTTACCAGGTTGACAAGAACAAAGATG | |
| | ACGAGCTGACGGGTTTC | |
| FcRn-99 | ATGATCCCGCGTGGCCTGTCTGAAGCTAAACCAGCAACTCCGGAAA | 986 |
| | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGTCTGGTAAACTGCTGAAACATCCATCCACCAA | |
| | CTATTACATTAAGGTTCGTGCCGGTGACAATAAGTATATGCACCTG | |
| | AAAGTGTTCAACGGCCCGCATATCGATTACAAAAACGGTCGTATCT | |
| | GGGCGGACCGTGTTCTGACCGGTTACCAGGTTGACAAGAACAAAG | |
| | ATGACGAGCTGACGGGTTTC | |
| FcRn-10 | ATGATCCCGCGTGGCCTGTCTGAAGCTAAACCAGCAACTCCGGAAA | 987 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCGTACCTCTTGGGATCATAAAAACTCCACCAA | |
| | CTATTACATTAAGGTTCGTGCCGGTGACAATAAGTATATGCACCTG | |
| | AAAGTGTTCAACGGCCCGGTTTTTCATCATCAGCGTGGTGGTCAGG | |
| | CGGACCGTGTTCTGACCGGTTACCAGGTTGACAAGAACAAAGATG | |
| | ACGAGCTGACGGGTTTC | |
| FcRn-10 | ATGATCCCGCGTGGCCTGTCTGAAGCTAAACCAGCAACTCCGGAAA | 988 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACATAAACAGAAACGTCATTTTTTTAACTCCAC | |
| | CAACTATTACATTAAGGTTCGTGCCGGTGACAATAAGTATATGCAC | |
| | CTGAAAGTGTTCAACGGCCCGTGGGGTCAGTCTAAACCAGCACATG | |
| | TTGCGGACCGTGTTCTGACCGGTTACCAGGTTGACAAGAACAAAGA | |
| | TGACGAGCTGACGGGTTTC | |
| FcRn-10 | ATGATCCCGCGTGGCCTGTCTGAAGCTAAACCAGCAACTCCGGAAA | 989 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATGATCAGCATAAACATGATTTTAAATCCACCAA | |
| | CTATTACATTAAGGTTCGTGCCGGTGACAATAAGTATATGCACCTG | |
| | AAAGTGTTCAACGGCCCGTTTCATCAGCGTTTTCCAGATCATAAAG | |
| | CGGACCGTGTTCTGACCGGTTACCAGGTTGACAAGAACAAAGATG | |
| | ACGAGCTGACGGGTTTC | |
| FcRn-10 | ATGATCCCGCGTGGCCTGTCTGAAGCTAAACCAGCAACTCCGGAAA | 990 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCGTGTTGTTCATCATTTTCATCACTCCACCAA | |
| | CTATTACATTAAGGTTCGTGCCGGTGACAATAAGTATATGCACCTG | |
| | AAAGTGTTCAAAGGCCCGATCCAGGCAGCAGAAGGTTACAAACAT | |
| | GCGGACCGTGTTCTGACCGGTTACCAGGTTGACAAGAACAAAGAT | |
| | GACGAGCTGACGGGTTTC | |
| FcRn-10 | ATGATCCCGCGTGGCCTGTCTGAAGCTAAACCAGCAACTCCGGAAA | 991 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCATAAAGCAATCCGTCAGCAGTTTTCCACCAA | |
| | CTATTACATTAAGGTTCGTGCCGGTGACAATAAGTATATGCACCTG | |
| | AAAGTGTTCAACGGCCCGTTTCATTACCAGTACCGTCATCAGCATG | |
| | CGGACCGTGTTCTGACCGGTTACCAGGTTGACAAGAACAAAGATG | |
| | ACGAGCTGACGGGTTTC | |
| FcRn-10 | ATGATCCCGCGTGGCCTGTCTGAAGCTAAACCAGCAACTCCGGAAA | 992 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCAAAGAATGGCATCAGCATATCAAATCCACCA | |
| | ACTATTACATTAAGGTTCGTGCCGGTGACAATAAGTATATGCACCT | |
| | GAAAGTGTTCAACGGCCCGAACAAATTTCTGCATGGTTTTGAAGTT | |
| | GCGGACCGTGTTCTGACCGGTTACCAGGTTGACAAGAACAAAGAT | |
| | GACGAGCTGACGGGTTTC | |
| FcRn-10 | ATGATCCCGCGTGGCCTGTCTGAAGCTAAACCAGCAACTCCGGAAA | 993 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGTACCATACCCATTTTGCAAACGCATCCACCAA | |
| | CTATTACATTAAGGTTCGTGCCGGTGACAATAAGTATATGCACCTG | |
| | AAAGTGTTCAACGGCCCGTTTAAACGTCATCAGCATGGTCATAAAG | |
| | CGGACCGTGTTCTGACCGGTTACCAGGTTGACAAGAACAAAGATG | |
| | ACGAGCTGACGGGTTTC | |
| FcRn-10 | ATGATCCCGCGTGGCCTGTCTGAAGCTAAACCAGCAACTCCGGAAA | 994 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCGTGTTCATAACCTGTCTGTTCTGTCCACCAA | |
| | CTATTACATTAAGGTTCGTGCCGGTGACAATAAGTATATGCACCTG | |
| | AAAGTGTTCAACGGCCCGCATTACGATCGTGCACATTACTTTAAAG | |
| | CGGACCGTGTTCTGACCGGTTACCAGGTTGACAAGAACAAAGATG | |
| | ACGAGCTGACGGGTTTC | |
| FcRn-10 | ATGATCCCGCGTGGCCTGTCTGAAGCTAAACCAGCAACTCCGGAAA | 995 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGAACCAGCCATACTGGACCACCTACTCCACCAA | |
| | CTATTACATTAAGGTTCGTGCCGGTGACAATAAGTATATGCACCTG | |
| | AAAGTGTTCAACGGCCCGTTTCGTTGGAAATTTCATGATTACAAAG | |
| | CGGACCGTGTTCTGACCGGTTACCAGGTTGACAAGAACAAAGATG | |
| | ACGAGCTGACGGGTTTC | |
| FcRn-10 | ATGATCCCGCGTGGCCTGTCTGAAGCTAAACCAGCAACTCCGGAAA | 996 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCCACATAACCGTGATTCTCATCGTTCCACCAA | |
| | CTATTACATTAAGGTTCGTGCCGGTGACAATAAGTATATGCACCTG | |
| | AAAGTGTTCAACGGCCCGGATCGTAAACATCGTAAACATTGGCATG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 997 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCATCCACGTCATCATTGGAAATACTCCACCAA | |
| | CTATTACATTAAGGTTCGTGCCGGTGACAATAAGTATATGCACCTG | |
| | AAAGTGTTCAACGGCCCGGCAACCTACAAATACCGTGTTGATTACG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 998 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCCAGGTCATCATCATGCACGTGATTCCACCAA | |
| | CTATTACATTAAGGTTCGTGCCGGTGACAATAAGTATATGCACCTG | |
| | AAAGTGTTCAACGGCCCGTACTTTTACCATCATCATTGGTTTAAAG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 999 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAATCGCAAAACATCATACCTGGCATCAGTCCACCAA | |
| | CTATTACATTAAGGTTCGTGCCGGTGACAATAAGTATATGCACCTG | |
| | AAAGTGTTCAACGGCCCGTACCGTAACCATCGTCATCATATCGTTG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 1000 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAACCATGGTCATTGGCATTTTCGTTCCACCAA | |
| | CTATTACATTAAGGTTCGTGCCGGTGACAATAAGTATATGCACCTG | |
| | AAAGTGTTCAACGGCCCGGTTCAGCATGCACGTCATAAACATTACG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 1001 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAAAATTTGATCATTACCATCAGAAATCCACCAA | |
| | CTATTACATTAAGGTTCGTGCCGGTGACAATAAGTATATGCACCTG | |
| | AAAGTGTTCAACGGCCCGAAAGATCGTCATCATCATAACCGTGCGG | |
| | ACCGTGTTCTGACCGGTTACCAGGTTGACAAGAACAAAGATGACG | |
| | AGCTGACGGGTTTC | |
| FcRn-11 | ATGATCCCGCGTGGCCTGTCTGAAGCTAAACCAGCAACTCCGGAAA | 1002 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTAAAGCACATCGTGTTGAACATAAATCCACCAA | |
| | CTATTACATTAAGGTTCGTGCCGGTGACAATAAGTATATGCACCTG | |
| | AAAGTGTTCAACGGCCCGAAACAGCATCATCTGTACCATTTTAAAG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 1003 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAAAAAAACATTACCATCATGGTATCTCCACCAA | |
| | CTATTACATTAAGGTTCGTGCCGGTGACAATAAGTATATGCACCTG | |
| | AAAGTGTTCAACGGCCCGGTTAACTCTTTTCAGGCACATCGTCATG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 1004 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAAAAAAACATTACCATCATGGTATCTCCACCAA | |
| | CTATTACATTAAGGTTCGTGCCGGTGACAATAAGTATATGCACCTG | |
| | AAAGTGTTCAACGGCCCGGTTAACTCTTTTCAGGCACATCGTCATG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 1005 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACATCATTCTCATCATCGTCTGGAATCCACCAA | |
| | CTATTACATTAAGGTTCGTGCCGGTGACAATAAGTATATGCACCTG | |
| | AAAGTGTTCAACGGCCCGCAGCCAACCTTTCGTCATCATTACACCG | |
| | CGGACCGTGTTCTGACCGGTTACCAGGTTGACAAGAACAAAGATG | |
| | ACGAGCTGACGGGTTTC | |
| FcRn-11 | ATGATCCCGCGTGGCCTGTCTGAAGCTAAACCAGCAACTCCGGAAA | 1006 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATGTTCATCATCATCGTGAAAAAGGTTCCACCAA | |
| | CTATTACATTAAGGTTCGTGCCGGTGACAATAAGTATATGCACCTG | |
| | AAAGTGTTCAACGGCCCGTACTCTAACTCTCGTGAACGTCAGTGGG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1007 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACATAAATACCATCATACCGGTCATTCCACCAA | |
| | CTATTACATTAAGGTTCGTGCCGGTGACAATAAGTATATGCACCTG | |
| | AAAGTGTTCAACGGCCCGGGTCAGATCCATAAAGTTCGTTCTACCG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1008 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAATACTTTGCACCACATGCACCACATTCCACCAA | |
| | CTATTACATTAAGGTTCGTGCCGGTGACAATAAGTATATGCACCTG | |
| | AAAGTGTTCAACGGCCCGCATTACCATCATCGTCATCAGCATTCTG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1009 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACTGCATCATCGTGCACATAAACATCTGTCCACCAA | |
| | CTATTACATTAAGGTTCGTGCCGGTGACAATAAGTATATGCACCTG | |
| | AAAGTGTTCAACGGCCCGTACTTTCATCGTGAACATGAACATCAGG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1010 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGCACATCATGGTCATTACGGTCGTGCATCCACCAA | |
| | CTATTACATTAAGGTTCGTGCCGGTGACAATAAGTATATGCACCTG | |
| | AAAGTGTTCAACGGCCCGTGGCATTACCATCATTCTCAGTGGCGTG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1011 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAGAACATTACTCTCTGTTTAAACCATCCACCAA | |
| | CTATTACATTAAGGTTCGTGCCGGTGACAATAAGTATATGCACCTG | |
| | AAAGTGTTCAACGGCCCTAAACATCATCGTAAACATCGTCATTGGG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1012 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCATCGTCCACGTCATCCAAAACATTCCACCAA | |
| | CTATTACATTAAGGTTCGTGCCGGTGACAATAAGTATATGCACCTG | |
| | AAAGTGTTCAACGGCCCGGCACATAAACATCATCTGGGTTTTAAAG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1013 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACATGAAGTTCATCATCATGGTAACTCCACCAA | |
| | CTATTACATTAAGGTTCGTGCCGGTGACAATAAGTATATGCACCTG | |
| | AAAGTGTTCAACGGCCCGTGGCATCGTCATGGTTCTGGTTTTCGTG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1014 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAATCTCACCATCATAAACATCGTGAATCCACCAA | |
| | CTATTACATTAAGGTTCGTGCCGGTGACAATAAGTATATGCACCTG | |
| | AAAGTGTTCAACGGCCCGGTTGATCGTTTTCTGCATGTTAAAAAAG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1015 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCGTCATCATACCCATAAATGGACCTCCACCAA | |
| | CTATTACATTAAGGTTCGTGCCGGTGACAATAAGTATATGCACCTG | |
| | AAAGTGTTCAACGGCCCGTGGCCACATTCTATCGATTACCGTCAGG | |
| | CGGACCGTGTTCTGACCGGTTACCAGGTTGACAAGAACAAAGATG | |
| | ACGAGCTGACGGGTTTC | |
| FcRn-12 | ATGATCCCGCGTGGCCTGTCTGAAGCTAAACCAGCAACTCCGGAAA | 1016 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTAAACATCCACATCATCATCAGAACTCCACCAA | |
| | CTATTACATTAAGGTTCGTGCCGGTGACAATAAGTATATGCACCTG | |
| | AAAGTGTTCAACGGCCCGAAAGGTCGTTACTCTCATCATCATGGTG | |
| | CGGACCGTGTTCTGACCGGTTACCAGGTTGACAAGAACAAAGATG | |
| | ACGAGCTGACGGGTTTC | |
| FcRn-13 | ATGATCCCGCGTGGCCTGTCTGAAGCTAAACCAGCAACTCCGGAAA | 1017 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCATAAACATCATCTGCGTTACCGTTCCACCAA | |
| | CTATTACATTAAGGTTCGTGCCGGTGACAATAAGTATATGCACCTG | |
| | AAAGTGTTCAACGGCCCGTACCCACAGGATAAACATAAAGTTCTG | |
| | GCGGACCGTGTTCTGACCGGTTACCAGGTTGACAAGAACAAAGAT | |
| | GACGAGCTGACGGGTTTC | |
| FcRn-13 | ATGATCCCGCGTGGCCTGTCTGAAGCTAAACCAGCAACTCCGGAAA | 1018 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAACCCATAAAGAATACCATCATTCTTCCACCAA | |
| | CTATTACATTAAGGTTCGTGCCGGTGACAATAAGTATATGCACCTG | |
| | AAAGTGTTCAACGGCCCGGGTTACCGTCGTCATCAGGGTCGTGGTG | |
| | CGGACCGTGTTCTGACCGGTTACCAGGTTGACAAGAACAAAGATG | |
| | ACGAGCTGACGGGTTTC | |
| FcRn-13 | ATGATCCCGCGTGGCCTGTCTGAAGCTAAACCAGCAACTCCGGAAA | 1019 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCGTCATCATCATCAGCATTGGTCTTCCACCAA | |
| | CTATTACATTAAGGTTCGTGCCGGTGACAATAAGTATATGCACCTG | |
| | AAAGTGTTCAACGGCCCGGCACTGCATGATACCCTGCATCCATCTG | |
| | CGGACCGTGTTCTGACCGGTTACCAGGTTGACAAGAACAAAGATG | |
| | ACGAGCTGACGGGTTTC | |
| FcRn-13 | ATGATCCCGCGTGGCCTGTCTGAAGCTAAACCAGCAACTCCGGAAA | 1020 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCATCGTTGGCATCAGGGTTCTCGTTCCACCAA | |
| | CTATTACATTAAGGTTCGTGCCGGTGACAATAAGTATATGCACCTG | |
| | AAAGTGTTCAACGGCCCGAAAAAACCACATAACCATCGTTACTAC | |
| | GCGGACCGTGTTCTGACCGGTTACCAGGTTGACAAGAACAAAGAT | |
| | GACGAGCTGACGGGTTTC | |
| FcRn-13 | ATGATCCCGCGTGGCCTGTCTGAAGCTAAACCAGCAACTCCGGAAA | 1021 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACGTGGTCATCATCATCCAAACCATTCCACCAA | |
| | CTATTACATTAAGGTTCGTGCCGGTGACAATAAGTATATGCACCTG | |
| | AAAGTGTTCAACGGCCCGGCAAAACATCATTGGGATACCTGGTCTG | |
| | CGGACCGTGTTCTGACCGGTTACCAGGTTGACAAGAACAAAGATG | |
| | ACGAGCTGACGGGTTTC | |
| FcRn-13 | ATGATCCCGCGTGGCCTGTCTGAAGCTAAACCAGCAACTCCGGAAA | 1022 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATACCGTTCCACTGCGTAAACATCAGTCCACCAA | |
| | CTATTACATTAAGGTTCGTGCCGGTGACAATAAGTATATGCACCTG | |
| | AAAGTGTTCAACGGCCCGGTTATCCATCATAAACATCGTCATCAGG | |
| | CGGACCGTGTTCTGACCGGTTACCAGGTTGACAAGAACAAAGATG | |
| | ACGAGCTGACGGGTTTC | |
| FcRn-13 | ATGATCCCGCGTGGCCTGTCTGAAGCTAAACCAGCAACTCCGGAAA | 1023 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCTACCGTTGGGGTCATCATTTTCATTCCACCAA | |
| | CTATTACATTAAGGTTCGTGCCGGTGACAATAAGTATATGCACCTG | |
| | AAAGTGTTCAACGGCCCGAAATACGAACAGATCGATCGTTGGCAT | |
| | GCGGACCGTGTTCTGACCGGTTACCAGGTTGACAAGAACAAAGAT | |
| | GACGAGCTGACGGGTTTC | |
| FcRn-13 | ATGATCCCGCGTGGCCTGTCTGAAGCTAAACCAGCAACTCCGGAAA | 1024 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATTTAAACATCATGATCGTGGTACCCATTCCACCAA | |
| | CTATTACATTAAGGTTCGTGCCGGTGACAATAAGTATATGCACCTG | |
| | AAAGTGTTCAACGGCCCGTACCGTAAACGTCATACCTGGTTTCAGG | |
| | CGGACCGTGTTCTGACCGGTTACCAGGTTGACAAGAACAAAGATG | |
| | ACGAGCTGACGGGTTTC | |
| FcRn-13 | ATGATCCCGCGTGGCCTGTCTGAAGCTAAACCAGCAACTCCGGAAA | 1025 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCGCAAAAAAACATCCAAAATCTCATTCCACCA | |
| | ACTATTACATTAAGGTTCGTGCCGGTGACAATAAGTATATGCACCT | |
| | GAAAGTGTTCAACGGCCCGAAAGTTAACTGGCATCACTACCGTCAT | |
| | GCGGACCGTGTTCTGACCGGTTACCAGGTTGACAAGAACAAAGAT | |
| | GACGAGCTGACGGGTTTC | |
| FcRn-13 | ATGATCCCGCGTGGCCTGTCTGAAGCTAAACCAGCAACTCCGGAAA | 1026 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATTACCATTTTTCTAAACATCATAACTCCACCAA | |
| | CTATTACATTAAGGTTCGTGCCGGTGACAATAAGTATATGCACCTG | |
| | AAAGTGTTCAACGGCCCGTCTTACCATCATAAACATTTTGTTAAAG | |
| | CGGACCGTGTTCTGACCGGTTACCAGGTTGACAAGAACAAAGATG | |
| | ACGAGCTGACGGGTTTC | |
| FcRn-14 | ATGATCCCGCGTGGCCTGTCTGAAGCTAAACCAGCAACTCCGGAAA | 1027 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACAAACATAAACATGGTAAATGGCGTTCCACCA | |
| | ACTATTACATTAAGGTTCGTGCCGGTGACAATAAGTATATGCACCT | |
| | GAAAGTGTTCAACGGCCCGTGGCATGGTCATTTTTCTAAAGGTGGT | |
| | GTTGCATACGCGGACCGTGTTCTGACCGGTTACCAGGTTGACAAGA | |
| | ACAAAGATGACGAGCTGACGGGTTTC | |
| FcRn-14 | ATGATCCCGCGTGGCCTGTCTGAAGCTAAACCAGCAACTCCGGAAA | 1028 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTCATCATAAACCACATAAAACCGAATCCACCA | |
| | ACTATTACATTAAGGTTCGTGCCGGTGACAATAAGTATATGCACCT | |
| | GAAAGTGTTCAACGGCCCGGCAACCCATCTGAAACATCATAACCAT | |
| | GCGGACCGTGTTCTGACCGGTTACCAGGTTGACAAGAACAAAGAT | |
| | GACGAGCTGACGGGTTTC | |
| FcRn-14 | ATGATCCCGCGTGGCCTGTCTGAAGCTAAACCAGCAACTCCGGAAA | 1029 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATGGTCAGCGTTACCATAACAAATCTTCCACCAA | |
| | CTATTACATTAAGGTTCGTGCCGGTGACAATAAGTATATGCACCTG | |
| | AAAGTGTTCAACGGCCCGAAACGTAAATGGGAACATTCTCATAAA | |
| | GCGGACCGTGTTCTGACCGGTTACCAGGTTGACAAGAACAAAGAT | |
| | GACGAGCTGACGGGTTTC | |
| FcRn-14 | ATGATCCCGCGTGGCCTGTCTGAAGCTAAACCAGCAACTCCGGAAA | 1030 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAAACATCATCGTCATGTTCCATCTTCCACCAA | |
| | CTATTACATTAAGGTTCGTGCCGGTGACAATAAGTATATGCACCTG | |
| | AAAGTGTTCAACGGCCCGGATCATCGTCATCGTCATTGGTACCTGG | |
| | CGGACCGTGTTCTGACCGGTTACCAGGTTGACAAGAACAAAGATG | |
| | ACGAGCTGACGGGTTTC | |
| FcRn-14 | ATGATCCCGCGTGGCCTGTCTGAAGCTAAACCAGCAACTCCGGAAA | 1031 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCGTAAACATTCTTGGTCTCGTCATTCCACCAA | |
| | CTATTACATTAAGGTTCGTGCCGGTGACAATAAGTATATGCACCTG | |
| | AAAGTGTTCAACGGCCCGACCAAACATTCTCATTCTCAGCTGTTTG | |
| | CGGACCGTGTTCTGACCGGTTACCAGGTTGACAAGAACAAAGATG | |
| | ACGAGCTGACGGGTTTC | |
| FcRn-14 | ATGATCCCGCGTGGCCTGTCTGAAGCTAAACCAGCAACTCCGGAAA | 1032 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCGTCATTACCATCAGGAATACAAATCCACCA | |
| | ACTATTACATTAAGGTTCGTGCCGGTGACAATAAGTATATGCACCT | |
| | GAAAGTGTTCAACGGCCCGGTTCATAAATCTAAACACTGGTTTTAC | |
| | GCGGACCGTGTTCTGACCGGTTACCAGGTTGACAAGAACAAAGAT | |
| | GACGAGCTGACGGGTTTC | |
| FcRn-14 | ATGATCCCGCGTGGCCTGTCTGAAGCTAAACCAGCAACTCCGGAAA | 1033 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAATCAAACATCATCATTCTTTTAAATCCACCAA | |
| | CTATTACATTAAGGTTCGTGCCGGTGACAATAAGTATATGCACCTG | |
| | AAAGTGTTCAACGGCCCGTCTCAGGATCATCATTTTCATCGTCATG | |
| | CGGACCGTGTTCTGACCGGTTACCAGGTTGACAAGAACAAAGATG | |
| | ACGAGCTGACGGGTTTC | |
| FcRn-14 | ATGATCCCGCGTGGCCTGTCTGAAGCTAAACCAGCAACTCCGGAAA | 1034 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGCATAAACGTTCTCATCGTCAGTCTTCCACCAA | |
| | CTATTACATTAAGGTTCGTGCCGGTGACAATAAGTATATGCACCTG | |
| | AAAGTGTTCAACGGCCCGGGTCATAAATATTCTCATTGGTCTAAAG | |
| | CGGACCGTGTTCTGACCGGTTACCAGGTTGACAAGAACAAAGATG | |
| | ACGAGCTGACGGGTTTC | |
| FcRn-14 | ATGATCCCGCGTGGCCTGTCTGAAGCTAAACCAGCAACTCCGGAAA | 1035 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTGTTTACAAATGGAAAGCATCCACCAACTATTA | |
| | CATTAAGGTTCGTGCCGGTGACAATAAGTATATGCACCTGAAAGTG | |
| | TTCAACGGCCCGAACAAACATCATCATCATGCACATCATGCGGACC | |
| | GTGTTCTGACCGGTTACCAGGTTGACAAGAACAAAGATGACGAGC | |
| | TGACGGGTTTC | |
| FcRn-14 | ATGATCCCGCGTGGCCTGTCTGAAGCTAAACCAGCAACTCCGGAAA | 1036 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTAAACTGGAACGTACCAAATACCATTCCACCA | |
| | ACTATTACATTAAGGTTCGTGCCGGTGACAATAAGTATATGCACCT | |
| | GAAAGTGTTCAACGGCCCGCATAACAAATACCATCCACATAACAA | |
| | AGCGGACCGTGTTCTGACCGGTTACCAGGTTGACAAGAACAAAGA | |
| | TGACGAGCTGACGGGTTTC | |
| FcRn-15 | ATGATCCCGCGTGGCCTGTCTGAAGCTAAACCAGCAACTCCGGAAA | 1037 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCGGTCATAAACATCAGTTTCATCAGTCCACCAA | |
| | CTATTACATTAAGGTTCGTGCCGGTGACAATAAGTATATGCACCTG | |
| | AAAGTGTTCAACGGCCCGAAACATAAACATGGTTGGTTTCATTCTG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 1038 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCAGGAACTGGGTCATCGTGTTTACTCCACCAA | |
| | CTATTACATTAAGGTTCGTGCCGGTGACAATAAGTATATGCACCTG | |
| | AAAGTGTTCAACGGCCCGTACCGTCGTCATCATGATAAAAAACATG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 1039 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCCACATCATACCGATCAGCGTCATTCCACCAA | |
| | CTATTACATTAAGGTTCGTGCCGGTGACAATAAGTATATGCACCTG | |
| | AAAGTGTTCAACGGCCCGGAAGGTCATCGTCAGCATGCAAAATTTG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 1040 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATTTCATAACCATGGTCATCCACATCTGTCCACCAA | |
| | CTATTACATTAAGGTTCGTGCCGGTGACAATAAGTATATGCACCTG | |
| | AAAGTGTTCAACGGCCCGAACTCTCGTGGTCATCATCATCATAAAG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 1041 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGAACCATCATCATCGTAACAAACAGTCCACCA | |
| | ACTATTACATTAAGGTTCGTGCCGGTGACAATAAGTATATGCACCT | |
| | GAAAGTGTTCAACGGCCCGCCACATAAACGTCCACATCTGTACCAT | |
| | GCGGACCGTGTTCTGACCGGTTACCAGGTTGACAAGAACAAAGAT | |
| | GACGAGCTGACGGGTTTC | |
| FcRn-15 | ATGATCCCGCGTGGCCTGTCTGAAGCTAAACCAGCAACTCCGGAAA | 1042 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCGTCATGGTCATCGTCATTACCGTTCCACCAA | |
| | CTATTACATTAAGGTTCGTGCCGGTGACAATAAGTATATGCACCTG | |
| | AAAGTGTTCAACGGCCCGTTTTACGATCTGCATCCAAAACTGTCTG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 1043 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACATCATCGTTGGCATCGTCAGCATTCCACCAA | |
| | CTATTACATTAAGGTTCGTGCCGGTGACAATAAGTATATGCACCTG | |
| | AAAGTGTTCAACGGCCCGATCCATCAGCATTCTCAGAAAAAATCTG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 1044 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCTGCGTCATCAGACCGAACATCGTTCCACCAA | |
| | CTATTACATTAAGGTTCGTGCCGGTGACAATAAGTATATGCACCTG | |
| | AAAGTGTTCAACGGCCCGAAACGTCATCATCGTCATTCTCATGTTG | |
| | CGGACCGTGTTCTGACCGGTTACCAGGTTGACAAGAACAAAGATG | |
| | ACGAGCTGACGGGTTTC | |
| FcRn-15 | ATGATCCCGCGTGGCCTGTCTGAAGCTAAACCAGCAACTCCGGAAA | 1045 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCATCGTAAACATACCCATCTGCTGTCCACCAA | |
| | CTATTACATTAAGGTTCGTGCCGGTGACAATAAGTATATGCACCTG | |
| | AAAGTGTTCAACGGCCCGAAAAAATCTCATAAAGCATGGGCATGG | |
| | GCGGACCGTGTTCTGACCGGTTACCAGGTTGACAAGAACAAAGAT | |
| | GACGAGCTGACGGGTTTC | |
| FcRn-15 | ATGATCCCGCGTGGCCTGTCTGAAGCTAAACCAGCAACTCCGGAAA | 1046 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCATTCTAAACCACAGCATTGGCCATCCACCAA | |
| | CTATTACATTAAGGTTCGTGCCGGTGACAATAAGTATATGCACCTG | |
| | AAAGTGTTCAACGGCCCGAAAGGTCATAAACAGCATCATCATTAC | |
| | GCGGACCGTGTTCTGACCGGTTACCAGGTTGACAAGAACAAAGAT | |
| | GACGAGCTGACGGGTTTC | |
| FcRn-16 | ATGATCCCGCGTGGCCTGTCTGAAGCTAAACCAGCAACTCCGGAAA | 1047 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACATCGTTCTCGTTTTCATAAACAGTCCACCAA | |
| | CTATTACATTAAGGTTCGTGCCGGTGACAATAAGTATATGCACCTG | |
| | AAAGTGTTCAACGGCCCGTGGAAAGCAGAACGTCATAAACATTAC | |
| | GCGGACCGTGTTCTGACCGGTTACCAGGTTGACAAGAACAAAGAT | |
| | GACGAGCTGACGGGTTTC | |
| FcRn-16 | ATGATCCCGCGTGGCCTGTCTGAAGCTAAACCAGCAACTCCGGAAA | 1048 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGCGTAAACATTTTCATTGGGATCATTCCACCAA | |
| | CTATTACATTAAGGTTCGTGCCGGTGACAATAAGTATATGCACCTG | |
| | AAAGTGTTCAACGGCCCGCAGCATCGTTACACCCATCATCATACCG | |
| | CGGACCGTGTTCTGACCGGTTACCAGGTTGACAAGAACAAAGATG | |
| | ACGAGCTGACGGGTTTC | |
| FcRn-16 | ATGATCCCGCGTGGCCTGTCTGAAGCTAAACCAGCAACTCCGGAAA | 1049 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACAAACATCATGGTCAGCAGCATAACTCCACCA | |
| | ACTATTACATTAAGGTTCGTGCCGGTGACAATAAGTATATGCACCT | |
| | GAAAGTGTTCAACGGCCCGTCTCATAAAGTTCATACCCATTCTAAA | |
| | GCGGACCGTGTTCTGACCGGTTACCAGGTTGACAAGAACAAAGAT | |
| | GACGAGCTGACGGGTTTC | |
| FcRn-16 | ATGATCCCGCGTGGCCTGTCTGAAGCTAAACCAGCAACTCCGGAAA | 1050 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAATACCATCATAAATACAAATCTTACTCCACCAA | |
| | CTATTACATTAAGGTTCGTGCCGGTGACAATAAGTATATGCACCTG | |
| | AAAGTGTTCAACGGCCCGAAACATCTGGATCAGTACCATCCATCTG | |
| | CGGACCGTGTTCTGACCGGTTACCAGGTTGACAAGAACAAAGATG | |
| | ACGAGCTGACGGGTTTC | |
| FcRn-16 | ATGATCCCGCGTGGCCTGTCTGAAGCTAAACCAGCAACTCCGGAAA | 1051 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTGAATGGCATCATCAGACCTACTACTCCACCAA | |
| | CTATTACATTAAGGTTCGTGCCGGTGACAATAAGTATATGCACCTG | |
| | AAAGTGTTCAACGGCCCGTCTGCACATAAACATCATCATAACCATG | |
| | CGGACCGTGTTCTGACCGGTTACCAGGTTGACAAGAACAAAGATG | |
| | ACGAGCTGACGGGTTTC | |
| FcRn-16 | ATGATCCCGCGTGGCCTGTCTGAAGCTAAACCAGCAACTCCGGAAA | 1052 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCATTACCATGATCATCATTACCGTTCCACCAA | |
| | CTATTACATTAAGGTTCGTGCCGGTGACAATAAGTATATGCACCTG | |
| | AAAGTGTTCAACGGCCCGAAATACAAACATCAGGTTAAACAGCAT | |
| | GCGGACCGTGTTCTGACCGGTTACCAGGTTGACAAGAACAAAGAT | |
| | GACGAGCTGACGGGTTTC | |
| FcRn-16 | ATGATCCCGCGTGGCCTGTCTGAAGCTAAACCAGCAACTCCGGAAA | 1053 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCATACCTACCGTCATTCTACCGGTTCCACCAA | |
| | CTATTACATTAAGGTTCGTGCCGGTGACAATAAGTATATGCACCTG | |
| | AAAGTGTTCAACGGCCCGATCTCTCATCGTCATCGTCATGATATCG | |
| | CGGACCGTGTTCTGACCGGTTACCAGGTTGACAAGAACAAAGATG | |
| | ACGAGCTGACGGGTTTC | |
| FcRn-16 | ATGATCCCGCGTGGCCTGTCTGAAGCTAAACCAGCAACTCCGGAAA | 1054 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCATCGTCATCATCATCCACATTTTTCCACCAA | |
| | CTATTACATTAAGGTTCGTGCCGGTGACAATAAGTATATGCACCTG | |
| | AAAGTGTTCAACGGCCCGAACTACCATGCACATCGTTCTTTTTACG | |
| | CGGACCGTGTTCTGACCGGTTACCAGGTTGACAAGAACAAAGATG | |
| | ACGAGCTGACGGGTTTC | |
| FcRn-16 | ATGATCCCGCGTGGCCTGTCTGAAGCTAAACCAGCAACTCCGGAAA | 1055 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATGCAAAAACCCGTCATCATGAACATTCCACCAA | |
| | CTATTACATTAAGGTTCGTGCCGGTGACAATAAGTATATGCACCTG | |
| | AAAGTGTTCAACGGCCCGTGGTTTAAACATCATTTTTGGCATCGTG | |
| | CGGACCGTGTTCTGACCGGTTACCAGGTTGACAAGAACAAAGATG | |
| | ACGAGCTGACGGGTTTC | |
| FcRn-16 | ATGATCCCGCGTGGCCTGTCTGAAGCTAAACCAGCAACTCCGGAAA | 1056 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAACCACATCAGAAACATAAACGTCATTCCACCA | |
| | ACTATTACATTAAGGTTCGTGCCGGTGACAATAAGTATATGCACCT | |
| | GAAAGTGTTCAACGGCCCGAAACGTAAAGGTGATTTTCTGAACTAC | |
| | GCGGACCGTGTTCTGACCGGTTACCAGGTTGACAAGAACAAAGAT | |
| | GACGAGCTGACGGGTTTC | |
| FcRn-17 | ATGATCCCGCGTGGCCTGTCTGAAGCTAAACCAGCAACTCCGGAAA | 1057 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCGTCGTCATCAGCATGGTCGTCATTCCACCAA | |
| | CTATTACATTAAGGTTCGTGCCGGTGACAATAAGTATATGCACCTG | |
| | AAAGTGTTCAACGGCCCGCATAAACCATGGGGTCATCATAAACTG | |
| | GCGGACCGTGTTCTGACCGGTTACCAGGTTGACAAGAACAAAGAT | |
| | GACGAGCTGACGGGTTTC | |
| FcRn-17 | ATGATCCCGCGTGGCCTGTCTGAAGCTAAACCAGCAACTCCGGAAA | 1058 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCAGCATCGTCATAACCTGCAGCAGTCCACCAA | |
| | CTATTACATTAAGGTTCGTGCCGGTGACAATAAGTATATGCACCTG | |
| | AAAGTGTTCAACGGCCCGCAGTACAAACATAAACATTGGCTGTGG | |
| | GCGGACCGTGTTCTGACCGGTTACCAGGTTGACAAGAACAAAGAT | |
| | GACGAGCTGACGGGTTTC | |
| FcRn-17 | ATGATCCCGCGTGGCCTGTCTGAAGCTAAACCAGCAACTCCGGAAA | 1059 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACGTATCCATACCTGGCATACCGATTCCACCAA | |
| | CTATTACATTAAGGTTCGTGCCGGTGACAATAAGTATATGCACCTG | |
| | AAAGTGTTCAACGGCCCGTTTAAACGTCATCATTCTTGGCATCATG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 1060 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCATCATCAGCCACGTTACCAGCAGTCCACCAA | |
| | CTATTACATTAAGGTTCGTGCCGGTGACAATAAGTATATGCACCTG | |
| | AAAGTGTTCAACGGCCCGAAAGATCGTCATCATGAATTTCGTCATG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 1061 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTATCGGTCGTCATCGTCGTCGTCGTTCCACCAA | |
| | CTATTACATTAAGGTTCGTGCCGGTGACAATAAGTATATGCACCTG | |
| | AAAGTGTTCAACGGCCCGCATCATCATCATTTTCATAACCATCGTG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 1062 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCAGCATAAACAGCATTACCATTTTTCCACCAA | |
| | CTATTACATTAAGGTTCGTGCCGGTGACAATAAGTATATGCACCTG | |
| | AAAGTGTTCAACGGCCCGTCTGTTAACCAGCATTTTAAACATAAAG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 1063 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCGTCATCATGAATCTCATAAATCTTCCACCAA | |
| | CTATTACATTAAGGTTCGTGCCGGTGACAATAAGTATATGCACCTG | |
| | AAAGTGTTCAACGGCCCGTTTCAGCATAAACTGCATAAACATCATG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 1064 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACGTCATCATCATTGGCATTACTCTTCCACCAA | |
| | CTATTACATTAAGGTTCGTGCCGGTGACAATAAGTATATGCACCTG | |
| | AAAGTGTTCAACGGCCCGGATACCCGTTACGATAAATGGCATGGTG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 1065 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCGTAAAGGTGGTCATCGTTACCATTCCACCAA | |
| | CTATTACATTAAGGTTCGTGCCGGTGACAATAAGTATATGCACCTG | |
| | AAAGTGTTCAACGGCCCGCATGTTCATCGTGTTCAGCATTCTAAAG | |
| | CGGACCGTGTTCTGACCGGTTACCAGGTTGACAAGAACAAAGATG | |
| | ACGAGCTGACGGGTTTC | |
| FcRn-17 | ATGATCCCGCGTGGCCTGTCTGAAGCTAAACCAGCAACTCCGGAAA | 1066 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTAAATGGCATGGTCATTGGCATCGTTCCACCAA | |
| | CTATTACATTAAGGTTCGTGCCGGTGACAATAAGTATATGCACCTG | |
| | AAAGTGTTCAACGGCCCGTGGAACTACCAGTTTAAATCTGCATCTG | |
| | CGGACCGTGTTCTGACCGGTTACCAGGTTGACAAGAACAAAGATG | |
| | ACGAGCTGACGGGTTTC | |
| FcRn-18 | ATGATCCCGCGTGGCCTGTCTGAAGCTAAACCAGCAACTCCGGAAA | 1067 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACTGGAAACGTCATCATTACCATCGTTCCACCAA | |
| | CTATTACATTAAGGTTCGTGCCGGTGACAATAAGTATATGCACCTG | |
| | AAAGTGTTCAACGGCCCGCAGTGGTGGTTTCATAAACATGTTAAAG | |
| | CGGACCGTGTTCTGACCGGTTACCAGGTTGACAAGAACAAAGATG | |
| | ACGAGCTGACGGGTTTC | |
| FcRn-18 | ATGATCCCGCGTGGCCTGTCTGAAGCTAAACCAGCAACTCCGGAAA | 1068 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCGTCATCATCATCGTAACCGTTTTTCCACCAA | |
| | CTATTACATTAAGGTTCGTGCCGGTGACAATAAGTATATGCACCTG | |
| | AAAGTGTTCAACGGCCCGATCTCTCATAACCCAAACCATTACCATG | |
| | CGGACCGTGTTCTGACCGGTTACCAGGTTGACAAGAACAAAGATG | |
| | ACGAGCTGACGGGTTTC | |
| FcRn-18 | ATGATCCCGCGTGGCCTGTCTGAAGCTAAACCAGCAACTCCGGAAA | 1069 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTAAATGGGATTTTAAACATTTTTACTCCACCAA | |
| | CTATTACATTAAGGTTCGTGCCGGTGACAATAAGTATATGCACCTG | |
| | AAAGTGTTCAACGGCCCGACCAACCTGCATTCTCCAGATTCTCCAG | |
| | CGGACCGTGTTCTGACCGGTTACCAGGTTGACAAGAACAAAGATG | |
| | ACGAGCTGACGGGTTTC | |
| FcRn-18 | ATGATCCCGCGTGGCCTGTCTGAAGCTAAACCAGCAACTCCGGAAA | 1070 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTGATGATCTGTCTCCAGTTAAATGGTCCACCAA | |
| | CTATTACATTAAGGTTCGTGCCGGTGACAATAAGTATATGCACCTG | |
| | AAAGTGTTCAACGGCCCGTTTGATAAATACAACTCTCATTACCTGG | |
| | CGGACCGTGTTCTGACCGGTTACCAGGTTGACAAGAACAAAGATG | |
| | ACGAGCTGACGGGTTTC | |
| FcRn-18 | ATGATCCCGCGTGGCCTGTCTGAAGCTAAACCAGCAACTCCGGAAA | 1071 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCATCGTCAGAAATGGCCAATCCATTCCACCAA | |
| | CTATTACATTAAGGTTCGTGCCGGTGACAATAAGTATATGCACCTG | |
| | AAAGTGTTCAACGGCCCGTCTACCCATCAGCAGAAACATCAGTGG | |
| | GCGGACCGTGTTCTGACCGGTTACCAGGTTGACAAGAACAAAGAT | |
| | GACGAGCTGACGGGTTTC | |
| FcRn-18 | ATGATCCCGCGTGGCCTGTCTGAAGCTAAACCAGCAACTCCGGAAA | 1072 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCGTCATGCATACCATCGTCATTCCACCAACTA | |
| | TTACATTAAGGTTCGTGCCGGTGACAATAAGTATATGCACCTGAAA | |
| | GTGTTCAACGGCCCGTTTCATGAAGAAATCAAACATTGGCAGGCGG | |
| | ACCGTGTTCTGACCGGTTACCAGGTTGACAAGAACAAAGATGACG | |
| | AGCTGACGGGTTTC | |
| FcRn-18 | ATGATCCCGCGTGGCCTGTCTGAAGCTAAACCAGCAACTCCGGAAA | 1073 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCGTCATCATCAGAAACATGCATTTTCCACCAA | |
| | CTATTACATTAAGGTTCGTGCCGGTGACAATAAGTATATGCACCTG | |
| | AAAGTGTTCAACGGCCCGTGGCGTGATTGGAACCATCGTTTTCCAG | |
| | CGGACCGTGTTCTGACCGGTTACCAGGTTGACAAGAACAAAGATG | |
| | ACGAGCTGACGGGTTTC | |
| FcRn-18 | ATGATCCCGCGTGGCCTGTCTGAAGCTAAACCAGCAACTCCGGAAA | 1074 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGAAAGGTAAACATCATGATTACCGTTCCACCA | |
| | ACTATTACATTAAGGTTCGTGCCGGTGACAATAAGTATATGCACCT | |
| | GAAAGTGTTCAACGGCCCGAAACCACATCAGACCAAATGGCATCA | |
| | TGCGGACCGTGTTCTGACCGGTTACCAGGTTGACAAGAACAAAGAT | |
| | GACGAGCTGACGGGTTTC | |
| FcRn-18 | ATGATCCCGCGTGGCCTGTCTGAAGCTAAACCAGCAACTCCGGAAA | 1075 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGAACAAACATTTTTACAAACAGGGTTCCACCAA | |
| | CTATTACATTAAGGTTCGTGCCGGTGACAATAAGTATATGCACCTG | |
| | AAAGTGTTCAACGGCCCGCGTCATCATCGTCAGTCTCATCATTGGG | |
| | CGGACCGTGTTCTGACCGGTTACCAGGTTGACAAGAACAAAGATG | |
| | ACGAGCTGACGGGTTTC | |
| FcRn-18 | ATGATCCCGCGTGGCCTGTCTGAAGCTAAACCAGCAACTCCGGAAA | 1076 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACGTCGTCATAACCGTGAATTTGTTTCCACCAA | |
| | CTATTACATTAAGGTTCGTGCCGGTGACAATAAGTATATGCACCTG | |
| | AAAGTGTTCAACGGCCCGATCCGTCATTACCATGCAGATCGTGAAG | |
| | CGGACCGTGTTCTGACCGGTTACCAGGTTGACAAGAACAAAGATG | |
| | ACGAGCTGACGGGTTTC | |
| FcRn-19 | ATGATCCCGCGTGGCCTGTCTGAAGCTAAACCAGCAACTCCGGAAA | 1077 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCGTCATGTTCGTCATTGGACCCATTCCACCAA | |
| | CTATTACATTAAGGTTCGTGCCGGTGACAATAAGTATATGCACCTG | |
| | AAAGTGTTCAACGGCCCGGCATCTCAGGTTCCACCAAAACATCGTG | |
| | CGGACCGTGTTCTGACCGGTTACCAGGTTGACAAGAACAAAGATG | |
| | ACGAGCTGACGGGTTTC | |
| FcRn-19 | ATGATCCCGCGTGGCCTGTCTGAAGCTAAACCAGCAACTCCGGAAA | 1078 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCGTAAATGGCAGCAGAACCATCATTCCACCA | |
| | ACTATTACATTAAGGTTCGTGCCGGTGACAATAAGTATATGCACCT | |
| | GAAAGTGTTCAACGGCCCGAAACATAAACATTGGCATCATCAGCT | |
| | GGCGGACCGTGTTCTGACCGGTTACCAGGTTGACAAGAACAAAGA | |
| | TGACGAGCTGACGGGTTTC | |
| FcRn-19 | ATGATCCCGCGTGGCCTGTCTGAAGCTAAACCAGCAACTCCGGAAA | 1079 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCACCGTGAAAAACATCAGCCATACTCCACCA | |
| | ACTATTACATTAAGGTTCGTGCCGGTGACAATAAGTATATGCACCT | |
| | GAAAGTGTTCAACGGCCCGTGGGAACATCATCGTACCCGTTGGCAG | |
| | GCGGACCGTGTTCTGACCGGTTACCAGGTTGACAAGAACAAAGAT | |
| | GACGAGCTGACGGGTTTC | |
| FcRn-19 | ATGATCCCGCGTGGCCTGTCTGAAGCTAAACCAGCAACTCCGGAAA | 1080 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCATAAACATAACTCTAAACATTCTTCCACCAA | |
| | CTATTACATTAAGGTTCGTGCCGGTGACAATAAGTATATGCACCTG | |
| | AAAGTGTTCAACGGCCCGTTTAAAACCTTTAAAGAATGGCATGTTG | |
| | CGGACCGTGTTCTGACCGGTTACCAGGTTGACAAGAACAAAGATG | |
| | ACGAGCTGACGGGTTTC | |
| FcRn-19 | ATGATCCCGCGTGGCCTGTCTGAAGCTAAACCAGCAACTCCGGAAA | 1081 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAGCAGGTCAGCATAAACGTAAACATTCCACCA | |
| | ACTATTACATTAAGGTTCGTGCCGGTGACAATAAGTATATGCACCT | |
| | GAAAGTGTTCAACGGCCCGAAAGGTCATCGTTGGCATGATTTTAAA | |
| | GCGGACCGTGTTCTGACCGGTTACCAGGTTGACAAGAACAAAGAT | |
| | GACGAGCTGACGGGTTTC | |
| FcRn-19 | ATGATCCCGCGTGGCCTGTCTGAAGCTAAACCAGCAACTCCGGAAA | 1082 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCGTCATAAATACCCAGTTCGTGTTTCCACCAA | |
| | CTATTACATTAAGGTTCGTGCCGGTGACAATAAGTATATGCACCTG | |
| | AAAGTGTTCAACGGCCCGAAACATGCATGGCAGCATCATAAATCT | |
| | GCGGACCGTGTTCTGACCGGTTACCAGGTTGACAAGAACAAAGAT | |
| | GACGAGCTGACGGGTTTC | |
| FcRn-19 | ATGATCCCGCGTGGCCTGTCTGAAGCTAAACCAGCAACTCCGGAAA | 1083 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTAACAACAACCCACAGGGTCATGTTTCCACCA | |
| | ACTATTACATTAAGGTTCGTGCCGGTGACAATAAGTATATGCACCT | |
| | GAAAGTGTTCAACGGCCCGTACAAACATTTTAAACATCATTGGCGT | |
| | GCGGACCGTGTTCTGACCGGTTACCAGGTTGACAAGAACAAAGAT | |
| | GACGAGCTGACGGGTTTC | |
| FcRn-19 | ATGATCCCGCGTGGCCTGTCTGAAGCTAAACCAGCAACTCCGGAAA | 1084 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACAGCTGCATCATCATCATTACAAATCCACCAA | |
| | CTATTACATTAAGGTTCGTGCCGGTGACAATAAGTATATGCACCTG | |
| | AAAGTGTTCAACGGCCCGGCACATCGTAAATTTTTTCAGTGGCATG | |
| | CGGACCGTGTTCTGACCGGTTACCAGGTTGACAAGAACAAAGATG | |
| | ACGAGCTGACGGGTTTC | |
| FcRn-19 | ATGATCCCGCGTGGCCTGTCTGAAGCTAAACCAGCAACTCCGGAAA | 1085 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGAAACATAACTGGCATCGTTGGCATTCCACCAA | |
| | CTATTACATTAAGGTTCGTGCCGGTGACAATAAGTATATGCACCTG | |
| | AAAGTGTTCAACGGCCCGTGGACCCATCGTTCTCAGGTTAAAGTTG | |
| | CGGACCGTGTTCTGACCGGTTACCAGGTTGACAAGAACAAAGATG | |
| | ACGAGCTGACGGGTTTC | |
| FcRn-19 | ATGATCCCGCGTGGCCTGTCTGAAGCTAAACCAGCAACTCCGGAAA | 1086 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACAAACATCTGGGTTACTGGCAGAAATCCACCA | |
| | ACTATTACATTAAGGTTCGTGCCGGTGACAATAAGTATATGCACCT | |
| | GAAAGTGTTCAACGGCCCGTTTCAGTGGTTTAAAGTTGGTGTTCCA | |
| | GCGGACCGTGTTCTGACCGGTTACCAGGTTGACAAGAACAAAGAT | |
| | GACGAGCTGACGGGTTTC | |
| FcRn-20 | ATGATCCCGCGTGGCCTGTCTGAAGCTAAACCAGCAACTCCGGAAA | 1087 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCAGAAAAACTTTGAAGCATGGGAATCCACCA | |
| | ACTATTACATTAAGGTTCGTGCCGGTGACAATAAGTATATGCACCT | |
| | GAAAGTGTTCAACGGCCCGGTTCGTTACTACTCTAAATACCAGTGG | |
| | GCGGACCGTGTTCTGACCGGTTACCAGGTTGACAAGAACAAAGAT | |
| | GACGAGCTGACGGGTTTC | |
| FcRn-20 | ATGATCCCGCGTGGCCTGTCTGAAGCTAAACCAGCAACTCCGGAAA | 1088 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAACGTGTTCGTCGTCGTCATCCACCATCCACCAA | |
| | CTATTACATTAAGGTTCGTGCCGGTGACAATAAGTATATGCACCTG | |
| | AAAGTGTTCAACGGCCCGAACGGTTGGCATGTTGGTCATCATATCG | |
| | CGGACCGTGTTCTGACCGGTTACCAGGTTGACAAGAACAAAGATG | |
| | ACGAGCTGACGGGTTTC | |
| FcRn-20 | ATGATCCCGCGTGGCCTGTCTGAAGCTAAACCAGCAACTCCGGAAA | 1089 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAAAGTTCATATCTTTCGTGAACCATCCACCAA | |
| | CTATTACATTAAGGTTCGTGCCGGTGACAATAAGTATATGCACCTG | |
| | AAAGTGTTCAACGGCCCGACCCGTTTTCGTCATTACCTGGTTACCG | |
| | CGGACCGTGTTCTGACCGGTTACCAGGTTGACAAGAACAAAGATG | |
| | ACGAGCTGACGGGTTTC | |
| FcRn-20 | ATGATCCCGCGTGGCCTGTCTGAAGCTAAACCAGCAACTCCGGAAA | 1090 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTAAATCTTTTCATGTTCATTCTCATTCCACCAAC | |
| | TATTACATTAAGGTTCGTGCCGGTGACAATAAGTATATGCACCTGA | |
| | AAGTGTTCAACGGCCCGTCTTGGCGTAACGTTCGTCCAGAATTTGC | |
| | GGACCGTGTTCTGACCGGTTACCAGGTTGACAAGAACAAAGATGA | |
| | CGAGCTGACGGGTTTC | |
| FcRn-20 | ATGATCCCGCGTGGCCTGTCTGAAGCTAAACCAGCAACTCCGGAAA | 1091 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCATAAAGATCCACCACCACCATGGTCCACCA | |
| | ACTATTACATTAAGGTTCGTGCCGGTGACAATAAGTATATGCACCT | |
| | GAAAGTGTTCAACGGCCCGTTTGGTCATACCTTTTCTTGGCGTTACG | |
| | CGGACCGTGTTCTGACCGGTTACCAGGTTGACAAGAACAAAGATG | |
| | ACGAGCTGACGGGTTTC | |
| FcRn-20 | ATGATCCCGCGTGGCCTGTCTGAAGCTAAACCAGCAACTCCGGAAA | 1092 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCGTTACGCACATAACCATTTTCTGTCCACCAA | |
| | CTATTACATTAAGGTTCGTGCCGGTGACAATAAGTATATGCACCTG | |
| | AAAGTGTTCAACGGCCCGTTTAAACATCAGAAATTTTACCGTGATG | |
| | CGGACCGTGTTCTGACCGGTTACCAGGTTGACAAGAACAAAGATG | |
| | ACGAGCTGACGGGTTTC | |
| FcRn-20 | ATGATCCCGCGTGGCCTGTCTGAAGCTAAACCAGCAACTCCGGAAA | 1093 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTTCTCATGCACTGAAAACCCATACCTCCACCAA | |
| | CTATTACATTAAGGTTCGTGCCGGTGACAATAAGTATATGCACCTG | |
| | AAAGTGTTCAACGGCCCGTGGCGTAACAAATGGCGTGCACAGGAT | |
| | GCGGACCGTGTTCTGACCGGTTACCAGGTTGACAAGAACAAAGAT | |
| | GACGAGCTGACGGGTTTC | |
| FcRn-20 | ATGATCCCGCGTGGCCTGTCTGAAGCTAAACCAGCAACTCCGGAAA | 1094 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCAGTCTCGTGCAATCTACGTTTACTCCACCAA | |
| | CTATTACATTAAGGTTCGTGCCGGTGACAATAAGTATATGCACCTG | |
| | AAAGTGTTCAACGGCCCGTACCAGAAATCTTACTTTCATCGTCATG | |
| | CGGACCGTGTTCTGACCGGTTACCAGGTTGACAAGAACAAAGATG | |
| | ACGAGCTGACGGGTTTC | |
| FcRn-20 | ATGATCCCGCGTGGCCTGTCTGAAGCTAAACCAGCAACTCCGGAAA | 1095 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATACCACCTACCATCAGCACCATTCCACCAA | |
| | CTATTACATTAAGGTTCGTGCCGGTGACAATAAGTATATGCACCTG | |
| | AAAGTGTTCAACGGCCCGTGGCGTCCACGTCCAGTTCATTGGAAAG | |
| | CGGACCGTGTTCTGACCGGTTACCAGGTTGACAAGAACAAAGATG | |
| | ACGAGCTGACGGGTTTC | |
| FcRn-20 | ATGATCCCGCGTGGCCTGTCTGAAGCTAAACCAGCAACTCCGGAAA | 1096 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCTGGTGGCGTAACGTTCAGCATCATTCCACCAA | |
| | CTATTACATTAAGGTTCGTGCCGGTGACAATAAGTATATGCACCTG | |
| | AAAGTGTTCAACGGCCCGGATCCACAGTACAAACGTCATGGTTACG | |
| | CGGACCGTGTTCTGACCGGTTACCAGGTTGACAAGAACAAAGATG | |
| | ACGAGCTGACGGGTTTC | |
| FcRn-21 | ATGATCCCGCGTGGCCTGTCTGAAGCTAAACCAGCAACTCCGGAAA | 1097 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGAACAAACATAACTACCAGCATCAGTCCACCA | |
| | ACTATTACATTAAGGTTCGTGCCGGTGACAATAAGTATATGCACCT | |
| | GAAAGTGTTCAACGGCCCGGTTCCACATTCTGTTGTTCATTACAAA | |
| | GCGGACCGTGTTCTGACCGGTTACCAGGTTGACAAGAACAAAGAT | |
| | GACGAGCTGACGGGTTTC | |
| FcRn-21 | ATGATCCCGCGTGGCCTGTCTGAAGCTAAACCAGCAACTCCGGAAA | 1098 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGCATACCCTGCGTGTTCATACCGTTTCCACCAA | |
| | CTATTACATTAAGGTTCGTGCCGGTGACAATAAGTATATGCACCTG | |
| | AAAGTGTTCAACGGCCCGGCATACTCTCAGTCTTTTATCCATCATG | |
| | CGGACCGTGTTCTGACCGGTTACCAGGTTGACAAGAACAAAGATG | |
| | ACGAGCTGACGGGTTTC | |
| FcRn-21 | ATGATCCCGCGTGGCCTGTCTGAAGCTAAACCAGCAACTCCGGAAA | 1099 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAACCAGCATTTTCATCAGGCAGGTCATTCCACCAA | |
| | CTATTACATTAAGGTTCGTGCCGGTGACAATAAGTATATGCACCTG | |
| | AAAGTGTTCAACGGCCCGTTTTCTCATTCTACCTGGCGTTACCATGC | |
| | GGACCGTGTTCTGACCGGTTACCAGGTTGACAAGAACAAAGATGA | |
| | CGAGCTGACGGGTTTC | |
| FcRn-21 | ATGATCCCGCGTGGCCTGTCTGAAGCTAAACCAGCAACTCCGGAAA | 1100 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCAGTGGACCGATCGTGTTTGGGTTTCCACCAA | |
| | CTATTACATTAAGGTTCGTGCCGGTGACAATAAGTATATGCACCTG | |
| | AAAGTGTTCAACGGCCCGTCTAAAAAACATCAGCAGCATTGGGCG | |
| | GACCGTGTTCTGACCGGTTACCAGGTTGACAAGAACAAAGATGAC | |
| | GAGCTGACGGGTTTC | |
| FcRn-21 | ATGATCCCGCGTGGCCTGTCTGAAGCTAAACCAGCAACTCCGGAAA | 1101 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCATGATTACTTTCATCATAACAAATCCACCAA | |
| | CTATTACATTAAGGTTCGTGCCGGTGACAATAAGTATATGCACCTG | |
| | AAAGTGTTCAACGGCCCGGCAAAACATCCACGTATCCATGTTACCG | |
| | CGGACCGTGTTCTGACCGGTTACCAGGTTGACAAGAACAAAGATG | |
| | ACGAGCTGACGGGTTTC | |
| FcRn-21 | ATGATCCCGCGTGGCCTGTCTGAAGCTAAACCAGCAACTCCGGAAA | 1102 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACTGGGATGTTGGTCCAGGTTTTAACTCCACCAA | |
| | CTATTACATTAAGGTTCGTGCCGGTGACAATAAGTATATGCACCTG | |
| | AAAGTGTTCAACGGCCCGTCTCCATGGCATCATCCAACCCATTTTG | |
| | CGGACCGTGTTCTGACCGGTTACCAGGTTGACAAGAACAAAGATG | |
| | ACGAGCTGACGGGTTTC | |
| FcRn-21 | ATGATCCCGCGTGGCCTGTCTGAAGCTAAACCAGCAACTCCGGAAA | 1103 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTATCCATGGTCATCATGAATACTACTCCACCAA | |
| | CTATTACATTAAGGTTCGTGCCGGTGACAATAAGTATATGCACCTG | |
| | AAAGTGTTCAACGGCCCGTCTAACTGGTTTCATCATAAACATCGTG | |
| | CGGACCGTGTTCTGACCGGTTACCAGGTTGACAAGAACAAAGATG | |
| | ACGAGCTGACGGGTTTC | |
| FcRn-21 | ATGATCCCGCGTGGCCTGTCTGAAGCTAAACCAGCAACTCCGGAAA | 1104 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCAGCGTTCTCGTTACGGTAAATACTCCACCAA | |
| | CTATTACATTAAGGTTCGTGCCGGTGACAATAAGTATATGCACCTG | |
| | AAAGTGTTCAACGGCCCGGCATACTGGCCATACCAGAAACCAACC | |
| | GCGGACCGTGTTCTGACCGGTTACCAGGTTGACAAGAACAAAGAT | |
| | GACGAGCTGACGGGTTTC | |
| FcRn-21 | ATGATCCCGCGTGGCCTGTCTGAAGCTAAACCAGCAACTCCGGAAA | 1105 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCATCAGCAGCATTGGCGTGTTCATTCCACCAA | |
| | CTATTACATTAAGGTTCGTGCCGGTGACAATAAGTATATGCACCTG | |
| | AAAGTGTTCAACGGCCCGATCCTGGTTGGTTACAACTGGCATTACG | |
| | CGGACCGTGTTCTGACCGGTTACCAGGTTGACAAGAACAAAGATG | |
| | ACGAGCTGACGGGTTTC | |
| FcRn-21 | ATGATCCCGCGTGGCCTGTCTGAAGCTAAACCAGCAACTCCGGAAA | 1106 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGCAACCCGTAACTCTTACCCACGTCATTCCACCAA | |
| | CTATTACATTAAGGTTCGTGCCGGTGACAATAAGTATATGCACCTG | |
| | AAAGTGTTCAACGGCCCGGTTCATTCTCATCTGCCACGTCATCCAG | |
| | CGGACCGTGTTCTGACCGGTTACCAGGTTGACAAGAACAAAGATG | |
| | ACGAGCTGACGGGTTTC | |
| FcRn-22 | ATGATCCCGCGTGGCCTGTCTGAAGCTAAACCAGCAACTCCGGAAA | 1107 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAACATCATCATGCACATTGGGCAACCTCCACCAA | |
| | CTATTACATTAAGGTTCGTGCCGGTGACAATAAGTATATGCACCTG | |
| | AAAGTGTTCAACGGCCCGCTGTTTCTGCATGGTGTTCATATCTTTGC | |
| | GGACCGTGTTCTGACCGGTTACCAGGTTGACAAGAACAAAGATGA | |
| | CGAGCTGACGGGTTTC | |
| FcRn-22 | ATGATCCCGCGTGGCCTGTCTGAAGCTAAACCAGCAACTCCGGAAA | 1108 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACAGCATCAGCGTTCTTTTATCATCTCCACCAA | |
| | CTATTACATTAAGGTTCGTGCCGGTGACAATAAGTATATGCACCTG | |
| | AAAGTGTTCAACGGCCCGACCTCTCTGCCATCTGAATGGTTTCAGG | |
| | CGGACCGTGTTCTGACCGGTTACCAGGTTGACAAGAACAAAGATG | |
| | ACGAGCTGACGGGTTTC | |
| FcRn-22 | ATGATCCCGCGTGGCCTGTCTGAAGCTAAACCAGCAACTCCGGAAA | 1109 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACAGTTTTGGGGTCATCGTGTTGAACATTCCACCAA | |
| | CTATTACATTAAGGTTCGTGCCGGTGACAATAAGTATATGCACCTG | |
| | AAAGTGTTCAACGGCCCGACCCGTCATTACCATCAGCGTAACCGTG | |
| | CGGACCGTGTTCTGACCGGTTACCAGGTTGACAAGAACAAAGATG | |
| | ACGAGCTGACGGGTTTC | |
| FcRn-22 | ATGATCCCGCGTGGCCTGTCTGAAGCTAAACCAGCAACTCCGGAAA | 1110 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATTTCCATCTTCTCATCGTACCTCTTACTCCACCAAC | |
| | TATTACATTAAGGTTCGTGCCGGTGACAATAAGTATATGCACCTGA | |
| | AAGTGTTCAACGGCCCGTACTCTGCACATCATATCCGTTGGCATGC | |
| | GGACCGTGTTCTGACCGGTTACCAGGTTGACAAGAACAAAGATGA | |
| | CGAGCTGACGGGTTTC | |
| FcRn-22 | ATGATCCCGCGTGGCCTGTCTGAAGCTAAACCAGCAACTCCGGAAA | mi |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTTCTAAATACATCGATCATCGTCAGTCCACCAA | |
| | CTATTACATTAAGGTTCGTGCCGGTGACAATAAGTATATGCACCTG | |
| | AAAGTGTTCAACGGCCCGGAACGTGCACAGCATCATACCCATCCA | |
| | GCGGACCGTGTTCTGACCGGTTACCAGGTTGACAAGAACAAAGAT | |
| | GACGAGCTGACGGGTTTC | |
| FcRn-22 | ATGATCCCGCGTGGCCTGTCTGAAGCTAAACCAGCAACTCCGGAAA | 1112 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACTGGCGTCATGAACATTCTTCTCCATCCACCAA | |
| | CTATTACATTAAGGTTCGTGCCGGTGACAATAAGTATATGCACCTG | |
| | AAAGTGTTCAACGGCCCGTGGAAAAAACATCATTACGGTCATTACG | |
| | CGGACCGTGTTCTGACCGGTTACCAGGTTGACAAGAACAAAGATG | |
| | ACGAGCTGACGGGTTTC | |
| FcRn-22 | ATGATCCCGCGTGGCCTGTCTGAAGCTAAACCAGCAACTCCGGAAA | 1113 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAACGTGCACATTACGATCATCATTACTCCACCAA | |
| | CTATTACATTAAGGTTCGTGCCGGTGACAATAAGTATATGCACCTG | |
| | AAAGTGTTCAACGGCCCGTCTCATCATGCACATCATTCTGTTCAGG | |
| | CGGACCGTGTTCTGACCGGTTACCAGGTTGACAAGAACAAAGATG | |
| | ACGAGCTGACGGGTTTC | |
| FcRn-22 | ATGATCCCGCGTGGCCTGTCTGAAGCTAAACCAGCAACTCCGGAAA | 1114 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCGTCATAAAGCATACATCTACGGTTCCACCAA | |
| | CTATTACATTAAGGTTCGTGCCGGTGACAATAAGTATATGCACCTG | |
| | AAAGTGTTCAACGGCCCGTGGAAACATTGGGAACATAAACCACAG | |
| | GCGGACCGTGTTCTGACCGGTTACCAGGTTGACAAGAACAAAGAT | |
| | GACGAGCTGACGGGTTTC | |
| FcRn-22 | ATGATCCCGCGTGGCCTGTCTGAAGCTAAACCAGCAACTCCGGAAA | 1115 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACAGATCAAAGAACAGTACAACGGTTCCACCA | |
| | ACTATTACATTAAGGTTCGTGCCGGTGACAATAAGTATATGCACCT | |
| | GAAAGTGTTCAACGGCCCGGCACAGGTTCCAGTTCTGCTGTGGTAC | |
| | GCGGACCGTGTTCTGACCGGTTACCAGGTTGACAAGAACAAAGAT | |
| | GACGAGCTGACGGGTTTC | |
| FcRn-22 | ATGATCCCGCGTGGCCTGTCTGAAGCTAAACCAGCAACTCCGGAAA | 1116 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATTTAAAAAAGTTGCACGTGATCATTGGTCCACCAA | |
| | CTATTACATTAAGGTTCGTGCCGGTGACAATAAGTATATGCACCTG | |
| | AAAGTGTTCAACGGCCCGTGGGTTCATTTTTACCCATGGCAGCAGG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1117 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGCACAGAAACATCATTGGCACAAAACCTCCACCA | |
| | ACTATTACATTAAGGTTCGTGCCGGTGACAATAAGTATATGCACCT | |
| | GAAAGTGTTCAACGGCCCGTGGCATCTGGCACATGTTTTTTACACC | |
| | GCGGACCGTGTTCTGACCGGTTACCAGGTTGACAAGAACAAAGAT | |
| | GACGAGCTGACGGGTTTC | |
| FcRn-23 | ATGATCCCGCGTGGCCTGTCTGAAGCTAAACCAGCAACTCCGGAAA | 1118 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTTCTCAGGGTCATCATTCTTGGGATTCCACCAA | |
| | CTATTACATTAAGGTTCGTGCCGGTGACAATAAGTATATGCACCTG | |
| | AAAGTGTTCAACGGCCCGTCTTCTCATCATCATAAAAACCATCATG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1119 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCATCTGCGTGGTCATCCACATTACTCCACCAA | |
| | CTATTACATTAAGGTTCGTGCCGGTGACAATAAGTATATGCACCTG | |
| | AAAGTGTTCAACGGCCCGACCAAACAGCCACATGGTGTTCATTACG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1120 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATTCTCATCATCATCAGCCATGGGAATCCACCAA | |
| | CTATTACATTAAGGTTCGTGCCGGTGACAATAAGTATATGCACCTG | |
| | AAAGTGTTCAACGGCCCGGAACATCGTACCCATCATCTGGGTAAAG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1121 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCGTTTTCGTGTTCATCTGCATCAGTCCACCAA | |
| | CTATTACATTAAGGTTCGTGCCGGTGACAATAAGTATATGCACCTG | |
| | AAAGTGTTCAACGGCCCGACCAACCATCGTCAGGATCATCCAGAA | |
| | GCGGACCGTGTTCTGACCGGTTACCAGGTTGACAAGAACAAAGAT | |
| | GACGAGCTGACGGGTTTC | |
| FcRn-23 | ATGATCCCGCGTGGCCTGTCTGAAGCTAAACCAGCAACTCCGGAAA | 1122 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCGTCAGACCAAATCTCATCAGCATTCCACCAA | |
| | CTATTACATTAAGGTTCGTGCCGGTGACAATAAGTATATGCACCTG | |
| | AAAGTGTTCAACGGCCCGCATCGTAAAACCAACTGGCATTCTTACG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1123 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCATACTCTCGTCATCATCATCAGCTGTCCACCAA | |
| | CTATTACATTAAGGTTCGTGCCGGTGACAATAAGTATATGCACCTG | |
| | AAAGTGTTCAACGGCCCGTCTGGTGTTCATCATGCAGCAGTTTGGG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1124 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTCATGGTGATCATACCCGTGCATGGTCCACCAA | |
| | CTATTACATTAAGGTTCGTGCCGGTGACAATAAGTATATGCACCTG | |
| | AAAGTGTTCAACGGCCCGCGTTACGCATCTTCTTACTGGGAATGGG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1125 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATTGGCAGAAACGCGGTCGTTCTTGGTCCACCAA | |
| | CTATTACATTAAGGTTCGTGCCGGTGACAATAAGTATATGCACCTG | |
| | AAAGTGTTCAACGGCCCGAACCAGTCTGGTGTTGTTGTTCAGGTTG | |
| | CGGACCGTGTTCTGACCGGTTACCAGGTTGACAAGAACAAAGATG | |
| | ACGAGCTGACGGGTTTC | |
| FcRn-23 | ATGATCCCGCGTGGCCTGTCTGAAGCTAAACCAGCAACTCCGGAAA | 1126 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACAACTGGGAACGTTTTCGTAAAGTTTCCACCAA | |
| | CTATTACATTAAGGTTCGTGCCGGTGACAATAAGTATATGCACCTG | |
| | AAAGTGTTCAACGGCCCGTACCATAACCATCAGCATACCATCCATG | |
| | CGGACCGTGTTCTGACCGGTTACCAGGTTGACAAGAACAAAGATG | |
| | ACGAGCTGACGGGTTTC | |
| FcRn-24 | ATGATCCCGCGTGGCCTGTCTGAAGCTAAACCAGCAACTCCGGAAA | 1127 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTTGGTCTCGTAACGTTTGGTTTTGGTCCACCAA | |
| | CTATTACATTAAGGTTCGTGCCGGTGACAATAAGTATATGCACCTG | |
| | AAAGTGTTCAACGGCCCGAAACAGGAACTGGGTACCAAAACCACC | |
| | GCGGACCGTGTTCTGACCGGTTACCAGGTTGACAAGAACAAAGAT | |
| | GACGAGCTGACGGGTTTC | |
| FcRn-24 | ATGATCCCGCGTGGCCTGTCTGAAGCTAAACCAGCAACTCCGGAAA | 1128 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCAGACCCAGCATCGTCGTCATCATTCCACCAA | |
| | CTATTACATTAAGGTTCGTGCCGGTGACAATAAGTATATGCACCTG | |
| | AAAGTGTTCAACGGCCCGCTTGTTCCACAGCATCATCAGCATCAGG | |
| | CGGACCGTGTTCTGACCGGTTACCAGGTTGACAAGAACAAAGATG | |
| | ACGAGCTGACGGGTTTC | |
| FcRn-24 | ATGATCCCGCGTGGCCTGTCTGAAGCTAAACCAGCAACTCCGGAAA | 1129 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAAACGTTAAACATAAACATCGTTGGTCCACCAA | |
| | CTATTACATTAAGGTTCGTGCCGGTGACAATAAGTATATGCACCTG | |
| | AAAGTGTTCAACGGCCCGTGGCATGATATCGCAGGTGGTCATTACG | |
| | CGGACCGTGTTCTGACCGGTTACCAGGTTGACAAGAACAAAGATG | |
| | ACGAGCTGACGGGTTTC | |
| FcRn-24 | ATGATCCCGCGTGGCCTGTCTGAAGCTAAACCGGCAACTCCGGAAA | 1130 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACATCCAGCATTTCATCAGCATTCTTCCACCAA | |
| | CTATTACATTAAGGTTCGTGCCGGTGACAATAAGTATATGCACCTG | |
| | AAAGTGTTCAACGGCCCGCGTCATGATCTGCATTACCATTACCCAG | |
| | CGGACCGTGTTCTGACCGGTTACCAGGTTGACAAGAACAAAGATG | |
| | ACGAGCTGACGGGTTTC | |
| FcRn-24 | ATGATCCCGCGTGGCCTGTCTGAAGCTAAACCAGCAACTCCGGAAA | 1131 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACATCATCATACCGATTGGCGTACCTCCACCAA | |
| | CTATTACATTAAGGTTCGTGCCGGTGACAATAAGTATATGCACCTG | |
| | AAAGTGTTCAACGGCCCGTACTGGCATTGGAAAGTTCGTCGTTTTG | |
| | CGGACCGTGTTCTGACCGGTTACCAGGTTGACAAGAACAAAGATG | |
| | ACGAGCTGACGGGTTTC | |
| FcRn-24 | ATGATCCCGCGTGGCCTGTCTGAAGCTAAACCAGCAACTCCGGAAA | 1132 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATACCCATAAAATCCTGCATTTTCATTCCACCAA | |
| | CTATTACATTAAGGTTCGTGCCGGTGACAATAAGTATATGCACCTG | |
| | AAAGTGTTCAACGGCCCGGATAAACAGCGTTACGAAGATAAACAG | |
| | GCGGACCGTGTTCTGACCGGTTACCAGGTTGACAAGAACAAAGAT | |
| | GACGAGCTGACGGGTTTC | |
| FcRn-24 | ATGATCCCGCGTGGCCTGTCTGAAGCTAAACCAGCAACTCCGGAAA | 1133 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAAACCATCATTTTTTTCTGCAGTTTTCCACCAAC | |
| | TATTACATTAAGGTTCGTGCCGGTGACAATAAGTATATGCACCTGA | |
| | AAGTGTTCAACGGCCCGCAGCATCATCATCCACATCGTCATCCAGC | |
| | GGACCGTGTTCTGACCGGTTACCAGGTTGACAAGAACAAAGATGA | |
| | CGAGCTGACGGGTTTC | |
| FcRn-24 | ATGATCCCGCGTGGCCTGTCTGAAGCTAAACCAGCAACTCCGGAAA | 1134 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCGTTACATCGGTCATAACTACTCTTCCACCAA | |
| | CTATTACATTAAGGTTCGTGCCGGTGACAATAAGTATATGCACCTG | |
| | AAAGTGTTCAACGGCCCGTGGCATCATTTTCATAACTCTTACGATG | |
| | CGGACCGTGTTCTGACCGGTTACCAGGTTGACAAGAACAAAGATG | |
| | ACGAGCTGACGGGTTTC | |
| FcRn-24 | ATGATCCCGCGTGGCCTGTCTGAAGCTAAACCAGCAACTCCGGAAA | 1135 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCATTACCATCATCAGTGGGATCCATCCACCAA | |
| | CTATTACATTAAGGTTCGTGCCGGTGACAATAAGTATATGCACCTG | |
| | AAAGTGTTCAACGGCCCGATCTGGTACTCTCATCGTCCACGTGCAG | |
| | CGGACCGTGTTCTGACCGGTTACCAGGTTGACAAGAACAAAGATG | |
| | ACGAGCTGACGGGTTTC | |
| FcRn-24 | ATGATCCCGCGTGGCCTGTCTGAAGCTAAACCAGCAACTCCGGAAA | 1136 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATAAAAAACATGGTCAGTACAAATCCACCAACT | |
| | ATTACATTAAGGTTCGTGCCGGTGACAATAAGTATATGCACCTGAA | |
| | AGTGTTCAACGGCCCGTGGGATGATCATACCCTGAAATGGTACGCG | |
| | GACCGTGTTCTGACCGGTTACCAGGTTGACAAGAACAAAGATGAC | |
| | GAGCTGACGGGTTTC | |
| FcRn-25 | ATGATCCCGCGTGGCCTGTCTGAAGCTAAACCAGCAACTCCGGAAA | 1137 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCATATCCAGGGTGTTTACTGGCGTTCCACCAA | |
| | CTATTACATTAAGGTTCGTGCCGGTGACAATAAGTATATGCACCTG | |
| | AAAGTGTTCAACGGCCCGATCGCATTTTGGGGTCCAAAACGTTTTG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 1138 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCGTTTTAAACATCATGTTCGTAACTCCACCAA | |
| | CTATTACATTAAGGTTCGTGCCGGTGACAATAAGTATATGCACCTG | |
| | AAAGTGTTCAACGGCCCGTTTCCACATCGTAACAAATCTGATGGTG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 1139 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGCATCATCAGCATCATCTGCTGGCATCCACCAA | |
| | CTATTACATTAAGGTTCGTGCCGGTGACAATAAGTATATGCACCTG | |
| | AAAGTGTTCAACGGCCCGTTTAAACGTTCTCAGCAGTGGGAATGGG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 1140 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAACAAACATCCATCTCCACGTGTTTCCACCAA | |
| | CTATTACATTAAGGTTCGTGCCGGTGACAATAAGTATATGCACCTG | |
| | AAAGTGTTCAACGGCCCGAAACATCGTTACCAGCCAACCCATTGGG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 1141 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCTGGTTTCATCAGCATGAACAGCAGTCCACCAA | |
| | CTATTACATTAAGGTTCGTGCCGGTGACAATAAGTATATGCACCTG | |
| | AAAGTGTTCAACGGCCCGTACCATGATATCTGGGCATGGCATGTTG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 1142 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGAAAGAATGGCGTTACCATCATCAGTCCACCA | |
| | ACTATTACATTAAGGTTCGTGCCGGTGACAATAAGTATATGCACCT | |
| | GAAAGTGTTCAACGGCCCGGATTTTGTTAAACATCATCTGCATGAT | |
| | GCGGACCGTGTTCTGACCGGTTACCAGGTTGACAAGAACAAAGAT | |
| | GACGAGCTGACGGGTTTC | |
| FcRn-25 | ATGATCCCGCGTGGCCTGTCTGAAGCTAAACCAGCAACTCCGGAAA | 1143 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATTTACCAAACATTGGGATCGTTGGTACTCCACCAA | |
| | CTATTACATTAAGGTTCGTGCCGGTGACAATAAGTATATGCACCTG | |
| | AAAGTGTTCAACGGCCCGATCTCTGATCATGTTCACTTTGGTTGGG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 1144 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCGTCTGTACGATCATTCTGTTTGGTCCACCAA | |
| | CTATTACATTAAGGTTCGTGCCGGTGACAATAAGTATATGCACCTG | |
| | AAAGTGTTCAACGGCCCGTACCATCATCGTGATCATTGGGGTTGGG | |
| | CGGACCGTGTTCTGACCGGTTACCAGGTTGACAAGAACAAAGATG | |
| | ACGAGCTGACGGGTTTC | |
| FcRn-25 | ATGATCCCGCGTGGCCTGTCTGAAGCTAAACCAGCAACTCCGGAAA | 1145 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATGGGAATACCAGACCCATCATCCAGCATCCACCA | |
| | ACTATTACATTAAGGTTCGTGCCGGTGACAATAAGTATATGCACCT | |
| | GAAAGTGTTCAACGGCCCGGAATGGTTTACCGTTGGTGGTATCGCA | |
| | GCGGACCGTGTTCTGACCGGTTACCAGGTTGACAAGAACAAAGAT | |
| | GACGAGCTGACGGGTTTC | |
| FcRn-25 | ATGATCCCGCGTGGCCTGTCTGAAGCTAAACCAGCAACTCCGGAAA | 1146 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTCATTTTCGTTCTCATCGTGATTTTTCCACCAAC | |
| | TATTACATTAAGGTTCGTGCCGGTGACAATAAGTATATGCACCTGA | |
| | AAGTGTTCAACGGCCCGGAACGTAAACATGCACATCAGCATCCAG | |
| | CGGACCGTGTTCTGACCGGTTACCAGGTTGACAAGAACAAAGATG | |
| | ACGAGCTGACGGGTTTC | |
| FcRn-26 | ATGATCCCGCGTGGCCTGTCTGAAGCTAAACCAGCAACTCCGGAAA | 1147 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTCGTCATACCCATCATCATCGTTCTTCCACCAAC | |
| | TATTACATTAAGGTTCGTGCCGGTGACAATAAGTATATGCACCTGA | |
| | AAGTGTTCAACGGCCCGGATTCTAACCTGTACAACGAATGGAACGC | |
| | GGACCGTGTTCTGACCGGTTACCAGGTTGACAAGAACAAAGATGA | |
| | CGAGCTGACGGGTTTC | |
| FcRn-26 | ATGATCCCGCGTGGCCTGTCTGAAGCTAAACCAGCAACTCCGGAAA | 1148 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCGCACGTTACGAACATGCACCAACCTCCACCA | |
| | ACTATTACATTAAGGTTCGTGCCGGTGACAATAAGTATATGCACCT | |
| | GAAAGTGTTCAACGGCCCGACCGCAAAACATTCTCATAAAAAACA | |
| | TGCGGACCGTGTTCTGACCGGTTACCAGGTTGACAAGAACAAAGAT | |
| | GACGAGCTGACGGGTTTC | |
| FcRn-26 | ATGATCCCGCGTGGCCTGTCTGAAGCTAAACCAGCAACTCCGGAAA | 1149 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTCATCGTAAAGAATCTTGGTACGTTTCCACCAA | |
| | CTATTACATTAAGGTTCGTGCCGGTGACAATAAGTATATGCACCTG | |
| | AAAGTGTTCAACGGCCCGAACTGGCCACATGGTATCGATCCAAAA | |
| | GCGGACCGTGTTCTGACCGGTTACCAGGTTGACAAGAACAAAGAT | |
| | GACGAGCTGACGGGTTTC | |
| FcRn-26 | ATGATCCCGCGTGGCCTGTCTGAAGCTAAACCAGCAACTCCGGAAA | 1150 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCATGGTTACGCACGTGGTCATCATTCCACCAA | |
| | CTATTACATTAAGGTTCGTGCCGGTGACAATAAGTATATGCACCTG | |
| | AAAGTGTTCAACGGCCCGAAACATATCCATGAACATAAATCTGAA | |
| | GCGGACCGTGTTCTGACCGGTTACCAGGTTGACAAGAACAAAGAT | |
| | GACGAGCTGACGGGTTTC | |
| FcRn-26 | ATGATCCCGCGTGGCCTGTCTGAAGCTAAACCAGCAACTCCGGAAA | 1151 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAACCCCACATAAAATCTGGCATTGGCATTCCACCAA | |
| | CTATTACATTAAGGTTCGTGCCGGTGACAATAAGTATATGCACCTG | |
| | AAAGTGTTCAACGGCCCGACCAAAAAATTTCATCAGCATGAACGT | |
| | GCGGACCGTGTTCTGACCGGTTACCAGGTTGACAAGAACAAAGAT | |
| | GACGAGCTGACGGGTTTC | |
| FcRn-26 | ATGATCCCGCGTGGCCTGTCTGAAGCTAAACCAGCAACTCCGGAAA | 1152 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATCTTACGCACAGCATACCCGTCTGCATTCCACCAA | |
| | CTATTACATTAAGGTTCGTGCCGGTGACAATAAGTATATGCACCTG | |
| | AAAGTGTTCAACGGCCCGACCCGTCATCATCAGCATTACTACCTGG | |
| | CGGACCGTGTTCTGACCGGTTACCAGGTTGACAAGAACAAAGATG | |
| | ACGAGCTGACGGGTTTC | |
| FcRn-26 | ATGATCCCGCGTGGCCTGTCTGAAGCTAAACCAGCAACTCCGGAAA | 1153 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAATCGATCATCGTTACCATTACCTGCATTCCACCAA | |
| | CTATTACATTAAGGTTCGTGCCGGTGACAATAAGTATATGCACCTG | |
| | AAAGTGTTCAACGGCCCGTGGTACTGGACCCAGCATCATCGTTGGG | |
| | CGGACCGTGTTCTGACCGGTTACCAGGTTGACAAGAACAAAGATG | |
| | ACGAGCTGACGGGTTTC | |
| FcRn-26 | ATGATCCCGCGTGGCCTGTCTGAAGCTAAACCAGCAACTCCGGAAA | 1154 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATGGTTACAACCATCGTAAAGTTCAGTCCACCAA | |
| | CTATTACATTAAGGTTCGTGCCGGTGACAATAAGTATATGCACCTG | |
| | AAAGTGTTCAACGGCCCGTACCATGTTTGGAACTGGCGTCTGAAAG | |
| | CGGACCGTGTTCTGACCGGTTACCAGGTTGACAAGAACAAAGATG | |
| | ACGAGCTGACGGGTTTC | |
| FcRn-26 | ATGATCCCGCGTGGCCTGTCTGAAGCTAAACCAGCAACTCCGGAAA | 1155 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTCATCTGAAAGCAGCACCATGGCATTCCACCA | |
| | ACTATTACATTAAGGTTCGTGCCGGTGACAATAAGTATATGCACCT | |
| | GAAAGTGTTCAACGGCCCGTTTCATCATTTTCGTCCACATCATCATG | |
| | CGGACCGTGTTCTGACCGGTTACCAGGTTGACAAGAACAAAGATG | |
| | ACGAGCTGACGGGTTTC | |
| FcRn-26 | ATGATCCCGCGTGGCCTGTCTGAAGCTAAACCAGCAACTCCGGAAA | 1156 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAGAAAAATACGCATCTTGGGAACGTTCCACCA | |
| | ACTATTACATTAAGGTTCGTGCCGGTGACAATAAGTATATGCACCT | |
| | GAAAGTGTTCAACGGCCCGTTTCTGAACGGTAAAAAACGTCATGTT | |
| | GCGGACCGTGTTCTGACCGGTTACCAGGTTGACAAGAACAAAGAT | |
| | GACGAGCTGACGGGTTTC | |
| FcRn-27 | ATGATCCCGCGTGGCCTGTCTGAAGCTAAACCAGCAACTCCGGAAA | 1157 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAAGGTCATCCACATGCACATCCACATTCCACCAA | |
| | CTATTACATTAAGGTTCGTGCCGGTGACAATAAGTATATGCACCTG | |
| | AAAGTGTTCAACGGCCCGTGGTGGAAAATCCATGGTTCTACCGTTG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1158 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCATACCGTCGTCATGAACATCATCAGTCCACCAA | |
| | CTATTACATTAAGGTTCGTGCCGGTGACAATAAGTATATGCACCTG | |
| | AAAGTGTTCAACGGCCCGAACTCTGATTTTCATCATAACCAGCAGG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1159 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGGTTTTCCACATTGGTTTGTTCATAACTCCACCAA | |
| | CTATTACATTAAGGTTCGTGCCGGTGACAATAAGTATATGCACCTG | |
| | AAAGTGTTCAACGGCCCGACCCATCATCTGCGTTACCATCATCAGG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1160 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATTTCGTCGTTACCAGTCTTTTCATTACTCCACCAAC | |
| | TATTACATTAAGGTTCGTGCCGGTGACAATAAGTATATGCACCTGA | |
| | AAGTGTTCAACGGCCCGTTTTACAAATACCATCAGGTTCGTTGGGC | |
| | GGACCGTGTTCTGACCGGTTACCAGGTTGACAAGAACAAAGATGA | |
| | CGAGCTGACGGGTTTC | |
| FcRn-27 | ATGATCCCGCGTGGCCTGTCTGAAGCTAAACCAGCAACTCCGGAAA | 1161 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACGTTACCGTCATCATGTTGATTACTCCACCAA | |
| | CTATTACATTAAGGTTCGTGCCGGTGACAATAAGTATATGCACCTG | |
| | AAAGTGTTCAACGGCCCGTACTCTTTTCGTGATCATCATTGGTGGG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1162 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATTACCTGAAACGTAACTTTCGTTACTCCACCAA | |
| | CTATTACATTAAGGTTCGTGCCGGTGACAATAAGTATATGCACCTG | |
| | AAAGTGTTCAACGGCCCGCCATTTTACCGTAACCATCATCATGAAG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1163 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACGTTCTCATCCAGGTAAACATGTTCATTCCACCAA | |
| | CTATTACATTAAGGTTCGTGCCGGTGACAATAAGTATATGCACCTG | |
| | AAAGTGTTCAACGGCCCGTTTCAGCTGAACCTGCGTTGGGGTCAGG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1164 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATCATCGTTGGGCAAAATGGCTGTCCACCAA | |
| | CTATTACATTAAGGTTCGTGCCGGTGACAATAAGTATATGCACCTG | |
| | AAAGTGTTCAACGGCCCGGTTCATAACTTTCATGATATCCGTCATG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1165 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGCAGCACATCATAACCATTGGCATATCTCCACCAA | |
| | CTATTACATTAAGGTTCGTGCCGGTGACAATAAGTATATGCACCTG | |
| | AAAGTGTTCAACGGCCCGGCACAGCATGGTCATGTTCCATTTTCTG | |
| | CGGACCGTGTTCTGACCGGTTACCAGGTTGACAAGAACAAAGATG | |
| | ACGAGCTGACGGGTTTC | |
| FcRn-27 | ATGATCCCGCGTGGCCTGTCTGAAGCTAAACCAGCAACTCCGGAAA | 1166 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCAGTTCAGAAACATGCAGGTTCTCATTCCACCAA | |
| | CTATTACATTAAGGTTCGTGCCGGTGACAATAAGTATATGCACCTG | |
| | AAAGTGTTCAACGGCCCGCCATGGCATAACGCAGAAATCAAACAT | |
| | GCGGACCGTGTTCTGACCGGTTACCAGGTTGACAAGAACAAAGAT | |
| | GACGAGCTGACGGGTTTC | |
| FcRn-28 | ATGATCCCGCGTGGCCTGTCTGAAGCTAAACCAGCAACTCCGGAAA | 1167 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATAACTGGCGTCATTGGCGTATCTGGTCCACCAA | |
| | CTATTACATTAAGGTTCGTGCCGGTGACAATAAGTATATGCACCTG | |
| | AAAGTGTTCAACGGCCCGGCAGGTTGGTCTTCTAACAAAGCAGATG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 1168 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACGTCATCATCATTGGGCATTTTCCACCAACTA | |
| | TTACATTAAGGTTCGTGCCGGTGACAATAAGTATATGCACCTGAAA | |
| | GTGTTCAACGGCCCGAAACGTCAGCATCATGATGTTGGTCAGGCGG | |
| | ACCGTGTTCTGACCGGTTACCAGGTTGACAAGAACAAAGATGACG | |
| | AGCTGACGGGTTTC | |
| FcRn-28 | ATGATCCCGCGTGGCCTGTCTGAAGCTAAACCAGCAACTCCGGAAA | 1169 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGTTTCTTACGATGATATCACCTGGGTTTCCACCAA | |
| | CTATTACATTAAGGTTCGTGCCGGTGACAATAAGTATATGCACCTG | |
| | AAAGTGTTCAACGGCCCGAACTCTTCTTACGGTTGGCTGTGGTGGG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 1170 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACCACCTCATCCACGTGTTCAGCATTACTCCACCAA | |
| | CTATTACATTAAGGTTCGTGCCGGTGACAATAAGTATATGCACCTG | |
| | AAAGTGTTCAACGGCCCGGCATTTCGTGATCATCGTGCACCACATG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 1171 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACAGTTTCGTCATCATCAGCATGAATCCACCAA | |
| | CTATTACATTAAGGTTCGTGCCGGTGACAATAAGTATATGCACCTG | |
| | AAAGTGTTCAACGGCCCGAAATGGTGGTCTACCCAGGGTATCGTTG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 1172 |
| 5 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAACATCATGAATACCATTACCGTTACTCCACCAA | |
| | CTATTACATTAAGGTTCGTGCCGGTGACAATAAGTATATGCACCTG | |
| | AAAGTGTTCAACGGCCCGTTTCGTCCAGTTCATCATATCCGTATCG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 1173 |
| 6 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATCATCATCATCGTCAGCATCCATCCACCAACTA | |
| | TTACATTAAGGTTCGTGCCGGTGACAATAAGTATATGCACCTGAAA | |
| | GTGTTCAACGGCCCGAAAGTTGGTCAGGGTGTTAACCTGGGTGCGG | |
| | ACCGTGTTCTGACCGGTTACCAGGTTGACAAGAACAAAGATGACG | |
| | AGCTGACGGGTTTC | |
| FcRn-28 | ATGATCCCGCGTGGCCTGTCTGAAGCTAAACCAGCAACTCCGGAAA | 1174 |
| 7 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAAAACTGCATCAGGCACATCATTGGCATTCCACCAA | |
| | CTATTACATTAAGGTTCGTGCCGGTGACAATAAGTATATGCACCTG | |
| | AAAGTTTTCAACGGCCCGGAATGGTCTAACAAACATTACCAGTGGG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 1175 |
| 8 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAATACCATCATTACGGTACCTCTCGTTCCACCAA | |
| | CTATTACATTAAGGTTCGTGCCGGTGACAATAAGTATATGCACCTG | |
| | AAAGTGTTCAACGGCCCGCGTCAGCTGAAACATCATACCAACTTTG | |
| | CGGACCGTGTTCTGACCGGTTACCAGGTTGACAAGAACAAAGATG | |
| | ACGAGCTGACGGGTTTC | |
| FcRn-28 | ATGATCCCGCGTGGCCTGTCTGAAGCTAAACCAGCAACTCCGGAAA | 1176 |
| 9 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATAACAAACATATCCCACAGCGTCAGTCCACCA | |
| | ACTATTACATTAAGGTTCGTGCCGGTGACAATAAGTATATGCACCT | |
| | GAAAGTGTTCAACGGCCCGCGTAACCATGTTGCAGAAAAATACTG | |
| | GGCGGACCGTGTTCTGACCGGTTACCAGGTTGACAAGAACAAAGA | |
| | TGACGAGCTGACGGGTTTC | |
| FcRn-29 | ATGATCCCGCGTGGCCTGTCTGAAGCTAAACCAGCAACTCCGGAAA | 1177 |
| 0 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATAAACAGTGGCAGTGGACCATCGTTTCCACCAA | |
| | CTATTACATTAAGGTTCGTGCCGGTGACAATAAGTATATGCACCTG | |
| | AAAGTGTTCAACGGCCCGGCATACAAATCTGATAAAATCCGTAAA | |
| | GCGGACCGTGTTCTGACCGGTTACCAGGTTGACAAGAACAAAGAT | |
| | GACGAGCTGACGGGTTTC | |
| FcRn-29 | ATGATCCCGCGTGGCCTGTCTGAAGCTAAACCAGCAACTCCGGAAA | 1178 |
| 1 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCATACCGTATCGGTCATGGTGTTCAGCATTCCACCAA | |
| | CTATTACATTAAGGTTCGTGCCGGTGACAATAAGTATATGCACCTG | |
| | AAAGTGTTCAACGGCCCGTACGATAAACCATACATCGTTTGGATCG | |
| | CGGACCGTGTTCTGACCGGTTACCAGGTTGACAAGAACAAAGATG | |
| | ACGAGCTGACGGGTTTC | |
| FcRn-29 | ATGATCCCGCGTGGCCTGTCTGAAGCTAAACCAGCAACTCCGGAAA | 1179 |
| 2 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGATCAGGTTCGTCGTATCCCACATCATTCCACCAA | |
| | CTATTACATTAAGGTTCGTGCCGGTGACAATAAGTATATGCACCTG | |
| | AAAGTGTTCAACGGCCCGCATGATAAACATCCACAGTCTTGGGCAG | |
| | CGGACCGTGTTCTGACCGGTTACCAGGTTGACAAGAACAAAGATG | |
| | ACGAGCTGACGGGTTTC | |
| FcRn-29 | ATGATCCCGCGTGGCCTGTCTGAAGCTAAACCAGCAACTCCGGAAA | 1180 |
| 3 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCAGAAGGTAAACATGAATTTCGTTTTCAGTCCACCAA | |
| | CTATTACATTAAGGTTCGTGCCGGTGACAATAAGTATATGCACCTG | |
| | AAAGTGTTCAACGGCCCGTGGGATAAACATCGTCAGCATCTGTGGG | |
| | CGGACCGTGTTCTGACCGGTTACCAGGTTGACAAGAACAAAGATG | |
| | ACGAGCTGACGGGTTTC | |
| FcRn-29 | ATGATCCCGCGTGGCCTGTCTGAAGCTAAACCAGCAACTCCGGAAA | 1181 |
| 4 | TTCAAGAGATCGTCGATAAGGTGAAACCGCAGCTGGAAGAGAAAA | |
| | CGAACGAAACCTACGGTAAGCTGGAAGCGGTCCAGTACAAAACCC | |
| | AAGTGCTAGCACATTACTGGGGTCGTTGGTACAAAATCTCCACCAA | |
| | CTATTACATTAAGGTTCGTGCCGGTGACAATAAGTATATGCACCTG | |
| | AAAGTGTTCAACGGCCCGTTTCATGCATTTTGGCATCTGGCATACG | |
| | CGGACCGTGTTCTGACCGGTTACCAGGTTGACAAGAACAAAGATG | |
| | ACGAGCTGACGGGTTTC | |

Anti-human FcRn AFFIMER® polypeptides provided herein, in some embodiments, are linked to another molecule and extend the half-life of that molecule (e.g., a therapeutic polypeptide). The term half-life refers to the amount of time it takes for a substance, such as an therapeutic AFFIMER® polypeptide, to lose half of its pharmacologic or physiologic activity or concentration. Biological half-life can be affected by elimination, excretion, degradation (e.g., enzymatic degradation) of the substance, or absorption and concentration in certain organs or tissues of the body. Biological half-life can be assessed, for example, by determining the time it takes for the blood plasma concentration of the substance to reach half its steady state level (“plasma half-life”).

In some embodiments, an anti-human FcRn AFFIMER® polypeptide extends the serum half-life of a molecule (e.g., a therapeutic polypeptide) in vivo. For example, an anti-human FcRn AFFIMER® polypeptide may extend the half-life of a molecule by at least 1.2-fold, relative to the half-life of the molecule not linked to an anti-human FcRn AFFIMER® polypeptide. In some embodiments, an anti-human FcRn AFFIMER® polypeptide extends the half-life of a molecule by at least 1.5-fold, at least 2-fold, at least 3-fold, at least 4-fold, at least 5-fold, at least 6-fold, at least 7-fold, at least 8-fold, at least 9-fold, at least 10-fold, at least 20-fold, or at least 30-fold, relative to the half-life of the molecule not linked to an anti-human FcRn AFFIMER® polypeptide. In some embodiments, an anti-human FcRn AFFIMER® polypeptide extends the half-life of a molecule by 1.2-fold to 5-fold, 1.2-fold to 10-fold, 1.5-fold to 5-fold, 1.5-fold to 10-fold, 2-fold to 5-fold, 2-fold to 10-fold, 3-fold to 5-fold, 3-fold to 10-fold, 15-fold to 5-fold, 4-fold to 10-fold, or 5-fold to 10-fold, relative to the half-life of the molecule not linked to an anti-human FcRn AFFIMER® polypeptide. In some embodiments, an anti-human FcRn AFFIMER® polypeptide extends the half-life of a molecule by at least 6 hours, at least 12 hours, at least 24 hours, at least 48 hours, at least 72 hours, at least 96 hours, for example, at least 1 week after in vivo administration, relative to the half-life of the molecule not linked to an anti-human FcRn AFFIMER® polypeptide.

### Polypeptides

A polypeptide is a polymer of amino acids (naturally-occurring or non-naturally occurring, e.g., amino acid analogs) of any length. The terms “polypeptide” and “peptide” are used interchangeably herein unless noted otherwise. A protein is one example of a polypeptide. It should be understood that a polypeptide may be linear or branched, it may comprise naturally-occurring and/or non-naturally-occurring (e.g., modified) amino acids, and/or it may include non-amino acids (e.g., interspersed throughout the polymer). A polypeptide, as provided herein, may be modified (e.g., naturally or non-naturally), for example, via disulfide bond formation, glycosylation, lipidation, acetylation, phosphorylation, or conjugation with a labeling component. Polypeptides, in some instances, may contain at least one analog of an amino acid (including, for example, unnatural amino acids) and/or other modifications.

An amino acid (also referred to as an amino acid residue) participates in peptide bonds of a polypeptide. In general, the abbreviations used herein for designating the amino acids are based on recommendations of the IUPAC-IUB Commission on Biochemical Nomenclature (see Biochemistry (1972) 11:1726-1732). For instance, Met, Ile, Leu, Ala and Gly represent “residues” of methionine, isoleucine, leucine, alanine and glycine, respectively. A residue is a radical derived from the corresponding α amino acid by eliminating the OH portion of the carboxyl group and the H portion of the α amino group. An amino acid side chain is that part of an amino acid exclusive of the —CH(NH2)COOH portion, as defined by K. D. Kopple, “Peptides and Amino Acids” W. A. Benjamin Inc., New York and Amsterdam, 1966, pages 2 and 33.

Amino acids used herein, in some embodiments, are naturally-occurring amino acids found in proteins, for example, or the naturally-occurring anabolic or catabolic products of such amino acids that contain amino and carboxyl groups. Examples of amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs that have been identified as constituents of peptidylglycan bacterial cell walls.

Amino acids having basic sidechains include Arg, Lys and His Amino acids having acidic sidechains include Glu and Asp Amino acids having neutral polar sidechains include Ser, Thr, Asn, Gln, Cys and Tyr Amino acids having neutral non-polar sidechains include Gly, Ala, Val, Ile, Leu, Met, Pro, Trp and Phe Amino acids having non-polar aliphatic sidechains include Gly, Ala, Val, Ile and Leu Amino acids having hydrophobic sidechains include Ala, Val, Ile, Leu, Met, Phe, Tyr and Trp Amino acids having small hydrophobic sidechains include Ala and Val. Amino acids having aromatic sidechains include Tyr, Trp and Phe.

The term amino acid includes analogs, derivatives and congeners of any specific amino acid referred to herein; for instance, the AFFIMER® polypeptides (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally-occurring amino acid metabolites or precursors having side chains that are suitable herein will be recognized by those skilled in the art and are included in the scope of the present disclosure.

Also included herein are the (D) and (L) stereoisomers of such amino acids when the structure of the amino acid admits of stereoisomeric forms. The configuration of the amino acids and amino acids herein are designated by the appropriate symbols (D), (L) or (DL); furthermore, when the configuration is not designated the amino acid or residue can have the configuration (D), (L) or (DL). It will be noted that the structure of some of the compounds of the present disclosure includes asymmetric carbon atoms. It is to be understood accordingly that the isomers arising from such asymmetry are included within the scope of the present disclosure. Such isomers can be obtained in substantially pure form by classical separation techniques and by sterically controlled synthesis. For the purposes of this disclosure, unless expressly noted to the contrary, a named amino acid shall be construed to include both the (D) or (L) stereoisomers.

Percent identity, in the context of two or more nucleic acids or polypeptides, refers to two or more sequences or subsequences that are the same (identical/100% identity) or have a specified percentage (e.g., at least 70% identity) of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the present disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A conservative amino acid substitution is one in which one amino acid residue is replaced with another amino acid residue having a similar side chain Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the present disclosure do not abrogate the binding of the polypeptide, soluble protein, or antibody containing the amino acid sequence, to the target binding site. Methods of identifying amino acid conservative substitutions that do not eliminate binding are well-known in the art.

Herein, it should be understood that an isolated molecule (e.g., polypeptide (e.g., soluble protein, antibody, etc.), polynucleotide (e.g., vector), cell, or other composition) is in a form not found in nature. Isolated molecules, for example, have been purified to a degree that is not possible in nature.

In some embodiments, an isolated molecule (e.g., polypeptide (e.g., soluble protein, antibody, etc.), polynucleotide (e.g., vector), cell, or other composition) is substantially pure, which refer to an isolated molecule that is at least 50% pure (e.g., free from 50% of contaminants associated with the unpurified form of the molecule), at least 90% pure, at least 95% pure, at least 98% pure, or at least 99% pure.

Conjugates, Including Polypeptide Fusions

The verb conjugate (used interchangeably with the verb link) herein refers to the joining together of two or more molecules (e.g., polypeptides and/or chemical moieties) to form another molecule. Thus, one molecule (e.g., an anti-FcRn AFFIMER® polypeptide) conjugated to another molecule (e.g., another AFFIMER® polypeptide, drug molecule, or other therapeutic protein or nucleic acid) forms a conjugate. The joining of two or more molecules can be, for example, through a non-covalent bond or a covalent bond. For example, an anti-FcRn AFFIMER® polypeptide linked directly or indirectly to an an FcRn affmier. For example, an anti-FcRn AFFIMER® polypeptide linked directly or indirectly to a chemical moiety or to another polypeptide (e.g., a heterologous polypeptide) forms a conjugate, as provided herein. Non-limiting examples of conjugates include chemical conjugates (e.g., joined through “click” chemistry or another chemical reaction) and fusions (two molecules linked by contiguous peptide bonds). In some embodiments, a conjugate is a fusion polypeptide, for example, a fusion protein. In some embodiments, an anti-FcRn AFFIMER® polypeptide is conjugated to two or more other molecules. For example, dual (or multi) mode of action drug conjugates may be conjugated to an anti-FcRn AFFIMER® polypeptide of the present disclosure. Such dual mode of action drug conjugates include those of the TMAC (Tumor Microenvironment-Activated Conjugates) platform (see, e.g., avacta.com/therapeutics/tmac-affimer-drug-conjugates).

A fusion polypeptide (e.g., fusion protein) is a polypeptide comprising at least two domains (e.g., protein domains) encoded by a polynucleotide comprising nucleotide sequences of at least two separate molecules (e.g., two genes). In some embodiments, a polypeptide comprises a heterologous polypeptide covalently linked (to an amino acid of the polypeptide) through an amide bond to form a contiguous fusion polypeptide (e.g., fusion protein). In some embodiments, the heterologous polypeptide comprises a therapeutic polypeptide. In some embodiments, an anti-FcRn AFFIMER® polypeptide is conjugated to a heterologous polypeptide through contiguous peptide bonds at the C-terminus or N-terminus of the anti-human FcRn AFFIMER® polypeptide.

A linker is a molecule inserted between a first polypeptide (e.g., as AFFIMER® polypeptide) and a second polypeptide (e.g., another AFFIMER® polypeptide, an Fc domain, a ligand binding domain, etc). A linker may be any molecule, for example, one or more nucleotides, amino acids, chemical functional groups. In some embodiments, the linker is a peptide linker (e.g., two or more amino acids). Linkers should not adversely affect the expression, secretion, or bioactivity of the polypeptides. In some embodiments, linkers are not antigenic and do not elicit an immune response. An immune response includes a response from the innate immune system and/or the adaptive immune system. Thus, an immune response may be a cell-mediate response and/or a humoral immune response. The immune response may be, for example, a T cell response, a B cell response, a natural killer (NK) cell response, a monocyte response, and/or a macrophage response. Other cell responses are contemplated herein.

In some embodiments, linkers are non-protein-coding.

In some embodiments, a conjugate comprises an AFFIMER® polypeptide linked to a therapeutic or diagnostic molecule. In some embodiments, a conjugate comprises an AFFIMER® polypeptide linked to another protein, a nucleic acid, a drug, or other small molecule or macromolecule.

Any conjugation method may be used, or readily adapted, for joining a molecule to an AFFIMER® polypeptide of the present disclosure, including, for example, the methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407.

### Therapeutics

In some embodiments, an AFFIMER® polypeptide is linked to a therapeutic molecule. Herein, a therapeutic molecule may be used, for example, to prevent and/or treat a disease in a subject, such as a human subject or other animal subject.

In some embodiments, the therapeutic molecule is for the treatment of an autoimmune disease (a condition in which a subject's immune system mistaken attacks his/her body). Non-limiting examples of autoimmune diseases include myasthenia gravis, pemphigus vulgaris, neuromyelitis optica, Guillain-Barre syndrome, rheumatoid arthritis, systemic lupus erythematosus (lupus), idiopathic thrombocytopenic purpura, thrombotic thrombocytopenic purpura, antiphospholipid syndrome (APS), autoimmune urticarial, chronic inflammatory demyelinating polyneuropathy (CIDP), psoriasis, Goodpasture's syndrome, Graves' disease, inflammatory bowel disease, Crohn's disease, Sjorgren's syndrome, hemolytic anemia, neutropenia, paraneoplastic cerebellar degeneration, paraproteinemic polyneuropathies, primary biliary cirrhosis, stiff person syndrome, vitiligo, warm idiopathic haemolytic anaemia, multiple sclerosis, type 1 diabetes mellitus, Hashimoto's thyroiditis, Myasthenia gravis, autoimmune vasculitis, pernicus anemia, and celiac disease. Other autoimmune diseases are contemplated herein.

In some embodiments, the therapeutic molecule is for the treatment of a cancer. Non-limiting examples of cancers include skin cancer (e.g., melanoma or non-melanoma, such as basal cell or squamous cell), lung cancer, prostate cancer, breast cancer, colorectal cancer, kidney (renal) cancer, bladder cancer, non-Hodgkin's lymphoma, thyroid cancer, endometrial cancer, exocrine cancer, and pancreatic cancer. Other cancers are contemplated herein.

In some embodiments, the therapeutic molecule is for the treatment of an inflammatory disease or disorder (a disease, disorder or condition characterized by inflammation of body tissue or having an inflammatory component). These include local inflammatory responses and systemic inflammation. Non-limiting examples of inflammatory disorders include: transplant rejection, including skin graft rejection; chronic inflammatory disorders of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome, and chronic obstructive airway disease; inflammatory disorders of the eye including corneal dystrophy, trachoma, onchocerciasis, uveitis, sympathetic ophthalmitis and endophthalmitis; chronic inflammatory disorders of the gums, including gingivitis and periodontitis; tuberculosis; leprosy; inflammatory diseases of the kidney including uremic complications, glomerulonephritis and nephrosis; inflammatory disorders of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune disorders, immune-complex vasculitis, systemic lupus and erythematodes; systemic lupus erythematosus (SLE); and inflammatory diseases of the heart such as cardiomyopathy, ischemic heart disease hypercholesterolemia, atherosclerosis; as well as various other diseases with significant inflammatory components, including preeclampsia; chronic liver failure, brain and spinal cord trauma. There may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent used in cancer chemotherapy.

In some embodiments, the therapeutic molecule is for the treatment of a cardiovascular disease or disorder. Cardiovascular disorders include, but are not limited to, abnormal heart rhythms, or arrhythmias, aorta disease and Marfan syndrome, congenital heart disease, coronary artery disease (e.g., narrowing of the arteries), deep vein thrombosis and pulmonary embolism, heart attack, heart failure, heart muscle disease (e.g., cardiomyopathy), heart valve disease, pericardial disease, peripheral vascular disease, rheumatic heart disease, stroke, and vascular disease (e.g., blood vessel disease).

In some embodiments, the therapeutic molecule is for the treatment of a metabolic disease or disorder. Examples of metabolic disorders include the following: glycogen storage diseases (also referred to as glycogenosis or dextrinosis), which include disorders that affect carbohydrate metabolism; fatty oxidation disorders, which affect fat metabolism and metabolism of fat components; and mitochondrial disorders, which affect mitochondria. Examples of glycogen storage diseases (GSD) include at least GSD type I (glucose-6-phosphatase deficiency; von Gierke's disease); GSD type II (acid maltase deficiency; Pompe's disease); GSD type III (glycogen debrancher deficiency; Cori's disease or Forbe's disease); GSD type IV (glycogen branching enzyme deficiency; Andersen disease); GSD type V (muscle glycogen phosphorylase deficiency; McArdle disease); GSD type VI (liver phosphorylase deficiency, Hers's disease); GSD type VII (muscle phosphofructokinase deficiency; Tarui's disease); GSD type IX (phosphorylase kinase deficiency); and GSD type XI (glucose transporter deficiency; Fanconi-Bickel disease). Examples of fatty acid metabolism deficiencies include at least coenzyme A dehydrogenase deficiencies; other coenzyme A enzyme deficiencies; carnitine-related disorders; or lipid storage disorders. Examples of coenzyme A dehydrogenase deficiencies include at least very long-chain acyl-coenzyme A dehydrogenase deficiency (VLCAD); long-chain 3-hydroxyacyl-coenzyme A dehydrogenase deficiency (LCHAD); medium-chain acyl-coenzyme A dehydrogenase deficiency (MCAD); short-chain acyl-coenzyme A dehydrogenase deficiency (SCAD); and short chain L-3-hydroxyacyl-coA dehydrogenase deficiency (SCHAD). Examples of other coenzyme A enzyme deficiencies include at least 2,4 Dienoyl-CoA reductase deficiency; 3-hydroxy-3-methylglutaryl-CoA lyase deficiency; and malonyl-CoA decarboxylase deficiency. Examples of carnitine-related deficiencies include at least primary carnitine deficiency; carnitine-acylcarnitine translocase deficiency; carnitine palmitoyltransferase I deficiency (CPT); and carnitine palmitoyltransferase II deficiency (CPT). Examples of lipid storage diseases include acid lipase diseases; Wolman disease; cholesteryl ester storage disease; Gaucher disease; Niemann-Pick disease; Fabry disease; Farber's disease; gangliosidoses; Krabbe disease; and metachromatic leukodystrophy. Other fatty acid metabolism disorders include at least mitochondrial trifunctional protein deficiency; electron transfer flavoprotein (ETF) dehydrogenase deficiency (GAII & MADD); Tangier disease; and acute fatty liver of pregnancy. Examples of mitochondrial diseases include at least progressive external ophthalmoplegia (PEO); Diabetes mellitus and deafness (DAD); Leber hereditary optic neuropathy (LHON) Mitochondrial encephalomyopathy, lactic acidosis, and stroke-like syndrome (MELAS); Myoclonic epilepsy and ragged-red fibers (MERRF); Leigh syndrome; subacute sclerosing encephalopathy; Neuropathy, ataxia, retinitis pigmentosa, and ptosis (NARP); Kearns-Sayre syndrome (KSS); Myoneurogenic gastrointestinal encephalopathy (MNGIE).

The term treat, as known in the art, refers to the process of alleviating at least one symptom associated with a disease. A symptom may be a physical, mental, or pathological manifestation of a disease. Symptoms associated with various diseases are known. To treat or prevent a particular condition, a conjugate as provided herein (e.g., an anti-human FcRn AFFIMER® polypeptide linked to a therapeutic molecule) should be administered in an effective amount, which can be any amount used to treat or prevent the condition. Thus, in some embodiments, an effective amount is an amount used to alleviate a symptom associated with the particular disease being treated. Methods are known for determining effective amounts of various therapeutic molecules, for example.

A subject may be any animal (e.g., a mammal), including, but not limited to, humans, non-human primates, canines, felines, and rodents. A “patient” refers to a human subject.

In some embodiments, an anti-human FcRn AFFIMER® polypeptide is linked to an agonist of a particular molecule (e.g., receptor) of interest. In other embodiments, an anti-human FcRn AFFIMER® polypeptide is linked to an antagonist of a particular molecule of interest. An agonist herein refers to a molecule that binds to and activates another molecule to produce a biological response. By contrast, an antagonist blocks the action of the agonist, and an inverse agonist causes an action opposite to that of the agonist. Thus, an antagonist herein refers to a molecule that binds to and deactivates or prevents activation of another molecule.

In some embodiments, an AFFIMER® polypeptide is considered “pharmaceutically acceptable”, and in some embodiments, is formulated with a pharmaceutically-acceptable excipient. A molecule or other substance/agent is considered pharmaceutically acceptable?if it is approved or approvable by a regulatory agency of the Federal government or a state government or listed in the U.S. Pharmacopeia or other generally recognized pharmacopeia for use in animals, including humans. An excipient may be any inert (inactive), non-toxic agent, administered in combination with an AFFIMER® polypeptide. Non-limiting examples of excipients include buffers (e.g., sterile saline), salts, carriers, preservatives, fillers, coloring agents.

Therapeutic molecules for use herein include, for example, those recognized in the official United States Pharmacopeia, official Homeopathic Pharmacopeia of the United States, official National Formulary, or any supplement thereof, and include, but are not limited, to small molecules chemicals/drugs, polynucleotides (e.g., RNA interference molecules, such as miRNA, siRNA, shRNA, and antisense RNA), and polypeptides (e.g., antibodies). Classes of therapeutic molecules that may be used as provided herein include, but are not limited to, recombinant proteins, antibodies, cytotoxic agents, anti-metabolites, alkylating agents, antibiotics, growth factors (e.g., erythropoietin, granulocyte colony-stimulating factor (G-CSF), granulocyte-macrophage colony-stimulating factor (GM-CSF), keratinocyte growth factor)), cytokines, chemokines, interferons (e.g., interferon-alpha, interferon-beta, interferon-gamma), blood factors (e.g., factor VIII, factor Vila, factor IX, thrombin, antithrombin), anti-mitotic agents, toxins, apoptotic agents, (e.g., DNA alkylating agents), topoisomerase inhibitors, endoplasmic reticulum stress inducing agents, platinum compounds, antimetabolites, vincalkaloids, taxanes, epothilones, enzyme inhibitors, receptor antagonists, tyrosine kinase inhibitors, radiosensitizers, chemotherapeutic combination therapies, receptor traps, receptor ligands, angiogenic agents, anti-angiogenic agents, anti-coagulants and thrombolytics (e.g., tissue plasminogen activator, hirudin, protein C), neurotransmitters, erythropoiesis-stimulating agents, insulin, growth hormones (e.g., human growth hormone (hGH), follicle-stimulating hormone), metabolic hormones (e.g., incretins), recombinant IL-1 receptor antagonists, and bispecific T-cell engaging molecules (BITEs®).

Specific examples of therapeutic molecules to which an anti-human FcRn AFFIMER® polypeptide may be linked (e.g., to extend the half-life of the molecules) includes fibroblast growth factor 21 (FGF21), insulin, insulin receptor peptide, GIP (glucose-dependent insulinotropic polypeptide), bone morphogenetic protein 9 (BMP-9), amylin, peptide YY (PYY3-36), pancreatic polypeptide (PP), interleukin 21 (IL-21), glucagon-like peptide 1 (GLP-1), Plectasin, Progranulin, Osteocalcin (OCN), Apelin, GLP-1, Exendin 4, adiponectin, IL-1Ra (Interleukin 1 Receptor Antagonist), VIP (vasoactive intestinal peptide), PACAP (Pituitary adenylate cyclase-activating polypeptide), leptin, INGAP (islet neogenesis associated protein), BMP (bone morphogenetic protein), and osteocalcin (OCN).

Antibodies

In some embodiments, a heterologous polypeptide to which an anti-human FcRn AFFIMER® polypeptide is linked is an antibody (e.g., a variable region of an antibody). Thus, the present disclosure, in some embodiments, provides an AFFIMER® polypeptide-antibody fusion protein. In some embodiments, an AFFIMER® polypeptide-antibody fusion protein comprises a full length antibody comprising, for example, at least one AFFIMER® polypeptide sequence appended to the C-terminus or N-terminus of at least one of its VH and/or VL chains (at least one chain of the assembled antibody forms a fusion protein with an AFFIMER® polypeptide). AFFIMER® polypeptide-antibody fusion proteins, in some embodiments, comprise at least one AFFIMER® polypeptide and an antigen binding site or variable region of an antibody fragment.

An antibody is an immunoglobulin molecule that recognizes and specifically binds a target, such as a polypeptide (e.g., peptide or protein), polynucleotide, carbohydrate, lipid, or a combination of any of the foregoing, through at least one antigen-binding site. The antigen-binding site, in some embodiments, is within the variable region of the immunoglobulin molecule. Antibodies include polyclonal antibodies, monoclonal antibodies, antibody fragments (such as Fab, Fab′, F(ab′)2, and Fv fragments), single chain Fv (scFv) antibodies provided those fragments have been formatted to include an Fc or other FcγIII binding domain, multispecific antibodies, bispecific antibodies, monospecific antibodies, monovalent antibodies, chimeric antibodies, humanized antibodies, human antibodies, fusion proteins comprising an antigen-binding site of an antibody (formatted to include an Fc or other FcγIII binding domain), and any other modified immunoglobulin molecule comprising an antigen-binding site as long as the antibodies exhibit the desired biological activity.

An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu.

A variable region of an antibody can be a variable region of an antibody light chain or a variable region of an antibody heavy chain, either alone or in combination. Generally, the variable region of heavy and light chains each consist of four framework regions (FR) and three complementarity determining regions (CDRs), also known as hypervariable regions. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

Humanized antibodies are forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the Fv framework region residues of a human immunoglobulin are replaced with the corresponding residues in an antibody from a non-human species. A humanized antibody can be further modified by the substitution of additional residues either in the Fv framework region and/or within the replaced non-human residues to refine and optimize antibody specificity, affinity, and/or binding capability. A humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

An epitope (also referred to as an antigenic determinant) is a portion of an antigen capable of being recognized and specifically bound by a particular antibody, a particular AFFIMER® polypeptide, or other particular binding domain. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds to” or is “specific for” refers to measurable and reproducible interactions such as binding between a target and an AFFIMER® polypeptide, antibody or other binding partner, which is determinative of the presence of the target in the presence of a heterogeneous population of molecules including biological molecules. For example, an AFFIMER® polypeptide that specifically binds to a target is an AFFIMER® polypeptide that binds this target with greater affinity, avidity (if multimeric formatted), more readily, and/or with greater duration than it binds to other targets.

Non-limiting examples of antibodies that may be conjugated to an FcRn-HSA an AFFIMER® polypeptide of the present disclosure 3F8, 8H9, abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, anatumomab mafenatox, andecaliximab, anetumab ravtansine, anifrolumab, anrukinzumab (IMA-638), apolizumab, aprutumab ixadotin, arcitumomab, ascrinvacumab, aselizumab, atezolizumab, atidortoxumab, atinumab, atorolimumab, avelumab, azintuxizumab vedotin, bapineuzumab, basiliximab, bavituximab, BCD-100, bectumomab, begelomab, belantamab mafodotin, belimumab, bemarituzumab, benralizumab, berlimatoxumab, bermekimab, bersanlimab, bertilimumab, besilesomab, bevacizumab, bezlotoxumab, biciromab, bimagrumab, bimekizumab, birtamimab, bivatuzumab mertansine, bleselumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, capromab pendetide, carlumab, carotuximab, catumaxomab, cBR96-doxorubicin immunoconjugate, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, CR6261, crenezumab, crizanlizumab, crotedumab, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, diridavumab, domagrozumab, dorlimomab aritox, dostarlimab, drozitumab, DS-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab vedotin, enavatuzumab, enfortumab vedotin, enlimomab pegol, enoblituzumab, enokizumab, enoticumab, ensituximab, epitumomab cituxetan, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, 1-BTA05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IBI308, ibritumomab tiuxetan, icrucumab, idarucizumab, ifabotuzumab, igovomab, iladatuzumab vedotin, IMAB362, imalumab, imaprelimab, imciromab, imgatuzumab, inclacumab, indatuximab ravtansine, indusatumab vedotin, inebilizumab, infliximab, inolimomab, inotuzumab ozogamicin, intetumumab, iomab-b, ipilimumab, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, lilotomab satetraxetan, lintuzumab, lirilumab, lodelcizumab, lokivetmab, loncastuximab tesirine, lorvotuzumab mertansine, losatuxizumab vedotin, lucatumumab, lulizumab pegol, lumiliximab, lumretuzumab, lupartumab amadotin, lutikizumab, mapatumumab, margetuximab, marstacimab, maslimomab, matuzumab, mavrilimumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-CD3, nacolomab tafenatox, namilumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odulimomab, ofatumumab, olaratumab, oleclumab, olendalizumab, olokizumab, omalizumab, omburtamab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, pdr001, pembrolizumab, pemtumomab, perakizumab, pertuzumab, pexelizumab, pidilizumab, pinatuzumab vedotin, pintumomab, placulumab, plozalizumab, pogalizumab, polatuzumab vedotin, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab, ravagalimab, ravulizumab, raxibacumab, refanezumab, regavirumab, relatlimab, remtolumab, reslizumab, rilotumumab, rinucumab, risankizumab, rituximab, rivabazumab pegol, rmab, robatumumab, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sarilumab, satralizumab, satumomab pendetide, secukinumab, selicrelumab, seribantumab, setoxaximab, setrusumab, sevirumab, SGN-CD19A, SHP647, sibrotuzumab, sifalimumab, siltuximab, simtuzumab, siplizumab, sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab tetraxetan, tadocizumab, talacotuzumab, talizumab, tamtuvetmab, tanezumab, taplitumomab paptox, tarextumab, tavolimab, tefibazumab, telimomab aritox, telisotuzumab vedotin, tenatumomab, teneliximab, teplizumab, tepoditamab, teprotumumab, tesidolumab, tetulomab, tezepelumab, TGN1412, tibulizumab, tigatuzumab, tildrakizumab, timigutuzumab, timolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab (IMAB362, claudiximab), and zolimomab aritox.

Other Therapeutic Molecules

Non-limiting examples of cytokines include IL-2, IL-12, TNF-alpha, IFN alpha, IFN beta, IFN gamma, IL-10, IL-15, IL-24, GM-CSF, IL-3, IL-4, IL-5, IL-6, IL-7, IL-9, IL-11, IL-13, LIF, CD80, B70, TNF beta, LT-beta, CD-40 ligand, Fas-ligand, TGF-beta, IL-1alpha and IL-1 beta.

Non-limiting examples of chemokines include IL-8, GRO alpha, GRO beta, GRO gamma, ENA-78, LDGF-PBP, GCP-2, PF4, Mig, IP-10, SDF-1alpha/beta, BUNZO/STRC33, I-TAC, BLC/BCA-1, MIP-1 alpha, MIP-1 beta, MDC, TECK, TARC, RANTES, HCC-1, HCC-4, DC-CK1, MIP-3 alpha, MIP-3 beta, MCP-1-5, eotaxin, Eotaxin-2, 1-309, MPIF-1, 6Ckine, CTACK, MEC, lymphotactin and fractalkine.

Non-limiting examples of DNA alkylating agents include nitrogen mustards, such as mechlorethamine, cyclophosphamide (ifosfamide, trofosfamide), chlorambucil (melphalan, prednimustine), bendamustine, uramustine and estramustine; nitrosoureas, such as carmustine (bcnu), lomustine (semustine), fotemustine, nimustine, ranimustine and streptozocin; alkyl sulfonates, such as busulfan (mannosulfan, treosulfan); aziridines, such as carboquone, thiotepa, triaziquone, triethylenemelamine; hydrazines (procarbazine); triazenes such as dacarbazine and temozolomide; altretamine and mitobronitol.

Non-limiting examples of topoisomerase I inhibitors include campothecin derivatives including CPT-11 (irinotecan), SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier Y. (2006) Nat. Rev. Cancer 6(10):789-802 and U.S. Patent Publication No. 200510250854; protoberberine alkaloids and derivatives thereof including berberrubine and coralyne as described in Li et al. (2000) Biochemistry 39(24):7107-7116 and Gatto et al. (1996) Cancer Res. 15(12):2795-2800; phenanthroline derivatives including benzo[i]phenanthridine, nitidine, and fagaronine as described in Makhey et al. (2003) Bioorg. Med. Chem. 11 (8): 1809-1820; terbenzimidazole and derivatives thereof as described in Xu (1998) Biochemistry 37(10):3558-3566; and anthracycline derivatives including doxorubicin, daunorubicin, and mitoxantrone as described in Foglesong et al. (1992) Cancer Chemother. Pharmacol. 30(2):123-]25, Crow et al. (1994) J. Med. Chem. 37(19):31913194, and Crespi et al. (1986) Biochem. Biophys. Res. Commun. 136(2):521-8. Topoisomerase II inhibitors include, but are not limited to Etoposide and teniposide. Dual topoisomerase I and II inhibitors include, but are not limited to, saintopin and other naphthecenediones, DACA and other Acridine-4-carboxamindes, intoplicine and other benzopyridoindoles, tas-103 and other 7h-indeno[2,1-c]quinoline-7-ones, pyrazoloacridine, XR 11576 and other benzophenazines, XR 5944 and other Dimeric compounds, 7-oxo-7H-dibenz[f,ij]Isoquinolines and 7-oxo-7H-benzo[e]perimidines, and anthracenyl-amino Acid Conjugates as described in Denny and Baguley (2003) Curr. Top. Med. Chem. 3(3):339-353. Some agents inhibit topoisomerase II and have DNA intercalation activity such as, but not limited to, anthracyclines (aclarubicin, daunorubicin, doxorubicin, epirubicin, idarubicin, amrubicin, pirarubicin, valrubicin, zorubicin) and antracenediones (mitoxantrone and pixantrone).

Non-limiting examples of endoplasmic reticulum stress inducing agents include dimethyl-celecoxib (DMC), nelfinavir, celecoxib, and boron radiosensitizers (i.e. velcade (bortezomib).

Non-limiting examples of platinum-based compound include carboplatin, cisplatin, nedaplatin, oxaliplatin, triplatin tetranitrate, satraplatin, aroplatin, lobaplatin, and JM-216. (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Non-limiting examples of antimetabolite agents include folic acid-based, e.g., dihydrofolate reductase inhibitors, such as aminopterin, methotrexate and pemetrexed; thymidylate synthase inhibitors, such as raltitrexed, pemetrexed; purine based, e.g., an adenosine deaminase inhibitor, such as pentostatin, a thiopurine, such as thioguanine and mercaptopurine, a halogenated/ribonucleotide reductase inhibitor, such as cladribine, clofarabine, fludarabine, or a guanine/guanosine: thiopurine, such as thioguanine; or pyrimidine based, e.g., cytosine/cytidine: hypomethylating agent, such as azacitidine and decitabine, a dna polymerase inhibitor, such as cytarabine, a ribonucleotide reductase inhibitor, such as gemcitabine, or a thymine/thymidine: thymidylate synthase inhibitor, such as a fluorouracil (5-FU). Equivalents to 5-FU include prodrugs, analogs and derivative thereof such as 5′deoxy 5 fluorouridine(doxifluoroidine), 1-tetrahydrofuranyl-5-fluorouracil (FTORAFUR®), capecitabine (XELODA®), S-I (MBMS-247616, consisting of tegafur and two modulators, a 5-chloro-2,4-dihydroxypyridine and potassium oxonate), ralititrexed (TOMUDEX®), no latrexed (Thymitaq, AG337), LY231514 and ZD9331, as described for example in Papamicheal (1999) The Oncologist 4:478-487.

Non-limiting examples of vincalkaloids vinblastine, vincristine, vinflunine, vindesine and vinorelbine.

Non-limiting examples of taxanes include docetaxel, larotaxel, ortataxel, paclitaxel and tesetaxel. an example of an epothilone is iabepilone.

Non-limiting examples of enzyme inhibitors include farnesyltransferase inhibitors (tipifamib); CDK inhibitor (alvocidib, seliciclib); proteasome inhibitor (bortezomib); phosphodiesterase inhibitor (anagrelide; rolipram); IMP dehydrogenase inhibitor (tiazofurine); and lipoxygenase inhibitor (masoprocol). Examples of receptor antagonists include, but are not limited to ERA (atrasentan); retinoid X receptor (bexarotene); and a sex steroid (testolactone).

Non-limiting examples of tyrosine kinase inhibitors include inhibitors to ErbB: HER1/EGFR (erlotinib, gefitinib, lapatinib, vandetanib, sunitinib, neratinib); HER2/neu (lapatinib, neratinib); RTK class III: C-kit (axitinib, sunitinib, sorafenib), FLT3 (lestaurtinib), PDGFR (axitinib, sunitinib, sorafenib); and VEGFR (vandetanib, semaxanib, cediranib, axitinib, sorafenib); bcr-abl (imatinib, nilotinib, dasatinib); Src (bosutinib) and Janus kinase 2 (lestaurtinib).

Non-limiting examples of chemotherapeutic agents include amsacrine, Trabectedin, retinoids (alitretinoin, tretinoin), arsenic trioxide, asparagine depleter asparaginase/pegaspargase), celecoxib, demecolcine, elesclomol, elsamitrucin, etoglucid, lonidamine, lucanthone, mitoguazone, mitotane, oblimersen, temsirolimus, and vorinostat.

Non-limiting examples of additional therapeutic molecules that can be linked to AFFIMER® polypeptides of the disclosure include flomoxef; fortimicin(s); gentamicin(s); glucosulfone solasulfone; gramicidin S; gramicidin(s); grepafloxacin; guamecycline; hetacillin; isepamicin; josamycin; kanamycin(s); flomoxef; fortimicin(s); gentamicin(s); glucosulfone solasulfone; gramicidin S; gramicidin(s); grepafloxacin; guamecycline; hetacillin; isepamicin; josamycin; kanamycin(s); bacitracin; bambermycin(s); biapenem; brodimoprim; butirosin; capreomycin; carbenicillin; carbomycin; carumonam; cefadroxil; cefamandole; cefatrizine; cefbuperazone; cefclidin; cefdinir; cefditoren; cefepime; cefetamet; cefixime; cefinenoxime; cefininox; cladribine; apalcillin; apicycline; apramycin; arbekacin; aspoxicillin; azidamfenicol; aztreonam; cefodizime; cefonicid; cefoperazone; ceforamide; cefotaxime; cefotetan; cefotiam; cefozopran; cefpimizole; cefpiramide; cefpirome; cefprozil; cefroxadine; cefteram; ceftibuten; cefuzonam; cephalexin; cephaloglycin; cephalosporin C; cephradine; chloramphenicol; chlortetracycline; clinafloxacin; clindamycin; clomocycline; colistin; cyclacillin; dapsone; demeclocycline; diathymosulfone; dibekacin; dihydrostreptomycin; 6-mercaptopurine; thioguanine; capecitabine; docetaxel; etoposide; gemcitabine; topotecan; vinorelbine; vincristine; vinblastine; teniposide; melphalan; methotrexate; 2-p-sulfanilyanilinoethanol; 4,4′sulfinydianilin; 4-sulfanilamidosalicylic acid; butorphanol; nalbuphine. streptozocin; doxorubicin; daunorubicin; plicamycin; idarubicin; mitomycin C; pentostatin; mitoxantrone; cytarabine; fludarabine phosphate; butorphanol; nalbuphine. streptozocin; doxorubicin; daunorubicin; plicamycin; idarubicin; mitomycin C; pentostatin; mitoxantrone; cytarabine; fludarabine phosphate; acediasulfone; acetosulfone; amikacin; amphotericin B; ampicillin; atorvastatin; enalapril; ranitidine; ciprofloxacin; pravastatin; clarithromycin; cyclosporin; famotidine; leuprolide; acyclovir; paclitaxel; azithromycin; lamivudine; budesonide; albuterol; indinavir; metformin; alendronate; nizatidine; zidovudine; carboplatin; metoprolol; amoxicillin; diclofenac; lisinopril; ceftriaxone; captopril; salmeterol; xinafoate; imipenem; cilastatin; benazepril; cefaclor; ceftazidime; morphine; dopamine; bialamicol; fluvastatin; phenamidine; podophyllinic acid 2-ethylhydrazine; acriflavine; chloroazodin; arsphenamine; amicarbilide; aminoquinuride; quinapril; oxymorphone; buprenorphine; floxuridine; dirithromycin; doxycycline; enoxacin; enviomycin; epicillin; erythromycin; leucomycin(s); lincomycin; lomefloxacin; lucensomycin; lymecycline; meclocycline; meropenem; methacycline; micronomicin; midecamycin(s); minocycline; moxalactam; mupirocin; nadifloxacin; natamycin; neomycin; netilmicin; norfloxacin; oleandomycin; oxytetracycline; p-sulfanilylbenzylamine; panipenem; paromomycin; pazufloxacin; penicillin N; pipacycline; pipemidic acid; polymyxin; primycin; quinacillin; ribostamycin; rifamide; rifampin; rifamycin SV; rifapentine; rifaximin; ristocetin; ritipenem; rokitamycin; rolitetracycline; rosaramycin; roxithromycin; salazosulfadimidine; sancycline; sisomicin; sparfloxacin; spectinomycin; spiramycin; streptomycin; succisulfone; sulfachrysoidine; sulfaloxic acid; sulfamidochrysoidine; sulfanilic acid; sulfoxone; teicoplanin; temafloxacin; temocillin; tetroxoprim; thiamphenicol; thiazolsulfone; thiostrepton; ticarcillin; tigemonam; tobramycin; tosufloxacin; trimethoprim; trospectomycin; trovafloxacin; tuberactinomycin; vancomycin; azaserine; candicidin(s); chlorphenesin; dermostatin(s); filipin; fungichromin; mepartricin; nystatin; oligomycin(s); perimycin A; tubercidin; 6-azauridine; 6-diazo-5-oxo-L-norleucine; aclacinomycin(s); ancitabine; anthramycin; azacitadine; azaserine; bleomycin(s); ethyl biscoumacetate; ethylidene dicoumarol; iloprost; lamifiban; taprostene; tioclomarol; tirofiban; amiprilose; bucillamine; gusperimus; gentisic acid; glucamethacin; glycol salicylate; meclofenamic acid; mefenamic acid; mesalamine; niflumic acid; olsalazine; oxaceprol; S-enosylmethionine; salicylic acid; salsalate; sulfasalazine; tolfenamic acid; carubicin; carzinophillin A; chlorozotocin; chromomycin(s); denopterin; doxifluridine; edatrexate; eflornithine; elliptinium; enocitabine; epirubicin; mannomustine; menogaril; mitobronitol; mitolactol; mopidamol; mycophenolic acid; nogalamycin; olivomycin(s); peplomycin; pirarubicin; piritrexim; prednimustine; procarbazine; pteropterin; puromycin; ranimustine; streptonigrin; thiamiprine; mycophenolic acid; procodazole; romurtide; sirolimus (rapamycin); tacrolimus; butethamine; fenalcomine; hydroxytetracaine; naepaine; orthocaine; piridocaine; salicyl alcohol; 3-amino-4-hydroxybutyric acid; aceclofenac; alminoprofen; amfenac; bromfenac; bromosaligenin; bumadizon; carprofen; diclofenac; diflunisal; ditazol; enfenamic acid; etodolac; etofenamate; fendosal; fepradinol; flufenamic acid; Tomudex (N-[[5-[[(1,4-Dihydro-2-methyl-4-oxo-6-quinazolinyemethyl]methylamino]-2-thienyl]carbonyl]-L-glutamic acid), trimetrexate, tubercidin, ubenimex, vindesine, zorubicin; argatroban; coumetarol and dicoumarol.

Non-limiting examples of cytotoxic factors include diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Non-limiting examples of neurotransmitters include arginine, aspartate, glutamate, gamma-aminobutyric acid, glycine, D-serine, acetylcholine, dopamine, norepinephrine (noradrenaline), epinephrine (adrenaline), serotonin (5-hydroxytryptamine), histamine, phenethylamine, N-methylphenethylamine, tyramine, octopamine, synephrine, tryptamine, N-methyltryptamine, anandamide, 2-arachidonoylglycerol, 2-arachidonyl glyceryl ether, N-arachidonoyl dopamine, virodhamine, adenosine, adenosine triphosphate, bradykinin, corticotropin-releasing hormone, urocortin, galanin, galanin-like peptide, gastrin, cholecystokinin, adrenocorticotropic hormone, proopiomelanocortin, melanocyte-stimulating hormones, vasopressin, oxytocin, Neurophysin I, Neurophysin II, Neuromedin U, Neuropeptide B, Neuropeptide S, Neuropeptide Y, Pancreatic polypeptide, Peptide YY, enkephalin, dynorphin, endorphin, endomorphin, nociceptin/orphanin FQ, Orexin A, Orexin B, kisspeptin, Neuropeptide FP, prolactin-releasing peptide, pyroglutamylated rfamide peptide, secretin, motilin, glucagon, glucagon-like peptide-1, glucagon-like peptide-2, vasoactive intestinal peptide, growth hormone-releasing hormone, pituitary adenylate cyclase-activating peptide, somatostatin, Neurokinin A, Neurokinin B, Substance P, Neuropeptide K, agouti-related peptide, N-acetylaspartylglutamate, cocaine- and amphetamine-regulated transcript, bombesin, gastrin releasing peptide, gonadotropin-releasing hormone, melanin-concentrating hormone, nitric oxide, carbon monoxide, and hydrogen sulfide.

Non-limiting examples of metabolic hormones, such as incretins (which stimulate a decrease in blood glucose levels), include glucagon-like peptide-1 (GLP-1) and gastric inhibitory peptide (GIP) and anologs thereof, such as dulaglutide (TRULICITY®), exenatide (BYETTA®), liraglutide (VICTOZA®), and exenatide extended-release (BYDUREON®).

### Pharmaceutical Compositions/Formulations

The present disclosure also provides pharmaceutical compositions comprising an anti-human FcRn AFFIMER® polypeptide (“AFFIMER® polypeptide”) described herein and a pharmaceutically acceptable vehicle. In some embodiments, the pharmaceutical compositions find use in immunotherapy. In some embodiments, the pharmaceutical compositions find use in immuno-oncology. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer, an inflammatory disorder, a cardiovascular disorder, a metabolic disorder, or an autoimmune disorder in a subject (e.g., a human patient).

Formulations are prepared for storage and use by combining a purified AFFIMER® polypeptide of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, a AFFIMER® polypeptide described herein is lyophilized and/or stored in a lyophilized form. In some embodiments, a formulation comprising a AFFIMER® polypeptide described herein is lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22nd Edition, 2012, Pharmaceutical Press, London).

The pharmaceutical compositions of the present disclosure can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

In some embodiments, a composition is formulated for topical delivery such that the when applied to the skin, for example, the AFFIMER® polypeptide penetrates the skin (crosses epithelial and mucosal barriers) to function systemically.

The therapeutic formulation can be in unit dosage form. Such formulations include tablets, pills, capsules, powders, granules, solutions or suspensions in water or non-aqueous media, or suppositories. In solid compositions, such as tablets, the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The AFFIMER® polypeptides described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In some embodiments, pharmaceutical formulations include an AFFIMER® polypeptide of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In some embodiments, sustained-release preparations comprising AFFIMER® polypeptides described herein can be produced. Suitable examples of sustained-release preparations include semi-permeable matrices of solid hydrophobic polymers containing a AFFIMER® polypeptide, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

For the treatment of a disease, the appropriate dosage of an AFFIMER® polypeptide of the present disclosure depends on the type of disease to be treated, the severity and course of the disease, the responsiveness of the disease, whether the AFFIMER® polypeptide is administered for therapeutic or preventative purposes, previous therapy, the patient's clinical history, and so on, all at the discretion of the treating physician. The AFFIMER® polypeptide can be administered one time or over a series of treatments lasting from several days to several months, or until a cure is affected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates. In some embodiments, dosage is from 0.01 mg to 100 mg/kg of body weight, from 0.1 mg to 100 mg/kg of body weight, from 1 mg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In some embodiments, the dosage of the AFFIMER® polypeptide is from about 0.1 mg to about 20 mg/kg of body weight. In some embodiments, the dosage of the AFFIMER® polypeptide is about 0.1 mg/kg of body weight. In some embodiments, the dosage of the AFFIMER® polypeptide is about 0.25 mg/kg of body weight. In some embodiments, the dosage of the AFFIMER® polypeptide is about 0.5 mg/kg of body weight. In some embodiments, the dosage of the AFFIMER® polypeptide is about 1 mg/kg of body weight. In some embodiments, the dosage of the AFFIMER® polypeptide is about 1.5 mg/kg of body weight. In some embodiments, the dosage of the AFFIMER® polypeptide is about 2 mg/kg of body weight. In some embodiments, the dosage of the AFFIMER® polypeptide is about 2.5 mg/kg of body weight. In some embodiments, the dosage of the AFFIMER® polypeptide is about 5 mg/kg of body weight. In some embodiments, the dosage of the AFFIMER® polypeptide is about 7.5 mg/kg of body weight. In some embodiments, the dosage of the AFFIMER® polypeptide is about 10 mg/kg of body weight. In some embodiments, the dosage of the AFFIMER® polypeptide is about 12.5 mg/kg of body weight. In some embodiments, the dosage of the AFFIMER® polypeptide is about 15 mg/kg of body weight. In some embodiments, the dosage can be given once or more daily, weekly, monthly, or yearly. In some embodiments, the AFFIMER® polypeptide is given once every week, once every two weeks, once every three weeks, or once every four weeks.

In some embodiments, an AFFIMER® polypeptide may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the AFFIMER® polypeptide is administered for 3, 4, 5, 6, 7, 8, or more cycles. For example, the AFFIMER® polypeptide is administered every 2 weeks for 6 cycles, the AFFIMER® polypeptide is administered every 3 weeks for 6 cycles, the AFFIMER® polypeptide is administered every 2 weeks for 4 cycles, the AFFIMER® polypeptide is administered every 3 weeks for 4 cycles, etc. Dosing schedules can be decided upon and subsequently modified by those skilled in the art.

Thus, the present disclosure provides methods of administering to a subject the polypeptides or agents described herein comprising using an intermittent dosing strategy for administering one or more agents, which may reduce side effects and/or toxicities associated with administration of an AFFIMER® polypeptide, therapeutic agent, etc. In some embodiments, a method for treating cancer in a human subject comprises administering to the subject a therapeutically effective dose of an AFFIMER® polypeptide in combination with a therapeutically effective dose of a therapeutic agent, wherein one or both of the agents are administered according to an intermittent dosing strategy. In some embodiments, the intermittent dosing strategy comprises administering an initial dose of an AFFIMER® polypeptide to the subject and administering subsequent doses of the AFFIMER® polypeptide about once every 2 weeks. In some embodiments, the intermittent dosing strategy comprises administering an initial dose of an AFFIMER® polypeptide to the subject and administering subsequent doses of the AFFIMER® polypeptide about once every 3 weeks. In some embodiments, the intermittent dosing strategy comprises administering an initial dose of an AFFIMER® polypeptide to the subject and administering subsequent doses of the AFFIMER® polypeptide about once every 4 weeks. In some embodiments, the AFFIMER® polypeptide is administered using an intermittent dosing strategy and the therapeutic agent is administered weekly.

### Polynucleotides

A polynucleotide (also referred to as a nucleic acid) is a polymer of nucleotides of any length, and may include deoxyribonucleotides, ribonucleotides, modified nucleotides or bases, and/or their analogs, or any substrate that can be incorporated into a polymer by DNA or RNA polymerase. In some embodiments, a polynucleotide herein encodes a polypeptide, such as an anti-human FcRn AFFIMER® polypeptide. As known in the art, the order of deoxyribonucleotides in a polynucleotide determines the order of amino acids along the encoded polypeptide (e.g., protein).

A polynucleotide sequence may be any sequence of deoxyribonucleotides and/or ribonucleotides, may be single-stranded, double-stranded, or partially double-stranded. The length of a polynucleotide may vary and is not limited. Thus, a polynucleotide may comprise, for example, 2 to 1,000,000 nucleotides. In some embodiments, a polynucleotide has a length of 100 to 100,000, a length of 100 to 10,000, a length of 100 to 1,000, a length of 100 to 500, a length of 200 to 100,000, a length of 200 to 10,000, a length of 200 to 1,000, or a length of 200 to 500 nucleotides.

A vector herein refers to a vehicle for delivering a molecule to a cell. In some embodiments, a vector is an expression vector comprising a promoter (e.g., inducible or constitutive) operably linked to a polynucleotide sequence encoding a polypeptide. Non-limiting examples of vectors include viral vectors (e.g., adenoviral vectors, adeno-associated virus vectors, and retroviral vectors), naked DNA or RNA expression vectors, plasmids, cosmids, phage vectors, DNA and/or RNA expression vectors associated with cationic condensing agents, and DNA and/or RNA expression vectors encapsulated in liposomes. Vectors may be transfected into a cell, for example, using any transfection method, including, for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics technology (biolistics).

### Gene Delivery

An alternative approach to the delivery of therapeutic anti-human FcRn AFFIMER® polypeptide would be to leave the production of the therapeutic polypeptide to the body itself. A multitude of clinical studies have illustrated the utility of in vivo gene transfer into cells using a variety of different delivery systems. In vivo gene transfer seeks to administer to patients the nucleotide sequence of the anti-human FcRn AFFIMER® polypeptide, rather than the anti-human FcRn AFFIMER® polypeptide itself. This allows the patient's body to produce the anti-human FcRn AFFIMER® polypeptide of interest for a prolonged period of time, and secrete it either systemically or locally, depending on the production site. Gene-based nucleotides encoding anti-human FcRn AFFIMER® polypeptides can present a labor- and cost-effective alternative to the conventional production, purification and administration of the polypeptide version of the anti-human FcRn AFFIMER® polypeptide. A number of antibody expression platforms have been pursued in vivo to which delivery of polynucleotides anti-human FcRn AFFIMER® polypeptide can be adapted: these include viral vectors, naked DNA and RNA. The use of gene transfer with polynucleotides encoding anti-human FcRn AFFIMER® polypeptide cannot only enable cost-savings by reducing the cost of goods and of production but may also be able to reduce the frequency of drug administration. Overall, a prolonged in vivo production of the therapeutic anti-human FcRn AFFIMER® polypeptides by expression of the polynucleotides encoding anti-human FcRn AFFIMER® polypeptides can contribute to (i) a broader therapeutic or prophylactic application of anti-human FcRn AFFIMER® polypeptides in price-sensitive conditions, (ii) an improved accessibility to therapy in both developed and developing countries, and (iii) more effective and affordable treatment modalities. In addition to in vivo gene transfer, cells can be harvested from the host (or a donor), engineered with polynucleotides encoding anti-human FcRn AFFIMER® polypeptides to produce anti-human FcRn AFFIMER® polypeptides and re-administered to patients.

The tumor presents a site for the transfer of polynucleotides encoding anti-human FcRn AFFIMER® polypeptides, targeted either via intravenous or direct injection/electroporation. Indeed, intratumoral expression of polynucleotides encoding anti-human FcRn AFFIMER® polypeptides can allow for a local production of the therapeutic anti-human FcRn AFFIMER® polypeptides, waiving the need for high systemic anti-human FcRn AFFIMER® polypeptide levels that might otherwise be required to penetrate and impact solid tumors. See, for example, Beckman et al. (2015) “Antibody constructs in cancer therapy: protein engineering strategies to improve exposure in solid tumors” Cancer 109(2):170-9 and Dronca et al. (2015) “Immunomodulatory antibody therapy of cancer: the closer, the better” Clin Cancer Res. 21(5):944-6.

The success of gene therapy has largely been driven by improvements in nonviral and viral gene transfer vectors. An array of physical and chemical nonviral methods have been used to transfer DNA and mRNA to mammalian cells and a substantial number of these have been developed as clinical stage technologies for gene therapy, both ex vivo and in vivo, and are readily adapted for delivery of the polynucleotides encoding anti-human FcRn AFFIMER® polypeptides of the present disclosure. To illustrate, cationic liposome technology can be employed, which is based on the ability of amphipathic lipids, possessing a positively charged head group and a hydrophobic lipid tail, to bind to negatively charged DNA or RNA and form particles that generally enter cells by endocytosis. Some cationic liposomes also contain a neutral co-lipid, thought to enhance liposome uptake by mammalian cells. See, for example, Feigner et al. (1987) Lipofection: a highly efficient, lipid-mediated DNA-transfection procedure. MNAS 84:7413-7417; San et al. (1983) “Safety and short-term toxicity of a novel cationic lipid formulation for human gene therapy” Hum. Gene Ther. 4:781-788; Xu et al. (1996) “Mechanism of DNA release from cationic liposome/DNA complexes used in cell transfection” Biochemistry 35:5616-5623; and Legendre et al. (1992) “Delivery of plasmid DNA into mammalian cell lines using pH-sensitive liposomes: comparison with cationic liposomes” Pharm. Res. 9, 1235-1242.

Similarly, other polycations, such as poly-1-lysine and polyethylene-imine, can be used to deliver polynucleotides encoding anti-human FcRn AFFIMER® polypeptides. These polycations complex with nucleic acids via charge interaction and aid in the condensation of DNA or RNA into nanoparticles, which are then substrates for endosome-mediated uptake. Several of these cationic nucleic acid complex technologies have been developed as potential clinical products, including complexes with plasmid DNA, oligodeoxynucleotides, and various forms of synthetic RNA. Modified (and unmodified or “naked”) DNA and RNA have also been shown to mediate successful gene transfer in a number of circumstances and can also be used as systems for delivery of polynucleotides encoding anti-human FcRn AFFIMER® polypeptides. These include the use of plasmid DNA by direct intramuscular injection, the use of intratumoral injection of plasmid DNA. See, for example, Rodrigo et al. (2012) “De novo automated design of small RNA circuits for engineering synthetic riboregulation in living cells” PNAS 109:15271-15276; Oishi et al. (2005) “Smart polyion complex micelles for targeted intracellular delivery of PEGylated antisense oligonucleotides containing acid-labile linkages” Chembiochem. 6:718-725; Bhatt et al. (2015) “Microbeads mediated oral plasmid DNA delivery using polymethacrylate vectors: an effectual groundwork for colorectal cancer” Drug Deliv. 22:849-861; Ulmer et al. (1994) Protective immunity by intramuscular injection of low doses of influenza virus DNA vaccines” Vaccine 12: 1541-1544; and Heinzerling et al. (2005) “Intratumoral injection of DNA encoding human interleukin 12 into patients with metastatic melanoma: clinical efficacy” Hum. Gene Ther. 16:35-48.

Viral vectors are currently used as a delivery vehicle in the vast majority of pre-clinical and clinical gene therapy trials and in the first to be approved directed gene therapy. See Gene Therapy Clinical Trials Worldwide 2017 (abedia.com/wiley/). The main driver thereto is their exceptional gene delivery efficiency, which reflects a natural evolutionary development; viral vector systems are attractive for gene delivery, because viruses have evolved the ability to cross through cellular membranes by infection, thereby delivering nucleic acids such as polynucleotides encoding anti-human FcRn AFFIMER® polypeptides to target cells. Pioneered by adenoviral systems, the field of viral vector-mediated antibody gene transfer made significant strides in the past decades. The myriad of successfully evaluated administration routes, pre-clinical models and disease indications puts the capabilities of antibody gene transfer at full display through which the skilled artisan would readily be able to identify and adapt antibody gene transfer systems and techniques for in vivo delivery of polynucleotides constructs encoding anti-human FcRn AFFIMER® polypeptides. In the context of vectored intratumoral polynucleotides encoding anti-human FcRn AFFIMER® polypeptides gene transfer, oncolytic viruses have a distinct advantage, as they can specifically target tumor cells, boost anti-human FcRn AFFIMER® polypeptide expression, and amplify therapeutic responses—such as to anti-human FcRn AFFIMER® polypeptides.

In vivo gene transfer of polynucleotides encoding anti-human FcRn AFFIMER® polypeptides can also be accomplished by use of nonviral vectors, such as expression plasmids. Nonviral vectors are easily produced and do not seem to induce specific immune responses. Muscle tissue is most often used as target tissue for transfection, because muscle tissue is well vascularized and easily accessible, and myocytes are long-lived cells. Intramuscular injection of naked plasmid DNA results in transfection of a certain percentage of myocytes. Using this approach, plasmid DNA encoding cytokines and cytokine/IgG1 chimeric proteins has been introduced in vivo and has positively influenced (autoimmune) disease outcome.

In some instances, in order to increase transfection efficiency via so-called intravascular delivery in which increased gene delivery and expression levels are achieved by inducing a short-lived transient high pressure in the veins. Special blood-pressure cuffs that may facilitate localized uptake by temporarily increasing vascular pressure and can be adapted for use in human patients for this type of gene delivery. See, for example, Zhang et al. (2001) “Efficient expression of naked DNA delivered intraarterially to limb muscles of nonhuman primates” Hum. Gene Ther., 12:427-438

Increased efficiency can also be gained through other techniques, such as in which delivery of the nucleic acid is improved by use of chemical carriers—cationic polymers or lipids—or via a physical approach—gene gun delivery or electroporation. See Tranchant et al. (2004) “Physicochemical optimisation of plasmid delivery by cationic lipids” J. Gene Med., 6 (Suppl. 1): S24-S35; and Niidome et al. (2002) “Gene therapy progress and prospects: nonviral vectors” Gene Ther., 9:1647-1652. Electroporation is especially regarded as an interesting technique for nonviral gene delivery. Somiari, et al. (2000) “Theory and in vivo application of electroporative gene delivery” Mol. Ther. 2:178-187; and Jaroszeski et al. (1999) “In vivo gene delivery by electroporation” Adv. Drug Delivery Rev., 35:131-137. With electroporation, pulsed electrical currents are applied to a local tissue area to enhance cell permeability, resulting in gene transfer across the membrane. Research has shown that in vivo gene delivery can be at least 10-100 times more efficient with electroporation than without. See, for example, Aihara et al. (1998) “Gene transfer into muscle by electroporation in vivo” Nat. Biotechnol. 16:867-870; Mir, et al. (1999) “High-efficiency gene transfer into skeletal muscle mediated by electric pulses” PNAS 96:4262-4267; Rizzuto, et al. (1999) “Efficient and regulated erythropoietin production by naked DNA injection and muscle electroporation” PNAS 96: 6417-6422; and Mathiesen (1999) “Electropermeabilization of skeletal muscle enhances gene transfer in vivo” Gene Ther., 6:508-514.

Encoded anti-human FcRn AFFIMER® polypeptides can be delivered by a wide range of gene delivery system commonly used for gene therapy including viral, non-viral, or physical. See, for example, Rosenberg et al., Science, 242:1575-1578, 1988, and Wolff et al., Proc. Natl. Acad. Sci. USA 86:9011-9014 (1989). Discussion of methods and compositions for use in gene therapy include Eck et al., in Goodman & Gilman's The Pharmacological Basis of Therapeutics, Ninth Edition, Hardman et al., eds., McGraw-Hill, New York, (1996), Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, and the references cited therein. U.S. Pat. No. 6,080,728 also provides a discussion of a wide variety of gene delivery methods and compositions. The routes of delivery include, for example, systemic administration and administration in situ.

An effective gene transfer approach should be directed to the specific tissues/cells where it is needed, and the resulting transgene expression should be at a level that is appropriate to the specific application. Promoters are a major cis-acting element within the vector genome design that can dictate the overall strength of expression as well as cell-specificity.

In some embodiments, a viral vector is used to deliver a nucleic acid encoding a anti-human FcRn AFFIMER® polypeptide of the present disclosure. Non-limiting examples of viral vectors include adenoviral vectors, adeno-associated viral (AAV) vectors, and retroviral vectors. In other embodiments, a non-viral vector is used to deliver a nucleic acid encoding a anti-human FcRn AFFIMER® polypeptide of the present disclosure. Non-limiting examples of non-viral vectors include plasmid vectors (e.g., plasmid DNA (pDNA) delivered via, e.g., hydrodynamic-based transfection or electroporation), minicircle DNA, and RNA-mediate gene transfer (e.g., delivery of messenger RNA (mRNA) encoding a anti-human FcRn AFFIMER® polypeptide of the present disclosure).

Exemplary nucleic acids or polynucleotides for the encoded anti-human FcRn AFFIMER® polypeptides of the present disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo

o configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof.

mRNA presents an emerging platform for antibody gene transfer that can be adapted by those skilled in the art for delivery of polynucleotide constructs encoding anti-human FcRn AFFIMER® polypeptides of the present disclosure. Although current results differ considerably, in certain instances the mRNA constructs appear to be able to rival viral vectors in terms of generated serum mAb titers. Levels were in therapeutically relevant ranges within hours after mRNA administration, a marked shift in speed compared to DNA. The use of lipid nanoparticles (LNP) for mRNA transfection, rather than the physical methods typically required for DNA, can provide significant advantages in some embodiments towards application range.

Nucleic acids encoding anti-human FcRn AFFIMER® polypeptides may be delivered by, for example, intravenously, intramuscularly, or intratumorally (e.g., by injection, electroporation or other means).

Nucleic acids encoding anti-human FcRn AFFIMER® polypeptides may be formulated, for example, in lipid nanoparticles or liposomes (e.g., cationic lipid nanoparticles or liposomes), biodegradable microsphere, or other nano- or microparticle. Other lipid-based (e.g., PEG lipid) and polymeric-based formulations and delivery vehicles are contemplated herein.

### EXAMPLES

### Example 1. AFFIMER® Selections

Process Overview

### Phage Selections

Biopanning on captured Human (HFcRn)

Solution selection on biotinylated FcRn

Two (2) rounds of selection on FcRn

Enrichment monitored by output size and polyclonal Phage ELISA

### Primary Screening

Monoclonal Crude extract ELISA against captured FcRn at pH6

### Secondary Screening

ELISA on FcRn at pH 6.0 and 7.4

### General Methods

Selection of huFcRn binding phage from the AFFIMER® library was carried out as described below using approximately 1×10¹² phage added from a library of size approximately 6×10¹⁰ diversity.

A peptide of the present disclosure, for example, a huFcRn binding component, may be identified by selection from a library of AFFIMER® polypeptides with two random loops, for example, generally but not exclusively of the same length of 9 amino acids.

As indicated above, the huFcRn binding peptides of the disclosure were identified by selection from a phage display library comprising random loop sequences nine amino acids in length displayed in a constant AFFIMER® framework backbone based upon the sequence for SQT. Such selection procedures are generally known. According to such procedures, suspensions of phage are incubated with target antigen (either biotinylated antigen captured on streptavidin beads or unbiotinylated antigen captured on a plate). Unbound phage are then washed away and, subsequently, bound phage are eluted either by incubating the antigen with low pH, high pH or trypsin. E. coli are then infected with released, pH neutralised phage or trypsin-inactivated phage and a preparation of first round phage is obtained. The cycle is performed repeatedly, for example, two or three times and, in order to enrich for targeting phage, the stringency conditions may be increased in the later rounds of selection, for example by increasing the number of wash steps, reducing the antigen concentration, and preselecting with blocked streptavidin beads or wells coated with blocking reagent.

Antigens used herein were human FcRn (BPS #71285), and biotinylated human FcRn (BPS #71283). Following selection by successive rounds of phage amplification, huFcRn binding clones were identified by a crude extract ELISA as described below.

Following phage selections, individual bacterial clones containing the phagemid vector were picked from titration plates into 96 well cell culture format. Soluble AFFIMER® in crude cell extract was prepared from lysis of bacterial cells overexpressing the AFFIMER® with a C-terminal myc tag and used in a primary screening ELISA. These AFFIMER® polypeptides in extract were screened for binding to antigen at pH 6 and later also at pH 7.4, detecting AFFIMER® bound to antigen immobilized on a plate with an HRP labelled anti-myc tag antibody (Abcam #ab1261), developing the ELISA using 1-step Ultra TMB-ELISA substrate (Thermo Scientific). The screening was also carried out against non-target or related target molecules captured on the plate (eg blocking molecule, neutravidin or b-2microglobulin (Sigma #M4890) The non-target and target binding data were compared to identify library members that specifically bind to the target.

### Example 2. huFcRn Binding ELISA Assay at pH 6

The binding of AFFIMER® to Hu-FcRn was evaluated by enzyme linked immunosorbent assay (ELISA) in 384 well plate format. Hu FcRn (BPS Bioscience) was coated at 5 μg/ml on the plate in 40 mM MES, pH 6. Plates were washed 3 times with 100 μl of washing buffer (PBS, Tween 20 0.05%, pH 6) with a plate washer and saturated with Casein 5% (Sigma) in MES pH6 for 60 minutes at room temperature (25±1° C.). Plates were washed as described previously. AFFIMER® and negative controls (mAb anti hFcRn (clone ADM31), negative controls) were then diluted in duplicate, and loaded on the plate for 90 minutes at room temperature (25±1° C.). Plates were washed 3 times as described previously. Biotinylated polyclonal antibody anti Cystatin (R&D Systems) was then diluted in dilution Buffer (1% casein, 0.05% Tween 20, and 8 mM MES. It is in pH6) and incubated 60 minutes at room temperature (25±1° C.). Plates were washed 3 times as described previously and Streptavidin HRP (N200, thermo-Fisher) was incubated for 30 minutes at room temperature (25±1° C.). Plates were washed and the substrate (TMB, Pierce Thermo-Scientific) was added in the plate for 8±1 minute. The reaction was stopped using an acidic solution and plates were read at 450-630 nm. The EC50 was then calculated using the interpolated non-linear four-parameters standard curve (Table 4).

### Example 3. huFcRn Binding ELISA Assay at pH 7.4

The binding of AFFIMER® to hu-FcRn was evaluated by enzyme linked immunosorbent assay (ELISA) in 384 well plate format. Hu FcRn (BPS Bioscience) was coated at 5 μg/ml on the plate in PBS, pH 7.4. Plates were washed 3 times with 100 μl of washing buffer (PBS, Tween 20 0.05%, pH 7.4) with a plate washer and saturated with Casein 5% (Sigma) in MES pH 7.4 for 60 minutes at room temperature (25±1° C.). Plates were washed as described previously. AFFIMER® and controls (mAb anti hFcRn (ADM31), blank) were then diluted in duplicate, and loaded on the plate for 90 minutes at room temperature (25±1° C.). Plates were washed 3 times as described previously. Biotinylated polyclonal antibody anti Cystatin (R&D Systems) was then diluted in dilution Buffer (1% casein, 0.01% Tween 20, and 8 mM MES. It is in pH 7.4) and incubated 60 minutes at room temperature (25±1° C.). Plates were washed 3 times as described previously and Streptavidin HRP (N200, thermo-Fisher) was incubated for 30 minutes at room temperature (25±1° C.). Plates were washed and the substrate (TMB, Pierce Thermo-Scientific) was added in the plate for 8±1 minute. The reaction was stopped using an acidic solution and plates were read at 450-630 nm. The EC50 was then calculated using the interpolated non-linear four-parameters standard curve, and the results are shown below in Table 4.

TABLE 4
EC₅₀ Values at pH 6 and pH 7.4

| AFFIMER ® | EC50 nM | EC50 nM |
|---|---|---|
| Clone | (pH 6) | (pH 7.4) |
| LGC01-15 | 74.09 | >500 |
| LGC01-35 | 47.92 | 225 |
| LGC01-38 | 0.14 | 0.895 |

In the present invention, LGC01 can be used interchangeably with FcRn. For example, LGC01-15 refers to FcRn-15.

### Example 4: AFFIMER® Expression and Purification

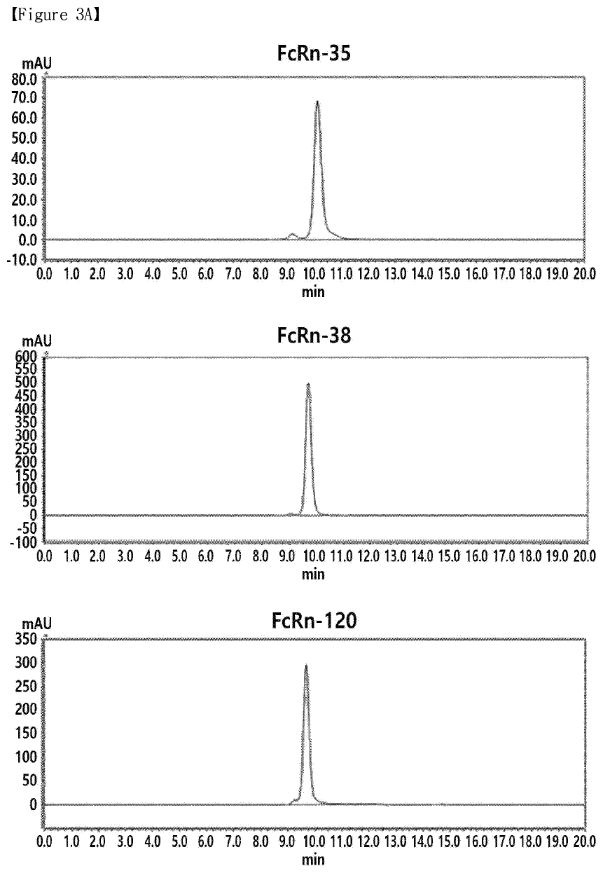

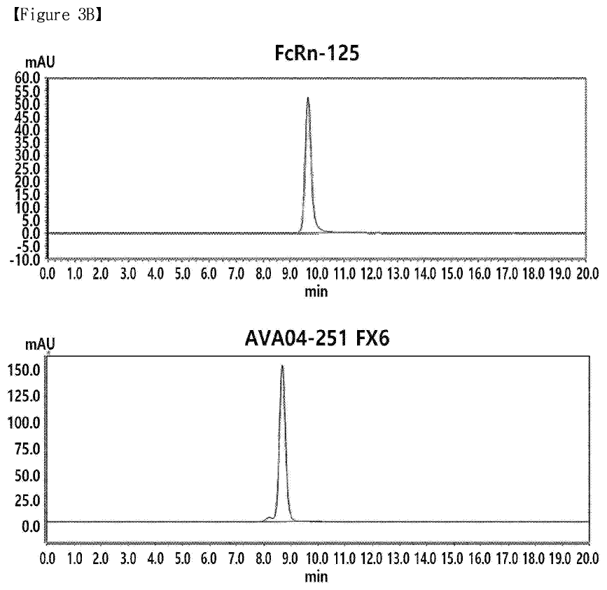

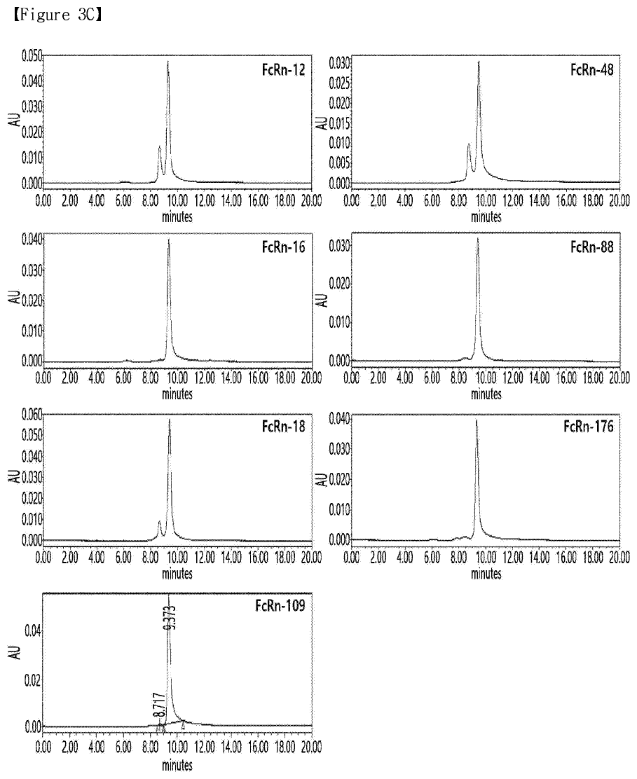

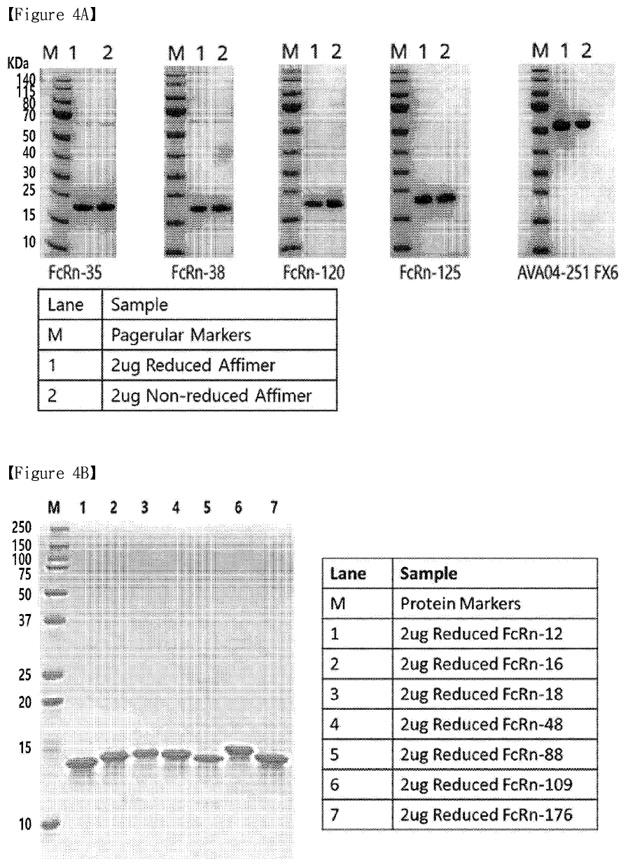

All AFFIMER® constructs expressed in E. coli have been cloned with a C-terminal hexa-HIS tag (HHHHHH (SEQ ID NO: 1185)) to simplify protein purification with immobilized metal affinity chromatography resin (IMAC resin). When required, additional peptide sequences can be added between the AFFIMER® and the HIS tag such as MYC (EQKLISEEDL (SEQ ID NO: 1186)) for detection or a TEV protease cleavage site (ENLYFQ(G/S) (SEQ ID NO: 1187)) to allow for the removal of tags. AFFIMER® analzed in FIG. 4A have MYC (EQKLISEEDL (SEQ ID NO: 1186)) and a TEV protease cleavage site (ENLYFQ(G/S) (SEQ ID NO: 1187)) and AFFIMER® analzed in FIG. 4B does not have MYC (EQKLISEEDL (SEQ ID NO: 1186)) and a TEV protease cleavage site (ENLYFQ(G/S) (SEQ ID NO: 1187)). AFFIMER® proteins were expressed from E. coli and purified using IMAC, a second stage purification to remove endotoxin, CHT (Ceramic hydroxyapatite, BioRad) type I resin or cation ion exchange (HiTrap, Cytiva) with a triton 114× wash step (Sigma), and size exclusion chromatography (SEC; Cytiva). AFFIMER® monomer purification from E. coli was performed by transforming the expression plasmid pD861 (Atum) into BL21 E. coli cells (Millipore) using the manufacturers protocol. The total transformed cell mixture was plated onto LB agar plates containing 50 μg/ml kanamycin (AppliChem) and incubated at 37° C. overnight. The following day, the lawn of transformed E. coli was transferred to a sterile flask of 1× terrific broth media (Melford) and 50 μg/ml kanamycin and incubated at 30° C. shaking at 250 rpm. Expression was induced with 10 mM rhamnose (Alfa Aesar) once the cells reached an optical density OD₆₀₀ of approximate 0.8-1.0. The culture was then incubated for a further 5 hours at 37° C. Cells were harvested by centrifuging and lysing the resulting cell pellet. AFFIMER® purification was performed using batch bind affinity purification of His-tagged protein. Specifically, nickel agarose affinity resin (Super-NiNTA500; Generon) was used. The resin was washed with NPI20 buffer (50 mM sodium phosphate, 0.5 M NaCl, 20 mM imidazole) and the bound protein was eluted with 5 column volumes (CV) of NPI400 buffer. Eluted protein was buffer exchanged for a second stage purification using CHT type I resin in running buffer 10 mM sodium phosphate pH 6.4-6.5 buffer, eluting with the addition of 2 M NaCl over a linear gradient (SEQ ID NO: 628, 631, 713 and 1184). Alternatively, a second stage purification using cation exchange was used with a SP HP ion exchange column (Cytiva) in running buffer 50 mM MES pH 6.2 for clone FcRn-125 included a 0.1% triton 114× (Sigma) wash step and the protein was eluted with a 1M NaCl linear gradient (SEQ ID NO: 718). A third stage polishing purification was performed on a preparative SEC performed using the HiLoad 26/600 Superdex 75 pg (Cytiva) run in PBS 1× buffer. Expression and purity of clones was analysed using SEC-HPLC (FIGS. 3A-3C) with an Acclaim SEC-300 column (Thermo) using a PBS 1× mobile phase. The protein yield was estimated using Nanodrop (Thermo) A280 readings and the final product was run on an SDS-PAGE Bolt Bis Tris plus 4-12% gel (Thermo)(FIG. 4A) and SDS-PAGE precast gel 20% (Komabiotech) (FIG. 4B) in Novex?20X Bolt?MES SDS running buffer (Thermo) at 200 volts, with samples heated in reducing buffer at 95° C. for 5 minutes. Protein bands on the gel were stained with Quick Commassie (Generon). PageRuler prestained protein molecular weight marker (Thermo) (FIG. 4A) and Precision Plus Protein™ Dual color standard (Bio-rad)(FIG. 4B) were run on the gel to estimate the molecular weight of the fusion proteins following the three-stage purification. Endotoxin levels of final protein batches were measured using a LAL test on an Endosafe® Nexgen MCS system (Charles River) and were between 1-0.1 EU/mg for all protein batches.

### Example 5. huFcRn Binding ELISA Assay at pH 6 for AFFIMER® Characterization

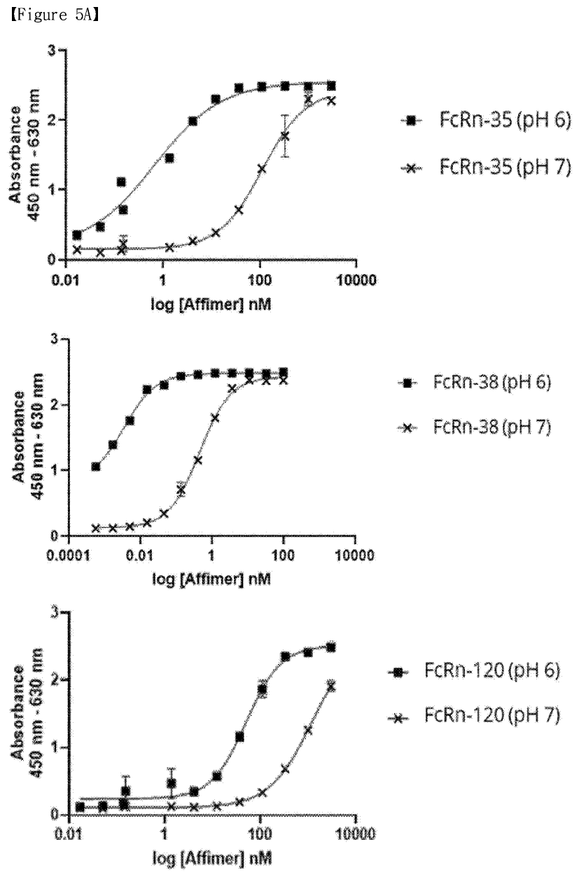

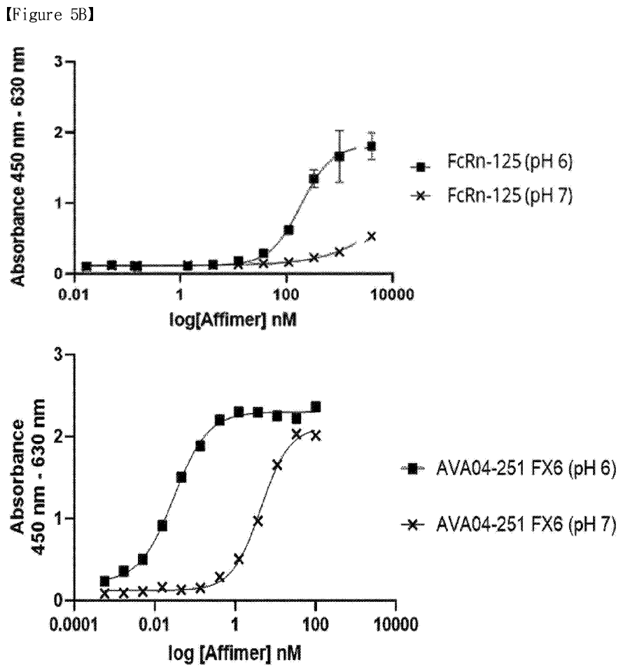

The binding of AFFIMER® to hu-FcRn was evaluated by enzyme linked immunosorbent assay (ELISA) in 384 well plate format. Hu FcRn (BPS Bioscience) was coated at 5 μg/ml on the plate in 40 mM MES, pH 6. Plates were washed 3 times with 100 ul of washing buffer (PBS, Tween 20 0.05%, pH 6) with a plate washer and saturated with Casein 5% (Sigma) in MES pH 6 for 60 minutes at room temperature (25±1° C.). Plates were washed as described previously. AFFIMER® and negative controls (mAb anti hFcRn (clone ADM31), negative controls) were then diluted in duplicate, and loaded on the plate for 90 minutes at room temperature (25±1° C.). Plates were washed 3 times as described previously. Biotinylated polyclonal antibody anti Cystatin (R&D Systems) was then diluted in dilution buffer (1% casein, 0.05% Tween 20, and 8 mM MES, pH 6) and incubated 60 minutes at room temperature (25±1° C.). Plates were washed 3 times as described previously and Streptavidin HRP (N200, Thermo-Fisher) was incubated for 30 minutes at room temperature (25±1° C.). Plates were washed and the substrate (TMB, Pierce Thermo-Scientific) was added in the plate for 8±1 minute. The reaction was stopped using an acidic solution and plates were read at 450-630 nm. The EC₅₀ was then calculated using the interpolated non-linear four-parameters standard curve (FIGS. 5A-5B and Table 5A).

In order to quantitatively compare the affinity for FcRn at pH 6.0 and pH 7.4, a slightly more optimized ELISA method was developed. After finding the optimal conditions by testing temperature and time, the binding affinity of the AFFIMER® was measured. (Table 5B).

### Example 6. huFcRn Binding ELISA Assay at pH 7.4 for AFFIMER® Characterization

The binding of AFFIMER® to hu-FcRn was evaluated by enzyme linked immunosorbent assay (ELISA) in 384 well plate format. Hu FcRn (BPS Bioscience) was coated at 5 μg/ml on the plate in PBS, pH 7.4. Plates were washed 3 times with 100 ul of washing buffer (PBS, Tween 20 0.05%, pH 7.4) with a plate washer and saturated with Casein 5% (Sigma) in MES pH 7.4 for 60 minutes at room temperature (25±1° C.). Plates were washed as described previously. AFFIMER® and controls (mAb anti hFcRn (ADM31), blank) were then diluted in duplicate, and loaded on the plate for 90 minutes at room temperature (25±1° C.). Plates were washed 3 times as described previously. Biotinylated polyclonal antibody anti Cystatin (R&D Systems) was then diluted in dilution Buffer (1% casein, 0.01% Tween 20, and 8 mM MES. It is in pH7.4) and incubated 60 minutes at room temperature (25±1° C.). Plates were washed 3 times as described previously and Streptavidin HRP (N200, thermo-Fisher) was incubated for 30 minutes at room temperature (25±1° C.). Plates were washed and the substrate (TMB, Pierce Thermo-Scientific) was added in the plate for 8±1 minute. The reaction was stopped using an acidic solution and plates were read at 450-630 nm. The EC₅₀ was then calculated using the interpolated non-linear four-parameters standard curve (FIGS. 5A-5B, Table 5A).

In order to quantitatively compare the affinity for FcRn at pH 6.0 and pH 7.4, a slightly more optimized ELISA method was developed. After finding the optimal conditions by testing temperature and time, the binding affinity of the AFFIMER® was measured. (Table 5B).

The most suitable FcRn AFFIMER® for FcRn cell recycling is advantageous if the difference in binding affinity at pH 6.0 and pH 7.4 is large, so the EC50 ratio at the measured pH 6.0 and pH 7.4 was calculated in Table 5A-B.

TABLE 5A
EC₅₀ at pH 6 and pH 7.4

| | EC50 (nM) | | pH 6/ |
|---|---|---|---|
| Clone name | pH 6 | pH 7.4 | pH 7.4 |
| FcRn-35 | 0.673 | 113.0 | 167.9049 |
| FcRn-38 | 0.003 | 0.5 | 166.6667 |
| FcRn-120 | 50.5 | NA | — |
| FcRn-125 | 187.2 | NA | — |
| AVA04-251 FX6 | 0.03 | 4.3 | 143.3333 |

TABLE 5B
EC₅₀ at pH 6 and pH 7.4

| | EC50 (nM) | | pH 6/ |
|---|---|---|---|
| Clone name | pH 6 | pH 7.4 | pH 7.4 |
| FcRn-12 | 262 | 15700 | 59.92 |
| FcRn-16 | 1020 | 48600 | 47.65 |
| FcRn-18 | 327 | 19700 | 60.24 |
| FcRn-48 | 1500 | 79900 | 53.27 |
| FcRn-88 | 967 | 23300 | 24.1 |
| FcRn-109 | 570 | 15700 | 27.54 |
| FcRn-176 | 4480 | 78900 | 17.61 |

### Example 7. BLI-Based FcRn AFFIMER® Screening

A BLI (Bio-Layer Interferometry)-based binding assay was performed for AFFIMER® screening in which the affinity to FcRn varies depending on the pH. hFcRn with a His-tag was fixed to a Ni-NTA biosensor. Thereafter, in the hFcRn and the AFFIMER® candidate group, Ni²⁺ not bound to the hFcRn was blocked using His-SQT-gly with a high concentration, in which reactivity is absent. Then, the AFFIMER® candidate group diluted to the same concentration was reacted with the hFcRn. All affimers were analyzed at pH 6.0 and pH 7.4, and K_{D} was determined with a 1:1 binding model. The results of Octet Kinetic Assay at pH 6.0 and 7.4 are shown in Table 6 below.

TABLE 6
Binding Affinity (K_{D}) at pH 6 and pH 7.4

| | Octet Kinetic Assay | | | | |
|---|---|---|---|---|---|
| | 40 nM, pH 6.0 | | 40 nM, pH 7.4 | | |
| | K_{D} | | K_{D} | | pH 7.4/ |
| Affimer | (nM) | Response | (nM) | Response | pH 6.0 |
| FcRn-12 | 28.4 | 0.836 | 123 | 0.5815 | 4.3 |
| FcRn-16 | 20.5 | 1.0713 | 83.3 | 0.6309 | 4.1 |
| FcRn-18 | 16 | 1.2006 | 65 | 0.7937 | 4.1 |
| FcRn-48 | 8.76 | 1.4376 | 59.3 | 0.8034 | 6.8 |
| FcRn-88 | 18.7 | 1.1172 | 82.8 | 0.7102 | 4.4 |
| FcRn-109 | 9.27 | 1.1567 | 61.2 | 0.6019 | 6.6 |
| FcRn-176 | 10.5 | 1.0154 | 57.9 | 0.5954 | 5.5 |

### Example 8. FcRn Competition ELISA

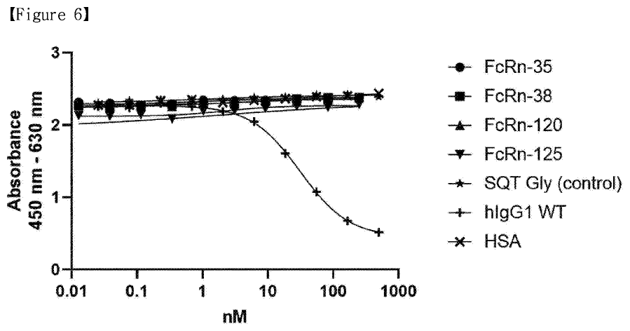

To evaluate if the AFFIMER® was competiting with IgG1, a competitive ELISA (huIgGl/huFcRn) was performed. Briefly, huIgG1 isotype control (BioXcell) was coated overnight on the plate at 5 μg/ml in 40 mM MES, pH 6. Then plates were saturated using 40 mM MES+5% casein, pH 6. In the meantime, huFcRn (His tagged molecule, BPS) was pre-incubated with a dilution of FcRn Binding AFFIMER® and its control (human IgG1 and HuSA. After saturation, plates were washed in PBS, 0.05% Tween at pH 6, the mix was added to the plates and incubated for minimum an hour. Plates were then washed as previously and the detection monoclonal antibody, anti-B2M HRP (Biolegend), was added and incubated for minimum 1 hour. After a final wash, development of the reaction was performed using TMB (Pierce) and the plates were read using a plate reader at 450 nm and absorbance were plotted against log of AFFIMER® and control concentration using a four-parameter fit. FIG. 6 shows FcRn binding AFFIMER® do not compete with huIgGl.

### Example 9. FcRn Cell Binding Protocol

1 μL of 100 μM AFFIMER® was placed in a 96-well V-bottom Plate, and 200 μL of CHO-Kl-FcRn, which was resuspended with washing buffer (PBS pH 6.0 or pH 7.4+2% FBS) at a concentration of 1×10⁶ cells/mL, was added thereto to react at room temperature for 20 min. 200 μL of washing buffer was added, and the resultants were centrifuged at 4° C. at 1,000 rpm for 3 min to remove the supernatant (3 times). Anti Cystatin Monoclonal Ab (Novus, NBP2-79882AF488), which is conjugated with AF488, was diluted with washing buffer to add 0.2 μL of the Anti Cystatin Monoclonal Ab per 2×10⁵ cells, and then the reaction was performed at 4° C. for 1 h. 200 μL of washing buffer was added, and the resultants were centrifuged at 4° C. at 1,000 rpm for 3 min to remove the supernatant (3 times). The resultants were resuspended with 200 μL of washing buffer, and the value was measured using Flow Cytometry.

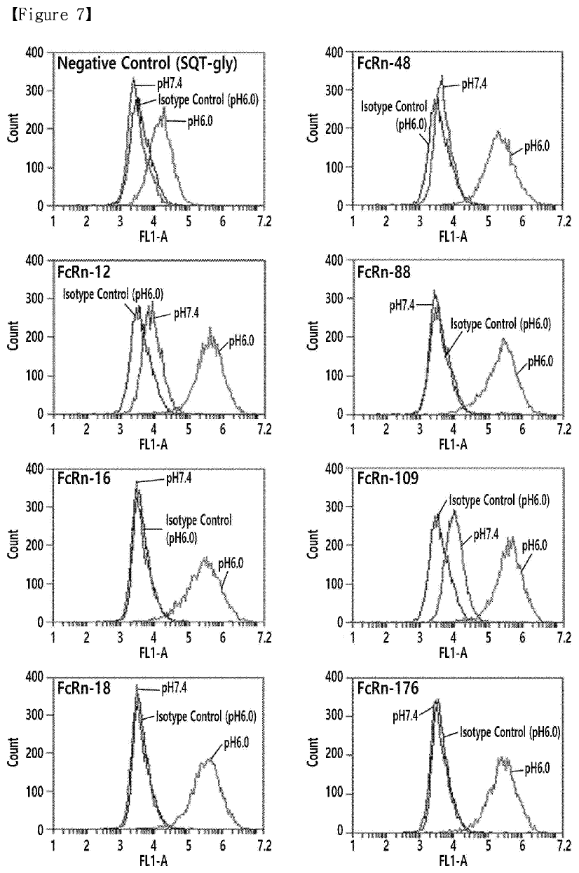

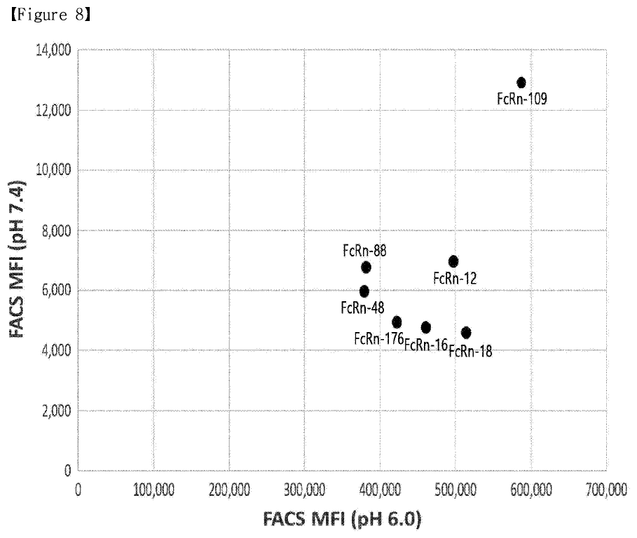

In FIG. 7 and FIG. 8, Affimer's cell binding using hFcRn over-expression CHO single clone cell line (pH6.0 & pH7.4) was confirmed.

### Example 10. Screening of Lead FcRn Binding AFFIMER® Polypeptides for Receptor Mediated Recycling in a Human Endothelial Cell-Based Recycling Assay

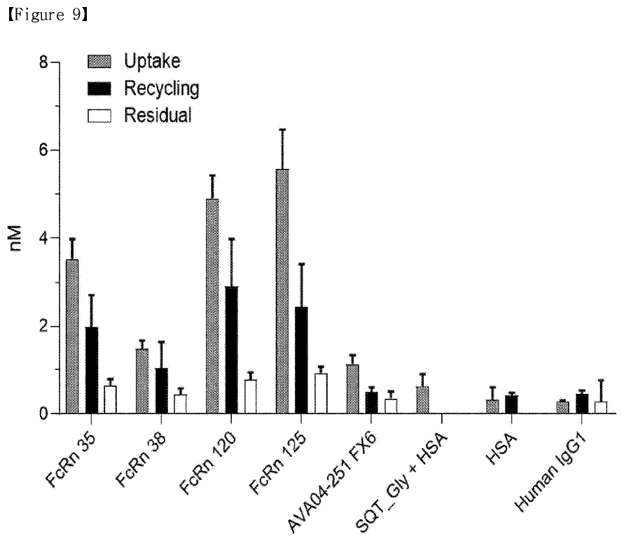

7.5×10⁵ endothelial cell line (HMEC1) stably expressing HA-hFcRn-EGFP were seeded into 24-well plates per well (Costar) and cultured for 2 days in growth medium. The cells were washed twice and starved for 1 hour in Hank's balanced salt solution (HBSS) (ThermoFisher). Then, 800 nM of either hIgG1 or AFFIMER® polypeptides were diluted in 125 μl HBSS (pH 7.4) and added to the cells followed by 4 h incubation. The media was removed and the cells were washed four times with ice cold HBSS (pH 7.4), before fresh warm HBSS (pH 7.4) or growth medium without FCS and supplemented with MEM non-essential amino acids (ThermoFisher) was added. The cells were incubated for 4 hours before sample were collected. The wells with uptake samples and residual amounts were then lysed prior to collection. Total protein lysates were obtained using RIPA lysis buffer (ThermoFisher) supplied with complete protease inhibitor tablets (Roche). The mixture was incubated (220 ul) with the cells on ice and a shaker for 10 min followed by centrifugation for 15 min at 10,000×g to remove cellular debris. Rescued AFFIMER® polypeptides and controls were quantified by quantitative ELISA anti-cystatin (see Example 11) or anti-human IgG (FIG. 9).

### Example 11. AFFIMER® Quantification by ELISA Following HERA Assay

96-well plates (Corning Costar, 3590) were coated with 50 ul of 1 ug/ml of Anti-His MAB050 diluted in coating buffer (Carbonate/bicarbonate) for 16 hours (+/−2h) at 4° C. The plates were further washed 2× with 150 ul wash buffer (1×PBS+0.05% Tween) and blocked with 100 ul 1×PBS+5% casein blocking buffer for 90 min (+/−15 min) at room temperature (RT). Next, the HERA samples were added to the plates, diluted 1:1 in 6 steps in dilution buffer (PBS+1% casein+0.01% Tween) and matching AFFIMER® polypeptides were used as standard for each variant (3.5 nM-0.0017 nM). The HERA samples were incubated for 90 min (+/−15 min) at RT. Plates were washed 3× with wash buffer. Binding was detected by using 0.05 mg/ml BAF1470 1:1000 and 1 mg/ml poly streptavidin-HRP 1:5000. The two antibodies were pre-incubated in a small volume for 20 min, before diluted in dilution buffer and added to the plates in 50 ul volume and incubated for 90 min (+/−15 min) at RT. Plates were washed 3× and binding was visualized by adding 50 ul of RT TMB to each well. The reaction was stopped by adding 50 ul 1M HCl (after 20-30 min). Absorbance was read at 450 nm and 620 nm. Control IgG1 was quantified using similar protocol using a goat polyclonal anti human Fc for capture and an alkaline phosphatase conjugated polyclonal antibody anti huIgGFc for detection.

All references, patents and patent applications disclosed herein are incorporated by reference with respect to the subject matter for which each is cited, which in some cases may encompass the entirety of the document.

The indefinite articles “a” and “an” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. A polypeptide comprising an FcRn binding recombinantly engineered variant of stefin sequence that binds to human FcRn with a K_{d} of 1×10⁻⁶M or less at pH 6.0, and optionally, a K_{d} for binding human FcRn at pH 7.4 that is at least half a log greater than the K_{d} for binding at pH 6.0.

2. (canceled)

3. (canceled)

4. The polypeptide of claim 1, wherein the polypeptide comprising (i) an FcRn binding recombinantly engineered variant of stefin polypeptide sequence which binds to human FcRn, and (ii) a heterologous polypeptide covalently associated to the FcRn binding recombinantly engineered variant of stefin polypeptide sequence Optionally as a fusion protein or chemically conjugated, which confers a therapeutic activity in human patients.

5. (canceled)

6. The polypeptide of claim 1, wherein the polypeptide A protein comprising an FcRn binding recombinantly engineered variant of stefin polypeptide sequence which binds to human FcRn and has an amino acid sequence that can be encoded by a nucleic acid having a coding sequence that hybridizes to any one of SEQ ID NOs: 888 to 1181 under stringent conditions of 6× sodium chloride/sodium citrate (SSC) at 45° C. followed by a wash in 0.2×SSC at 65° C.

7. The polypeptide of claim 1, wherein the FcRn binding recombinantly engineered variant of stefin sequence binds to FcRn with a K_{d} of 1×10⁻⁷ M or less at pH 6.0, a K_{d} of 1×10⁻⁸ M or less at pH 6.0, or K_{d} of 1×10⁻⁹ M or less at pH 6.0.

8. The polypeptide of claim 1, wherein the FcRn binding recombinantly engineered variant of stefin sequence binds to FcRn at pH 7.4 with a K_{d} that is at least one log greater than the K_{d} for binding to FcRn at pH 6.0, at least 1.5 logs greater than the K_{d} for binding to FcRn at pH 6, at least 2 logs greater than the K_{d} for binding to FcRn at pH 6, or at least 2.5 log greater than the K_{d} for binding to FcRn at pH 6.

9. The polypeptide of claim 1, wherein the polypeptide has a serum half-life in human patients of greater than 10 hours, greater than 24 hours, greater than 48 hours, greater than 72 hours, greater than 96 hours, greater than 120 hours, greater than 144 hours, greater than 168 hours, greater than 192 hours, greater than 216 hours, greater than 240 hours, greater than 264 hours, greater than 288 hours, greater than 312 hours, greater than 336 hours or, greater than 360 hours.

10. The polypeptide of claim 1, wherein the polypeptide has a serum half-life in human patients of greater than 50%, greater than 60%, greater than 70%, or greater than 80% of the serum half-life of IgG and/or
wherein the polypeptide has a serum half-life in human patients of greater than 50%, greater than 60%, greater than 70%, or greater than 80% of the serum half-life of serum albumin.

11. (canceled)

12. The polypeptide of claim 1, wherein the polypeptide does not inhibit binding of human serum albumin to human FcRn.

13. (canceled)

14. (canceled)

15. The polypeptide of claim 1 comprising an amino acid sequence represented in general formula (I)
FR1-(Xaa)ₙ-FR2-(Xaa)ₘ-FR3 (I),
wherein
FR1 is an amino acid sequence having at least 70% identity to MIPGGLSEAK PATPEIQEIV DKVKPQLEEK TNETYGKLEA VQYKTQVLA (SEQ ID NO: 1);
FR2 is an amino acid sequence having at least 70% identity to GTNYYIKVRA GDNKYMHLKV FKSL (SEQ ID NO: 2);
FR3 is an amino acid sequence having at least 70% identity to EDLVLTGYQV DKNKDDELTG F (SEQ ID NO: 3); and
Xaa, individually for each occurrence, is an amino acid,
n is an integer from 3 to 20, and m is an integer from 3 to 20.

16. The polypeptide of claim 15, wherein:
FR1 has at least 80%, at least 82%, at least 84%, at least 86%, at least 88%, at least 90%, at least 92%, at least 94%, at least 96%, or at least 98% identity to SEQ ID NO: 1;
FR2 has at least 80%, at least 84%, at least 88%, at least 92%, or at least 96% identity to SEQ ID NO: 2; and/or.
FR3 has at least 80%, at least 85%, at least 90%, or at least 95% identity to SEQ ID NO: 3.

17. The polypeptide of claim 15, wherein:
FR1 comprises the amino acid sequence of SEQ ID NO: 1;
FR2 comprises the amino acid sequence of SEQ ID NO: 2; and/or
FR3 comprises the amino acid sequence of SEQ ID NO: 3.

18. The polypeptide of claim 15, wherein (Xaa)ₙ is an amino acid sequence represented in the general formula
-Xaa-Xaa2-Xaa3-Xaa4-Xaa5-Xaa6-Xaa7-Xaa-Xaa- (SEQ ID NO: 4)
wherein Xaa, Xaa2, Xaa3, Xaa4, Xaa5, Xaa6 and Xaa7, individually for each occurrence, is an amino acid residue, with the caveat that (i) at least two of Xaa2, Xaa3, Xaa4 or Xaa5 are selected from His, Lys or Arg, or (ii) at least two of Xaa4, Xaa5, Xaa6 or Xaa7 are selected from His, Lys or Arg.

19. The polypeptide of claim 18, wherein at least three, and preferably four of Xaa2, Xaa3, Xaa4, Xaa5, Xaa6 or Xaa7 are selected from His, Lys or Arg.

20. The polypeptide of claim 15, wherein (Xaa)ₙ is at least 75% identical to the Loop 2 sequence selected from SEQ ID NOs: 6-299 and 1182.

21. The polypeptide of claim 15, wherein (Xaa)ₙ, is an amino acid sequence represented in the general formula
-Xaa-Xaa8-Xaa9-Xaa10-Xaa11-Xaa12-Xaa13-Xaa14-Xaa- (SEQ ID NO: 5)
wherein Xaa, Xaa8, Xaa9, Xaa10, Xaa11, Xaa12, Xaa13 and Xaa14, individually for each occurrence, is an amino acid residue, with the caveat that at least three of Xaa8, Xaa9, Xaa10, Xaa11, Xaa12, Xaa13 and Xaa14 are selected from His, Lys or Arg, and at least an additional two of Xaa8, Xaa9, Xaa10, Xaa11, Xaa12, Xaa13 and Xaa14 are selected from His, Lys, Arg, Phe, Tyr or Trp.

22. The polypeptide of claim 15, wherein (Xaa)ₘ is at least 75% identical to the Loop 4 sequence selected from SEQ ID NOs: 300-593 and 1183.

23. The polypeptide of claim 1, wherein the polypeptide includes at least one cysteine, which is (optionally) available for chemical conjugation, and which (optionally) is located at the C-terminal end or the N-terminal end of the polypeptide, and/or
wherein the polypeptide further comprising a heterologous polypeptide covalently linked through an amide bond to form a contiguous fusion protein.

24. (canceled)

25. The polypeptide of claim 23, wherein the heterologous polypeptide comprises a therapeutic polypeptide.

26. The polypeptide of claim 25, wherein the therapeutic polypeptide is selected from the group consisting of polypeptide hormones, polypeptide cytokines, polypeptide chemokines, growth factors, hemostasis active polypeptides, enzymes, and toxins,
wherein the therapeutic polypeptide is selected from the group consisting of receptor traps and receptor ligands,
wherein the therapeutic polypeptide sequence is selected from the group consisting of angiogenic agents and anti-angiogenic agents,
wherein the therapeutic polypeptide sequence is a neurotransmitter, and optionally wherein the neurotransmitter is Neuropeptide Y,
wherein the therapeutic polypeptide sequence is an erythropoiesis-stimulating agent, and optionally wherein the erythropoiesis-stimulating agent is erythropoietin or an erythropoietin mimetic,
wherein the therapeutic polypeptide is an incretin, and optionally wherein the incretin is selected from the group consisting of glucagon, gastric inhibitory peptide (GIP), glucagon-like peptide-1 (GLP-1), glucagon-like peptide-2 (GLP-2), peptide YY (PYY), and oxyntomodulin (OXM),
wherein the therapeutic polypeptide is an anticancer immune enhancing agent, such as a checkpoint inhibitor, a costimulatory receptor agonist or an iducer of innate immunity, and/or
wherein the therapeutic polypeptide is an anti-inflammatory immune inhibiting agent, such as a checkpoint agonist, a costimulatory receptor antagonist or an inhibitor of innate immunity.

27.-33. (canceled)

34. A pharmaceutical composition suitable for therapeutic use in a human patient, comprising a polypeptide of claim 1, and a pharmaceutically acceptable excipient.

35. The pharmaceutical composition of claim 34, wherein the pharmaceutical composition is formulated for pulmonary delivery or topical application.

36. The pharmaceutical composition of claim 35, wherein the pulmonary delivery is intranasal delivery.

37. A polynucleotide comprising a sequence encoding the polypeptide of claim 1.

38.-45. (canceled)

46. A viral vector comprising the polynucleotide of claim 37.

47. A plasmid or minicircle comprising the polynucleotide claim 37.

48. A cell comprising the polypeptide of claim 1, the polynucleotide of claim 37, the viral vector of claim 46, or the plasmid or minicircle of claim 47.

49. A method of increasing serum half-life of a therapeutic molecule, the method comprising conjugating the polypeptide of claim 1 to the therapeutic molecule.

50. A polypeptide of claim 1 for use in a method for treating an autoimmune disease and/or an inflammatory disease.

51. A polypeptide of claim 1 for use in a method for treating cancer.

52. A polypeptide of claim 1 for use in a method for treating cardiovascular or metabolic disease or disorder.

53. A method of producing the polypeptide of claim 1, the method comprising expressing in a host cell a nucleic acid encoding the polypeptide, and optionally isolating the polypeptide from the host cell.

54. A protein comprising an FcRn binding recombinantly engineered variant of stefin polypeptide sequence which binds to human FcRn and inhibits the binding of human IgG to human FcRn.

55. The protein of claim 54 for use in a method for treating an autoimmune or inflammatory disorder or disease.

56. A pharmaceutical composition suitable for therapeutic use in a human patient, comprising a protein of claim 54, and a pharmaceutically acceptable excipient.

57. The polypeptide of claim 1 comprising a loop 2 amino acid sequence of any one of SEQ ID NOs: 6-299 and 1182.

58. The polypeptide of claim 1 comprising a loop 4 amino acid sequence of any one of SEQ ID NOs: 300-593 and 1183.

59. The polypeptide of claim 1 comprising an amino acid sequence having at least 80%, at least 85%, at least 90%, at least 95%, at least 98%, or 100% identity to the sequence of any one of SEQ ID NOs: 594-887 or 1184.

60. The polypeptide of claim 1 encoded by a nucleic acid sequence having at least 70%, at least 75%, at least 80%, at least 85%, at least 90%, at least 95%, at least 98%, or 100% identity to the sequence of any one of SEQ ID NOs: 888-1181.

61. A use of the polynucleotide of claim 1 for targeting FcRn.

62. A use of the polynucleotide of claim 1 for increasing serum half-life of a therapeutic molecule.
